(19) United States
(12) Patent Application Publication
(10) Pub. No.: US 2025/0236589 A1
(43) Pub. Date: Jul. 24, 2025

(54) THERAPEUTIC PHENETHYLAMINE COMPOSITIONS AND METHODS OF USE

(71) Applicant: Cybin IRL Limited, Dublin (IE)

(72) Inventors: Alex Nivorozhkin, West Roxbury, MA (US); Joshua A. Hartsel, Lake Forest, CA (US); Clinton E. Canal, Atlanta, GA (US); Francesco G. Salituro, Marlborough, MA (US); Tina A. Mueller, West Palm Beach, FL (US); Brett J. Greene, Fitchburg, MA (US); Alex Belser, Brooklyn, NY (US); Kenneth L. Avery, Merrimack, NH (US); Amy Claire Reichelt, Ontario (CA); Geoffrey B. Varty, Annandale, NJ (US); Michael Palfreyman, St. Petersburg, FL (US)

(73) Assignee: Cybin IRL Limited, Dublin (IE)

(21) Appl. No.: 18/730,397

(22) PCT Filed: Feb. 15, 2023
(86) PCT No.: PCT/EP2023/053744

§ 371 Date: Jul. 19, 2024

Related U.S. Application Data
(60) Provisional application No. 63/386,375, filed on Dec. 7, 2022.
(60) Provisional application No. 63/268,022, filed on Feb. 15, 2022.
(60) Provisional application No. 63/268,019, filed on Feb. 15, 2022.

(51) Int. Cl. C07C 323/32; C07C 217/60; C07C 391/02

(52) U.S. Cl. CPC C07C 323/32; C07C 217/60; C07C 391/02

(57) ABSTRACT

There are disclosed phenethylamine compounds, the use of such compounds in the treatment of diseases associated with a serotonin 5-HT₂ receptor, pharmaceutical compositions such as tablet compositions and kits containing the compounds, methods of delivering the compounds in a mist via inhalation, and methods of treating diseases or disorders associated with a serotonin 5-HT₂ receptor, such as central nervous system (CNS) disorders or psychological disorders with the disclosed compounds.

### CROSS-REFERENCE

This application claims priority to U.S. Provisional Application No. 63/268,019, filed on Feb. 15, 2022, U.S. Provisional Application No. 63/268,022, filed on Feb. 15, 2022, and U.S. Provisional Application No. 63/386,375, filed on Dec. 7, 2022, each incorporated by reference herein in their entireties.

### FIELD OF THE INVENTION

The present disclosure relates generally to phenethylamine compounds and, in some embodiments, to serotonin 5-HT₂ receptor agonists and uses in the treatment of diseases associated with a 5-HT₂ receptor.

### BACKGROUND OF THE INVENTION

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

There are three, closely related subtypes of serotonin 5-HT₂ receptors (5-HT₂Rs), 5-HT_{2A}, 5-HT_{2B}, and 5-HT_{2C}, and they are primary targets of classic serotonergic psychedelics, such as lysergic acid diethylamide (LSD), psilocybin, and 2,5-dimethoxy-4-bromoamphetamine (DOB). Classic serotonergic psychedelics and entactogens have been actively investigated by the research and medical community to alleviate a multitude of central nervous system (CNS) disorders (Reiff, C. M., Richman, E. E., Nemeroff, C. B., Carpenter, L. L., Widge, A. S., Rodriguez, C. I., Kalin, N. H., and McDonald, W. M., 2020, Psychedelics and Psychedelic-Assisted Psychotherapy, Am J Psychiatry 177, 391-410), such as: (i) post-traumatic stress disorder (PTSD)(Jerome, L., Feduccia, A. A., Wang, J. B., Hamilton, S., Yazar-Klosinski, B., Emerson, A., Mithoefer, M. C., and Doblin, R., 2020, Long-term follow-up outcomes of MDMA-assisted psychotherapy for treatment of PTSD: a longitudinal pooled analysis of six phase 2 trials, Psychopharmacology (Berl) 237, 2485-2497), (ii) major depressive disorder (MDD), (iii) treatment-resistant depression (TRD)(Goldberg, S. B., Pace, B. T., Nicholas, C. R., Raison, C. L., and Hutson, P. R., 2020, The experimental effects of psilocybin on symptoms of anxiety and depression: A meta-analysis, Psychiatry Res 284, 112749), (iv) obsessive-compulsive disorder (OCD)(Moreno, F. A., Wiegand, C. B., Taitano, E. K., and Delgado, P. L., 2006, Safety, tolerability, and efficacy of psilocybin in 9 patients with obsessive-compulsive disorder, J Clin Psychiatry 67, 1735-1740), (v) social anxiety disorder (ClinicalTrials.gov, number NCT02008396), (vi) substance use disorders, including but not limited to alcohol use disorder, opioid use disorder, amphetamine use disorder, nicotine use disorder, and cocaine use disorder, (vii) anorexia nervosa, (viii) bulimia nervosa (ClinicalTrials.gov, numbers NCT04454684 and NCT04052568), (ix) Alzheimer's disease (ClinicalTrials.gov, number NCTO4123314), and (x) cluster headache and migraine (Nichols, D. E., 2016, Psychedelics, Pharmacol Rev 68, 264-355; Johnson, M. W., Hendricks, P. S., Barrett, F. S., and Griffiths, R. R., 2019, Classic psychedelics: An integrative review of epidemiology, therapeutics, mystical experience, and brain network function, Pharmacol Ther 197, 83-102; Sewell, R. A., Halpern, J. H., and Pope, H. G., Jr., 2006, Response of cluster headache to psilocybin and LSD, Neurology 66, 1920-1922; ClinicalTrials.gov, number NCTO4218539).

These drugs have also been investigated to alleviate conditions of the autonomic nervous system (ANS), including pulmonary disorders (e.g., asthma and chronic obstructive pulmonary disorder (COPD) and cardiovascular disorders (e.g., atherosclerosis), among others (Nichols, D. E., Johnson, M. W., and Nichols, C. D., 2017, Psychedelics as Medicines: An Emerging New Paradigm, Clin Pharmacol Ther 101, 209-219; Flanagan, T. W., Sebastian, M. N., Battaglia, D. M., Foster, T. P., Cormier, S. A., and Nichols, C. D., 2019, 5-HT2 receptor activation alleviates airway inflammation and structural remodeling in a chronic mouse asthma model, Life Sci 236, 116790; Flanagan, T. W., Sebastian, M. N., Battaglia, D. M., Foster, T. P., Maillet, E. L., and Nichols, C. D., 2019, Activation of 5-HT2 Receptors Reduces Inflammation in Vascular Tissue and Cholesterol Levels in High-Fat Diet-Fed Apolipoprotein E Knockout Mice, Sci Rep 9, 13444; Sexton, J. D., Nichols, C. D., and Hendricks, P. S., 2019, Population Survey Data Informing the Therapeutic Potential of Classic and Novel Phenethylamine, Tryptamine, and Lysergamide Psychedelics, Front Psychiatry 10, 896).

Some studies have advanced into Phase III trials, for example the use of 3,4-methylenedioxymethamphetamine (MDMA) for the treatment of PTSD (Feduccia, A. A., Jerome, L., Yazar-Klosinski, B., Emerson, A., Mithoefer, M. C., and Doblin, R., 2019, Breakthrough for Trauma Treatment: Safety and Efficacy of MDMA-Assisted Psychotherapy Compared to Paroxetine and Sertraline, Front Psychiatry 10, 650), and phase 1 trials of 3,4,5-trimethoxyphenethylamine (mescaline) have begun (ClinicalTrials.gov, number NCT04227756).

Mechanistically, the therapeutic effects of psychedelic phenethylamines are thought to be mediated by their interaction with serotonin (5-HT) receptors, particularly 5-HT_{2A} receptors, though other targets, including the 5-HT_{1A} receptor may also be involved (Nichols, D. E., 2016, Psychedelics, Pharmacol Rev 68, 264-355; Canal, C. E., 2018, Serotonergic Psychedelics: Experimental Approaches for Assessing Mechanisms of Action, Handb Exp Pharmacol 252, 227-260). A contribution from the 5-HT_{2C} receptor may be responsible for the reported anti-addictive properties of classic psychedelics (Canal, C. E., and Murnane, K. S., 2017, The serotonin 5-HT2C receptor and the non-addictive nature of classic hallucinogens, J Psychopharmacol 31, 127-143). The effects of entactogen phenethylamines are mediated primarily by their interaction with monoamine transporters, particular the serotonin (SERT) and dopamine (DAT) transporters (Jayanthi, L. D., and Ramamoorthy, S., 2005, Regulation of monoamine transporters: influence of psychostimulants and therapeutic antidepressants, AAPS J 7, E728-738).

Safety aspects of psychedelics and entactogens remain a key challenge for clinical applications (Hasler, F., Grimberg, U., Benz, M. A., Huber, T., and Vollenweider, F. X., 2004, Acute psychological and physiological effects of psilocybin in healthy humans: a double-blind, placebo-controlled dose-effect study, Psychopharmacology (Berl) 172, 145-156; Carbonaro, T. M., Bradstreet, M. P., Barrett, F. S., MacLean, K. A., Jesse, R., Johnson, M. W., and Griffiths, R. R., 2016, Survey study of challenging experiences after ingesting psilocybin mushrooms: Acute and enduring positive and negative consequences, J Psychopharmacol 30, 1268-1278; Garcia-Romeu, A., Kersgaard, B., and Addy, P. H., 2016, Clinical applications of hallucinogens: A review, Exp Clin Psychopharmacol 24, 229-268; Morgan, L., 2020, MDMA-assisted psychotherapy for people diagnosed with treatment-resistant PTSD: what it is and what it isn't, Ann Gen Psychiatry 19, 33; Schenk, S., and Newcombe, D., 2018, Methylenedioxymethamphetamine (MDMA) in Psychiatry: Pros, Cons, and Suggestions, J Clin Psychopharmacol 38, 632-638).

Clearly, a safe therapeutic window for this class of drugs is very narrow, because of cardiovascular complications associated with prolonged, increased serotonin release and 5-HT_{2B} stimulation (Huang, X.-P., Setola, V., Yadav, P. N., Allen, J. A., Rogan, S. C., Hanson, B. J., Revankar, C., Robers, M., Doucette, C., and Roth, B. L., 2009, Parallel Functional Activity Profiling Reveals Valvulopathogens Are Potent 5-Hydroxytryptamine(2B) Receptor Agonists: Implications for Drug 25 Safety Assessment, Molecular Pharmacology 76, 710-722; Rothman, R. B., and Baumann, M. H., 2009, Serotonergic drugs and valvular heart disease, Expert Opin Drug Saf 8, 317-329), depressive after-effects associated with depletion of central serotonin levels (Parrott, A. C., 2014, The potential dangers of using MDMA for psychotherapy, J Psychoactive Drugs 46, 37-43), and numerous other acute adverse effects, including anxiety, fear, tachycardia, hypertension, increased body temperature, nausea and vomiting, with many acute adverse effects being attributed to high drug concentrations (“spiking”) in the blood shortly after oral administration (Meyer, J. S., 2013, 3,4-methylenedioxymethamphetamine (MDMA): current perspectives, Subst Abuse Rehabil 4, 83-99; Baylen, C. A., and Rosenberg, H., 2006, A review of the acute subjective effects of MDMA/ecstasy, Addiction 101, 933-947; Shulgin, A., and Shulgin, Ann., 1991, Pihkal: a chemical love story, Transform Press, Berkeley, CA; Barrett, F. S., Bradstreet, M. P., Leoutsakos, J. S., Johnson, M. W., and Griffiths, R. R., 2016, The Challenging Experience Questionnaire: Characterization of challenging experiences with psilocybin mushrooms, J Psychopharmacol 30, 1279-1295). Many psychedelics and entactogens are also long acting, and thus require full day supervision considering their narrow therapeutic window, which is a major impediment to their clinical use.

One class of psychedelic phenethylamines is the 2C—X family of phenethylamines (phenethylamines containing 2,4,5 substitution, with methoxy groups at the 2 and 5 positions of the phenyl group). Members of this class, such as 2,5-dimethoxy-4-bromophenethylamine (2C—B), may be used to treat sexual dysfunctions (Shulgin, A., and Shulgin, Ann., 1991, Pihkal: a chemical love story, Transform Press, Berkeley, CA), as well as neuropsychiatric conditions, and induce changes in perception, cognition, emotion, and mood that may underlie its reported neuropsychotherapeutic benefits (Johnson, M. W., Hendricks, P. S., Barrett, F. S., and Griffiths, R. R., 2019, Classic psychedelics: An integrative review of epidemiology, therapeutics, mystical experience, and brain network function, Pharmacol Ther 197, 83-102).

However, wider clinical testing and finding practical treatment protocols for 2C—X compounds is challenged by the following factors: 1) poor oral bioavailability; 2) low brain penetration; 3) delayed effects from oral administration; 4) therapeutic effects requiring high doses; 5) acute psychiatric adverse events (AE), such as fear, anxiety, and paranoia, cardiovascular events including tachycardia and hypertension, and gastrointestinal effects including nausea; and 6) toxicity. These properties are thought to be due to rapid first-pass metabolism through deamination/oxidation by monoamine oxidases (MAO), MAO-A and MAO-B and O-dealkylation by cytochrome P450 enzymes such as CYP2D6, and also to their relatively hydrophilic nature (e.g., 2,4,5-trimethoxphenethylamine (2C—O) possesses a logP value of 0.98; ChemDraw) which limits distribution to the brain (Suzuki, O., Katsumata, Y., and Oya, M., 1981, Oxidation of beta-phenylethylamine by both types of monoamine oxidase: examination of enzymes in brain and liver mitochondria of eight species, J Neurochem 36, 1298-1301; Monte, A. P., Marona-Lewicka, D., Parker, M. A., Wainscott, D. B., Nelson, D. L., and Nichols, D. E., 1996, Dihydrobenzofuran analogues of hallucinogens. 3. Models of 4-substituted (2,5-dimethoxyphenyl)alkylamine derivatives with rigidified methoxy groups, J Med Chem 39, 2953-2961; Monte, A. P., Waldman, S. R., Marona-Lewicka, D., Wainscott, D. B., Nelson, D. L., Sanders-Bush, E., and Nichols, D. E., 1997, Dihydrobenzofuran analogues of hallucinogens. 4. Mescaline derivatives, J Med Chem 40, 2997-3008). For example, 2,4,5-trimethoxphenethylamine (2C—O) is inactive after oral administration (Shulgin, A. T., 1978, Psychotomimetic Drugs: Structure-activity relationships. Chapter 6, In Handbook of psychopharmacology, V.11-Stimulants, pp 243-333, Plenum Press, New York).

The limitations of current 2C—X compounds and formulations thereof, and other psychedelic and entactogen phenethylamines, are apparent—and it has proven difficult to control drug exposure and maintain drug concentrations in the safe and efficacious range.

### SUMMARY OF THE INVENTION

In view of the forgoing, there is a need for novel phenethylamine compounds, e.g., of the 2C—X variety, which have improved pharmacokinetic properties—that are bioavailable, brain penetrable, have fast onset, and are short acting—and which demonstrate enhanced activity while minimizing psychiatric adverse events and toxicity. There is a further need for efficient, more convenient, and controllable phenethylamine formulations that afford no neurologically toxic (e.g., psychotomimetic toxic) plasma concentration.

Accordingly, it is one object of the present invention to provide novel compounds that meet these criteria.

It is another object of the present disclosure to provide novel pharmaceutical compositions which contain the compounds.

It is another object of the present disclosure to provide novel methods of treating a subject with a disease or disorder e.g., those associated with a serotonin 5-HT₂ receptor, with the compounds.

It is another object of the present disclosure to provide novel tablet compositions, such as single-layer orally administered tablet compositions, containing the compounds.

It is another object of the present disclosure to provide novel kits containing formulations of the compounds for use in treatment.

It is yet another object of the present disclosure to provide novel methods of delivering the compounds in an aerosol, preferably a mist, via inhalation, such as for the treatment of a central nervous system (CNS) disorder or psychological disorder.

It is yet another object of the invention to provide a novel use of the compounds for treating a subject with a disease or disorder associated with a serotonin 5-HT₂ receptor, such as a central nervous system (CNS) disorder or psychological disorder.

These and other objects, which will become apparent during the following detailed description, have been achieved by the inventors' discovery of novel 2C—X type phenethylamine compounds (e.g., compounds of Formula (I) through (VII)), including those with site-specific modifications (e.g., deuteration/fluorination) which maintain preferential binding to G-protein coupled receptors (GPCRs), e.g., 5-HT₂ receptors, have improved exposure (e.g., prevent high drug concentrations (spiking) observed acutely after administration), and possess advantageous enzymatic degradation profiles for improved bioavailability, brain penetration, and prevention/reduction of toxic metabolite formation.

Thus, the present invention provides:

(1) A compound having a structure of Formula (I):

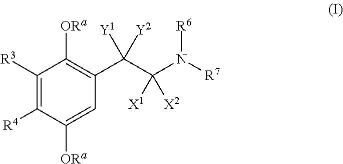

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R³ is hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b};
R⁶ and R⁷ are independently hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl; or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl;
with the proviso that at least one of X¹, X², Y¹, Y², R³, R⁴, R⁶, R⁷, and R^{a} comprises deuterium and/or R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N.

(2) The compound of (1), wherein Y¹ and Y² are hydrogen.

(3) The compound of (1) or (2), wherein R³ is hydrogen.

(4) The compound of any one of (1) to (3), wherein X¹ and X² are hydrogen.

(5) The compound of any one of (1) to (3), wherein X¹ and X² are deuterium.

(6) The compound of any one of (1) to (5), wherein each R^{a} is —CH₃ or —CD₃.

(7) The compound of any one of (1) to (6), wherein R⁴ is —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br.

(8) The compound of (1), having a structure of Formula (II):

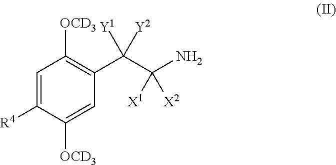

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b}; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl.

(9) The compound of (8), wherein X¹ and X² are hydrogen.

(10) The compound of (8) or (9), wherein R⁴ is —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br.

(11) The compound of any one of (8) to (10), wherein R⁴ is —SMe, —SCF₃, —SCF₂H, -Me, —OCD₃, —CF₃, -t-Bu, or -cyclopentyl.

(12) The compound of (1), having a structure of Formula (III):

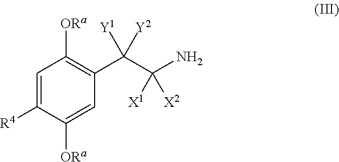

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁴ is a C₁-C₆ alkyl substituted with one or more deuterium, a C₃-C₁₀ cycloalkyl substituted with one or more deuterium, —OR^{b}, or —SR^{b};
each R^{a} is independently a substituted or unsubstituted C₁-C₆ alkyl; and
R^{b} is a C₁-C₆ alkyl substituted with one or more deuterium or a C₃-C₁₀ cycloalkyl substituted with one or more deuterium.

(13) The compound of (12), wherein X¹ and X² are hydrogen and each R^{a} is —CH₃.

(14) The compound of (12) or (13), wherein R⁴ is —SCD₃, —CD₃, —C(CD₃)₃, or —OCD₃.

(15) The compound of (1), having a structure of Formula (IV):

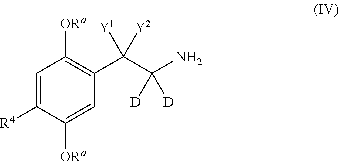

or a pharmaceutically acceptable salt, solvate, stereoisomer, or prodrug thereof,
wherein:
Y¹ and Y² are independently hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b};
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl.

(16) The compound of (1), having a structure of Formula (V):

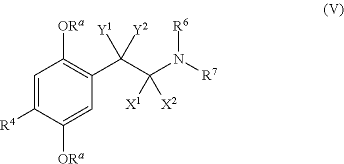

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b};
R⁶ is hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl;
with the proviso that at least one of X¹, X², Y¹, Y², R³, R⁴, R⁶, R⁷, and R^{a} comprises deuterium and/or R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N.

(17) The compound of (16), wherein R⁶ is hydrogen, and R⁷ is an unsubstituted C₁-C₆ alkyl, a C₁-C₆ alkyl substituted with one or more deuterium atoms, a C₁-C₆ alkyl substituted with one or more fluorine atoms, or a C₁-C₆ alkyl substituted with a substituted or unsubstituted cycloalkyl.

(18) The compound of any one of (1) to (17), which is selected from the group consisting of

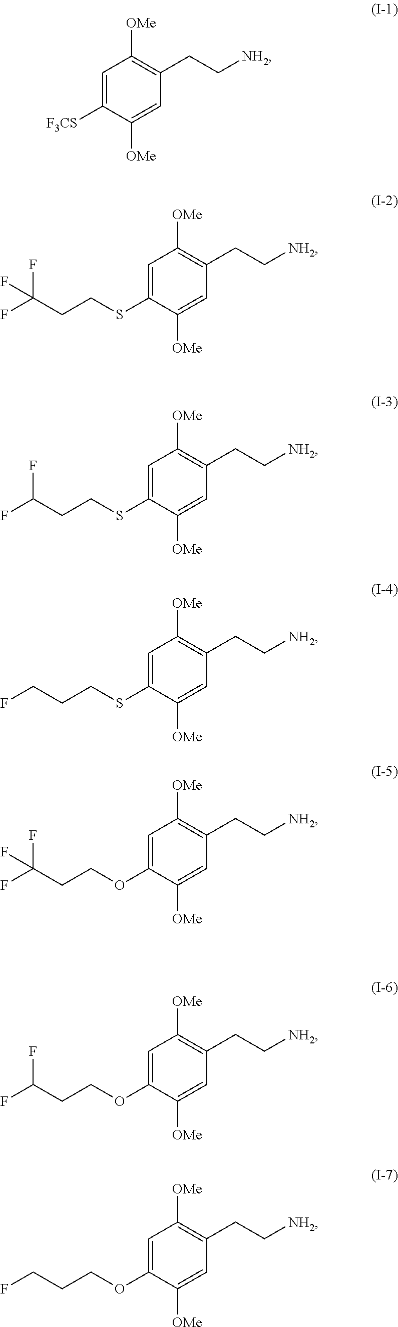

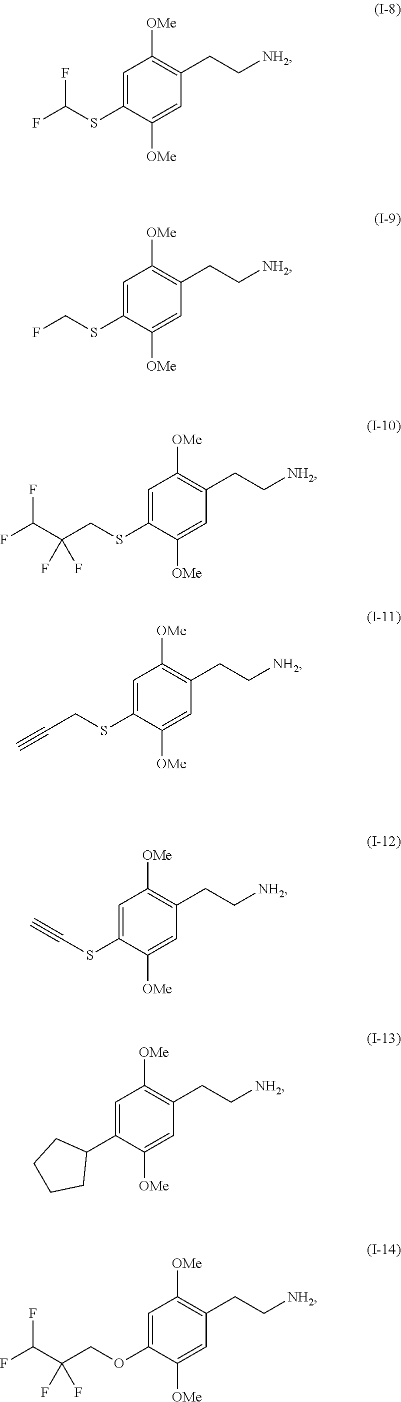

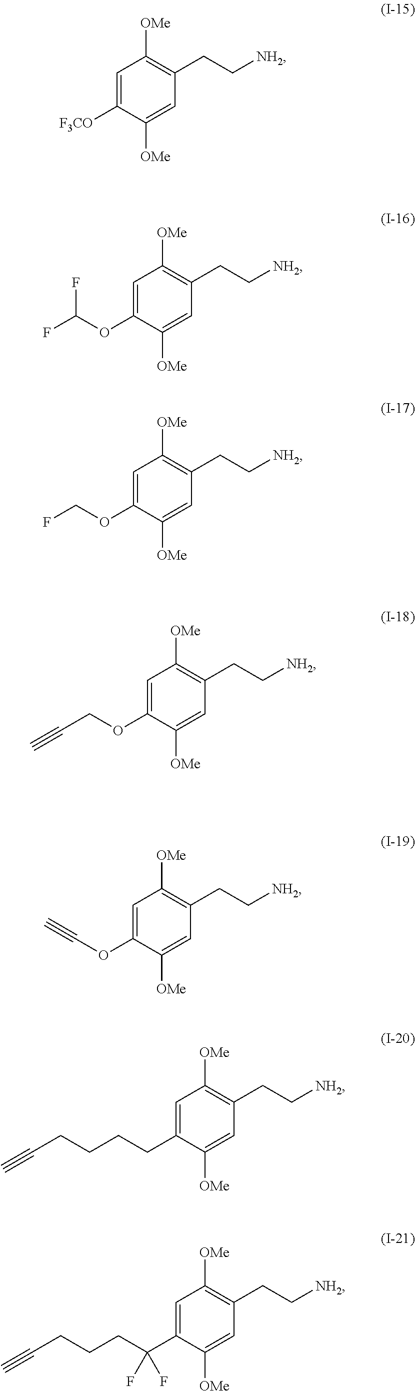

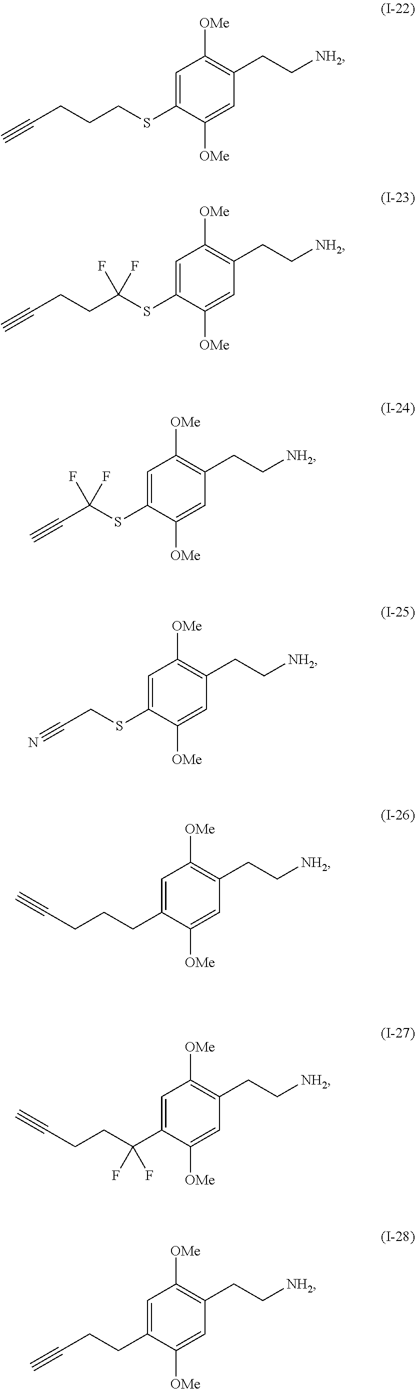

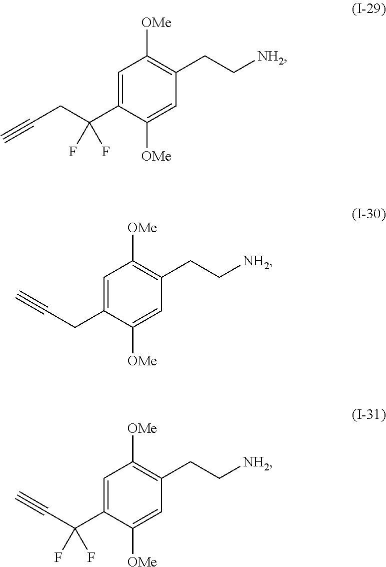 [SMILES: C#CCC(F)(F)c1cc(OC)c(CCN)cc1OC ; C#CCc1cc(OC)c(CCN)cc1OC ; C#CC(F)(F)c1cc(OC)c(CCN)cc1OC]

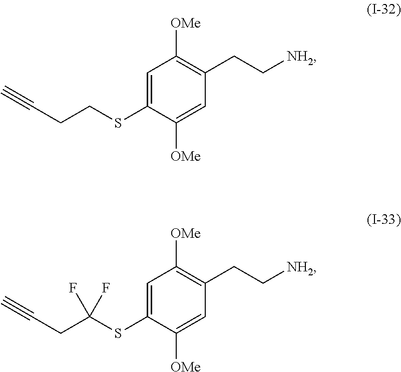 [SMILES: C#CCCSc1cc(OC)c(CCN)cc1OC ; C#CCC(F)(F)Sc1cc(OC)c(CCN)cc1OC]

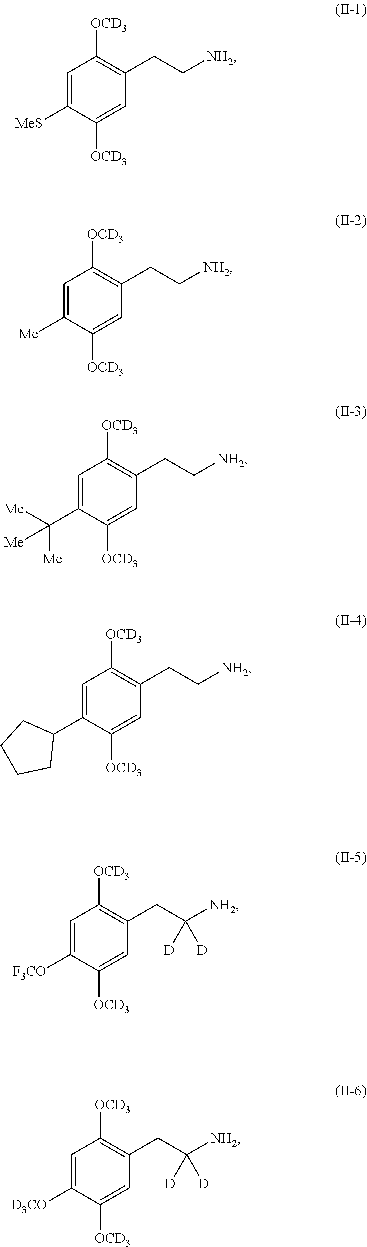

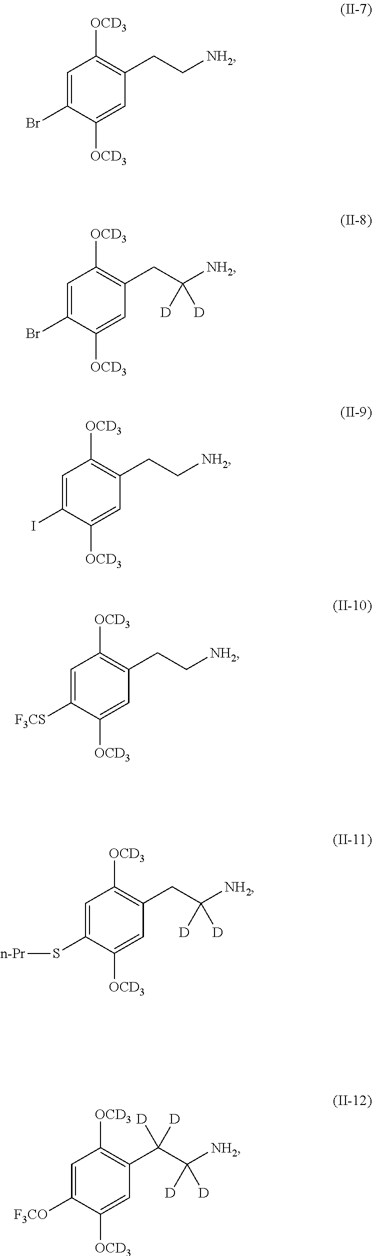

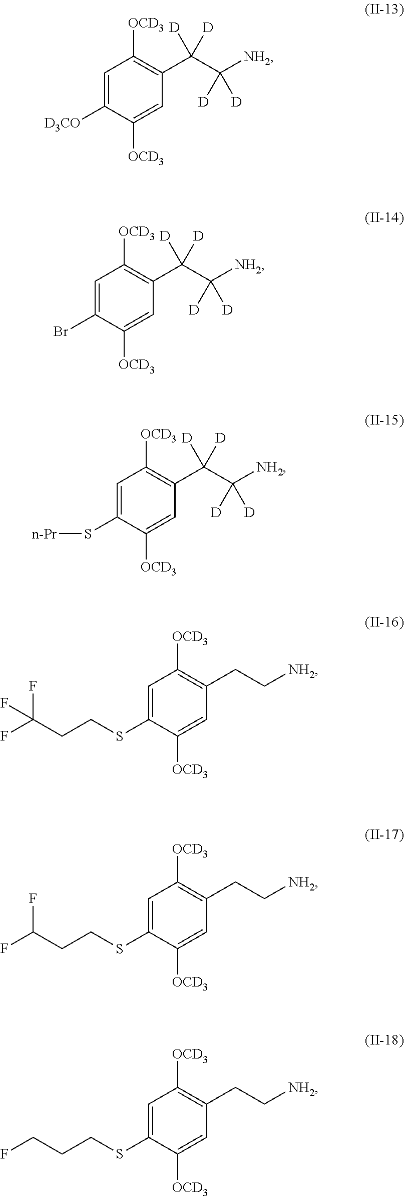

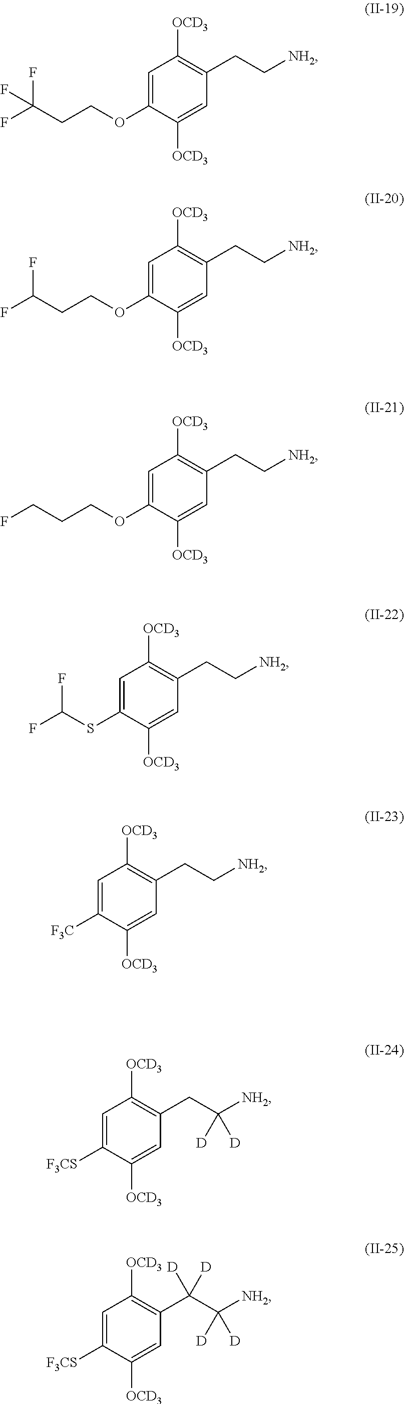

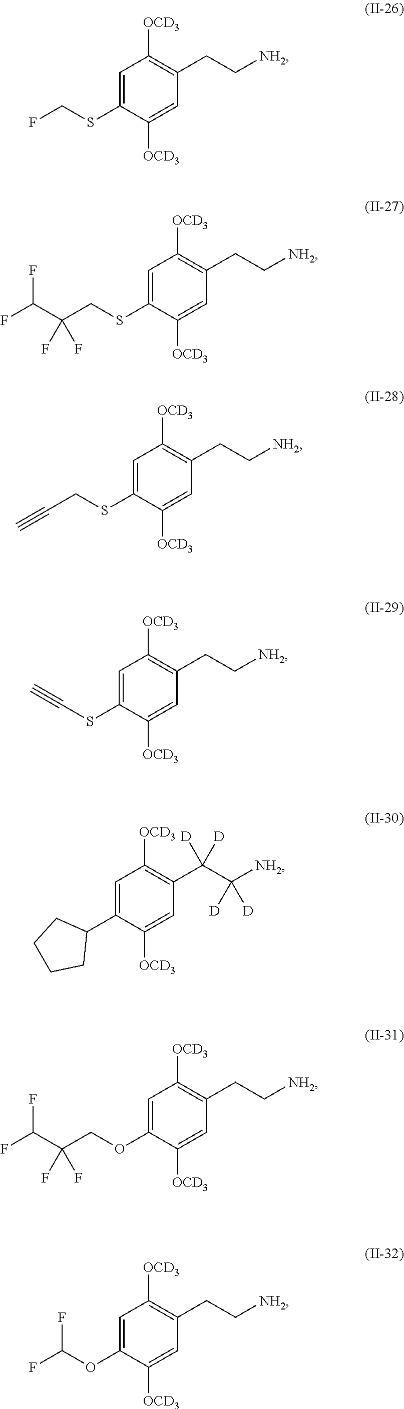

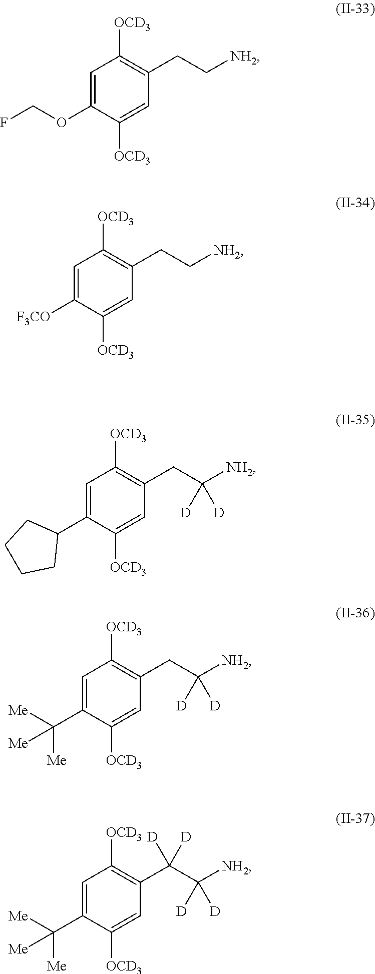

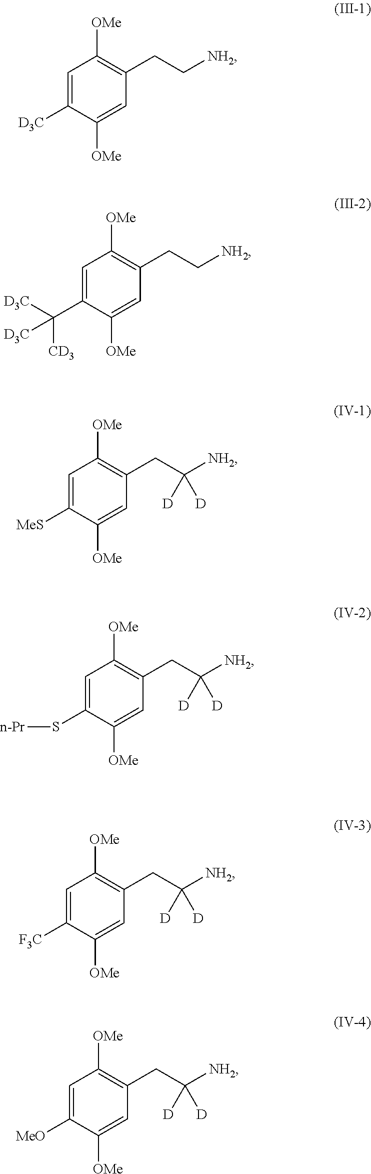

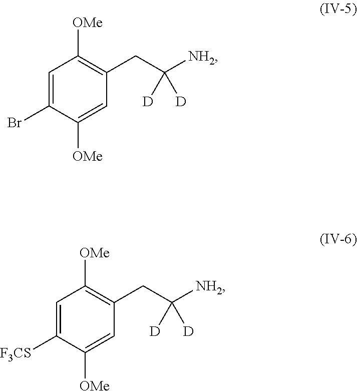

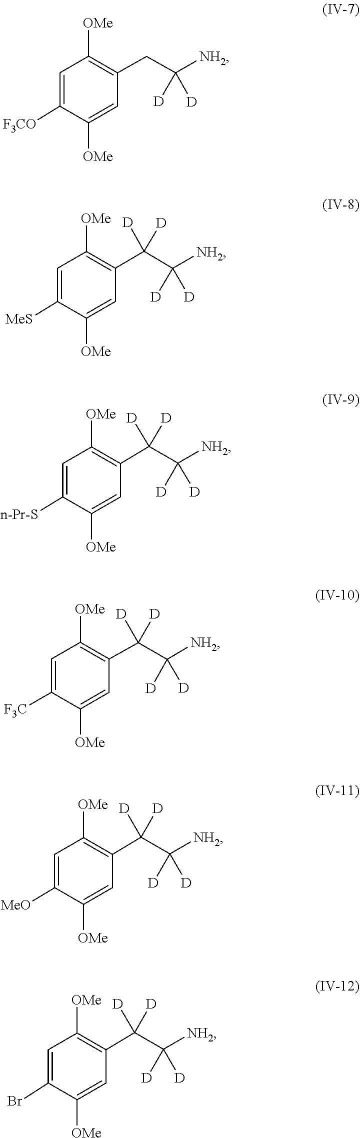

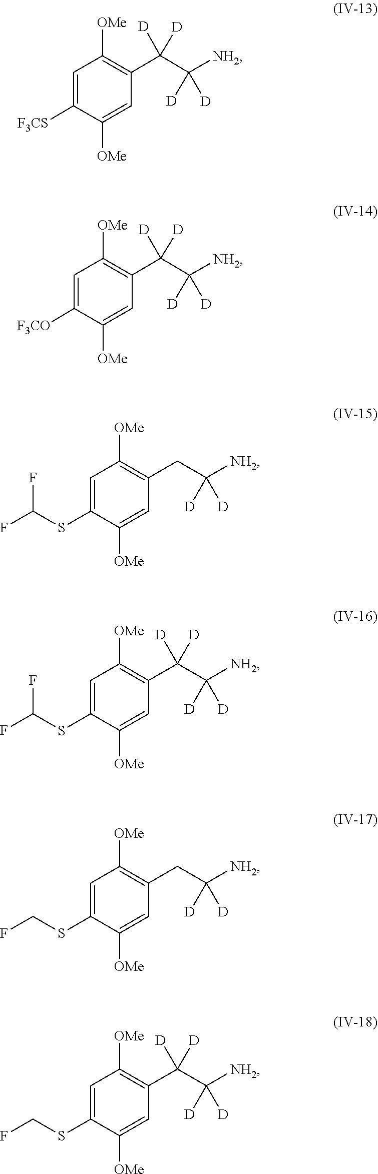

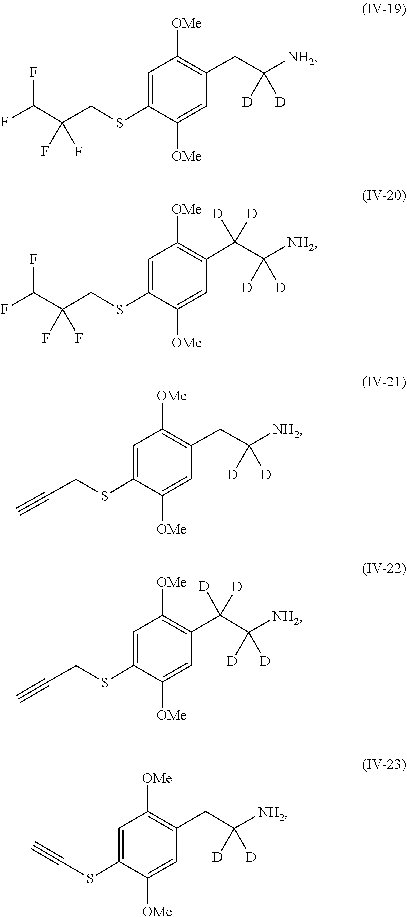 [SMILES: [2H]C([2H])(N)Cc1cc(OC)c(SCC(F)(F)C(F)F)cc1OC ; [2H]C([2H])(N)C([2H])([2H])c1cc(OC)c(SCC(F)(F)C(F)F)cc1OC ; [2H]C([2H])(N)Cc1cc(OC)c(SCC#C)cc1OC ; [2H]C([2H])(N)C([2H])([2H])c1cc(OC)c(SCC#C)cc1OC ; [2H]C([2H])(N)Cc1cc(OC)c(SC#C)cc1OC]

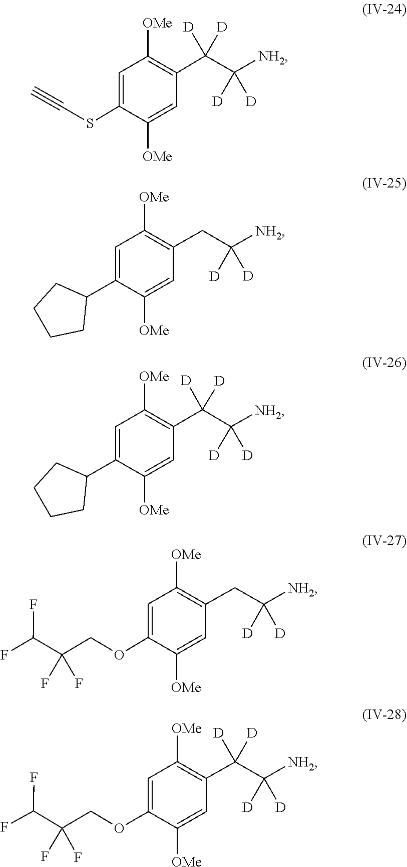

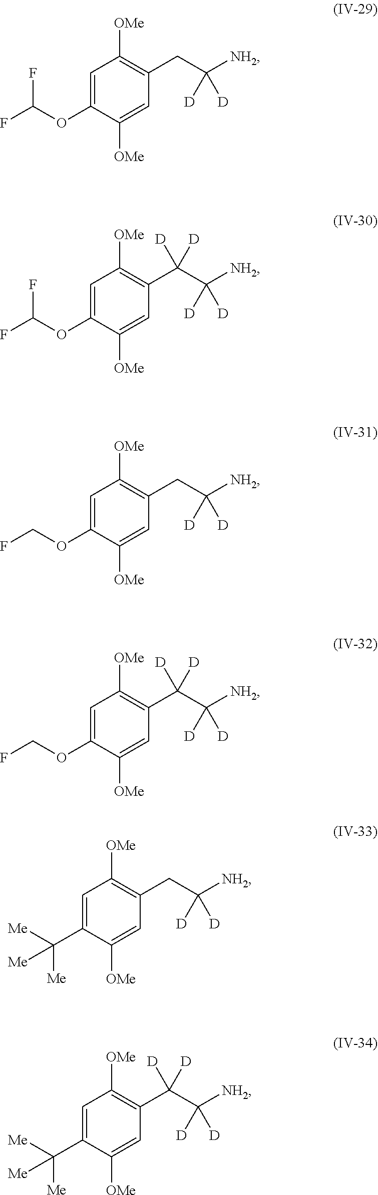 [SMILES: [2H]C([2H])(N)Cc1cc(OC)c(OC(F)F)cc1OC ; [2H]C([2H])(N)C([2H])([2H])c1cc(OC)c(OC(F)F)cc1OC ; [2H]C([2H])(N)Cc1cc(OC)c(OCF)cc1OC ; [2H]C([2H])(N)C([2H])([2H])c1cc(OC)c(OCF)cc1OC ; [2H]C([2H])(N)Cc1cc(OC)c(C(C)(C)C)cc1OC ; [2H]C([2H])(N)C([2H])([2H])c1cc(OC)c(C(C)(C)C)cc1OC]

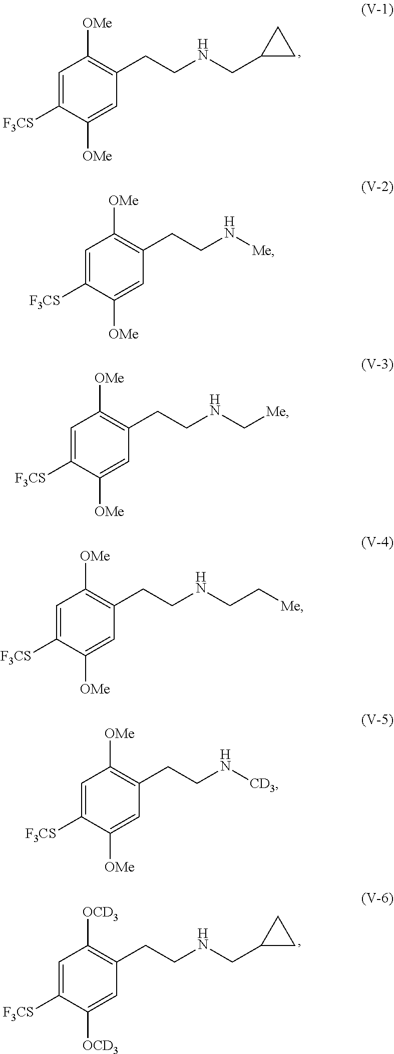

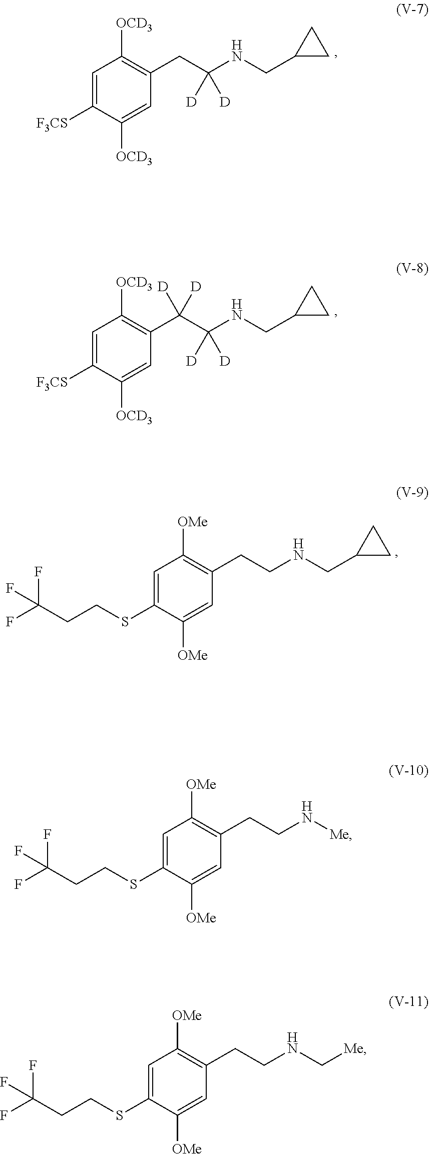

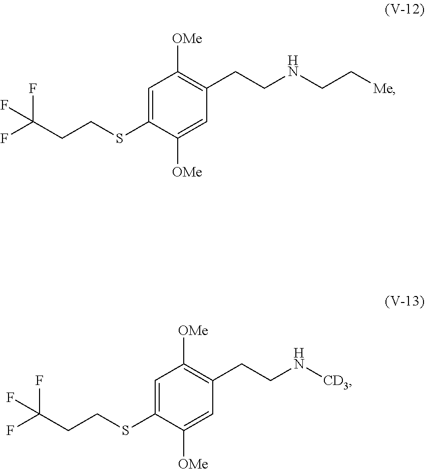 [SMILES: CCCNCCc1cc(OC)c(SCCC(F)(F)F)cc1OC ; [2H]C([2H])([2H])NCCc1cc(OC)c(SCCC(F)(F)F)cc1OC]

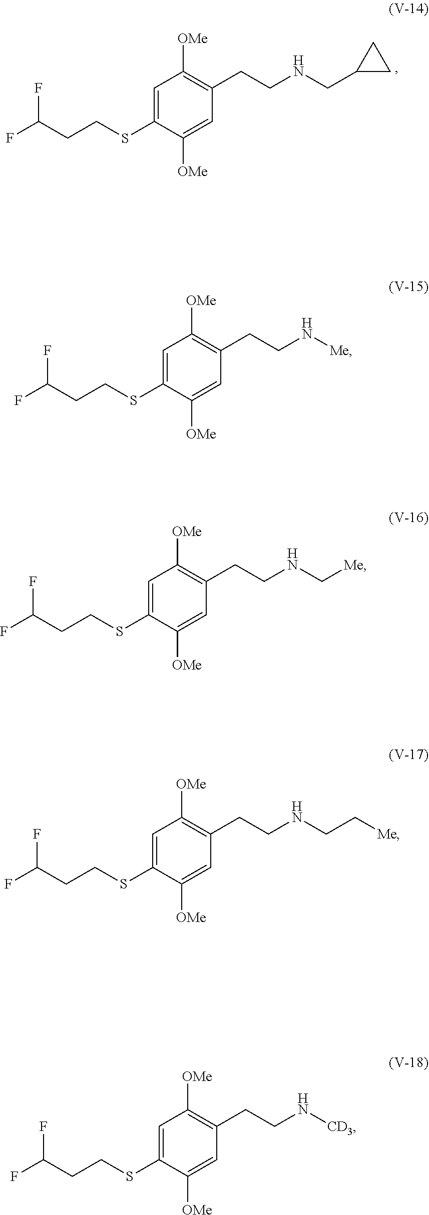 [SMILES: COc1cc(SCCC(F)F)c(OC)cc1CCNCC1CC1 ; CNCCc1cc(OC)c(SCCC(F)F)cc1OC ; CCNCCc1cc(OC)c(SCCC(F)F)cc1OC ; CCCNCCc1cc(OC)c(SCCC(F)F)cc1OC ; [2H]C([2H])([2H])NCCc1cc(OC)c(SCCC(F)F)cc1OC]

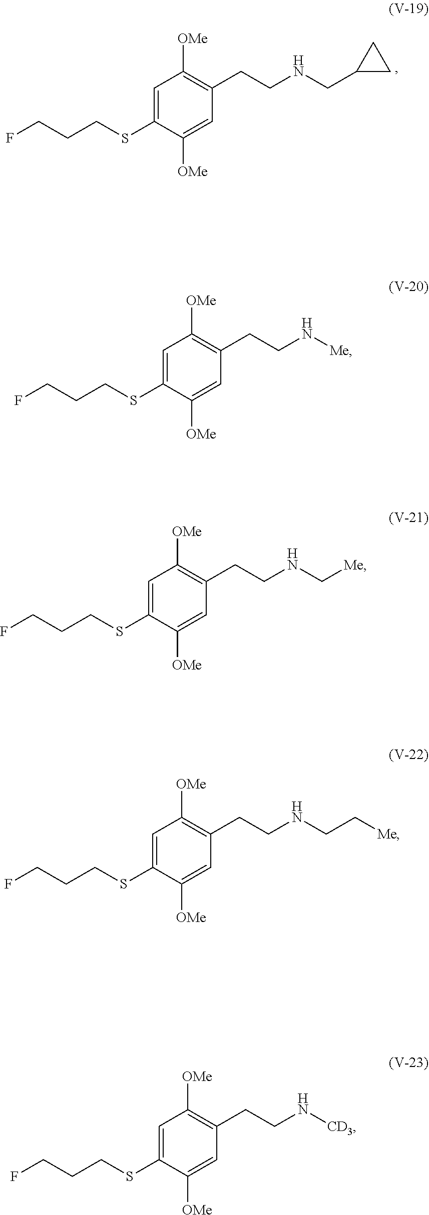 [SMILES: COc1cc(SCCCF)c(OC)cc1CCNCC1CC1 ; CNCCc1cc(OC)c(SCCCF)cc1OC ; CCNCCc1cc(OC)c(SCCCF)cc1OC ; CCCNCCc1cc(OC)c(SCCCF)cc1OC ; [2H]C([2H])([2H])NCCc1cc(OC)c(SCCCF)cc1OC]

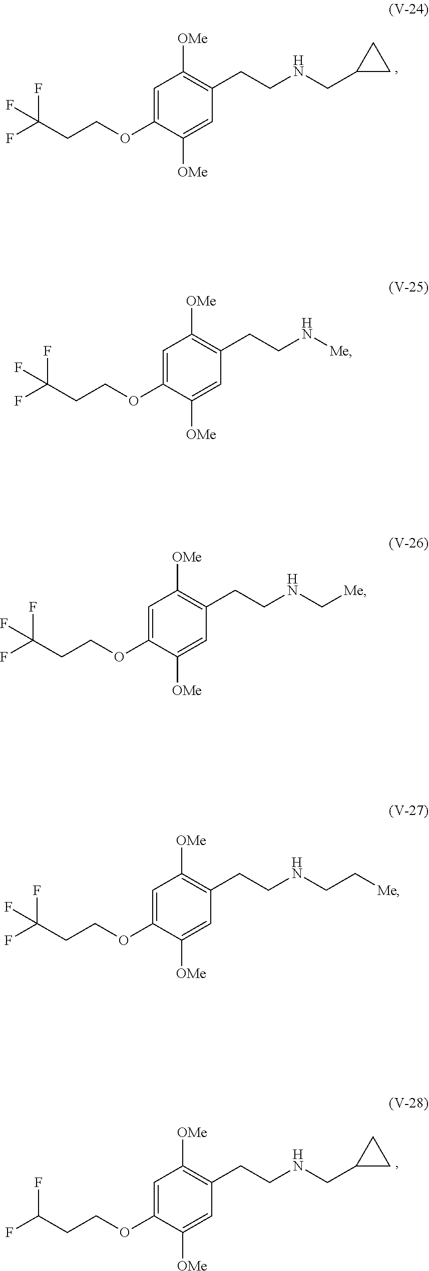 [SMILES: COc1cc(OCCC(F)(F)F)c(OC)cc1CCNCC1CC1 ; CNCCc1cc(OC)c(OCCC(F)(F)F)cc1OC ; CCNCCc1cc(OC)c(OCCC(F)(F)F)cc1OC ; CCCNCCc1cc(OC)c(OCCC(F)(F)F)cc1OC ; COc1cc(OCCC(F)F)c(OC)cc1CCNCC1CC1]

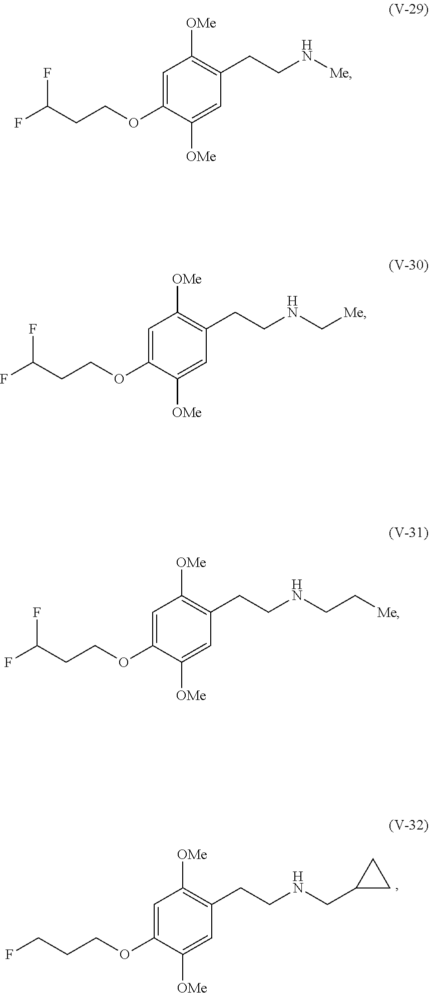 [SMILES: CNCCc1cc(OC)c(OCCC(F)F)cc1OC ; CCNCCc1cc(OC)c(OCCC(F)F)cc1OC ; CCCNCCc1cc(OC)c(OCCC(F)F)cc1OC ; COc1cc(OCCCF)c(OC)cc1CCNCC1CC1]

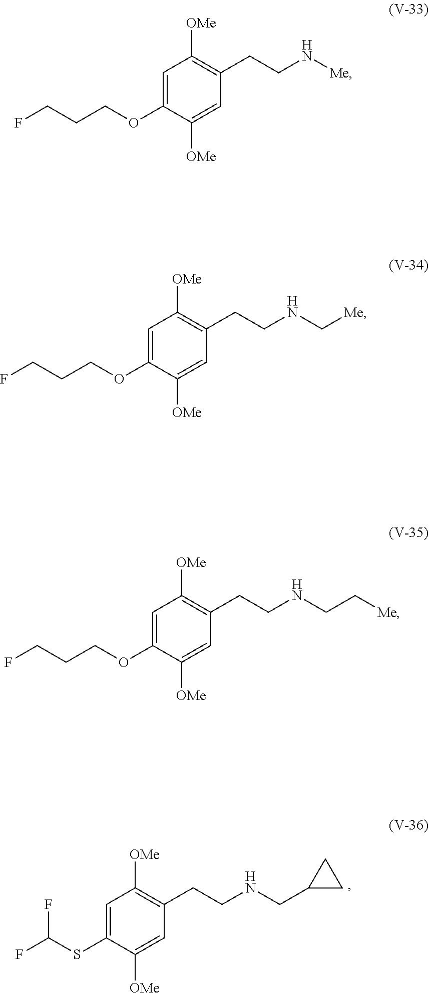 [SMILES: CNCCc1cc(OC)c(OCCCF)cc1OC ; CCNCCc1cc(OC)c(OCCCF)cc1OC ; CCCNCCc1cc(OC)c(OCCCF)cc1OC ; COc1cc(SC(F)F)c(OC)cc1CCNCC1CC1]

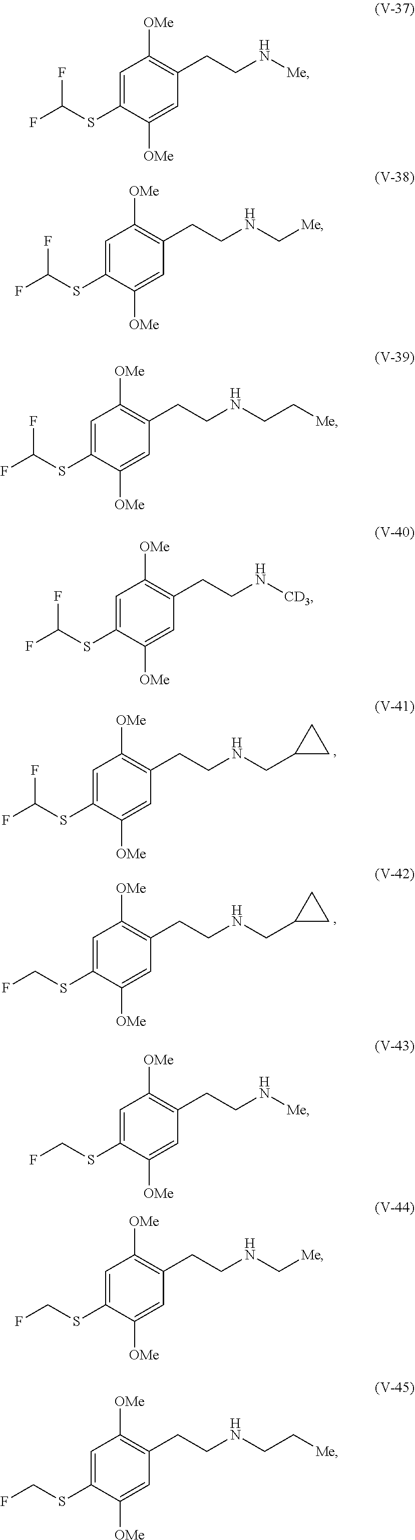

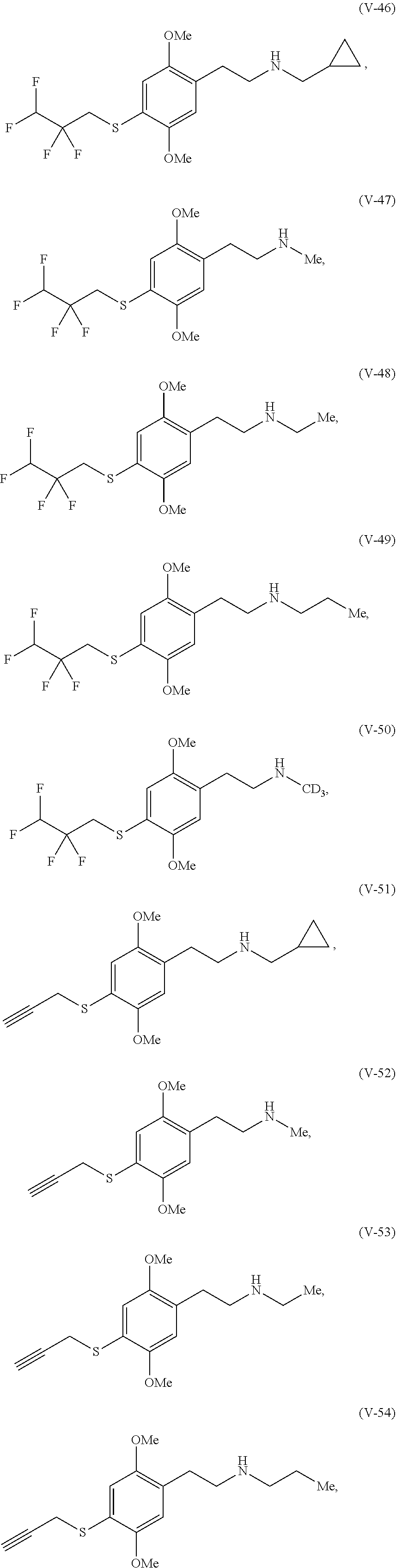

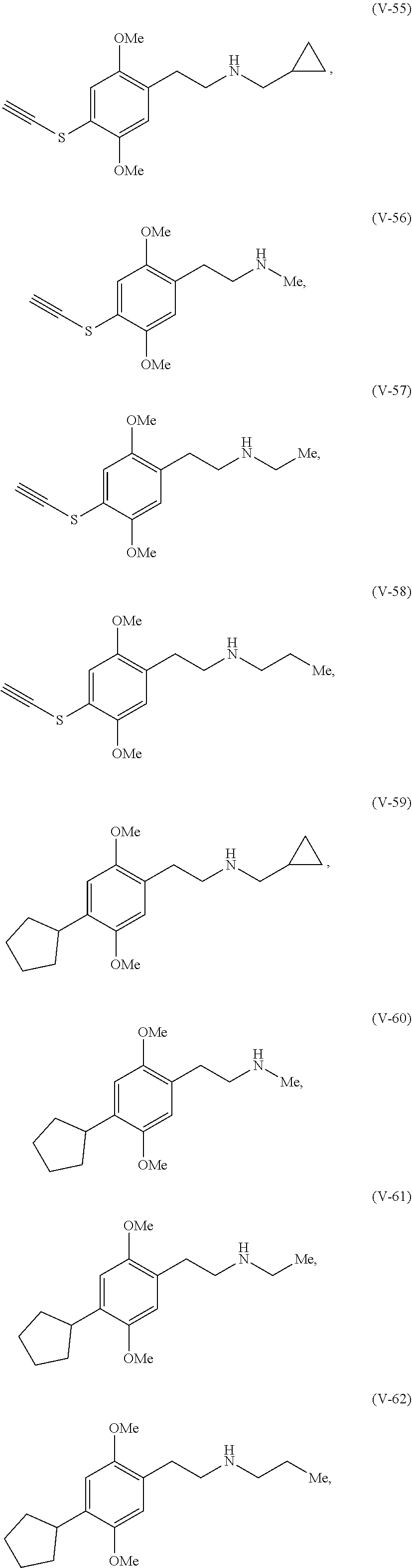

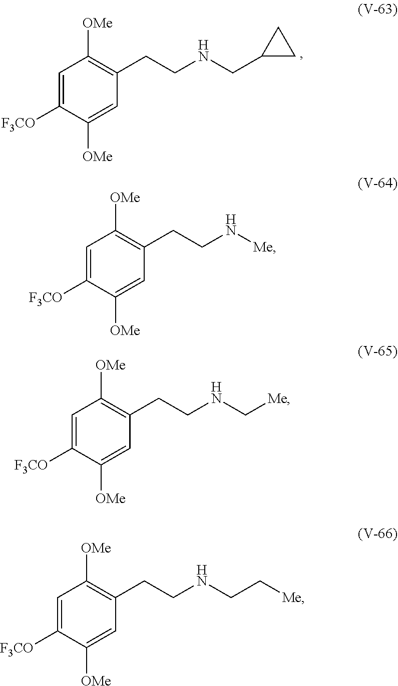

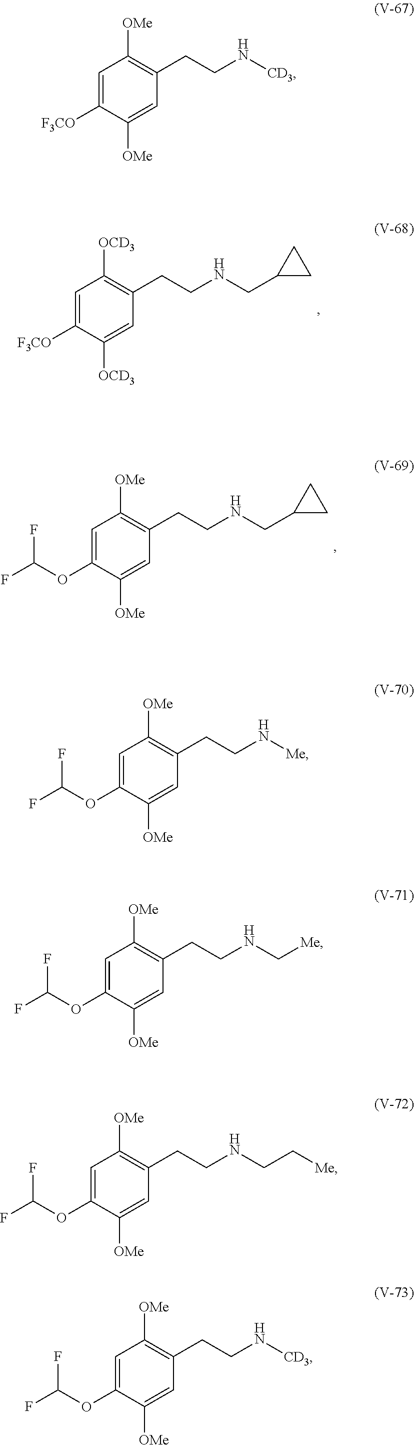

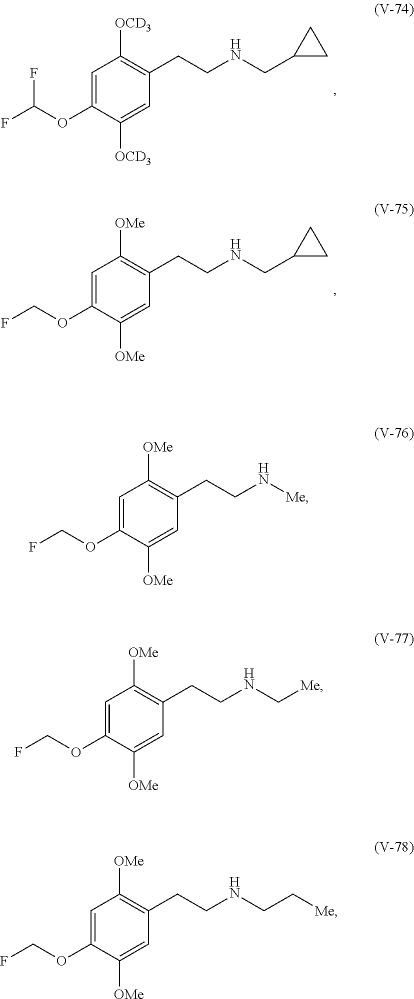

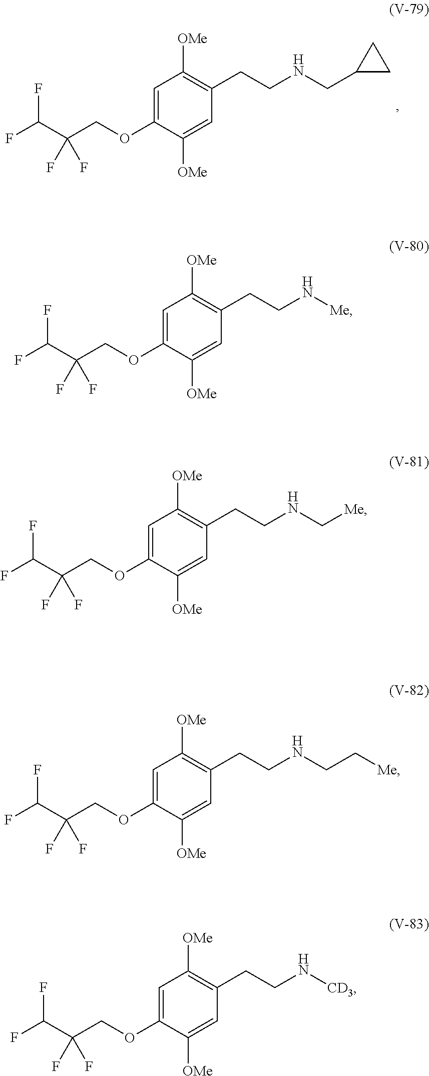 [SMILES: COc1cc(OCC(F)(F)C(F)F)c(OC)cc1CCNCC1CC1 ; CNCCc1cc(OC)c(OCC(F)(F)C(F)F)cc1OC ; CCNCCc1cc(OC)c(OCC(F)(F)C(F)F)cc1OC ; CCCNCCc1cc(OC)c(OCC(F)(F)C(F)F)cc1OC ; [2H]C([2H])([2H])NCCc1cc(OC)c(OCC(F)(F)C(F)F)cc1OC]

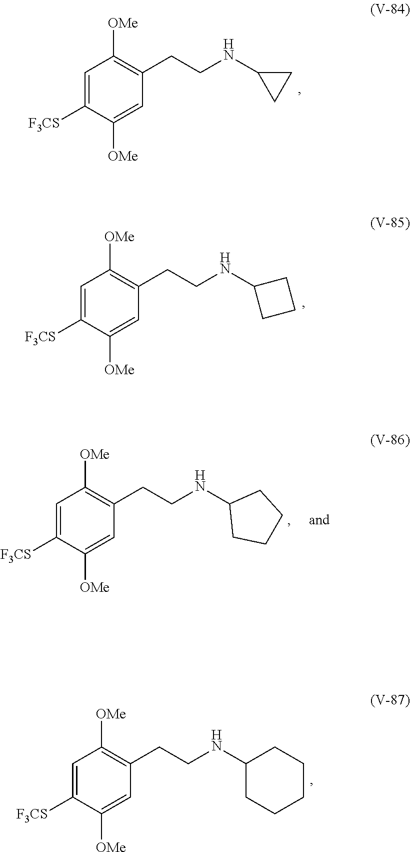

or a pharmaceutically acceptable salt, solvate, or prodrug thereof.

(19) The compound of any one of (1) to (18), wherein the compound is an agonist of a serotonin 5-HT₂ receptor.

(20) The compound of any one of (1) to (19), wherein the compound is an agonist of a serotonin 5-HT_{2A} receptor.

(21) A pharmaceutical composition, comprising the compound of any one of (1) to (20) and a pharmaceutically acceptable excipient.

(22) The pharmaceutical composition of (21), wherein the compound is present in the pharmaceutical composition at a purity of at least 50% by weight based on a total weight of isotopologues of the compound present in the pharmaceutical composition.

(23) The pharmaceutical composition of (21) or (22), wherein any position in the compound having deuterium has a minimum deuterium incorporation of at least 50 atom % at the site of deuteration.

(24) The pharmaceutical composition of any one of (21) to (23), which is substantially free of other isotopologues of the compound.

(25) The pharmaceutical composition of any one of (21) to (24), which is formulated for oral administration.

(26) The pharmaceutical composition of any one of (21) to (24), which is formulated for administration via inhalation.

(27) A method of treating a subject with a disease or disorder associated with a serotonin 5-HT₂ receptor, the method comprising:
administering to the subject a therapeutically effective amount of the compound of any one of (1) to (20).

(28) The method of (27), wherein the disease or disorder associated with a serotonin 5-HT₂ receptor is a neuropsychiatric disease or disorder or an inflammatory disease or disorder.

(29) The method of (27), wherein the disease or disorder associated with a serotonin 5-HT₂ receptor is a central nervous system (CNS) disorder.

(30) The method of (29), wherein the central nervous system (CNS) disorder is selected from the group consisting of post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), a bipolar disorder and related disorders, cyclothymic disorder, obsessive-compulsive disorder (OCD), generalized anxiety disorder (GAD), social anxiety disorder, a substance use disorder including alcohol use disorder, anorexia nervosa, bulimia nervosa, binge eating disorder, Alzheimer's disease, cluster headache and migraine, attention deficit hyperactivity disorder (ADHD), pain, aphantasia, childhood-onset fluency disorder, major neurocognitive disorder, mild neurocognitive disorder, sexual dysfunction, chronic fatigue syndrome, Lyme disease, and obesity.

(31) The method of (29), wherein the central nervous system (CNS) disorder is pain.

(32) The method of (29), wherein the central nervous system (CNS) disorder is sexual dysfunction.

(33) The method of (27), wherein the disease or disorder associated with a serotonin 5-HT₂ receptor is an autonomic nervous system (ANS) disorder.

(34) The method of (33), wherein the autonomic nervous system (ANS) disorder is a pulmonary disorder or a cardiovascular disorder.

(35) The method of any one of (27) to (34), wherein the compound is administered orally, sublingually, buccally, topically, via injection, or via inhalation.

(36) A single-layer orally administered tablet composition comprising the compound of any one of (1) to (20), and a polymer.

(37) The single-layer orally administered tablet composition of (36), wherein the composition is adapted for maximum sustained release.

(38) The single-layer orally administered tablet composition of (36) or (37), wherein the tablet composition comprises a combination of (i) a water-insoluble neutrally charged non-ionic matrix; (ii) a polymer carrying one or more negatively charged groups; and (iii) the compound.

(39) The single-layer orally administered tablet composition of (38), wherein the water-insoluble neutrally charged non-ionic matrix is selected from a cellulose-based polymer, alone or enhanced by mixing with components selected from the group consisting of starches; waxes; neutral gums; polymethacrylates; PVA; PVA/PVP blends; and mixtures thereof.

(40) The single-layer orally administered tablet composition of (39), wherein the cellulose-based polymer is hydroxypropyl methylcellulose (HPMC).

(41) The single-layer orally administered tablet composition of (38), wherein the polymer carrying one or more negatively charged groups is selected from the group consisting of polyacrylic acid, polylactic acid, polyglycolic acid, polymethacrylate carboxylate, a cation-exchange resin, a clay, a zeolite, hyaluronic acid, an anionic gum, salts thereof, and mixtures thereof.

(42) The single-layer orally administered tablet composition of (41), wherein the anionic gum is selected from the group consisting of a naturally occurring material and a semi-synthetic material.

(43) The single-layer orally administered tablet composition of (42), wherein the naturally occurring material is selected from the group consisting of alginic acid, pectin, xanthan gum, carrageenan, locust bean gum, gum arabic, gum karaya, guar gum, and gum tragacanth.

(44) The single-layer orally administered tablet composition of (42) or (43), wherein the semi-synthetic material is selected from the group consisting of carboxymethyl-chitin and cellulose gum.

(45) The single-layer orally administered tablet composition of any one of (36) to (44), comprising a therapeutically effective amount of the compound for the treatment of pain.

(46) The single-layer orally administered tablet composition of any one of (36) to (44), comprising a therapeutically effective amount of the compound for the treatment of brain injury.

(47) The single-layer orally administered tablet composition of any one of (36) to (44), comprising a therapeutically effective amount of the compound for the treatment of depression.

(48) The single-layer orally administered tablet composition of any one of (36) to (44), comprising a therapeutically effective amount of the compound for use in treating a disease or disorder associated with a serotonin 5-HT₂ receptor.

(49) The single-layer orally administered tablet composition of (48), wherein the disease or disorder is a central nervous system (CNS) disorder selected from the group consisting of post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), bipolar and related disorders including bipolar I disorder, bipolar II disorder, cyclothymic disorder, obsessive-compulsive disorder (OCD), generalized anxiety disorder (GAD), social anxiety disorder, substance use disorders including alcohol use disorder, opioid use disorder, amphetamine use disorder, nicotine use disorder, and cocaine use disorder, anorexia nervosa, bulimia nervosa, binge eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, sexual dysfunction, chronic fatigue syndrome, Lyme disease, and obesity.

(50) The single-layer orally administered tablet composition of (48), wherein the disease or disorder is a condition of the autonomic nervous system (ANS).

(51) The single-layer orally administered tablet composition of (48), wherein the disease or disorder is a pulmonary disorder.

(52) The single-layer orally administered tablet composition of (48), wherein the disease or disorder is a cardiovascular disorder.

(53) The single-layer orally administered tablet composition of any one of (36) to (52), wherein the composition achieves a combined concentration of the compound in plasma in the range of 10-500 ng/ml, and maintains this concentration for a duration of release.

(54) The single-layer orally administered tablet composition of any one of (36) to (53), wherein the polymer comprises one or more negatively charged groups.

(55) A tablet composition formulated for oral administration comprising the compound of any one of (1) to (20), and a polymer.

(56) The tablet composition of (55), wherein the polymer comprises one or more negatively charged groups.

(57) The tablet composition of (55), wherein the polymer comprises one or more acid groups.

(58) The tablet composition of any one of (55) to (57), wherein the polymer comprises a water-insoluble neutrally charged non-ionic matrix.

(59) The tablet composition of (58), wherein the water-insoluble neutrally charged non-ionic matrix is selected from a cellulose-based polymer, alone or enhanced by mixing with components selected from the group consisting of starches; waxes; neutral gums; polymethacrylates; PVA; PVA/PVP blends; and mixtures thereof.

(60) The tablet composition of (59), wherein the cellulose-based polymer is hydroxypropyl methylcellulose (HPMC).

(61) A kit for the treatment of a subject comprising 1) a single-layer orally administered tablet composition of any one of (36) to (54), and 2) instructions for use in the treatment of pain.

(62) The kit of (61), wherein the polymer comprises one or more negatively charged groups.

(63) A kit for the treatment of a subject comprising 1) a single-layer orally administered tablet composition of any one of (36) to (54), and 2) instructions for use in the treatment of brain injury.

(64) The kit of (63), wherein the polymer comprises one or more negatively charged groups.

(65) A kit for the treatment of a subject comprising 1) a single-layer orally administered tablet composition of any one of (36) to (54) and 2) instructions for use in the treatment of depression.

(66) The kit of (65), wherein the polymer comprises one or more negatively charged groups.

(67) A kit for the treatment of a subject comprising 1) a single-layer orally administered tablet composition of any one of (36) to (54) and 2) instructions for use in the treatment of a disease or disorder associated with a serotonin 5-HT₂ receptor.

(68) The kit of (67), wherein the polymer comprises one or more negatively charged groups.

(69) A method of delivering a compound of any one of (1) to (20) to a subject in need thereof comprising administering the compound dissolved in a liquid phase of a mist via inhalation.

(70) The method of (69), wherein the compound is delivered to the subject's central nervous system.

(71) The method of (69) or (70), wherein the compound is delivered with air, oxygen, or a mixture of helium and oxygen.

(72) The method of any one of (69) to (71), wherein the compound is delivered with a mixture of helium and oxygen.

(73) The method of (72), wherein the mixture of helium and oxygen is heated to about 50° C. to about 60° C.

(74) The method of (72) or (73), wherein the helium is present in the mixture of helium and oxygen at about 50 to 90% and the oxygen is present in the mixture of helium and oxygen at about 10 to 50%.

(75) The method of any one of (72) to (74), further comprising administering a pretreatment inhalation therapy prior to administration of the mixture of helium and oxygen and the compound.

(76) The method of (75), wherein the pretreatment comprises administering via inhalation a mixture of helium and oxygen heated to about 90° C. to about 120° C. to the subject.

(77) The method of (69) to (76), further comprising (i) administering via inhalation a mixture of helium and oxygen heated to about 90° C. to about 120° C. to the subject, and (ii) administering via inhalation to the subject a mist comprising helium and oxygen heated to about 50° C. to about 60° C. and the compound.

(78) The method of (77), further comprising repeating steps (i) and (ii) at least one time.

(79) The method of any one of (69) to (78), wherein the compound is delivered to the subject's central nervous system with an improvement in drug bioavailability by at least 25% as compared to oral delivery, increased Cₘₐₓ by at least 25% as compared to oral delivery, reduced Tₘₐₓ by at least 50% as compared to oral delivery, or a combination thereof.

(80) A method of treating a central nervous system (CNS) disorder or psychological disorder comprising administering, via inhalation, a compound of any one of (1) to (20) dissolved in a mist.

(81) The method of (80), wherein the compound is delivered with air, oxygen, or a mixture of helium and oxygen.

(82) The method of (80) or (81), wherein the compound is delivered with the mixture of helium and oxygen, and the mixture of helium and oxygen is heated to about 50° C. to about 60° C. prior to administering the compound to the subject.

(83) The method of any one of (80) to (82), wherein the CNS disorder is post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), bipolar and related disorders including bipolar I disorder, bipolar II disorder, cyclothymic disorder, obsessive-compulsive disorder (OCD), generalized anxiety disorder (GAD), social anxiety disorder, substance use disorders including alcohol use disorder, opioid use disorder, amphetamine use disorder, nicotine use disorder, and cocaine use disorder, anorexia nervosa, bulimia nervosa, binge eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, sexual dysfunction, chronic fatigue syndrome, Lyme disease, or obesity.

(84) A transdermal patch, comprising the compound of any one of (1) to (20).

(85) The transdermal patch of (84), further comprising a pressure sensitive adhesive layer, a backing, and a release liner.

(86) The transdermal patch of (85), wherein the compound is uniformly distributed throughout the pressure sensitive adhesive layer.

(87) The transdermal patch of any one of (84) to (86), comprising 5 mg to 25 mg of the compound.

(88) A method of treating a subject with a disease or disorder associated with a serotonin 5-HT₂ receptor, the method comprising: administering to the subject, via the transdermal patch of any one of (84) to (87), a therapeutically effective amount of the compound.

(89) The method of (88), wherein the disease or disorder associated with a serotonin 5-HT₂ receptor is a neuropsychiatric disease or disorder or an inflammatory disease or disorder.

(90) The method of (88), wherein the disease or disorder associated with a serotonin 5-HT₂ receptor is a central nervous system (CNS) disorder.

(91) The method of (90), wherein the central nervous system (CNS) disorder is selected from the group consisting of post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), a bipolar disorder and related disorders, cyclothymic disorder, obsessive-compulsive disorder (OCD), generalized anxiety disorder (GAD), social anxiety disorder, a substance use disorder, anorexia nervosa, bulimia nervosa, binge eating disorder, Alzheimer's disease, cluster headache and migraine, attention deficit hyperactivity disorder (ADHD), pain, aphantasia, childhood-onset fluency disorder, major neurocognitive disorder, mild neurocognitive disorder, sexual dysfunction, chronic fatigue syndrome, Lyme disease, and obesity.

(92) The method of (88), wherein the disease or disorder associated with a serotonin 5-HT₂ receptor is an autonomic nervous system (ANS) disorder.

(93) The method of (92), wherein the autonomic nervous system (ANS) disorder is a pulmonary disorder or a cardiovascular disorder.

(94) The method of any one of (88) to (93), wherein 5 mg to 25 mg of the compound is administered to the subject over a 4 to 72 hour period.

(95) The method of any one of (88) to (94), wherein a serotonergic, but sub-psychoactive concentration of the compound is administered.

(96) A method of treating a subject with a disease or disorder associated with a serotonin 5-HT₂ receptor, the method comprising: administering to the subject transdermally, subcutaneously, or intramuscularly, via an automatic injection device, a therapeutically effective amount of the compound of any one of (1) to (20).

(97) A compound having a structure of Formula (VI):

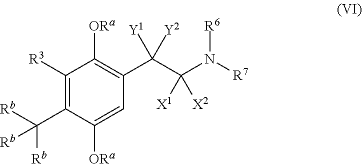

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R³ is hydrogen or deuterium;
R⁶ and R⁷ are independently hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl; or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
each R^{b} is independently a substituted or unsubstituted C₁-C₂ alkyl; or alternatively two R^{b}s together with the carbon atom attached thereto are optionally joined to form a substituted or unsubstituted cycloalkyl.

(98) The compound of (97), which is selected from the group consisting of

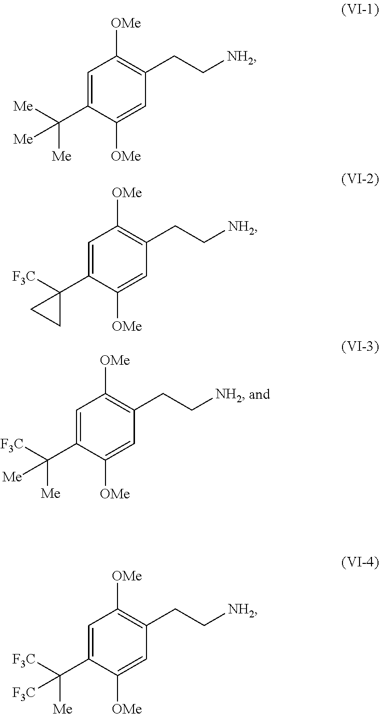

or a pharmaceutically acceptable salt, solvate, or prodrug thereof.

(99) A pharmaceutical composition, comprising the compound of (97) or (98) and a pharmaceutically acceptable excipient.

(100) A method of treating a subject with a disease or disorder associated with a serotonin 5-HT₂ receptor, the method comprising:
administering to the subject a therapeutically effective amount of the compound of (97) or (98).

(101) A compound having a structure of Formula (VII):

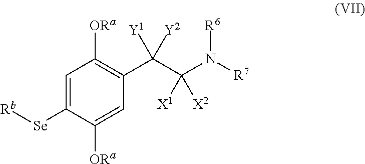

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁶ is hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl.

(102) The compound of (101), which is selected from the group consisting of

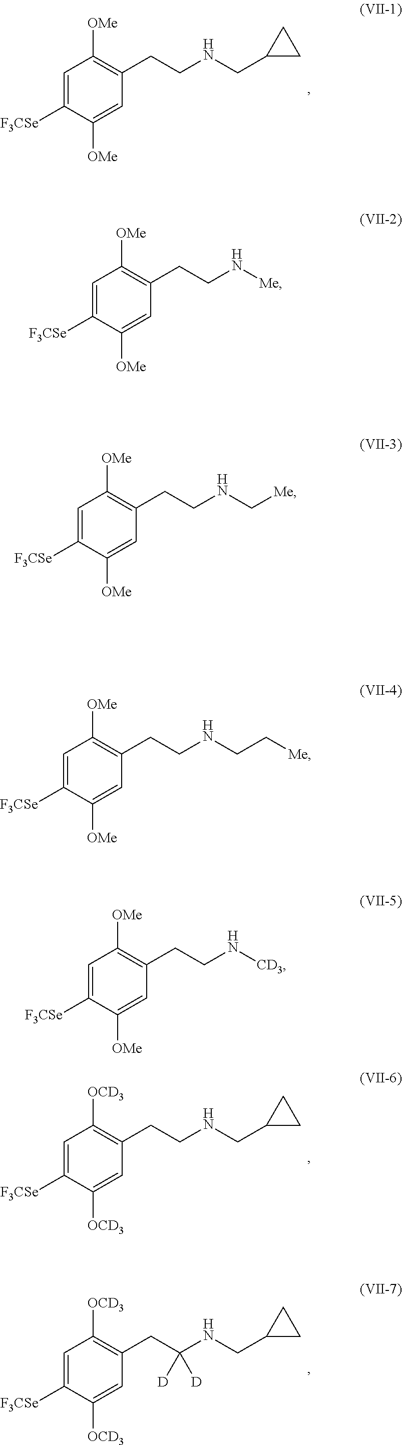

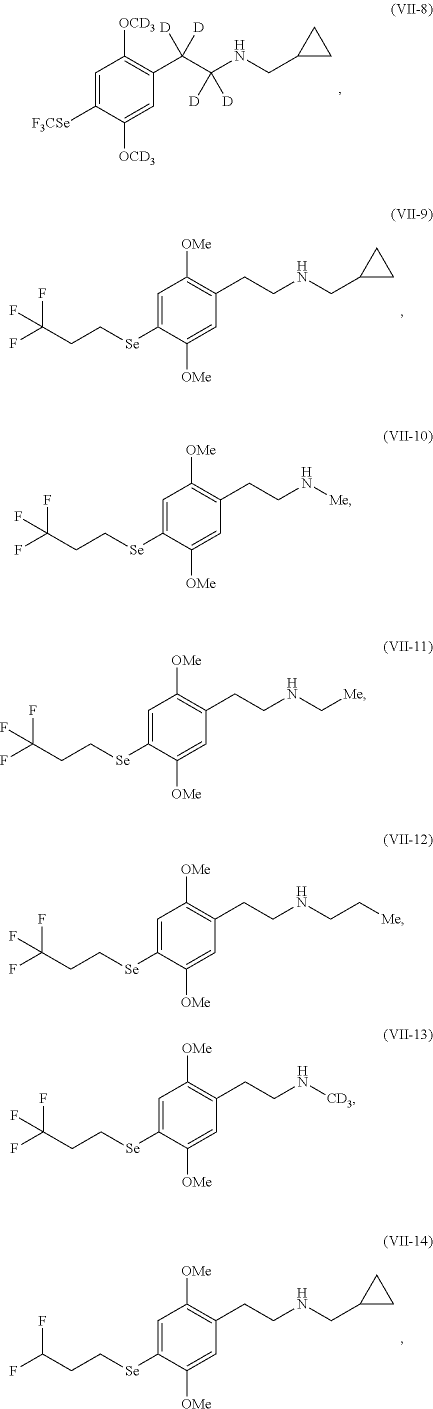

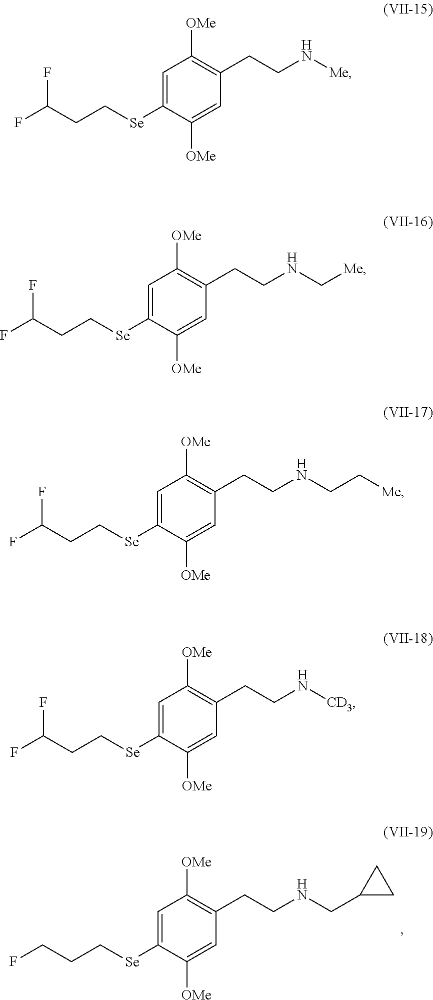 [SMILES: CNCCc1cc(OC)c([Se]CCC(F)F)cc1OC ; CCNCCc1cc(OC)c([Se]CCC(F)F)cc1OC ; CCCNCCc1cc(OC)c([Se]CCC(F)F)cc1OC ; [2H]C([2H])([2H])NCCc1cc(OC)c([Se]CCC(F)F)cc1OC ; COc1cc([Se]CCCF)c(OC)cc1CCNCC1CC1]

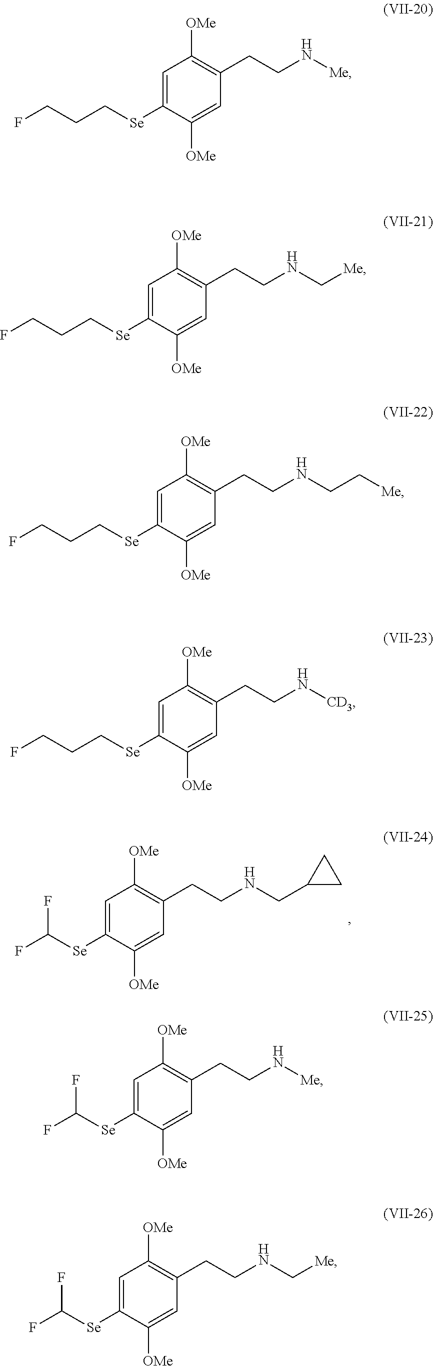

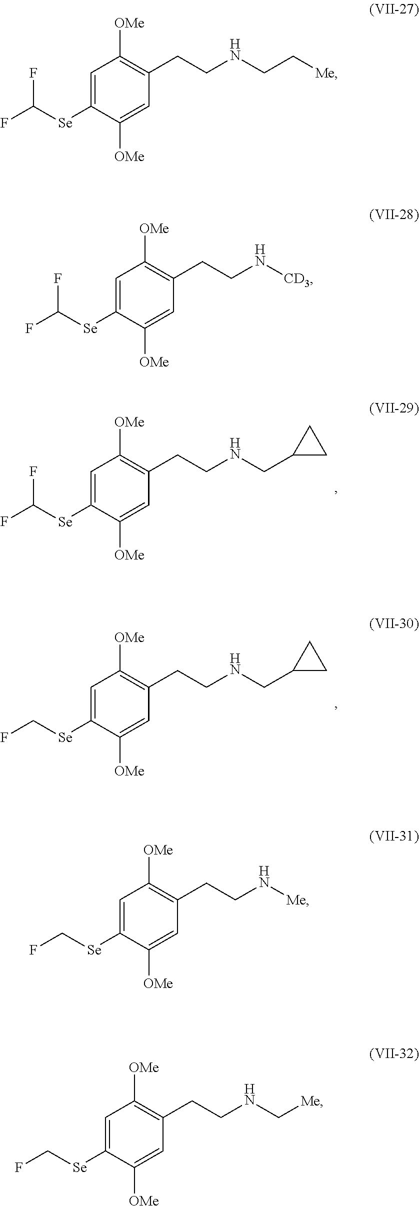 [SMILES: CCCNCCc1cc(OC)c([Se]C(F)F)cc1OC ; [2H]C([2H])([2H])NCCc1cc(OC)c([Se]C(F)F)cc1OC ; COc1cc([Se]C(F)F)c(OC)cc1CCNCC1CC1 ; COc1cc([Se]CF)c(OC)cc1CCNCC1CC1 ; CNCCc1cc(OC)c([Se]CF)cc1OC ; CCNCCc1cc(OC)c([Se]CF)cc1OC]

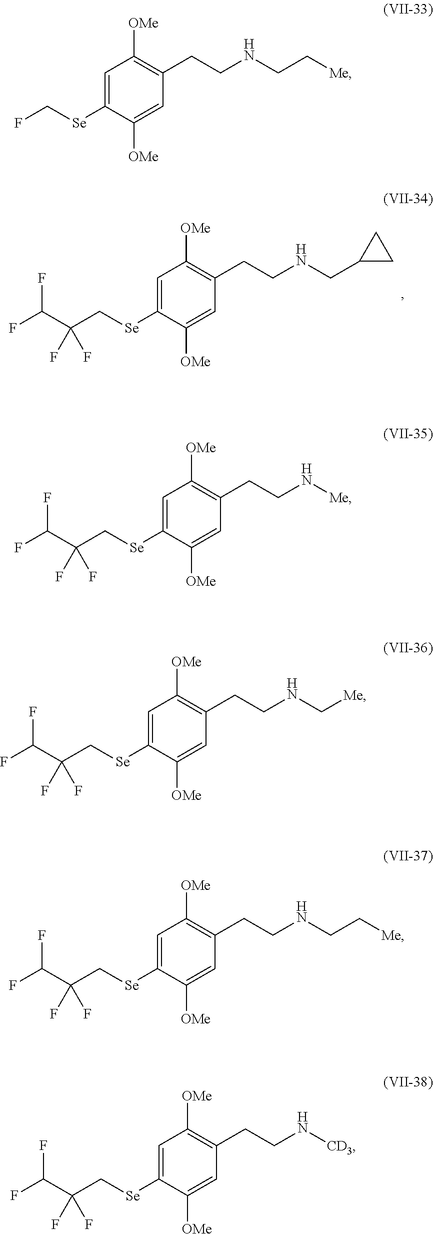 [SMILES: CCCNCCc1cc(OC)c([Se]CF)cc1OC ; COc1cc([Se]CC(F)(F)C(F)F)c(OC)cc1CCNCC1CC1 ; CNCCc1cc(OC)c([Se]CC(F)(F)C(F)F)cc1OC ; CCNCCc1cc(OC)c([Se]CC(F)(F)C(F)F)cc1OC ; CCCNCCc1cc(OC)c([Se]CC(F)(F)C(F)F)cc1OC ; [2H]C([2H])([2H])NCCc1cc(OC)c([Se]CC(F)(F)C(F)F)cc1OC]

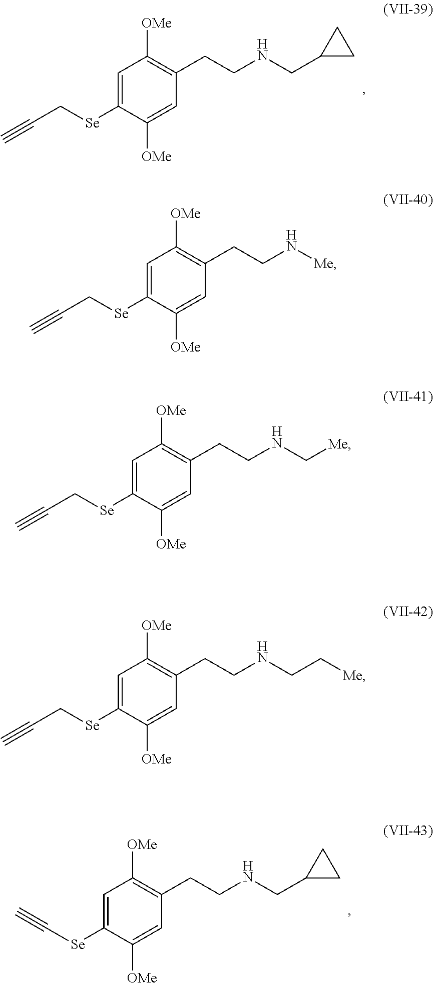 [SMILES: C#CC[Se]c1cc(OC)c(CCNCC2CC2)cc1OC ; C#CC[Se]c1cc(OC)c(CCNC)cc1OC ; C#CC[Se]c1cc(OC)c(CCNCC)cc1OC ; C#CC[Se]c1cc(OC)c(CCNCCC)cc1OC ; C#C[Se]c1cc(OC)c(CCNCC2CC2)cc1OC]

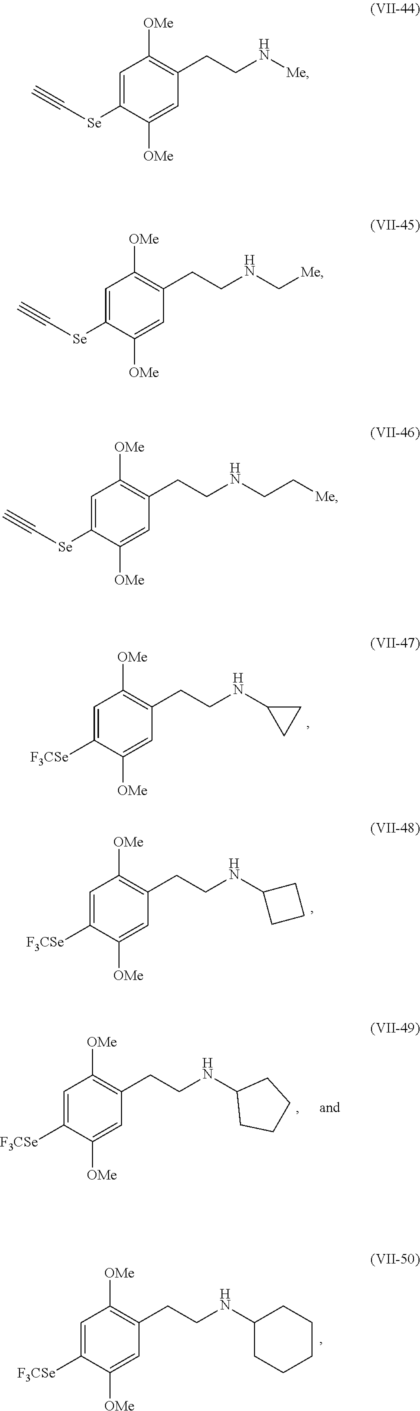

or a pharmaceutically acceptable salt, solvate, or prodrug thereof.

(103) A pharmaceutical composition, comprising the compound of (101) or (102) and a pharmaceutically acceptable excipient.

(104) A method of treating a subject with a disease or disorder associated with a serotonin 5-HT₂ receptor, the method comprising:
administering to the subject a therapeutically effective amount of the compound of (101) or (102).

(105) Use of a compound of any one of (1) to (20) for treating a subject with a disease or disorder, including a disease or disorder associated with a serotonin 5-HT₂ receptor.

(106) Use of a compound of (97) or (98) for treating a subject with a disease or disorder, including a disease or disorder associated with a serotonin 5-HT₂ receptor.

(107) Use of a compound of (101) or (102) for treating a subject with a disease or disorder, including a disease or disorder associated with a serotonin 5-HT₂ receptor.

### BRIEF DESCRIPTION OF THE DRAWINGS

The forgoing paragraphs have been provided by way of general introduction and are not intended to limit the scope of the following claims. The described embodiments, together with further advantages, will be best understood by reference to the following detailed description when considered in conjunction with the accompanying drawings, wherein:

FIG. 1 illustrates the synthetic route for making compound II-1;

FIG. 2 illustrates the synthetic route for making compound II-2;

FIG. 3 illustrates the synthetic route for making compound II-3;

FIG. 4 illustrates the synthetic route for making compound II-4;

FIG. 5 illustrates the synthetic route for making compound II-14;

FIG. 6 illustrates the synthetic route for making compound III-1;

FIG. 7 illustrates the synthetic route for making compound III-2;

FIG. 8 illustrates the synthetic route for making compound IV-1;

FIG. 9 illustrates the synthetic route for making compound IV-2;

FIG. 10 illustrates the synthetic route for making compound IV-3;

FIG. 11 illustrates the synthetic route for making compound IV-5;

FIG. 12 illustrates the synthetic route for making compound IV-12;

FIG. 13 illustrates the synthetic route for making compound I-1;

FIG. 14 illustrates the synthetic route for making compound I-2;

FIG. 15 illustrates the synthetic route for making compound I-3;

FIG. 16 illustrates the synthetic route for making compound I-4;

FIG. 17 illustrates the synthetic route for making compound I-5;

FIG. 18 illustrates the synthetic route for making compound I-6;

FIG. 19 illustrates the synthetic route for making compound I-7;

FIG. 20 illustrates the synthetic route for making compounds of Formula (I) with a fluoropropoxy or fluorothiopropoxy substituent as R⁴, e.g., compounds II-16, II-17, II-18, II-19, II-20 and II-21;

FIG. 21 illustrates the synthetic route for making compound I-8;

FIG. 22 illustrates the synthetic route for making compound I-10;

FIG. 23 illustrates the synthetic route for making compound I-11;

FIG. 24 illustrates the synthetic route for making compound I-13;

FIG. 25 illustrates the synthetic route for making compound II-10;

FIG. 26 illustrates the synthetic route for making compound II-23;

FIG. 27 illustrates the synthetic route for making compound V-1;

FIG. 28 illustrates the synthetic route for making compound VI-1;

FIG. 29 illustrates the synthetic route for making compound IV-33;

FIG. 30 illustrates the synthetic route for making Reference Compound 1;

FIG. 31 is a graph showing agonist-labeled 5-HT_{2A} radioligand ([³H]ketanserin) competition binding using compound II-1 and IV-1; at least three independent experiments, with compounds tested in duplicate were performed with data shown being the average results from all experiments combined; K_{d} for [³H]ketanserin was set to 1.57 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 32 is a graph showing agonist-labeled 5-HT_{2A} radioligand ([³H]ketanserin) competition binding using compound II-23 and IV-3; at least three independent experiments, with compounds tested in duplicate were performed with data shown being the average results from all experiments combined; K_{d} for [³H]ketanserin was set to 1.57 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 33 is a graph showing agonist-labeled 5-HT_{2A} radioligand ([³H]ketanserin) competition binding using compound I-1 and II-10 and 5-HT as positive control; data shown are the average results from 2-6 experiments combined (N=6-12 replicates per concentration); K_{d} for [³H]ketanserin was set to 1.57 nM, and data were analyzed using a two-site, fit Kᵢ model (GraphPad Prism 9); the data point for −10 samples is total, specific binding (no compound present) and −4 was interpolated to complete the binding curves to 0 specific binding;

FIG. 34 is a graph showing agonist-labeled 5-HT_{2A} radioligand ([³H]LSD) competition binding using compounds I-13 and II-4; three independent experiments with compounds tested in duplicate (5-HT, control) or triplicate (I-13, II-4) were performed with data shown being the average results from all experiments combined (N=6-12 replicates per concentration); K_{d} for [³H]LSD was set to 0.78 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 35 is a graph showing agonist-labeled 5-HT_{2A} radioligand ([³H]LSD) competition binding using compounds VI-1, II-3, and III-2; up to 11 independent experiments with 5-HT tested in duplicate and compounds II-3, III-2, VI-1 in triplicate were performed with data shown being the average results from all experiments combined (N=up to 33 replicates); K_{d} for [³H]LSD was set to 0.78 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 36 is a graph showing agonist-labeled 5-HT_{2A} radioligand ([³H]LSD) competition binding using compounds I-4 and I-7; four independent experiments with 5-HT tested in duplicate (N=8 replicates) and compounds I-4 and I-7 in triplicate (N=12) were performed with data shown being the average results from all experiments combined; K_{d} for [³H]LSD was set to 0.78 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 37 is a graph showing agonist-labeled 5-HT_{2A} radioligand ([³H]LSD) competition binding using compound I-3; four independent experiments with 5-HT tested in duplicate (N=8 replicates) and compound I-3 in triplicate (N=12) were performed with data shown being the average results from all experiments combined; K_{d} for [³H]LSD was set to 0.78 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 38 is a graph showing agonist-labeled 5-HT_{2A} radioligand ([³H]LSD) competition binding using compound I-2; four independent experiments with 5-HT tested in duplicate (N=8 replicates) and compound I-2 in triplicate (N=12) were performed with data shown being the average results from all experiments combined; K_{d} for [³H]LSD was set to 0.78 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 39 is a graph showing agonist-labeled 5-HT_{2A} radioligand ([³H]LSD) competition binding using compounds I-5 and I-6; seven independent experiments with 5-HT tested in duplicate (N=14 replicates) and compounds I-5 and I-6 in triplicate (N=21) were performed with data shown being the average results from all experiments combined; K_{d} for [³H]LSD was set to 0.78 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 40 is a graph showing agonist-labeled 5-HT_{2A} radioligand ([³H]LSD) competition binding using compound I-8; three independent experiments with 5-HT tested in duplicate (N=6 replicates) and compound I-8 in triplicate (N=9) were performed with data shown being the average results from all experiments combined; K_{d} for [³H]LSD was set to 0.78 nM, and data were analyzed using a one-site, fit Kᵢ model (GraphPad Prism 9) the data point for −10 samples is total, specific binding (no compound present) and −4 was interpolated to complete the binding curves to 0 specific binding;

FIG. 41 is a graph showing agonist-labeled 5-HT_{2A} radioligand ([³H]LSD) competition binding using compound V-1; two independent experiments with 5-HT tested in duplicate (N=4 replicates) and compound V-1 in triplicate (N=6) were performed with data shown being the average results from all experiments combined; K_{d} for [³H]LSD was set to 0.78 nM, and data were analyzed using a one-site, fit Kᵢ model (GraphPad Prism 9); the data point for −10 samples is total, specific binding (no compound present) and −4 was interpolated to complete the binding curves to 0 specific binding;

FIG. 42 is a graph showing a dose-response of 5-HT_{2A} functional assay as a percent of control agonist response (5-HT); compound I-1 was tested in duplicate at the indicated concentrations and the EC₅₀ and Eₘₐₓ values were determined by non-linear regression analysis of the concentration-response curves generated with the mean replicate values using Hill equation curve fitting;

FIG. 43 is a graph showing a dose-response of 5-HT_{2A} functional assay as a percent of control agonist response (5-HT); compounds I-3, I-8, and VI-1 were tested in duplicate at the indicated concentrations and the EC₅₀ and Eₘₐₓ values were determined by non-linear regression analysis of the concentration-response curves generated with the mean replicate values using Hill equation curve fitting;

FIG. 44 is a graph showing a dose-response of 5-HT_{2A} functional assay as a percent of control agonist response (5-HT); compound I-10 was tested in duplicate at the indicated concentrations and the EC₅₀ and Eₘₐₓ values were determined by non-linear regression analysis of the concentration-response curves generated with the mean replicate values using Hill equation curve fitting;

FIG. 45 is a graph showing a dose-response of 5-HT_{2A} functional assay as a percent of control agonist response (5-HT); compound V-1 was tested in duplicate at the indicated concentrations and the EC₅₀ and Eₘₐₓ values were determined by non-linear regression analysis of the concentration-response curves generated with the mean replicate values using Hill equation curve fitting;

FIG. 46 is a graph showing a dose-response of 5-HT_{2A} functional assay (TRUPATH) as a percent of control agonist response (5-HT); compound V-1 was tested in 11 replicates at the indicated concentrations and the EC₅₀ and Eₘₐₓ values were determined by non-linear regression analysis of the concentration-response curves generated with the mean replicate values using Hill equation curve fitting;

FIG. 47 is a graph showing a dose-response of 5-HT_{2B} functional assay as a percent of control agonist response (5-HT); compound I-1 was tested in duplicate at the indicated concentrations and the EC₅₀ and Eₘₐₓ values were determined by non-linear regression analysis of the concentration-response curves generated with the mean replicate values using Hill equation curve fitting;

FIG. 48 is a graph showing a dose-response of 5-HT_{2B} functional assay as a percent of control agonist response (5-HT); compounds I-3, I-8, and VI-1 were tested in duplicate at the indicated concentrations and the EC₅₀ and Eₘₐₓ values were determined by non-linear regression analysis of the concentration-response curves generated with the mean replicate values using Hill equation curve fitting;

FIG. 49 is a graph showing a dose-response of 5-HT_{2B} functional assay as a percent of control agonist response (5-HT); compound I-10 was tested in duplicate at the indicated concentrations and the EC₅₀ and Eₘₐₓ values were determined by non-linear regression analysis of the concentration-response curves generated with the mean replicate values using Hill equation curve fitting;

FIG. 50 is a graph showing a dose-response of 5-HT_{2B} functional assay as a percent of control agonist response (5-HT); compound V-1 was tested in duplicate at the indicated concentrations and the EC₅₀ and Eₘₐₓ values were determined by non-linear regression analysis of the concentration-response curves generated with the mean replicate values using Hill equation curve fitting;

FIG. 51 is a graph showing a dose-response of 5-HT_{2C} functional assay (TRUPATH) as a percent of control agonist response (5-HT); compound V-1 was tested in 9 replicates at the indicated concentrations and the EC₅₀ and Eₘₐₓ values were determined by non-linear regression analysis of the concentration-response curves generated with the mean replicate values using Hill equation curve fitting;

FIG. 52 is a graph showing the head-twitch response in adult, male C57BL/6J mice after administration of 0.1, 0.3, 1, and 3 mg/kg s.c. of compound I-1 and observed at the indicated time points; DOI was used as a positive control, vehicle as a negative control;

FIGS. 53A-53B are graphs showing oral administration of I-1 (1-10 mg/kg, p.o.) induced the HTR in C57BL/6 mice; I-1 at doses of 1, 3 and 10 mg/kg p.o. produced a dose-dependent increase in HTRs similar to I-1 3 mg/kg s.c. (n=10 per group) and DOI 1 mg/kg i.p. (n=4); the HTR is compared to saline vehicle (n=4) across minutes 1-30 (FIG. 53A) and 120-150 (FIG. 53B) post-dosing, showing that significant numbers of head-twitches were present in all test compound groups in minutes 1-30, and minutes 120-150; *p<0.05; **p<0.01 vs. vehicle;

FIGS. 54A-54B are graphs showing mean I-1 concentrations ±SD in mouse plasma and brain samples over time in C57BL/6J mice (n=3) administered orally (FIG. 54A) or subcutaneously (FIG. 54B) at 3 mg/kg and plasma and brain samples were collected at the indicated time points;

FIGS. 55A-55B are graphs showing the mean concentrations ±SD of I-1 in rat plasma samples and brain samples over time on a linear scale after intravenous administration at 1 mg/kg (FIG. 55A) and oral administration at 5 mg/kg (FIG. 55B) to male Wistar rats (n=3);

FIGS. 56A-56B are graphs showing the mean concentrations ±SD of I-1 in rat plasma, brain, and CSF samples over time on a linear scale (FIG. 56A) and a log scale (FIG. 56B) after intravenous administration (1 mg/kg, s.c.) to male Sprague-Dawley rats (n=4 per time point); and

FIGS. 57A-57J are graphs showing transcriptional changes in the frontal cortex of male C57BL/6J mice in the acute phase (2 h) after treatment with treatment groups A, B, C, and D in the following brain markers of neuroplasticity: bdnf (FIG. 57A), psd-95 (FIG. 57B), homer1 (FIG. 57C), Egr2 (FIG. 57D), cFos (FIG. 57E), mTOR (FIG. 57F), Creb1 (FIG. 57G), Syn1 (FIG. 57H), Nptn (FIG. 57I), and Igf1 (FIG. 57J); statistical analysis by one-way ANOVA followed by multiple comparisons against vehicle group on log transformed data, back transformed data presented, *=p<0.05, **=p<0.01, ****=p<0.001.

### DETAILED DESCRIPTION

In the following detailed description of the embodiments of the instant disclosure, numerous specific details are set forth in order to provide a thorough understanding of the disclosed embodiments. However, it will be obvious to one skilled in the art that the embodiments of this disclosure may be practiced without these specific details. In other instances, well known methods, procedures, components, and circuits have not been described in detail so as not to unnecessarily obscure aspects of the embodiments of the instant disclosure.

### Definitions

Unless defined otherwise, all technical and scientific terms used herein have the same meaning as is commonly understood by one of skill in the art to which this disclosure belongs.

“Alkyl” refers to monovalent saturated aliphatic hydrocarbyl groups having from 1 to 10 carbon atoms and such as 1 to 6 carbon atoms, or 1 to 5, or 1 to 4, or 1 to 3, or 1 to 2 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl (t-Bu)((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, −S—, —S(O)ₙ— (where n is 0 to 2), —NR— (where R is hydrogen or alkyl) and having from 1 to 10 substituents selected from the group consisting of deuterium, alkoxy, substituted alkoxy, unsubstituted cycloalkyl, substituted cycloalkyl, unsubstituted cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR′R″, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups having from 1 to 6, including, for example, 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms, for example 2 to 4 carbon atoms and having at least 1, for example from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 10 carbon atoms, for example, 2 to 3 carbon atoms, 3 to 6 carbon atoms, and having at least 1 and for example, from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO— heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclohexyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from deuterium, alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂—heteroaryl.

“Cycloalkenyl” refers to non-aromatic cyclic alkyl groups of from 3 to 10 carbon atoms having single or multiple rings and having at least one double bond and for example, from 1 to 2 double bonds.

The term “substituted cycloalkenyl” refers to cycloalkenyl groups having from 1 to 5 substituents, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂—alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In some embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO—alkyl, —SO-substituted alkyl, —SO-aryl, —SO-heteroaryl, —SO₂-alkyl, —SO₂-substituted alkyl, —SO₂-aryl and —SO₂—heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In some embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, benzo[d][1,3]oxathiole, benzo[d][1,3]dioxole, and the like.

Unless otherwise constrained by the definition for the heterocyclic substituent, such heterocyclic groups can be optionally substituted with 1 to 5, or from 1 to 3 substituents, selected from deuterium, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, and —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In some embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, deuterium, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O—)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the disclosure and the other a typical counter ion such as chloride, or two ionized compounds disclosed herein can serve as counter ions for such divalent alkali earth ions, or a doubly ionized compound of the disclosure can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, deuterium, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸OR⁸⁰, —C(NR⁷⁰)NR⁸OR⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O—)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein, unless specified otherwise. In such cases, the maximum number of such substitutions is three. For example, serial substitutions of substituted aryl groups specifically contemplated herein are limited to substituted aryl-(substituted aryl)-substituted aryl. However, substituent groups defined as e.g., polyethers may contain serial substitution greater than three, e.g., —O—(CH₂CH₂O)ₙ—H, where n can be 1, 2, 3, or greater.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

As used herein, the term “fatty” describes a compound with a long-chain (linear) hydrophobic portion made up of hydrogen and anywhere from 4 to 26 carbon atoms, which may be fully saturated or partially unsaturated.

When it is defined that a substituent or group “comprise(s) deuterium” or is “comprising deuterium,” it is to be understood that the substituent or group may itself be deuterium, or the substituent or group may contain at least one deuterium substitution in its chemical structure. For example, when substituent “—R” is defined to comprise deuterium, it is to be understood that —R may be -D (-deuterium), or a group such as —CD₃ that is consistent with the other requirements set forth of —R.

The phrases “pharmaceutically acceptable,” “physiologically acceptable,” and the like, are employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio. When referencing salts, the phrases “pharmaceutically acceptable salt,” “physiologically acceptable salt,” and the like, means a salt which is acceptable for administration to a patient, such as a mammal (salts with counterions having acceptable mammalian safety for a given dosage regime). As is well known in the art, such salts can be derived from pharmaceutically acceptable inorganic or organic bases, by way of example, sodium, potassium, calcium, magnesium, lithium, aluminum, zinc, and ammonium and tetraalkylammonium salts (e.g., salts formed with pharmaceutically acceptable amines such as ammonia, alkylamines, hydroxyalkylamines, lysine, arginine, N-methylglucamine, procaine, etc.), and the like, and when the molecule contains a basic functionality, addition salts with inorganic acids, such as hydrochloride, hydrobromide, sulfate, sulfamate, phosphate, nitrate, perchlorate salts, and the like, and addition salts with organic acids, such as formate, tartrate, besylate, mesylate, acetate, maleate, malonate, oxalate, fumarate, benzoate, salicylate, succinate, oxalate, glycolate, hemi-oxalate, hemi-fumarate, propionate, stearate, lactate, citrate, ascorbate, pamoate, hydroxymaleate, phenylacetate, glutamate, 2-acetoxybenzoate, tosylate, ethanedisulfonate, isethionate salts, and the like. The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a physical association of a compound or salt of the present disclosure with one or more solvent molecules, whether organic, inorganic, or a mixture of both. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Some examples of solvents include, but are not limited to, methanol, ethanol, isopropanol, N,N-dimethylformamide, tetrahydrofuran, dimethylsulfoxide, and water. When the solvent is water, the solvate formed is a hydrate (e.g., monohydrate, dihydrate, etc.). Exemplary solvates thus include, but are not limited to, hydrates, methanolates, ethanolates, isopropanolates, etc. Methods of solvation are generally known in the art.

“Stereoisomer” and “stereoisomers” refer to compounds that have same atomic connectivity but different atomic arrangement in space. Stereoisomers include cis-trans isomers, E and Z isomers, enantiomers, and diastereomers. All forms such as racemates and optically pure stereoisomers of the compounds are contemplated herein. Chemical formulas and compounds which possess at least one stereogenic center, but are drawn without reference to stereochemistry, are intended to encompass both the racemic compound, as well as the separate stereoisomers, e.g., R- and/or S-stereoisomers, each permutation of diastereomers so long as those diastereomers are geometrically feasible, etc.

“Tautomer” refers to alternate forms of a molecule that differ only in electronic bonding of atoms and/or in the position of a proton, such as enol-keto, imine-enamine, and neutral/zwitterionic tautomers, or the tautomeric forms of heteroaryl groups containing a —N═C(H)—NH— ring atom arrangement, such as pyrazoles, imidazoles, benzimidazoles, triazoles, and tetrazoles. Other tautomeric ring atom arrangements are also possible. For example, compounds containing an acid and a base group within the same molecule depicted in neutral form may exist also in a zwitterionic form, as is the case for amino acid/ammonium carboxylate tautomers. A given chemical formula or name shall encompass all tautomeric forms thereof, insofar as they exist.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, e.g., an ester, a phosphate ester, etc. but is converted in vivo to an active compound, for example, by hydrolysis to a free carboxylic acid or free hydroxyl group. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxyl, free amino or free mercapto group, respectively. Examples of prodrugs include, but are not limited to, ester (e.g., acetate, formate, benzoate, etc.), carbonate, carbamate, and dihydrogen phosphate derivatives of an alcohol, or amide (e.g., acetamide, formamide, benzamide, etc.), carbamate, etc. derivatives of an amine functional group in the active compound, and the like.

Compounds of the present disclosure may in some cases exist in a crystalline solid form or an amorphous solid form, and as such, these solid forms are contemplated herein. A “crystalline” solid is a type of solid whose fundamental three-dimensional structure contains a highly regular pattern of atoms or molecules—with long range order—forming a crystal lattice, and thus displays sharp characteristic crystalline peak(s) in its X-ray power diffraction (XRPD) pattern. In some instances, crystalline solids can exist in different crystalline forms known as “polymorphs,” which have the same chemical composition, but differ in packing, geometric arrangement, and other descriptive properties of the crystalline solid state. As such, polymorphs may have different solid-state physical properties to affect, for example, the solubility, dissolution rate, bioavailability, chemical and physical stability, flowability, and compressibility, etc. of the compound as well as the safety and efficacy of drug products based on the compound. In the process of preparing a polymorph, further purification, in terms of gross physical purity or optical purity, may be accomplished as well. As used herein, the term “amorphous” refers to a solid material having substantially no long range order in the position of its molecules—the molecules are arranged in a random manner so that there is effectively no well-defined arrangement, e.g., molecular packing, and no long range order. Amorphous solids are generally isotropic, i.e., exhibit similar properties in all directions and do not have definite melting points. For example, an amorphous material is a solid material having substantially no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. Thus, an “amorphous” subject compound/material is one characterized as having substantially no crystallinity—less than 10% crystallinity, less than 8% crystallinity, less than 6% crystallinity, less than 4% crystallinity, less than 2% crystallinity, less than 1% crystallinity, or 0% crystallinity—i.e., is at least 90%, at least 92%, at least 94%, at least 96%, at least 98%, or 100% amorphous, as determined for example by XRPD. For example, the % crystallinity can in some embodiments be determined by measuring the intensity of one or more peaks in the XRPD diffractogram compared to a reference peak, which may be that of an internal standard. Other characterization techniques, such as modulated differential scanning calorimetry (mDSC) analysis, Fourier transform infrared spectroscopy (FTIR), and other quantitative methods, may also be employed to determine the percent a subject compound/material is amorphous or crystalline, including quantitative methods which provide the above percentages in terms of weight percent.

It will be appreciated that the compounds herein can exist in different salt, solvate, stereoisomer, tautomer, crystalline/amorphous (including polymorphic) forms, and the present disclosure is intended to include all permutations thereof, such as a solvate of a pharmaceutically acceptable salt of a stereoisomer of the subject compound.

A “vapor” is a solid substance in the gas phase at a temperature lower than its critical temperature, meaning that the vapor can be condensed to a liquid by increasing the pressure on it without reducing the temperature.

An “aerosol”, as used herein, is a suspension of fine solid particles or liquid droplets in a gas phase (e.g., air, oxygen, helium, nitrous oxide, and other gases, as well as mixtures thereof). A “mist”, as used herein, is a subset of aerosols, differing from a vapor, and is a dispersion of liquid droplets (liquid phase) suspended in the gas phase (e.g., air, oxygen, helium, and mixtures thereof). The liquid droplets of an aerosol or mist can comprise a drug moiety dissolved in an aqueous liquid, organic solvent, or a mixture thereof. The gas phase of an aerosol or mist can comprise air, oxygen, helium, or other gases, including mixtures thereof. Mists do not comprise solid particulates. Aerosols and mists of the present disclosure can be generated by any suitable methods and devices, examples of which are set forth herein, e.g., through use of an inhaler or nebulizer.

As used herein, the term “inhalation session” describes a dosing event whereby the subject inhales a given dose of drug, irrespective of the number of breadths needed to inhale the given dose. For example, a subject prescribed to take 10 mg of a drug twice a day would undertake two inhalation sessions, each inhalation session providing 10 mg of the drug. The length of time and the number of breaths for each inhalation session would be dependent on factors such as the inhalation device used, the amount of drug that is drawn per breath, the concentration of the drug in the dosage form, the subject's breathing pattern, etc.

As used herein, the language “release period” describes the time window in which any compound described herein is released from the dosage form (e.g., the matrix) to afford plasma concentrations of compounds described herein. The start time of the release period is defined from the point of administration to a subject, which for oral administration is considered nearly equivalent to entry into the stomach, and initial dissolution by gastric enzymes and acid.

As used herein, the language “maximum sustained release” describes the release window for certain formulations of the present disclosure formulated to increase the release period to a maximum value, which for enteral routes is ultimately limited by the time the gastrointestinal tract naturally excretes all drugs with food.

The language “tamper resistance” is art-recognized to describe aspects of a drug formulation that make it more difficult to use the formulation to abuse the drug moiety of the formulation through extraction for intravenous use, or crushing for freebase use; and therefore reduce the risk for abuse of the drug.

As used herein, the term “steady” describes the stable or steady-state level of a molecule concentration, e.g., concentration of any compound described herein.

As used herein, the term “composition” is equivalent to the term “formulation.”

As used herein, the language “administration event” describes the administration of a given dose to a subject within a short window of time, e.g., less than 10 minutes. An oral administration event may be in the form of administration of, for example, one or more pills within a short window of time.

The term “treating” or “treatment” as used herein means the treating or treatment of a disease or medical condition in a patient, such as a mammal (particularly a human) that includes: ameliorating the disease or medical condition, such as, eliminating or causing regression of the disease or medical condition in a patient; suppressing the disease or medical condition, for example by, slowing or arresting the development of the disease or medical condition in a patient; or alleviating a symptom of the disease or medical condition in a patient. In some embodiments, prophylactic treatment can result in preventing the disease or medical condition from occurring, in a subject.

A “patient” or “subject,” used interchangeably herein, can be any mammal including, for example, a human or a non-human subject. A patient or subject can have a condition to be treated or can be susceptible to a condition to be treated.

As used herein, and unless otherwise specified, the terms “prevent,” “preventing” and “prevention” refer to the prevention of the onset, recurrence or spread of a disease, disorder, or condition, or of one or more symptoms thereof. The terms encompass the inhibition or reduction of a symptom of the particular disease, disorder, or condition. Subjects with familial history of a disease, disorder, or condition, in particular, are candidates for preventive regimens in some embodiments. In addition, subjects who have a history of recurring symptoms are also potential candidates for the prevention. In this regard, the term “prevention” may be interchangeably used with the term “prophylactic treatment.”

As used herein, and unless otherwise specified, the terms “manage,” “managing” and “management” refer to preventing or slowing the progression, spread or worsening of a disease, disorder, or condition, or of one or more symptoms thereof. Often, the beneficial effects that a subject derives from a prophylactic and/or therapeutic agent do not result in a cure of the disease, disorder, or condition. In this regard, the term “managing” encompasses treating a subject who had suffered from the particular disease, disorder, or condition in an attempt to prevent or minimize the recurrence of the disease, disorder, or condition, or of one or more symptoms thereof.

“Therapeutically effective amount” refers to an amount of a compound sufficient to treat a specified disorder or disease or one or more of its symptoms and/or to prevent the occurrence of the disease or disorder (prophylactically effective amount).

As used herein, and unless otherwise specified, a “prophylactically effective amount” of an active agent, is an amount sufficient to prevent a disease, disorder, or condition, or prevent its recurrence. The term “prophylactically effective amount” can encompass an amount that improves overall prophylaxis or enhances the prophylactic efficacy of another prophylactic agent.

The term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. A “continuous” administration schedule means administration every day without interruption during the treatment course. If the administration schedule follows an “intermittent” administration schedule, then days of administration may be followed by “rest days” or days of non-administration of drug within the course. A “drug holiday” indicates that the drug is not administered in a predetermined administration schedule. For example, after undergoing several courses of treatment, a subject may be prescribed a regulated drug holiday as part of the administration schedule, e.g., prior to re-recommencing active treatment.

The language “toxic spikes” is used herein to describe spikes in concentration of any compound described herein that would produce neurological side-effects of sedation or psychotomimetic effects, (e.g., hallucination, dizziness, and nausea), or any unwanted and/or unintended secondary effects caused by the administration of a medicament to an individual resulting in subjective experiences being qualitatively different from those of ordinary consciousness. These experiences can include derealization, depersonalization, hallucinations and/or sensory distortions in the visual, auditory, olfactory, tactile, proprioceptive and/or interoceptive spheres and/or any other perceptual modifications, and/or any other substantial subjective changes in cognition, memory, emotion and consciousness. Such side effects, when unwanted, unintended, and/or severe, can not only have immediate repercussions, but also effect treatment compliance. In particular, side effects may become more pronounced at blood concentration levels of about 250, 300, 400, 500 ng/L or more.

As used herein, and unless otherwise specified, a “neuropsychiatric disease or disorder” is a behavioral or psychological problem associated with a known neurological condition, and typically defined as a cluster of symptoms that co-exist. Examples of neuropsychiatric disorders include, but are not limited to, schizophrenia, cognitive deficits in schizophrenia, attention deficit disorder, attention deficit hyperactivity disorder, bipolar and manic disorders, depression or any combinations thereof.

“Inflammatory conditions” or “inflammatory disease,” as used herein, refers broadly to chronic or acute inflammatory diseases, including, but not limited to, rheumatic diseases (e.g., rheumatoid arthritis, osteoarthritis, psoriatic arthritis) spondyloarthropathies (e.g., ankylosing spondylitis, reactive arthritis, Reiter's syndrome), crystal arthropathies (e.g., gout, pseudogout, calcium pyrophosphate deposition disease), multiple sclerosis, Lyme disease, polymyalgia rheumatica; connective tissue diseases (e.g., systemic lupus erythematosus, systemic sclerosis, polymyositis, dermatomyositis, Sjogren's syndrome); vasculitides (e.g., polyarteritis nodosa, Wegener's granulomatosis, Churg-Strauss syndrome); inflammatory conditions including consequences of trauma or ischaemia, sarcoidosis; vascular diseases including atherosclerotic vascular disease, atherosclerosis, and vascular occlusive disease (e.g., atherosclerosis, ischaemic heart disease, myocardial infarction, stroke, peripheral vascular disease), and vascular stent restenosis; ocular diseases including uveitis, corneal disease, iritis, iridocyclitis, glaucoma, and cataracts.

All diseases and disorders listed herein may be defined as described in the Diagnostic and Statistical Manual of Mental Disorders (DSM-5), published by the American Psychiatric Association, or in International Classification of Diseases (ICD), published by the World Health Organization.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

### Compounds

The inventors have identified novel 2C—X type phenethylamine compounds, including those that demonstrate preferential binding to G-protein coupled receptors (GPCRs), e.g., 5-HT₂ receptors, that are bioavailable (e.g., orally bioavailable), are distributed to the brain, have improved exposure (i.e., prevention of high drug concentrations (spiking) observed acutely after administration), and that possess advantageous enzymatic degradation profiles and clearance. As a result, the disclosed compounds may have reduced side effects and/or toxicity, reduced interpatient variability, fast onset, and may be relatively short acting, thereby enabling practical use in clinical settings. In addition to oral administration routes, these novel compounds may also possess properties, such as desirable lipophilicity, which enable their administration via inhalation or through transdermal routes, e.g., in the form of a transdermal patch. The novel 2C—X compounds are based on specific molecular modifications, e.g., involving deuteration and/or fluorination, to slow or shunt enzymatic degradation at specific sites, and in many cases introducing/maintaining metabolic soft spots at other sites—modifications which have been identified only after significant studies.

### Formula (I)

Disclosed herein is a compound according to Formula (I):

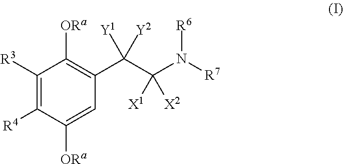

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R³ is hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b};
R⁶ and R⁷ are independently hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl; or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl;
with the proviso that at least one of X¹, X², Y¹, Y², R³, R⁴, R⁶, R⁷, and R^{a} comprises deuterium and/or R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N.
X¹ and X² may be the same, or different. In some embodiments, X¹ and X² are the same. In some embodiments, X¹ and X² are hydrogen. In some embodiments, X¹ and X² are deuterium.
Y¹ and Y² may be the same, or different. In some embodiments, Y¹ and Y² are the same. In some embodiments, Y¹ and Y² are hydrogen. In some embodiments, Y¹ and Y² are deuterium.

In some embodiments, R³ is deuterium. In some embodiments, R³ is hydrogen.

In some embodiments, R⁴ is halogen, for example —Br, —F, —Cl, or —I.

In some embodiments, R⁴ is a substituted or unsubstituted C₁-C₆ alkyl. In some embodiments, R⁴ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. Preferred unsubstituted alkyl groups are methyl and t-butyl. In some embodiments, R⁴ is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R⁴ is a substituted C₁ alkyl group (i.e., a substituted methyl group), examples of which include, but are not limited to, —CDH₂, —CD₂H, —CD₃, —CFH₂, —CF₂H, —CF₃, and —CH₂C≡N. In some embodiments, R⁴ is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R⁴ is not a substituted C₂ alkyl group such as a C₂ fluoroalkyl group. For example, in some embodiments, R⁴ is not —CH₂CFH₂, —CH₂CF₂H, or —CH₂CF₃. In some embodiments, R⁴ is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R⁴ is not —CH₂CH₂CFH₂.

In some embodiments, R⁴ is a substituted or unsubstituted C₃-C₁₀ cycloalkyl. In some embodiments, R⁴ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R⁴ is a substituted C₃-C₁₀ cycloalkyl. The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁴ is a substituted or unsubstituted alkenyl, e.g., a substituted or unsubstituted allyl, butenyl, crotyl, etc.

In some embodiments, R⁴ is a substituted or unsubstituted alkynyl, e.g., a substituted or unsubstituted acetylenyl, propargyl, homopropargyl, etc. In some embodiments, R⁴ is an unsubstituted alkynyl. In some embodiments, R⁴ is an unsubstituted acetylenyl (—C≡CH). In some embodiments, R⁴ is an unsubstituted propargyl (—CH₂C≡CH). In some embodiments, R⁴ is —CH₂CH₂C≡CH. In some embodiments, R⁴ is —CH₂CH₂CH₂C≡CH. In some embodiments, R⁴ is —CH₂CH₂CH₂CH₂C≡CH. In some embodiments, R⁴ is a substituted alkynyl. In some embodiments, R⁴ is a substituted propargyl (e.g., —CF₂C≡CH). In some embodiments, R⁴ is —CF₂CH₂C≡CH. In some embodiments, R⁴ is —CF₂CH₂CH₂C≡CH. In some embodiments, R⁴ is —CF₂CH₂CH₂CH₂C≡CH.

In some embodiments, R⁴ is —OR^{b} or —SR^{b}, wherein R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl. In some embodiments, R⁴ is —OR^{b}. In some embodiments, R⁴ is —SR^{b}. In some embodiments, R^{b} is a substituted or unsubstituted C₁-C₆ alkyl, or a substituted or unsubstituted C₃-C₁₀ cycloalkyl, such as those substituted C₁-C₆ alkyl groups, unsubstituted C₁-C₆ alkyl groups, substituted C₃-C₁₀ cycloalkyl groups, or unsubstituted C₃-C₁₀ cycloalkyl groups defined and exemplified above.

In some embodiments, R^{b} is a substituted or unsubstituted C₁-C₆ alkyl. In some embodiments, R^{b} is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R^{b} is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R^{b} is a substituted C₁ alkyl group, examples of which may include, but are not limited to, —CDH₂, —CD₂H, —CD₃, —CFH₂, —CF₂H, —CF₃, and —CH₂C≡N. In some embodiments, R^{b} is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R^{b} is not a substituted C₂ alkyl group such as a C₂ fluoroalkyl group. For example, in some embodiments when R⁴ is —OR^{b} or —SR^{b}, R^{b} is not —CH₂CFH₂, —CH₂CF₂H, or —CH₂CF₃. In some embodiments, R^{b} is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R^{b} is not —CH₂CH₂CFH₂.

In some embodiments, R^{b} is a substituted or unsubstituted alkenyl, e.g., a substituted or unsubstituted allyl, butenyl, crotyl, etc.

In some embodiments, R^{b} is a substituted or unsubstituted alkynyl, e.g., a substituted or unsubstituted acetylenyl, propargyl, homopropargyl, etc. In some embodiments, R^{b} is an unsubstituted alkynyl. In some embodiments, R^{b} is an unsubstituted acetylenyl (—C≡CH). In some embodiments, R^{b} is an unsubstituted propargyl (—CH₂C≡CH). In some embodiments, R^{b} is —CH₂CH₂C≡CH. In some embodiments, R^{b} is —CH₂CH₂CH₂C≡CH. In some embodiments, R^{b} is —CH₂CH₂CH₂CH₂C≡CH. In some embodiments, R^{b} is a substituted alkynyl. In some embodiments, R^{b} is a substituted propargyl (e.g., —CF₂C≡CH). In some embodiments, R^{b} is —CF₂CH₂C≡CH. In some embodiments, R^{b} is —CF₂CH₂CH₂C≡CH. In some embodiments, R^{b} is —CF₂CH₂CH₂CH₂C≡CH.

In some embodiments, R^{b} is a substituted or unsubstituted cycloalkyl, for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, R^{b} is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R^{b} is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁴ is -A(CF₂)ₘ(CH₂)ₙQ, wherein A is S, O, CH₂, or CF₂, m is 0 to 3, n is 0 to 6, and Q is —C≡CH or —C≡N. In some embodiments, A is S. In some embodiments, A is O. In some embodiments, A is CH₂ (methylene). In some embodiments, A is CF₂ (difluoromethylene). In some embodiments, m is 0. In some embodiments, m is 1. In some embodiments, m is 2. In some embodiments, m is 3. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, when A is S or O, the sum of m+n is no more than 6, or no more than 5, or no more than 4, or no more than 3, or no more than 2, or no more than 1, or 0. In some embodiments, when A is CH₂ or CF₂, the sum of m+n is no more than 5, or no more than 4, or no more than 3, or no more than 2, or no more than 1, or 0. In some embodiments, Q is —C≡CH. In some embodiments, Q is —C≡N. Representative examples of the group -A(CF₂)ₘ(CH₂)ₙQ include, but are not limited to, —SC≡CH, —SCH₂C≡CH, —SCF₂C≡CH, —SCH₂C≡N, —SCH₂CH₂C≡CH, —SCF₂CH₂C≡CH, —SCH₂CH₂CH₂C≡CH, —SCF₂CH₂CH₂C≡CH, —OCH₂C≡CH, —OC≡CH, —CH₂C≡CH, —CF₂C≡CH, —CH₂CH₂C≡CH, —CF₂CH₂C≡CH, —CH₂CH₂CH₂C≡CH, —CF₂CH₂CH₂C≡CH, —CH₂CH₂CH₂CH₂C≡CH, and —CF₂CH₂CH₂CH₂C≡CH.

In some embodiments, R⁴ is selected from the group consisting of —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br. In some embodiments, R⁴ is selected from the group consisting of —SMe, —SCF₃, —SCF₂H, -Me, —OCD₃, —CF₃, -t-Bu, or -cyclopentyl. In some embodiments, R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl. When R⁴ is —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), or -A(CF₂)ₘ(CH₂)ₙQ, wherein A is S, O, CH₂, or CF₂, m is 0 to 3, n is 0 to 6, and Q is —C≡CH or —C≡N, the other substituents (i.e., X¹, X², Y¹, Y², R³, R⁶, R⁷, and R^{a}) may, or may not, comprise deuterium. In some embodiments, R⁴ is selected from the group consisting of —SCF₂H, —SCFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl (—C₅H₉). When R⁴ is —SCF₂H, —SCFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CF₂CF₂H, —OCH₂C≡CH, —OC≡CH, —CH₂CH₂CH₂CH₂C≡CH, —CF₂CH₂CH₂CH₂C≡CH, or -cyclopentyl (—C₅H₉), the other substituents (i.e., X¹, X², Y¹, Y², R³, R⁶, R⁷, and R^{a}) may, or may not, comprise deuterium.

R⁶ and R⁷ may be the same, or different. In some embodiments, R⁶ and R⁷ are the same. For example, in some embodiments, both R⁶ and R⁷ are hydrogen. In some embodiments, R⁶ and R⁷ are different. For example, in some embodiments, R⁶ is hydrogen, and R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl.

In some embodiments, R⁶ and/or R⁷ is an unsubstituted C₁-C₆ alkyl, for example, an unsubstituted C₁ alkyl, an unsubstituted C₂ alkyl, an unsubstituted C₃ alkyl, an unsubstituted C₄ alkyl, an unsubstituted C₅ alkyl, or an unsubstituted C₆ alkyl. In some embodiments, R⁶ and/or R⁷ is an unsubstituted linear C₂-C₆ alkyl. In some embodiments, R⁶ and/or R⁷ is an unsubstituted branched C₃-C₁₀ alkyl. Examples of an unsubstituted C₁-C₆ alkyl include, but are not limited to, methyl, ethyl, n-propyl, isopropyl, butyl, isobutyl, t-butyl, n-pentyl, isopentyl, n-hexyl, and isohexyl.

In some embodiments, R⁶ and/or R⁷ is a substituted C₁-C₆ alkyl, e.g., a substituted C₁ alkyl, a substituted C₂ alkyl, a substituted C₃ alkyl, a substituted C₄ alkyl, a substituted C₅ alkyl, or a substituted C₆ alkyl. The alkyl group may contain one, or more than one, substituent. The alkyl group may be substituted with any one or more substituents recited herein, examples of those substituents may include, but are not limited to, deuterium, halogen (e.g., fluorine), polar substituents such as hydroxyl, oxo, unsubstituted alkoxy, substituted alkoxy (e.g., polyether groups), unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. In some embodiments, R⁶ and/or R⁷ is a C₁-C₆ alkyl substituted with one or more deuterium atoms, examples of which include, but are not limited to, —CDH₂, —CD₂H, —CD₃, —CD₂CD₃, and —CD₂CD₂CD₃. In some embodiments, R⁶ and/or R⁷ is a C₁-C₆ alkyl substituted with one or more fluorine atoms, i.e., is a fluoroalkyl group. Examples of fluoroalkyl groups include, but are not limited to, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, —CH₂CH₂CH₂CF₃, —CH₂CF₂CHF₂, —CH₂CF₂CF₃, —CH(CF₃)₂, and —CH(CH₃)CF₃.

In some embodiments, R⁶ and/or R⁷ is a C₁-C₆ alkyl substituted with one or more deuterium atoms and one or more fluorine atoms, examples of which include, but are not limited to, —CD₂CH₂F, —CD₂CHF₂, —CD₂CF₃, —CD₂CH₂CH₂F, —CD₂CH₂CHF₂, —CD₂CH₂CF₃, —CD₂CD₂CH₂, —CD₂CD₂CHF₂, —CD₂CD₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃.

In some embodiments, R⁶ and/or R⁷ is a C₁-C₆ alkyl substituted with a substituted or unsubstituted cycloalkyl. The C₁-C₆ alkyl may be substituted with, e.g., a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, the C₁-C₆ alkyl is substituted with an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, the C₁-C₆ alkyl is substituted with a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent. In some embodiments, R⁶ and/or R⁷ is a C₁ alkyl substituted with a substituted or unsubstituted cycloalkyl, with particular mention being made to cyclopropylmethyl (—CH₂C₃H₅).

In some embodiments, R⁶ and/or R⁷ is a substituted or unsubstituted alkenyl, e.g., a substituted or unsubstituted allyl, butenyl, crotyl, etc.

In some embodiments, R⁶ and/or R⁷ is a substituted or unsubstituted alkynyl, e.g., a substituted or unsubstituted propargyl.

In some embodiments, R⁶ and/or R⁷ is a substituted or unsubstituted cycloalkyl, for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, R⁶ and/or R⁷ is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R⁶ and/or R⁷ is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁶ and/or R⁷ is a substituted or unsubstituted heterocycloalkyl. In some embodiments, the unsubstituted or substituted heterocycloalkyl group may be a 3-membered ring, a 4-membered ring, a 5-membered ring, a 6-membered ring, a 7-membered ring, or an 8-membered ring. In some embodiments, R⁶ and/or R⁷ is an unsubstituted heterocycloalkyl, such as those set forth herein, examples of which include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, tetrahydrofuran, tetrahydropyran, and 1,3-dioxolane. In some embodiments, R⁶ and/or R⁷ is a substituted heterocycloalkyl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), oxo, and hydroxyl. The heterocycloalkyl group may contain one, or more than one, substituent.

In some embodiments, R⁶ and/or R⁷ is a substituted or unsubstituted aryl. In some embodiments, R⁶ and/or R⁷ is an unsubstituted aryl, examples of which include, but are not limited to, phenyl and naphthyl. In some embodiments, R⁶ and/or R⁷ is a substituted aryl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), hydroxyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The aryl group may contain one, or more than one, substituent.

In some embodiments, R⁶ and/or R⁷ is a substituted or unsubstituted heteroaryl. In some embodiments, R⁶ and/or R⁷ is an unsubstituted heteroaryl, examples of which include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothiophenyl, thiophenyl, benzimidazolyl, quinolinyl, isoquinolinyl, quinazolinyl, quinoxalinyl, and pyrazolyl. In some embodiments, R⁶ and/or R⁷ is a substituted heteroaryl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), hydroxyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The heteroaryl group may contain one, or more than one, substituent.

In some embodiments, R⁶ is hydrogen, and R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl. In some embodiments, R⁶ is hydrogen, and R⁷ is a substituted or unsubstituted C₁-C₆ alkyl. In some embodiments, R⁶ is hydrogen, and R⁷ is an unsubstituted C₁-C₆ alkyl, a C₁-C₆ alkyl substituted with one or more deuterium atoms, a C₁-C₆ alkyl substituted with one or more fluorine atoms, or a C₁-C₆ alkyl substituted with a substituted or unsubstituted cycloalkyl. For example, in some embodiments, R⁶ is hydrogen, and R⁷ is methyl, ethyl, propyl, —CD₃, or cyclopropylmethyl (—CH₂C₃H₅).

In some embodiments, R⁶ and R⁷ together with the nitrogen atom attached thereto are joined to form a substituted or unsubstituted heterocycloalkyl. In some embodiments, R⁶ and R⁷ together with the nitrogen atom attached thereto are joined to form an unsubstituted heterocycloalkyl. The unsubstituted heterocycloalkyl group may be, e.g., a 3-membered ring, a 4-membered ring, 5-membered ring, a 6-membered ring, a 7-membered ring, an 8-membered ring, etc., which may be optionally fused to other ring(s). The unsubstituted heterocycloalkyl group contains a minimum of one nitrogen ring atom (the nitrogen atom intervening R⁶ and R⁷), and may optionally contain at least one additional hetero-ring atom, which may be one or more of nitrogen, sulfur, or oxygen, for a total of 1, 2, 3, or 4 hetero-ring atoms (at least one of which is a nitrogen ring atom). Examples of unsubstituted heterocycloalkyl groups formed from joining R⁶ and R⁷ together with the nitrogen atom attached thereto include, but are not limited to,

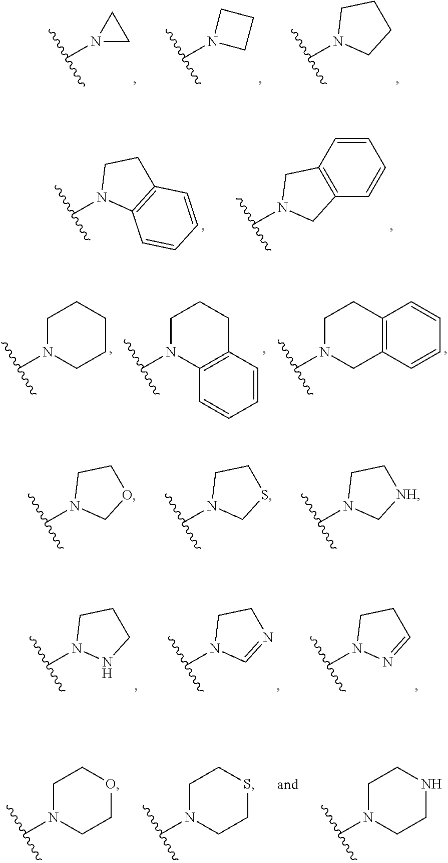

In some embodiments, R⁶ and R⁷ together with the nitrogen atom attached thereto are joined to form a substituted heterocycloalkyl. The substituted heterocycloalkyl group may be, e.g., a 3-membered ring, a 4-membered ring, a 5-membered ring, a 6-membered ring, a 7-membered ring, an 8-membered ring, etc., which may be optionally fused to other ring(s). The substituted heterocycloalkyl group contains a minimum of one nitrogen ring atom (the nitrogen atom intervening R⁶ and R⁷), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the substituted heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, 1,2,3,4-tetrahydroisoquinoline, thiazolidine, morpholine, or thiomorpholine, which is substituted with at least one substituent. The substituent(s) may be any recited herein, including, but not limited to, deuterium, halogen (e.g., fluorine), polar substituents such as hydroxyl, oxo, unsubstituted alkoxy, substituted alkoxy (e.g., polyether groups), unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The substituted heterocycloalkyl formed from joining R⁶ and R⁷ together with the nitrogen atom attached thereto contains a heterocycloalkyl group substituted with one, two, three, four, or more substituents. The substituent may be located on a carbon ring atom or on a hetero-ring atom.

Examples of substituted heterocycloalkyl groups formed from joining R⁶ and R⁷ together with the nitrogen atom attached thereto include, but are not limited to,

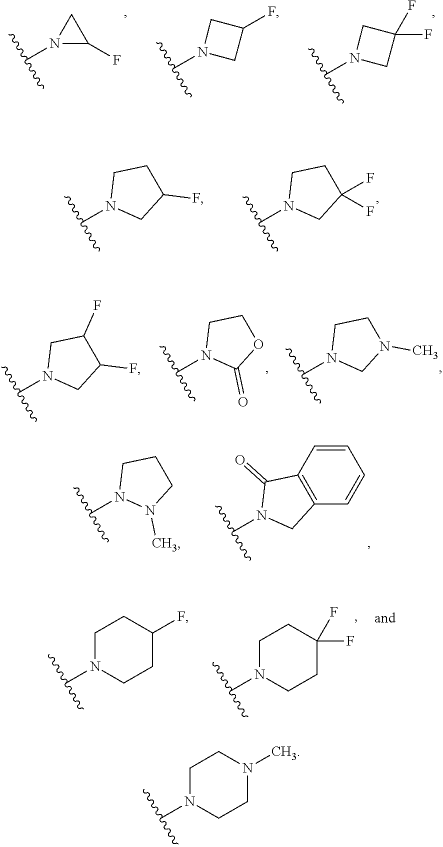

Each R^{a} may be the same, or different. In some embodiments, each R^{a} is the same. Each R^{a} may be, independently, a substituted or unsubstituted C₁-C₆ alkyl, preferably a substituted or unsubstituted C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, each R^{a} is different, e.g., one R^{a} is —CH₃, while another is —CD₃.

In some embodiments, Y¹ and Y² are each hydrogen or each deuterium; R³ is hydrogen; X¹ and X² are each hydrogen or each deuterium; each R^{a} is —CH₃ or —CD₃; R⁴ is —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br, preferably R⁴ is —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl; R⁶ is hydrogen; and R⁷ is hydrogen or a substituted or unsubstituted C₁-C₆ alkyl.

As stated above, any of the above embodiments of the compound of Formula (I) may be provided as long as (i) at least one of X¹, X², Y¹, Y², R³, R⁴, R⁶, R⁷, and R^{a} comprises deuterium and/or (ii) R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N. In some embodiments, in condition (ii), R⁴ is selected from the group consisting of —SCF₂H, —SCFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl (—C₅H₉). The above proviso written with “and/or” means that compounds fall within the scope when one of conditions (i) or (ii) is satisfied, or when both conditions (i) and (ii) are satisfied. For clarity, compounds in which condition (i) is satisfied, i.e., where at least one of X¹, X², Y¹, Y², R³, R⁴, R⁶, R⁷, and R^{a} comprises deuterium, do not require condition (ii) to be satisfied, e.g., R⁴ may or may not be selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N. Likewise compounds in which condition (ii) is satisfied, e.g., when R⁴ is —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), or -A(CF₂)ₘ(CH₂)ₙQ, wherein A is S, O, CH₂, or CF₂, m is 0 to 3, n is 0 to 6, and Q is —C≡CH or —C≡N, do not require condition (i) to be satisfied, i.e., the other substituents X¹, X², Y¹, Y², R³, R⁶, R⁷, and R^{a} may, or may not, comprise deuterium.

### Formula (II)

In some embodiments, the compound has a structure of Formula (II)

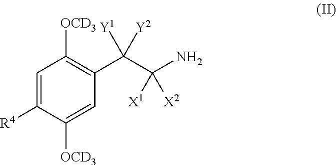

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b}; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl.
X¹ and X² may be the same, or different. In some embodiments, X¹ and X² are the same. In some embodiments, X¹ and X² are hydrogen. In some embodiments, X¹ and X² are deuterium.
Y¹ and Y² may be the same, or different. In some embodiments, Y¹ and Y² are the same. In some embodiments, Y¹ and Y² are hydrogen. In some embodiments, Y¹ and Y² are deuterium.

In some embodiments, R⁴ is halogen, for example —Br, —F, —Cl, or —I.

In some embodiments, R⁴ is a substituted or unsubstituted C₁-C₆ alkyl. In some embodiments, R⁴ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. Preferred unsubstituted alkyl groups are methyl and t-butyl. In some embodiments, R⁴ is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R⁴ is a substituted C₁ alkyl group (i.e., a substituted methyl group), examples of which include, but are not limited to, —CDH₂, —CD₂H, —CD₃, —CFH₂, —CF₂H, —CF₃, and —CH₂C≡N. In some embodiments, R⁴ is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R⁴ is not a substituted C₂ alkyl group such as a C₂ fluoroalkyl group. For example, in some embodiments, R⁴ is not —CH₂CFH₂, —CH₂CF₂H, or —CH₂CF₃. In some embodiments, R⁴ is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R⁴ is not —CH₂CH₂CFH₂.

In some embodiments, R⁴ is a substituted or unsubstituted C₃-C₁₀ cycloalkyl. In some embodiments, R⁴ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R⁴ is a substituted C₃-C₁₀ cycloalkyl. The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁴ is a substituted or unsubstituted alkenyl, e.g., a substituted or unsubstituted allyl, butenyl, crotyl, etc.

In some embodiments, R⁴ is a substituted or unsubstituted alkynyl, e.g., a substituted or unsubstituted acetylenyl, propargyl, homopropargyl, etc. In some embodiments, R⁴ is an unsubstituted alkynyl. In some embodiments, R⁴ is an unsubstituted acetylenyl (—C≡CH). In some embodiments, R⁴ is an unsubstituted propargyl (—CH₂C≡CH). In some embodiments, R⁴ is —CH₂CH₂C≡CH. In some embodiments, R⁴ is —CH₂CH₂CH₂C≡CH. In some embodiments, R⁴ is —CH₂CH₂CH₂CH₂C≡CH. In some embodiments, R⁴ is a substituted alkynyl. In some embodiments, R⁴ is a substituted propargyl (e.g., —CF₂C≡CH). In some embodiments, R⁴ is —CF₂CH₂C≡CH. In some embodiments, R⁴ is —CF₂CH₂CH₂C≡CH. In some embodiments, R⁴ is —CF₂CH₂CH₂CH₂C≡CH.

In some embodiments, R⁴ is —OR^{b} or —SR^{b}, wherein R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl. In some embodiments, R⁴ is —OR^{b}. In some embodiments, R⁴ is —SR^{b}. In some embodiments, R^{b} is a substituted or unsubstituted C₁-C₆ alkyl, or a substituted or unsubstituted C₃-C₁₀ cycloalkyl, such as those substituted C₁-C₆ alkyl groups, unsubstituted C₁-C₆ alkyl groups, substituted C₃-C₁₀ cycloalkyl groups, or unsubstituted C₃-C₁₀ cycloalkyl groups defined and exemplified above.

In some embodiments, R^{b} is a substituted or unsubstituted C₁-C₆ alkyl. In some embodiments, R^{b} is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R^{b} is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R^{b} is a substituted C₁ alkyl group, examples of which may include, but are not limited to, —CDH₂, —CD₂H, —CD₃, —CFH₂, —CF₂H, —CF₃, and —CH₂C≡N. In some embodiments, R^{b} is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R^{b} is not a substituted C₂ alkyl group such as a C₂ fluoroalkyl group. For example, in some embodiments when R⁴ is —OR^{b} or —SR^{b}, R^{b} is not —CH₂CFH₂, —CH₂CF₂H, or —CH₂CF₃. In some embodiments, R^{b} is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R^{b} is not —CH₂CH₂CFH₂.

In some embodiments, R^{b} is a substituted or unsubstituted alkenyl, e.g., a substituted or unsubstituted allyl, butenyl, crotyl, etc.

In some embodiments, R^{b} is a substituted or unsubstituted alkynyl, e.g., a substituted or unsubstituted acetylenyl, propargyl, homopropargyl, etc. In some embodiments, R^{b} is an unsubstituted alkynyl. In some embodiments, R^{b} is an unsubstituted acetylenyl (—C≡CH). In some embodiments, R^{b} is an unsubstituted propargyl (—CH₂C≡CH). In some embodiments, R^{b} is —CH₂CH₂C≡CH. In some embodiments, R^{b} is —CH₂CH₂CH₂C≡CH. In some embodiments, R^{b} is —CH₂CH₂CH₂CH₂C≡CH. In some embodiments, R^{b} is a substituted alkynyl. In some embodiments, R^{b} is a substituted propargyl (e.g., —CF₂C≡CH). In some embodiments, R^{b} is —CF₂CH₂C≡CH. In some embodiments, R^{b} is —CF₂CH₂CH₂C≡CH. In some embodiments, R^{b} is —CF₂CH₂CH₂CH₂C≡CH.

In some embodiments, R^{b} is a substituted or unsubstituted cycloalkyl, for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, R^{b} is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R^{b} is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁴ is -A(CF₂)ₘ(CH₂)ₙQ, wherein A is S, O, CH₂, or CF₂, m is 0 to 3, n is 0 to 6, and Q is —C≡CH or —C≡N. In some embodiments, A is S. In some embodiments, A is O. In some embodiments, A is CH₂ (methylene). In some embodiments, A is CF₂ (difluoromethylene). In some embodiments, m is 0. In some embodiments, m is 1. In some embodiments, m is 2. In some embodiments, m is 3. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, when A is S or O, the sum of m+n is no more than 6, or no more than 5, or no more than 4, or no more than 3, or no more than 2, or no more than 1, or 0. In some embodiments, when A is CH₂ or CF₂, the sum of m+n is no more than 5, or no more than 4, or no more than 3, or no more than 2, or no more than 1, or 0. In some embodiments, Q is —C≡CH. In some embodiments, Q is —C≡N. Representative examples of the group -A(CF₂)ₘ(CH₂)ₙQ include, but are not limited to, —SC≡CH, —SCH₂C≡CH, —SCF₂C≡CH, —SCH₂C≡N, —SCH₂CH₂C≡CH, —SCF₂CH₂C≡CH, —SCH₂CH₂CH₂C≡CH, —SCF₂CH₂CH₂C≡CH, —OCH₂C≡CH, —OC≡CH, —CH₂C≡CH, —CF₂C≡CH, —CH₂CH₂C≡CH, —CF₂CH₂C≡CH, —CH₂CH₂CH₂C≡CH, —CF₂CH₂CH₂C≡CH, —CH₂CH₂CH₂CH₂C≡CH, and —CF₂CH₂CH₂CH₂C≡CH.

In some embodiments, R⁴ is selected from the group consisting of —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br. In some embodiments, R⁴ is selected from the group consisting of —SMe, —SCF₃, —SCF₂H, -Me, —OCD₃, —CF₃, -t-Bu, or -cyclopentyl. In some embodiments, R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl. In some embodiments, R⁴ is selected from the group consisting of —SCF₂H, —SCFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl (—C₅H₉).

In some embodiments, Y¹ and Y² are each hydrogen or each deuterium, X¹ and X² are each hydrogen or each deuterium, and R⁴ is —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br, preferably R⁴ is —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, and -cyclopentyl.

The compounds of Formula (II) containing deuteration in the form of —OCD₃ groups at the 2- and 5-position of the phenyl ring can have beneficial effects by slowing or shunting O-demethylation (primarily mediated by CYP2D6 enzymes) at these positions, thereby improving the pharmacokinetics, specifically the bioavailability, and safety as a result of lower exposure to potentially toxic metabolites.

### Formula (III)

In some embodiments, the compound has a structure of Formula (III):

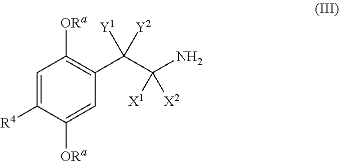

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁴ is a C₁-C₆ alkyl substituted with one or more deuterium, a C₃-C₁₀ cycloalkyl substituted with one or more deuterium, —OR^{b}, or —SR^{b};
each R^{a} is independently a substituted or unsubstituted C₁-C₆ alkyl; and
R^{b} is a C₁-C₆ alkyl substituted with one or more deuterium or a C₃-C₁₀ cycloalkyl substituted with one or more deuterium.
X¹ and X² may be the same, or different. In some embodiments, X¹ and X² are the same. In some embodiments, X¹ and X² are hydrogen. In some embodiments, X¹ and X² are deuterium.
Y¹ and Y² may be the same, or different. In some embodiments, Y¹ and Y² are the same. In some embodiments, Y¹ and Y² are hydrogen. In some embodiments, Y¹ and Y² are deuterium.

In some embodiments, R⁴ is a C₁-C₆ alkyl substituted with one or more deuterium, examples of which include, but are not limited to, methyl, ethyl, n-propyl, isopropyl, n-butyl, isobutyl, sec-butyl, t-butyl, n-pentyl, neopentyl, and hexyl groups containing one or more deuterium substitutions. The alkyl group may contain one, or more than one, deuterium substituent, such as 1, 2, 3, 4, 5, 6, 7, 8, or 9 deuterium substituents. Exemplary C₁-C₆ alkyl groups substituted with one or more deuterium include, but are not limited to, —CD₃ and —C(CD₃)₃.

In some embodiments, R⁴ is a C₃-C₁₀ cycloalkyl substituted with one or more deuterium, for example, adamantyl, cyclopropyl, cyclobutyl, cyclopentyl, cyclohexyl, or cyclooctyl groups substituted with one or more deuterium. The cycloalkyl group may contain one, or more than one, substituent, such as 1, 2, 3, 4, 5, 6, 7, 8, or 9 deuterium substituents.

In some embodiments, R⁴ is —OR^{b}, wherein R^{b} is a C₁-C₆ alkyl substituted with one or more deuterium, or a C₃-C₁₀ cycloalkyl substituted with one or more deuterium, such as those C₁-C₆ alkyl groups substituted with one or more deuterium or C₃-C₁₀ cycloalkyl groups substituted with one or more deuterium defined and exemplified above. In some embodiments, R^{b} is a C₁ alkyl group substituted with one or more deuterium, for example, —CDH₂, —CD₂H, and —CD₃. In some embodiments, R^{b} is a C₂ alkyl group substituted with one or more deuterium, examples of which may include, but are not limited to, —CDHCDH₂, —CDHCD₂H, —CD₂CD₃. In some embodiments, R^{b} is a C₃ alkyl group substituted with one or more deuterium. In some embodiments, R⁴ is —OCD₃.

In some embodiments, R⁴ is —SR^{b}, wherein R^{b} is a C₁-C₆ alkyl substituted with one or more deuterium, or a C₃-C₁₀ cycloalkyl substituted with one or more deuterium, such as those C₁-C₆ alkyl groups substituted with one or more deuterium or C₃-C₁₀ cycloalkyl groups substituted with one or more deuterium defined and exemplified above. In some embodiments, R^{b} is a C₁ alkyl group substituted with one or more deuterium, for example, —CDH₂, —CD₂H, and —CD₃. In some embodiments, R^{b} is a C₂ alkyl group substituted with one or more deuterium, examples of which may include, but are not limited to, —CDHCDH₂, —CDHCD₂H, —CD₂CD₃. In some embodiments, R^{b} is a C₃ alkyl group substituted with one or more deuterium. In some embodiments, R⁴ is —SCD₃.

In some embodiments, R⁴ is selected from the group consisting of R⁴ is —SCD₃, —CD₃, —C(CD₃)₃, and —OCD₃.

Each R^{a} may be the same, or different. In some embodiments, each R^{a} is the same. Each R^{a} may be, independently, a substituted or unsubstituted C₁-C₆ alkyl, preferably a substituted or unsubstituted C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, each R^{a} is different, e.g., one R^{a} is —CH₃, while another is —CD₃.

In some embodiments, X¹ and X² are each hydrogen or each deuterium, Y¹ and Y² are each hydrogen or each deuterium, each R^{a} is —CH₃, and R⁴ is —SCD₃, —CD₃, —C(CD₃)₃, or —OCD₃.

The compounds of Formula (III) containing deuterated substituents (e.g., deuterated alkyl/cycloalkyl groups) in the R⁴ position of the phenyl ring can have beneficial effects by allowing for the incorporation of lipophilic groups for improved brain penetrability, while at the same time slowing or shunting metabolism at this position, for improved pharmacokinetics, specifically bioavailability.

### Formula (IV)

In some embodiments, the compound has a structure of Formula (IV):

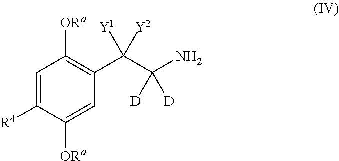

or a pharmaceutically acceptable salt, solvate, stereoisomer, or prodrug thereof,
wherein:
Y¹ and Y² are independently hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b};
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl.
Y¹ and Y² may be the same, or different. In some embodiments, Y¹ and Y² are the same. In some embodiments, Y¹ and Y² are hydrogen. In some embodiments, Y¹ and Y² are deuterium.

In some embodiments, R⁴ is halogen, for example —Br, —F, —Cl, or —I.

In some embodiments, R⁴ is a substituted or unsubstituted C₁-C₆ alkyl. In some embodiments, R⁴ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. Preferred unsubstituted alkyl groups are methyl and t-butyl. In some embodiments, R⁴ is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R⁴ is a substituted C₁ alkyl group (i.e., a substituted methyl group), examples of which include, but are not limited to, —CDH₂, —CD₂H, —CD₃, —CFH₂, —CF₂H, —CF₃, and —CH₂C≡N. In some embodiments, R⁴ is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R⁴ is not a substituted C₂ alkyl group such as a C₂ fluoroalkyl group. For example, in some embodiments, R⁴ is not —CH₂CFH₂, —CH₂CF₂H, or —CH₂CF₃. In some embodiments, R⁴ is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R⁴ is not —CH₂CH₂CFH₂.

In some embodiments, R⁴ is a substituted or unsubstituted C₃-C₁₀ cycloalkyl. In some embodiments, R⁴ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R⁴ is a substituted C₃-C₁₀ cycloalkyl. The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁴ is a substituted or unsubstituted alkenyl, e.g., a substituted or unsubstituted allyl, butenyl, crotyl, etc.

In some embodiments, R⁴ is a substituted or unsubstituted alkynyl, e.g., a substituted or unsubstituted acetylenyl, propargyl, homopropargyl, etc. In some embodiments, R⁴ is an unsubstituted alkynyl. In some embodiments, R⁴ is an unsubstituted acetylenyl (—C≡CH). In some embodiments, R⁴ is an unsubstituted propargyl (—CH₂C≡CH). In some embodiments, R⁴ is —CH₂CH₂C≡CH. In some embodiments, R⁴ is —CH₂CH₂CH₂C≡CH. In some embodiments, R⁴ is —CH₂CH₂CH₂CH₂C≡CH. In some embodiments, R⁴ is a substituted alkynyl. In some embodiments, R⁴ is a substituted propargyl (e.g., —CF₂C≡CH). In some embodiments, R⁴ is —CF₂CH₂C≡CH. In some embodiments, R⁴ is —CF₂CH₂CH₂C≡CH. In some embodiments, R⁴ is —CF₂CH₂CH₂CH₂C≡CH.

In some embodiments, R⁴ is —OR^{b} or —SR^{b}, wherein R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl. In some embodiments, R⁴ is —OR^{b}. In some embodiments, R⁴ is —SR^{b}. In some embodiments, R^{b} is a substituted or unsubstituted C₁-C₆ alkyl, or a substituted or unsubstituted C₃-C₁₀ cycloalkyl, such as those substituted C₁-C₆ alkyl groups, unsubstituted C₁-C₆ alkyl groups, substituted C₃-C₁₀ cycloalkyl groups, or unsubstituted C₃-C₁₀ cycloalkyl groups defined and exemplified above.

In some embodiments, R^{b} is a substituted or unsubstituted C₁-C₆ alkyl. In some embodiments, R^{b} is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R^{b} is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R^{b} is a substituted C₁ alkyl group, examples of which may include, but are not limited to, —CDH₂, —CD₂H, —CD₃, —CFH₂, —CF₂H, —CF₃, and —CH₂C≡N. In some embodiments, R^{b} is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R^{b} is not a substituted C₂ alkyl group such as a C₂ fluoroalkyl group. For example, in some embodiments when R⁴ is —OR^{b} or —SR^{b}, R^{b} is not —CH₂CFH₂, —CH₂CF₂H, or —CH₂CF₃. In some embodiments, R^{b} is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R^{b} is not —CH₂CH₂CFH₂.

In some embodiments, R^{b} is a substituted or unsubstituted alkenyl, e.g., a substituted or unsubstituted allyl, butenyl, crotyl, etc.

In some embodiments, R^{b} is a substituted or unsubstituted alkynyl, e.g., a substituted or unsubstituted acetylenyl, propargyl, homopropargyl, etc. In some embodiments, R^{b} is an unsubstituted alkynyl. In some embodiments, R^{b} is an unsubstituted acetylenyl (—C≡CH). In some embodiments, R^{b} is an unsubstituted propargyl (—CH₂C≡CH). In some embodiments, R^{b} is —CH₂CH₂C≡CH. In some embodiments, R^{b} is —CH₂CH₂CH₂C≡CH. In some embodiments, R^{b} is —CH₂CH₂CH₂CH₂C≡CH. In some embodiments, R^{b} is a substituted alkynyl. In some embodiments, R^{b} is a substituted propargyl (e.g., —CF₂C≡CH). In some embodiments, R^{b} is —CF₂CH₂C≡CH. In some embodiments, R^{b} is —CF₂CH₂CH₂C≡CH. In some embodiments, R^{b} is —CF₂CH₂CH₂CH₂C≡CH.

In some embodiments, R^{b} is a substituted or unsubstituted cycloalkyl, for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, R^{b} is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R^{b} is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁴ is -A(CF₂)ₘ(CH₂)ₙQ, wherein A is S, O, CH₂, or CF₂, m is 0 to 3, n is 0 to 6, and Q is —C≡CH or —C≡N. In some embodiments, A is S. In some embodiments, A is O. In some embodiments, A is CH₂ (methylene). In some embodiments, A is CF₂ (difluoromethylene). In some embodiments, m is 0. In some embodiments, m is 1. In some embodiments, m is 2. In some embodiments, m is 3. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, when A is S or O, the sum of m+n is no more than 6, or no more than 5, or no more than 4, or no more than 3, or no more than 2, or no more than 1, or 0. In some embodiments, when A is CH₂ or CF₂, the sum of m+n is no more than 5, or no more than 4, or no more than 3, or no more than 2, or no more than 1, or 0. In some embodiments, Q is —C≡CH. In some embodiments, Q is —C≡N. Representative examples of the group -A(CF₂)ₘ(CH₂)ₙQ include, but are not limited to, —SC≡CH, —SCH₂C≡CH, —SCF₂C≡CH, —SCH₂C≡N, —SCH₂CH₂C≡CH, —SCF₂CH₂C≡CH, —SCH₂CH₂CH₂C≡CH, —SCF₂CH₂CH₂C≡CH, —OCH₂C≡CH, —OC≡CH, —CH₂C≡CH, —CF₂C≡CH, —CH₂CH₂C≡CH, —CF₂CH₂C≡CH, —CH₂CH₂CH₂C≡CH, —CF₂CH₂CH₂C≡CH, —CH₂CH₂CH₂CH₂C≡CH, and —CF₂CH₂CH₂CH₂C≡CH.

In some embodiments, R⁴ is selected from the group consisting of —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br. In some embodiments, R⁴ is selected from the group consisting of —SMe, —SCF₃, —SCF₂H, -Me, —OCD₃, —CF₃, -t-Bu, or -cyclopentyl. In some embodiments, R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl. In some embodiments, R⁴ is selected from the group consisting of —SCF₂H, —SCFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl (—C₅H₉).

Each R^{a} may be the same, or different. In some embodiments, each R^{a} is the same. Each R^{a} may be, independently, a substituted or unsubstituted C₁-C₆ alkyl, preferably a substituted or unsubstituted C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, each R^{a} is different, e.g., one R^{a} is —CH₃, while another is —CD₃.

In some embodiments, Y¹ and Y² are each hydrogen or each deuterium, each R^{a} is —CH₃ or —CD₃; and R⁴ is —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br, preferably R⁴ is —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, and -cyclopentyl.

The compounds of Formula (IV) containing deuteration in the ethylene fragment that links the amino group with the benzene ring in phenethylamines, e.g., α-carbon deuteration, may advantageously slow enzymatic degradation compared to compounds which may otherwise be susceptible to MAO mediated deamination/oxidation processes, thereby improving bioavailability as well as enhancing brain levels of the active compound, with the objective to effectively reduce therapeutic doses and to prevent high drug concentrations (“spiking”) observed acutely after administration. As a result, such compounds may result in reduced side effects and toxicity, such as acute adverse effects, including anxiety, fear, tachycardia, hypertension, increased body temperature, nausea and vomiting, as well as toxicity caused by activation of 5-HT_{2B} receptors associated with valvular heart disease.

### Formula (V)

In some embodiments, the compound has a structure of Formula (V):

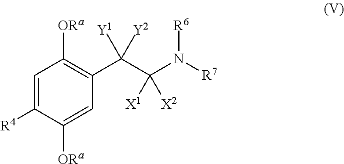

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b};
R⁶ is hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl;

In some embodiments, at least one of X¹, X², Y¹, Y², R³, R⁴, R⁶, R⁷, and R^{a} comprises deuterium and/or R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N.

X¹ and X² may be the same, or different. In some embodiments, X¹ and X² are the same. In some embodiments, X¹ and X² are hydrogen. In some embodiments, X¹ and X² are deuterium.

Y¹ and Y² may be the same, or different. In some embodiments, Y¹ and Y² are the same. In some embodiments, Y¹ and Y² are hydrogen. In some embodiments, Y¹ and Y² are deuterium.

In some embodiments, R⁴ is halogen, for example —Br, —F, —Cl, or —I.

In some embodiments, R⁴ is a substituted or unsubstituted C₁-C₆ alkyl. In some embodiments, R⁴ is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. Preferred unsubstituted alkyl groups are methyl and t-butyl. In some embodiments, R⁴ is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R⁴ is a substituted C₁ alkyl group (i.e., a substituted methyl group), examples of which include, but are not limited to, —CDH₂, —CD₂H, —CD₃, —CFH₂, —CF₂H, —CF₃, and —CH₂C≡N. In some embodiments, R⁴ is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R⁴ is not a substituted C₂ alkyl group such as a C₂ fluoroalkyl group. For example, in some embodiments, R⁴ is not —CH₂CFH₂, —CH₂CF₂H, or —CH₂CF₃. In some embodiments, R⁴ is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R⁴ is not —CH₂CH₂CFH₂.

In some embodiments, R⁴ is a substituted or unsubstituted C₃-C₁₀ cycloalkyl. In some embodiments, R⁴ is an unsubstituted C₃-C₁₀ cycloalkyl, examples of which may include, but are not limited to, adamantyl, cyclopropyl, cyclobutyl, cyclopentyl, cyclohexyl, and cyclooctyl. In some embodiments, R⁴ is a substituted C₃-C₁₀ cycloalkyl. The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁴ is a substituted or unsubstituted alkenyl, e.g., a substituted or unsubstituted allyl, butenyl, crotyl, etc.

In some embodiments, R⁴ is a substituted or unsubstituted alkynyl, e.g., a substituted or unsubstituted acetylenyl, propargyl, homopropargyl, etc. In some embodiments, R⁴ is an unsubstituted alkynyl. In some embodiments, R⁴ is an unsubstituted acetylenyl (—C≡CH). In some embodiments, R⁴ is an unsubstituted propargyl (—CH₂C≡CH). In some embodiments, R⁴ is —CH₂CH₂C≡CH. In some embodiments, R⁴ is —CH₂CH₂CH₂C≡CH. In some embodiments, R⁴ is —CH₂CH₂CH₂CH₂C≡CH. In some embodiments, R⁴ is a substituted alkynyl. In some embodiments, R⁴ is a substituted propargyl (e.g., —CF₂C≡CH). In some embodiments, R⁴ is —CF₂CH₂C≡CH. In some embodiments, R⁴ is —CF₂CH₂CH₂C≡CH. In some embodiments, R⁴ is —CF₂CH₂CH₂CH₂C≡CH.

In some embodiments, R⁴ is —OR^{b} or —SR^{b}, wherein R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl. In some embodiments, R⁴ is —OR^{b}. In some embodiments, R⁴ is —SR^{b}. In some embodiments, R^{b} is a substituted or unsubstituted C₁-C₆ alkyl, or a substituted or unsubstituted C₃-C₁₀ cycloalkyl, such as those substituted C₁-C₆ alkyl groups, unsubstituted C₁-C₆ alkyl groups, substituted C₃-C₁₀ cycloalkyl groups, or unsubstituted C₃-C₁₀ cycloalkyl groups defined and exemplified above.

In some embodiments, R^{b} is a substituted or unsubstituted C₁-C₆ alkyl. In some embodiments, R^{b} is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R^{b} is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R^{b} is a substituted C₁ alkyl group, examples of which may include, but are not limited to, —CDH₂, —CD₂H, —CD₃, —CFH₂, —CF₂H, —CF₃, and —CH₂C≡N. In some embodiments, R^{b} is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R^{b} is not a substituted C₂ alkyl group such as a C₂ fluoroalkyl group. For example, in some embodiments when R⁴ is —OR^{b} or —SR^{b}, R^{b} is not —CH₂CFH₂, —CH₂CF₂H, or —CH₂CF₃. In some embodiments, R^{b} is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R^{b} is not —CH₂CH₂CFH₂.

In some embodiments, R^{b} is a substituted or unsubstituted alkenyl, e.g., a substituted or unsubstituted allyl, butenyl, crotyl, etc.

In some embodiments, R^{b} is a substituted or unsubstituted alkynyl, e.g., a substituted or unsubstituted acetylenyl, propargyl, homopropargyl, etc. In some embodiments, R^{b} is an unsubstituted alkynyl. In some embodiments, R^{b} is an unsubstituted acetylenyl (—C≡CH). In some embodiments, R^{b} is an unsubstituted propargyl (—CH₂C≡CH). In some embodiments, R^{b} is —CH₂CH₂C≡CH. In some embodiments, R^{b} is —CH₂CH₂CH₂C≡CH. In some embodiments, R^{b} is —CH₂CH₂CH₂CH₂C≡CH. In some embodiments, R^{b} is a substituted alkynyl. In some embodiments, R^{b} is a substituted propargyl (e.g., —CF₂C≡CH). In some embodiments, R^{b} is —CF₂CH₂C≡CH. In some embodiments, R^{b} is —CF₂CH₂CH₂C≡CH. In some embodiments, R^{b} is —CF₂CH₂CH₂CH₂C≡CH.

In some embodiments, R^{b} is a substituted or unsubstituted cycloalkyl, for example a substituted or unsubstituted C₃-C₁₀ cycloalkyl, or a substituted or unsubstituted C₄-C₅ cycloalkyl, or a substituted or unsubstituted C₅-C₆ cycloalkyl. In some embodiments, R^{b} is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R^{b} is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁴ is -A(CF₂)ₘ(CH₂)ₙQ, wherein A is S, O, CH₂, or CF₂, m is 0 to 3, n is 0 to 6, and Q is —C≡CH or —C≡N. In some embodiments, A is S. In some embodiments, A is O. In some embodiments, A is CH₂ (methylene). In some embodiments, A is CF₂ (difluoromethylene). In some embodiments, m is 0. In some embodiments, m is 1. In some embodiments, m is 2. In some embodiments, m is 3. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, when A is S or O, the sum of m+n is no more than 6, or no more than 5, or no more than 4, or no more than 3, or no more than 2, or no more than 1, or 0. In some embodiments, when A is CH₂ or CF₂, the sum of m+n is no more than 5, or no more than 4, or no more than 3, or no more than 2, or no more than 1, or 0. In some embodiments, Q is —C≡CH. In some embodiments, Q is —C≡N. Representative examples of the group -A(CF₂)ₘ(CH₂)ₙQ include, but are not limited to, —SC≡CH, —SCH₂C≡CH, —SCF₂C≡CH, —SCH₂C≡N, —SCH₂CH₂C≡CH, —SCF₂CH₂C≡CH, —SCH₂CH₂CH₂C≡CH, —SCF₂CH₂CH₂C≡CH, —OCH₂C≡CH, —OC≡CH, —CH₂C≡CH, —CF₂C≡CH, —CH₂CH₂C≡CH, —CF₂CH₂C≡CH, —CH₂CH₂CH₂C≡CH, —CF₂CH₂CH₂C≡CH, —CH₂CH₂CH₂CH₂C≡CH, and —CF₂CH₂CH₂CH₂C≡CH.

In some embodiments, R⁴ is selected from the group consisting of —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br. In some embodiments, R⁴ is selected from the group consisting of —SMe, —SCF₃, —SCF₂H, -Me, —OCD₃, —CF₃, -t-Bu, or -cyclopentyl. In some embodiments, R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl. In embodiments where R⁴ is —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), or -A(CF₂)ₘ(CH₂)ₙQ, (wherein A is S, O, CH₂, or CF₂, m is 0 to 3, n is 0 to 6, and Q is —C≡CH or —C≡N), the other substituents (i.e., X¹, X², Y¹, Y², R⁶, R⁷, and R^{a}) may, or may not, comprise deuterium. In some embodiments, R⁴ is selected from the group consisting of —SCF₂H, —SCFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl (—C₅H₉). In embodiments where R⁴ is —SCF₂H, —SCFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CF₂CF₂H, —OCH₂C≡CH, —OC≡CH, —CH₂CH₂CH₂CH₂C≡CH, —CF₂CH₂CH₂CH₂C≡CH, or -cyclopentyl (—C₆H₉), the other substituents (i.e., X¹, X², Y¹, Y², R³, R⁶, R⁷, and R^{a}) may, or may not, comprise deuterium.

R⁶ and R⁷ may be the same, or different. In some embodiments, R⁶ and R⁷ are the same. In some embodiments, R⁶ and R⁷ are different. For example, in some embodiments, R⁶ is hydrogen, and R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl.

In some embodiments, R⁶ and/or R⁷ is an unsubstituted C₁-C₆ alkyl, for example, an unsubstituted C₁ alkyl, an unsubstituted C₂ alkyl, an unsubstituted C₃ alkyl, an unsubstituted C₄ alkyl, an unsubstituted C₅ alkyl, or an unsubstituted C₆ alkyl. In some embodiments, R⁶ and/or R⁷ is an unsubstituted linear C₂-C₆ alkyl. In some embodiments, R⁶ and/or R⁷ is an unsubstituted branched C₃-C₁₀ alkyl. Examples of an unsubstituted C₁-C₁₀ alkyl include, but are not limited to, methyl, ethyl, n-propyl, isopropyl, butyl, isobutyl, t-butyl, n-pentyl, isopentyl, n-hexyl, and isohexyl.

In some embodiments, R⁶ and/or R⁷ is a substituted C₁-C₆ alkyl, e.g., a substituted C₁ alkyl, a substituted C₂ alkyl, a substituted C₃ alkyl, a substituted C₄ alkyl, a substituted C₅ alkyl, or a substituted C₆ alkyl. The alkyl group may contain one, or more than one, substituent. The alkyl group may be substituted with any one or more substituents recited herein, examples of those substituents may include, but are not limited to, deuterium, halogen (e.g., fluorine), polar substituents such as hydroxyl, oxo, unsubstituted alkoxy, substituted alkoxy (e.g., polyether groups), unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. In some embodiments, R⁶ and/or R⁷ is a C₁-C₆ alkyl substituted with one or more deuterium atoms, examples of which include, but are not limited to, —CDH₂, —CD₂H, —CD₃, —CD₂CD₃, and —CD₂CD₂CD₃. In some embodiments, R⁶ and/or R⁷ is a C₁-C₆ alkyl substituted with one or more fluorine atoms, i.e., is a fluoroalkyl group. Examples of fluoroalkyl groups include, but are not limited to, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, —CH₂CH₂CH₂CF₃, —CH₂CF₂CHF₂, —CH₂CF₂CF₃, —CH(CF₃)₂, and —CH(CH₃)CF₃.

In some embodiments, R⁶ and/or R⁷ is a C₁-C₆ alkyl substituted with one or more deuterium atoms and one or more fluorine atoms, examples of which include, but are not limited to, —CD₂CH₂F, —CD₂CHF₂, —CD₂CF₃, —CD₂CH₂CH₂F, —CD₂CH₂CHF₂, —CD₂CH₂CF₃, —CD₂CD₂CH₂, —CD₂CD₂CHF₂, —CD₂CD₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃.

In some embodiments, R⁶ and/or R⁷ is a C₁-C₆ alkyl substituted with a substituted or unsubstituted cycloalkyl. The C₁-C₆ alkyl may be substituted with, e.g., a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, the C₁-C₆ alkyl is substituted with an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, the C₁-C₆ alkyl is substituted with a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent. In some embodiments, R⁶ and/or R⁷ is a C₁ alkyl substituted with a substituted or unsubstituted cycloalkyl, with particular mention being made to cyclopropylmethyl (—CH₂C₃H₅).

In some embodiments, R⁶ and/or R⁷ is a substituted or unsubstituted alkenyl, e.g., a substituted or unsubstituted allyl, butenyl, crotyl, etc.

In some embodiments, R⁶ and/or R⁷ is a substituted or unsubstituted alkynyl, e.g., a substituted or unsubstituted propargyl.

In some embodiments, R⁶ and/or R⁷ is a substituted or unsubstituted cycloalkyl, for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, R⁶ and/or R⁷ is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R⁶ and/or R⁷ is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁶ and/or R⁷ is a substituted or unsubstituted heterocycloalkyl. In some embodiments, the unsubstituted or substituted heterocycloalkyl group may be a 3-membered ring, a 4-membered ring, a 5-membered ring, a 6-membered ring, a 7-membered ring, or an 8-membered ring. In some embodiments, R⁶ and/or R⁷ is an unsubstituted heterocycloalkyl, such as those set forth herein, examples of which include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, tetrahydrofuran, tetrahydropyran, and 1,3-dioxolane. In some embodiments, R⁶ and/or R⁷ is a substituted heterocycloalkyl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), oxo, and hydroxyl. The heterocycloalkyl group may contain one, or more than one, substituent.

In some embodiments, R⁶ and/or R⁷ is a substituted or unsubstituted aryl. In some embodiments, R⁶ and/or R⁷ is an unsubstituted aryl, examples of which include, but are not limited to, phenyl and naphthyl. In some embodiments, R⁶ and/or R⁷ is a substituted aryl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), hydroxyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The aryl group may contain one, or more than one, substituent.

In some embodiments, R⁶ and/or R⁷ is a substituted or unsubstituted heteroaryl. In some embodiments, R⁶ and/or R⁷ is an unsubstituted heteroaryl, examples of which include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothiophenyl, thiophenyl, benzimidazolyl, quinolinyl, isoquinolinyl, quinazolinyl, quinoxalinyl, and pyrazolyl. In some embodiments, R⁶ and/or R⁷ is a substituted heteroaryl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), hydroxyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The heteroaryl group may contain one, or more than one, substituent.

In some embodiments, R⁶ is hydrogen, and R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl. In some embodiments, R⁶ is hydrogen, and R⁷ is a substituted or unsubstituted C₁-C₆ alkyl. In some embodiments, R⁶ is hydrogen, and R⁷ is an unsubstituted C₁-C₆ alkyl, a C₁-C₆ alkyl substituted with one or more deuterium atoms, a C₁-C₆ alkyl substituted with one or more fluorine atoms, or a C₁-C₆ alkyl substituted with a substituted or unsubstituted cycloalkyl. For example, in some embodiments, R⁶ is hydrogen, and R⁷ is methyl, ethyl, propyl, —CD₃, or cyclopropylmethyl (—CH₂C₃H₅).

In some embodiments, R⁶ and R⁷ together with the nitrogen atom attached thereto are joined to form a substituted or unsubstituted heterocycloalkyl. In some embodiments, R⁶ and R⁷ together with the nitrogen atom attached thereto are joined to form an unsubstituted heterocycloalkyl. The unsubstituted heterocycloalkyl group may be, e.g., a 3-membered ring, a 4-membered ring, 5-membered ring, a 6-membered ring, a 7-membered ring, an 8-membered ring, etc., which may be optionally fused to other ring(s). The unsubstituted heterocycloalkyl group contains a minimum of one nitrogen ring atom (the nitrogen atom intervening R⁶ and R⁷), and may optionally contain at least one additional hetero-ring atom, which may be one or more of nitrogen, sulfur, or oxygen, for a total of 1, 2, 3, or 4 hetero-ring atoms (at least one of which is a nitrogen ring atom). Examples of unsubstituted heterocycloalkyl groups formed from joining R⁶ and R⁷ together with the nitrogen atom attached thereto include, but are not limited to,

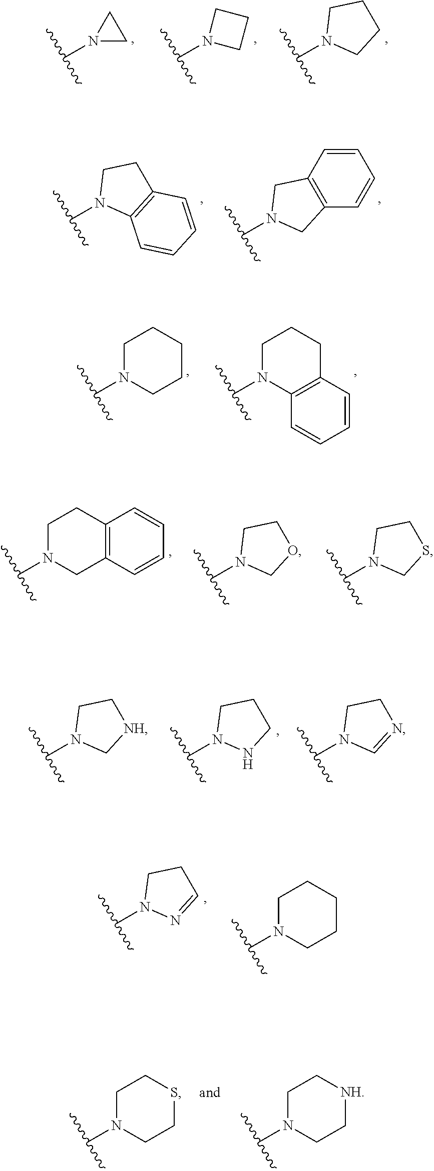

In some embodiments, R⁶ and R⁷ together with the nitrogen atom attached thereto are joined to form a substituted heterocycloalkyl. The substituted heterocycloalkyl group may be, e.g., a 3-membered ring, a 4-membered ring, a 5-membered ring, a 6-membered ring, a 7-membered ring, an 8-membered ring, etc., which may be optionally fused to other ring(s). The substituted heterocycloalkyl group contains a minimum of one nitrogen ring atom (the nitrogen atom intervening R⁶ and R⁷), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the substituted heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, 1,2,3,4-tetrahydroisoquinoline, thiazolidine, morpholine, or thiomorpholine, which is substituted with at least one substituent. The substituent(s) may be any recited herein, including, but not limited to, deuterium, halogen (e.g., fluorine), polar substituents such as hydroxyl, oxo, unsubstituted alkoxy, substituted alkoxy (e.g., polyether groups), unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The substituted heterocycloalkyl formed from joining R⁶ and R⁷ together with the nitrogen atom attached thereto contains a heterocycloalkyl group substituted with one, two, three, four, or more substituents. The substituent may be located on a carbon ring atom or on a hetero-ring atom.

Examples of substituted heterocycloalkyl groups formed from joining R⁶ and R⁷ together with the nitrogen atom attached thereto include, but are not limited to,

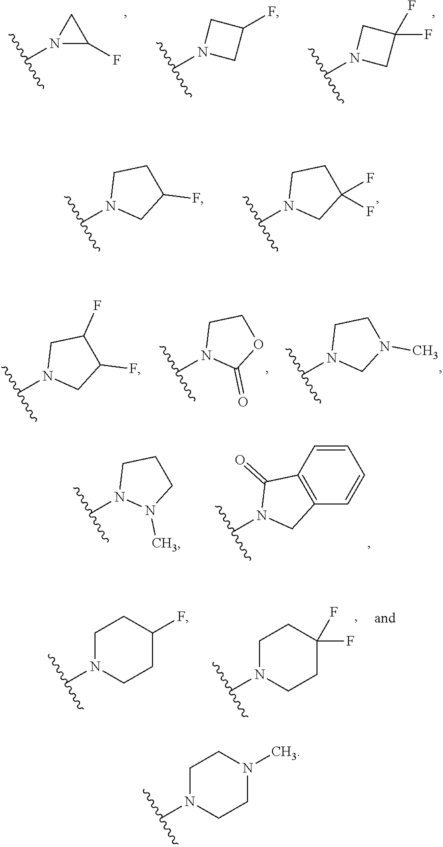

Each R^{a} may be the same, or different. In some embodiments, each R^{a} is the same. Each R^{a} may be, independently, a substituted or unsubstituted C₁-C₆ alkyl, preferably a substituted or unsubstituted C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, each R^{a} is different, e.g., one R^{a} is —CH₃, while another is —CD₃.

In some embodiments, Y¹ and Y² are each hydrogen or each deuterium; X¹ and X² are each hydrogen or each deuterium; each R^{a} is —CH₃ or —CD₃; R⁴ is —SMe, —SCD₃, —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —SEt, —Sn—Pr, -Me, —CD₃, —CF₃, -t-Bu, —C(CD₃)₃, -cyclopentyl, —OMe, —OCD₃, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, —CH₂CH₂CH₂CH₂C≡CH, —CF₂CH₂CH₂CH₂C≡CH, —Cl, —I, or —Br, preferably R⁴ is —SCF₃, —SCF₂H, —SCFH₂, —SCH₂CH₂CF₃, —SCH₂CH₂CF₂H, —SCH₂CH₂CFH₂, —SCH₂CF₂CF₂H, —SCH₂C≡CH, —SC≡CH, —OCF₃, —OCF₂H, —OCFH₂, —OCH₂CH₂CF₃, —OCH₂CH₂CF₂H, —OCH₂CH₂CFH₂, —OCH₂CF₂CF₂H, —OCH₂C≡CH, —OC≡CH, and -cyclopentyl; R⁶ is hydrogen; and R⁷ is a substituted or unsubstituted C₁-C₆ alkyl.

In some embodiments, the compound of Formula (V) may be provided with the following proviso: (i) at least one of X¹, X², Y¹, Y², R⁴, R⁶, R⁷, and R^{a} comprises deuterium and/or (ii) R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N. In some embodiments, in condition (ii), R⁴ is selected from the group consisting of —SCF₂H, —SCFH₂, —SCH₂CF₂CF₂H, —SCH₂C≡CH, —SC≡CH, —SCF₂C≡CH, —SCH₂CH₂CH₂C≡CH, —SCF₂CH₂CH₂C≡CH, —OCF₃, —OCF₂H, —OCFH₂, —OCH₂CF₂CF₂H, —OCH₂C≡CH, —OC≡CH, —CH₂CH₂CH₂CH₂C≡CH, —CF₂CH₂CH₂CH₂C≡CH, and -cyclopentyl (—C₅H₉). The above proviso written with “and/or” means that compounds fall within the scope when one of conditions (i) or (ii) is satisfied, or when both conditions (i) and (ii) are satisfied. For clarity, compounds in which condition (i) is satisfied, i.e., where at least one of X¹, X², Y¹, Y², R³, R⁴, R⁶, R⁷, and R^{a} comprises deuterium, do not require condition (ii) to be satisfied, e.g., R⁴ may or may not be selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N. Likewise compounds in which condition (ii) is satisfied, e.g., when R⁴ is —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), or -A(CF₂)ₘ(CH₂)ₙQ, wherein A is S, O, CH₂, or CF₂, m is 0 to 3, n is 0 to 6, and Q is —C≡CH or —C≡N, do not require condition (i) to be satisfied, i.e., the other substituents X¹, X², Y¹, Y², R³, R⁶, R⁷ and R^{a} may, or may not, comprise deuterium. In some embodiments, a compound of Formula (V) is provided without the above proviso.

In some embodiments, the compound, e.g., the compound of Formula (I) through (V) is selected from the group consisting of:

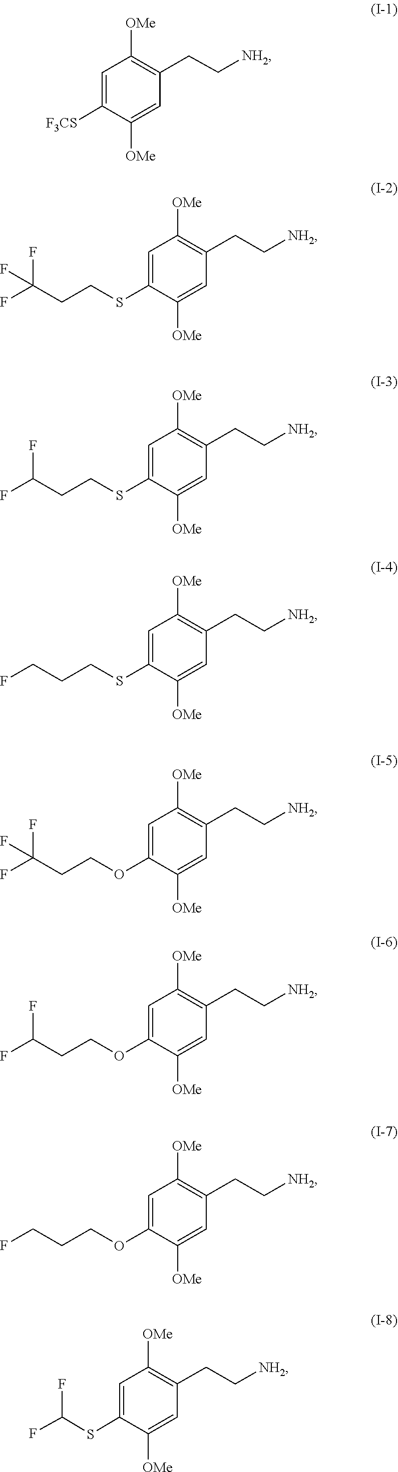

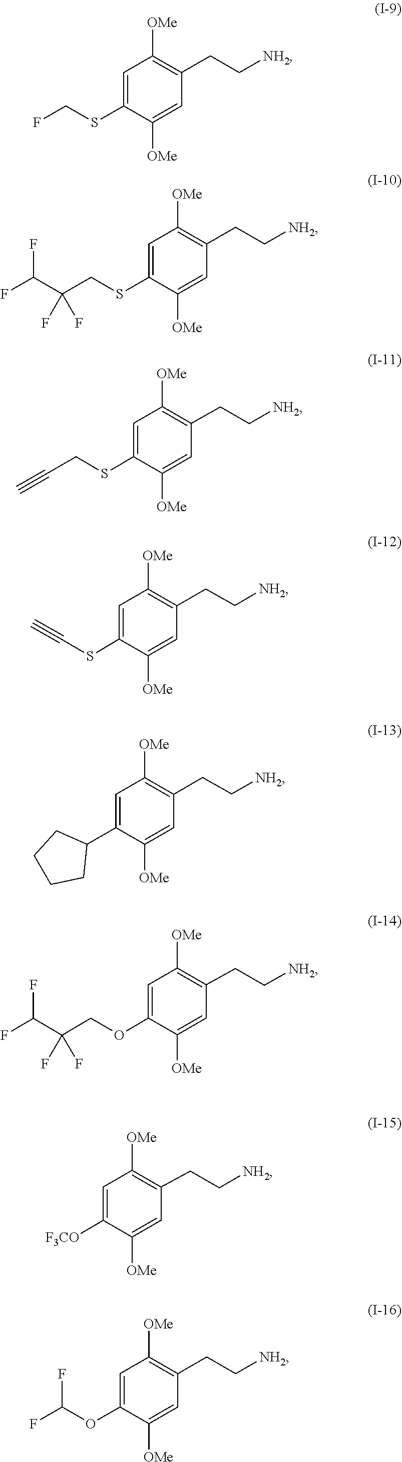

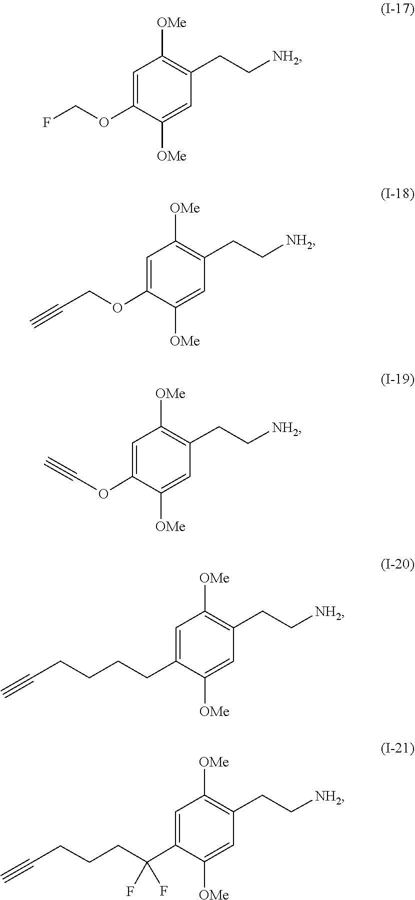 [SMILES: COc1cc(OCF)c(OC)cc1CCN ; C#CCOc1cc(OC)c(CCN)cc1OC ; C#COc1cc(OC)c(CCN)cc1OC ; C#CCCCCc1cc(OC)c(CCN)cc1OC ; C#CCCCC(F)(F)c1cc(OC)c(CCN)cc1OC]

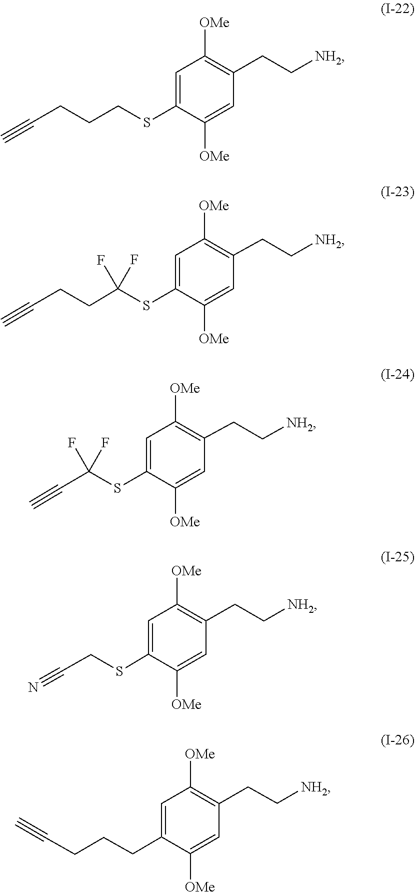 [SMILES: C#CCCCSc1cc(OC)c(CCN)cc1OC ; C#CCCC(F)(F)Sc1cc(OC)c(CCN)cc1OC ; C#CC(F)(F)Sc1cc(OC)c(CCN)cc1OC ; COc1cc(SCC#N)c(OC)cc1CCN ; C#CCCCc1cc(OC)c(CCN)cc1OC]

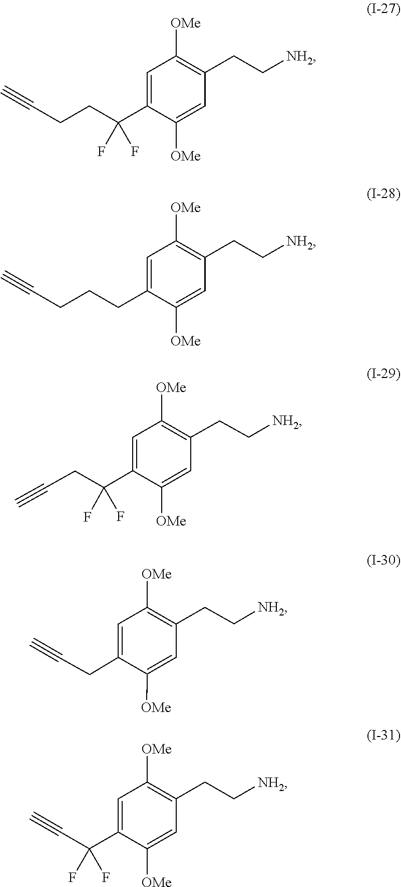

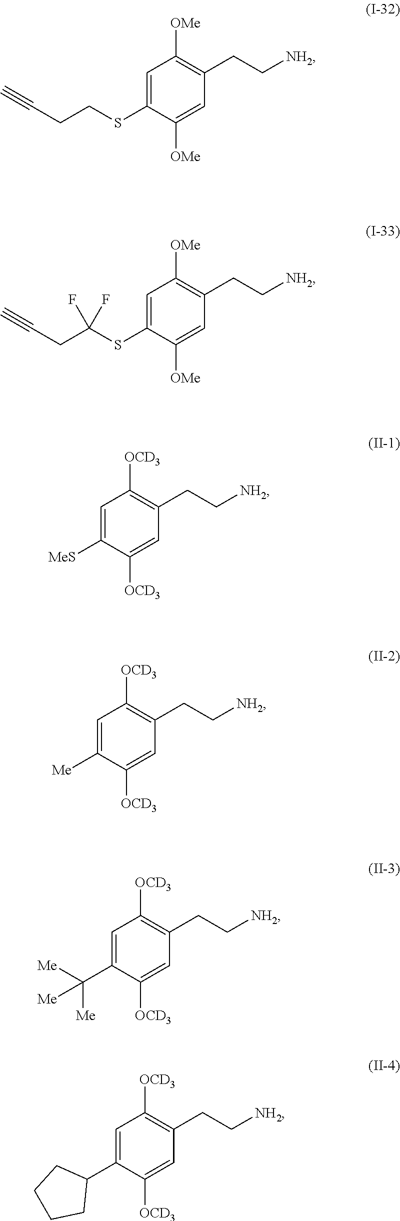

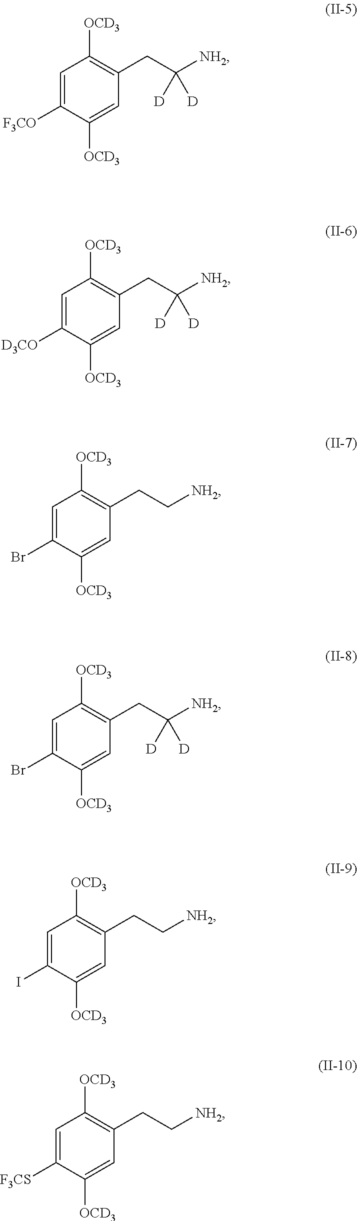

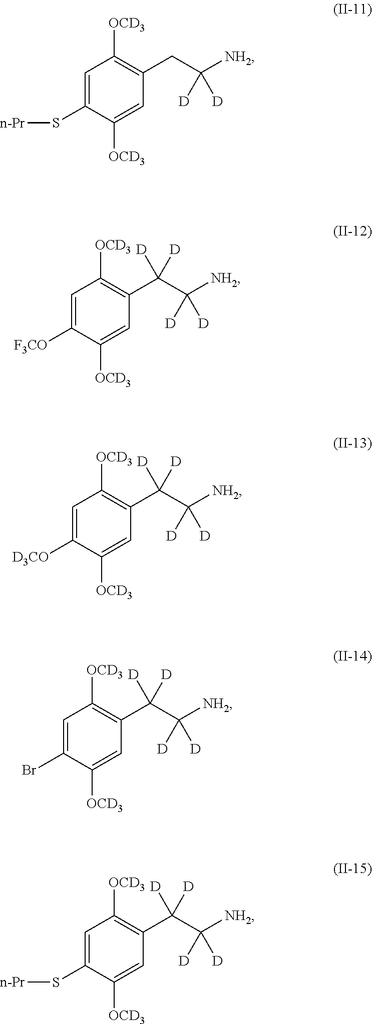

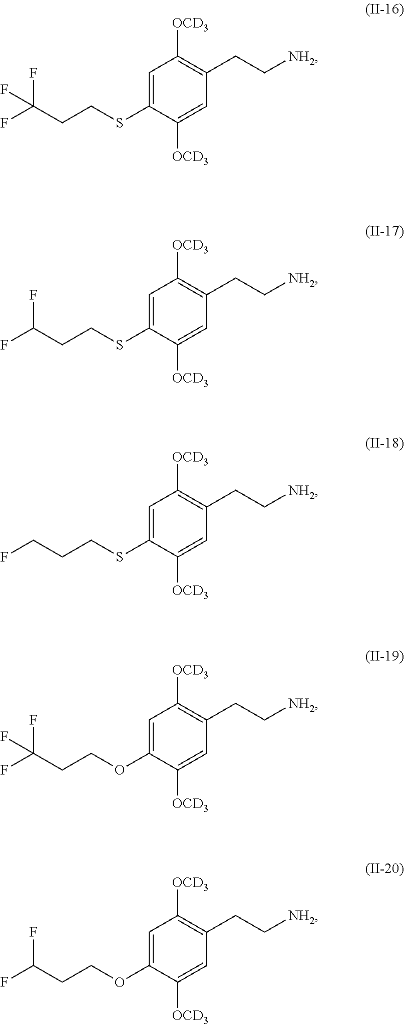

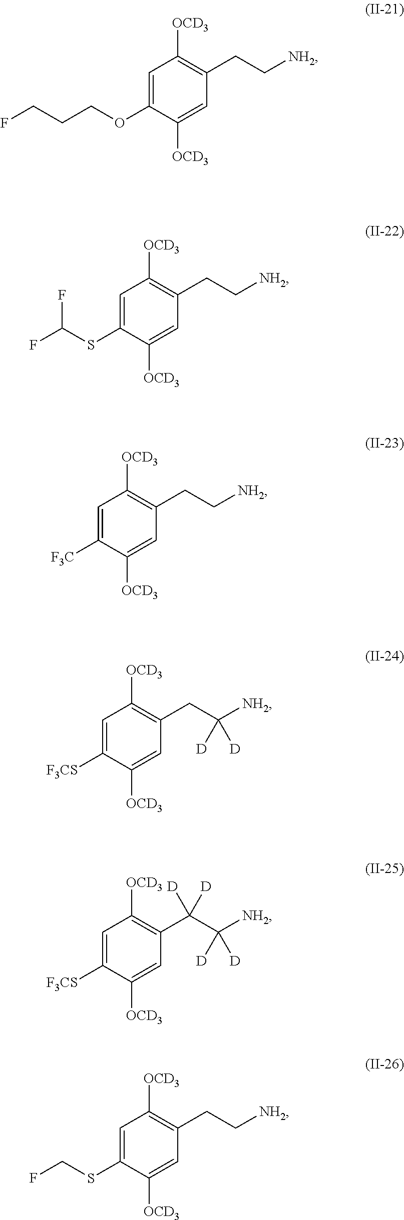

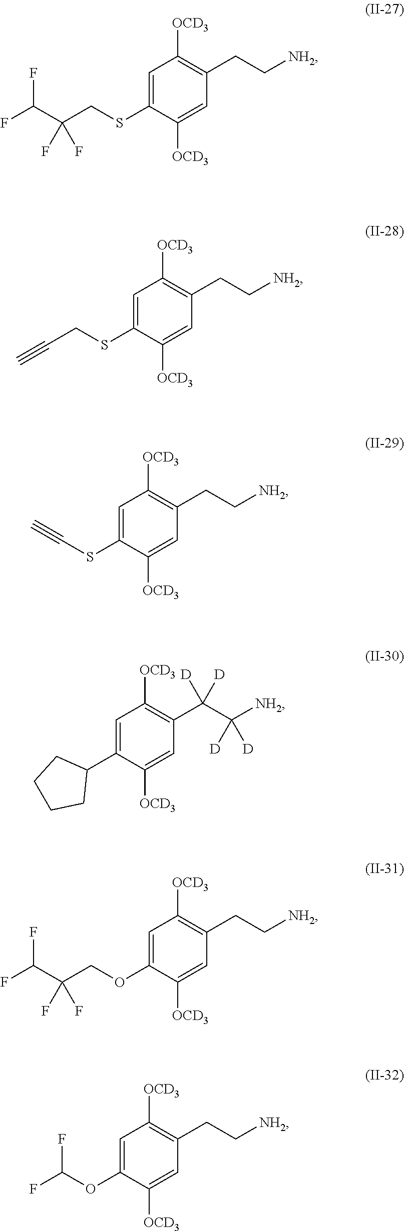

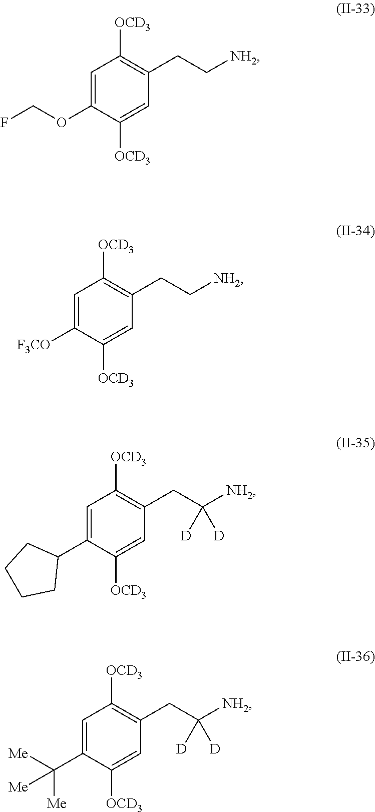

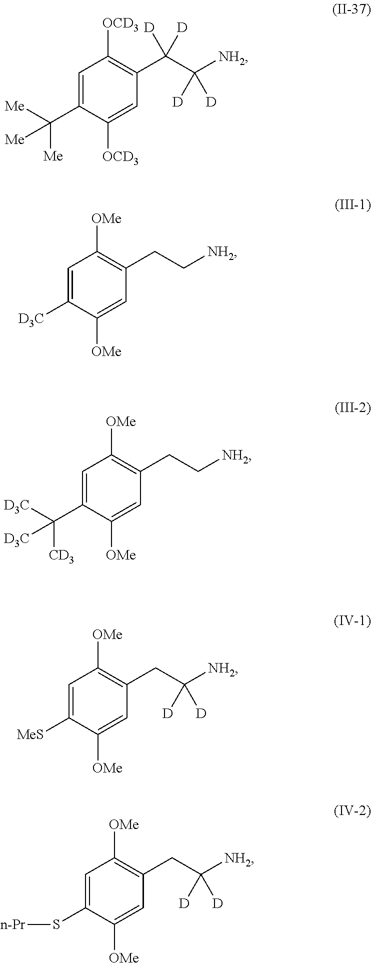

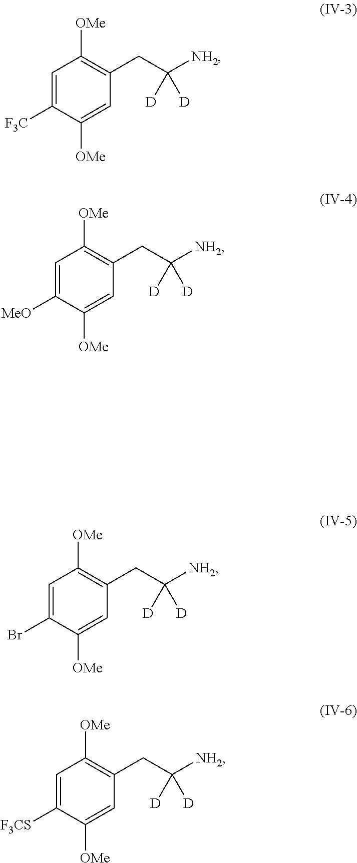

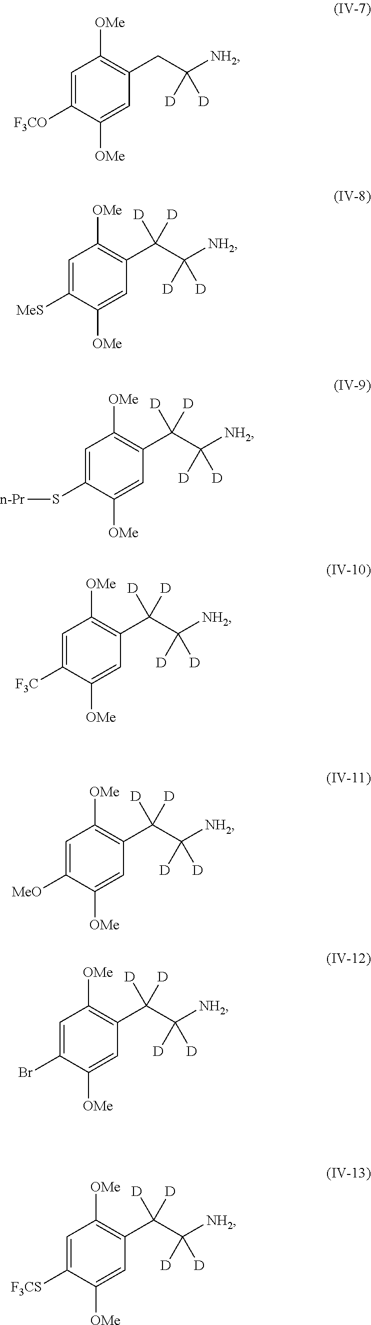

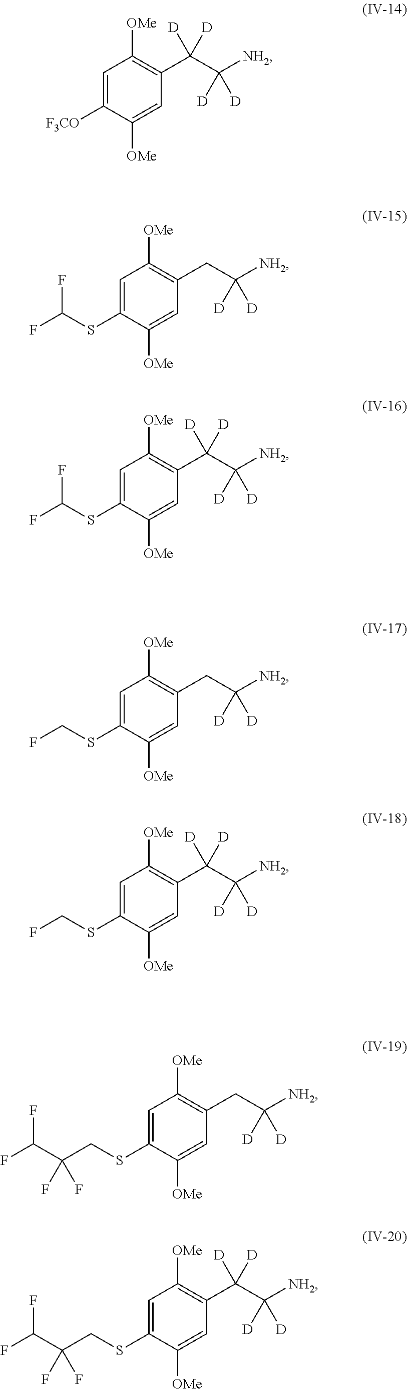

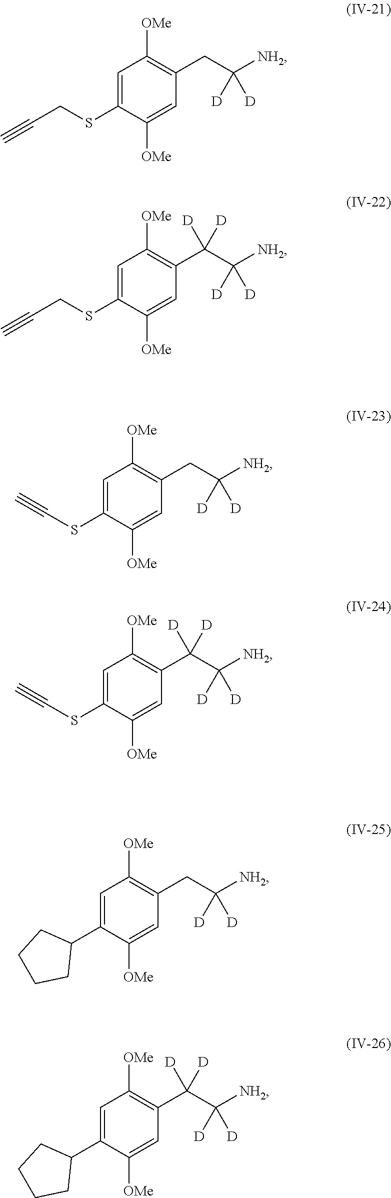 [SMILES: [2H]C([2H])(N)Cc1cc(OC)c(SCC#C)cc1OC ; [2H]C([2H])(N)C([2H])([2H])c1cc(OC)c(SCC#C)cc1OC ; [2H]C([2H])(N)Cc1cc(OC)c(SC#C)cc1OC ; [2H]C([2H])(N)C([2H])([2H])c1cc(OC)c(SC#C)cc1OC ; [2H]C([2H])(N)Cc1cc(OC)c(C2CCCC2)cc1OC ; [2H]C([2H])(N)C([2H])([2H])c1cc(OC)c(C2CCCC2)cc1OC]

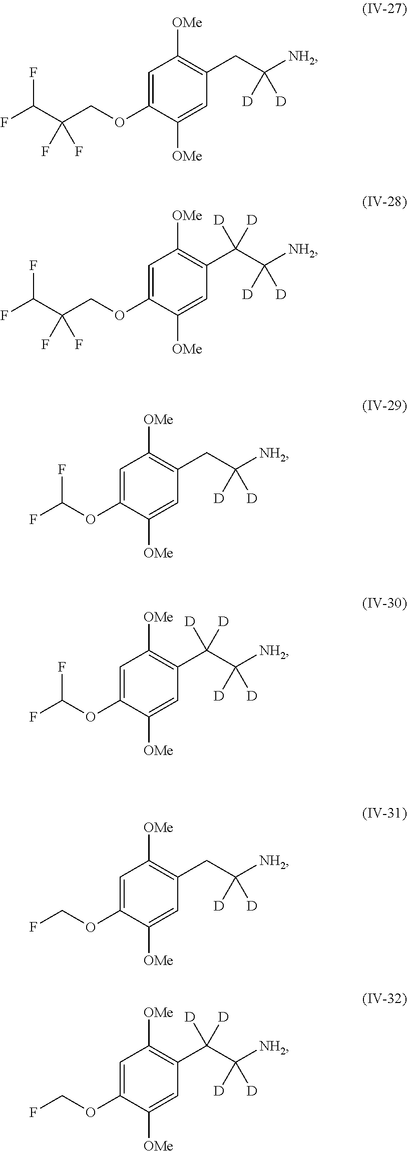 [SMILES: [2H]C([2H])(N)Cc1cc(OC)c(OCC(F)(F)C(F)F)cc1OC ; [2H]C([2H])(N)C([2H])([2H])c1cc(OC)c(OCC(F)(F)C(F)F)cc1OC ; [2H]C([2H])(N)Cc1cc(OC)c(OC(F)F)cc1OC ; [2H]C([2H])(N)C([2H])([2H])c1cc(OC)c(OC(F)F)cc1OC ; [2H]C([2H])(N)Cc1cc(OC)c(OCF)cc1OC ; [2H]C([2H])(N)C([2H])([2H])c1cc(OC)c(OCF)cc1OC]

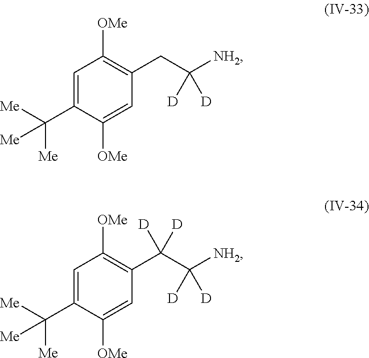 [SMILES: [2H]C([2H])(N)Cc1cc(OC)c(C(C)(C)C)cc1OC ; [2H]C([2H])(N)C([2H])([2H])c1cc(OC)c(C(C)(C)C)cc1OC]

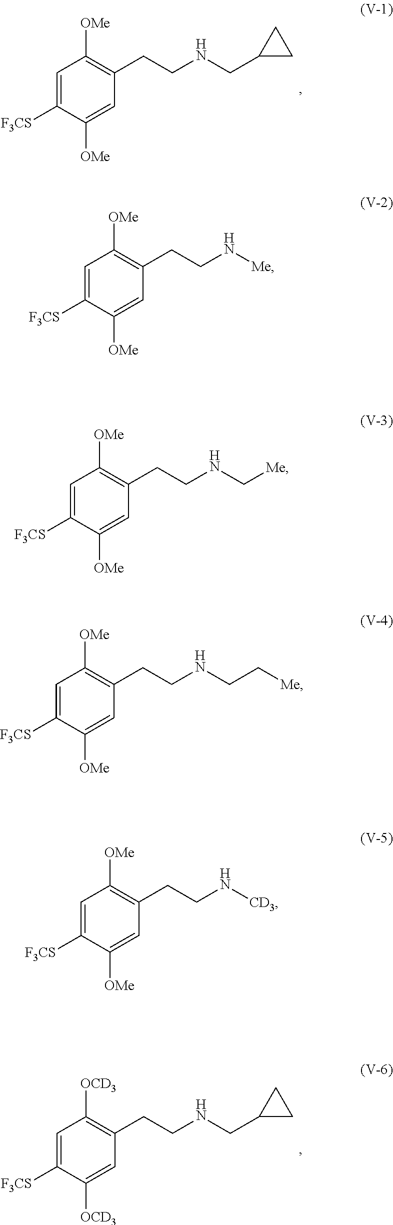

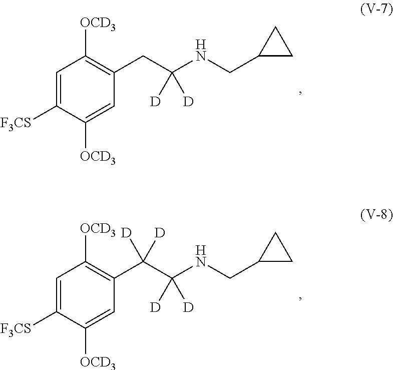

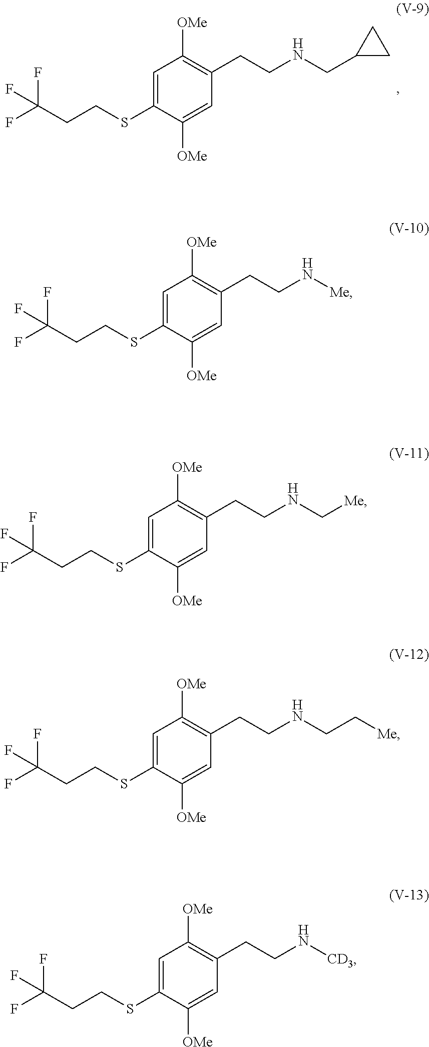 [SMILES: COc1cc(SCCC(F)(F)F)c(OC)cc1CCNCC1CC1 ; CNCCc1cc(OC)c(SCCC(F)(F)F)cc1OC ; CCNCCc1cc(OC)c(SCCC(F)(F)F)cc1OC ; CCCNCCc1cc(OC)c(SCCC(F)(F)F)cc1OC ; [2H]C([2H])([2H])NCCc1cc(OC)c(SCCC(F)(F)F)cc1OC]

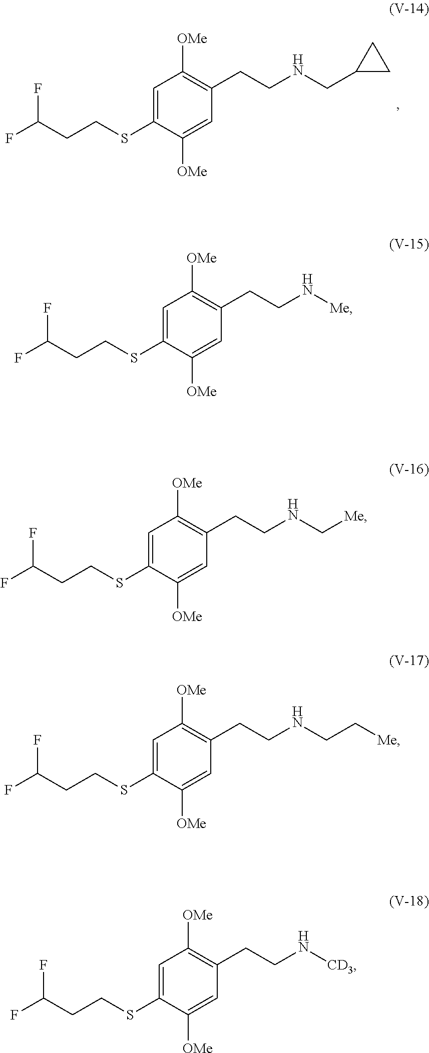 [SMILES: COc1cc(SCCC(F)F)c(OC)cc1CCNCC1CC1 ; CNCCc1cc(OC)c(SCCC(F)F)cc1OC ; CCNCCc1cc(OC)c(SCCC(F)F)cc1OC ; CCCNCCc1cc(OC)c(SCCC(F)F)cc1OC ; [2H]C([2H])([2H])NCCc1cc(OC)c(SCCC(F)F)cc1OC]

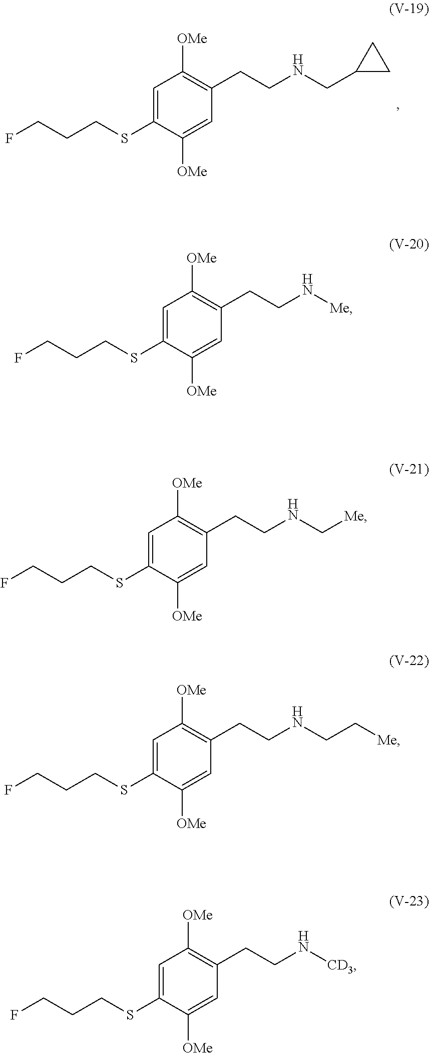 [SMILES: COc1cc(SCCCF)c(OC)cc1CCNCC1CC1 ; CNCCc1cc(OC)c(SCCCF)cc1OC ; CCNCCc1cc(OC)c(SCCCF)cc1OC ; CCCNCCc1cc(OC)c(SCCCF)cc1OC ; [2H]C([2H])([2H])NCCc1cc(OC)c(SCCCF)cc1OC]

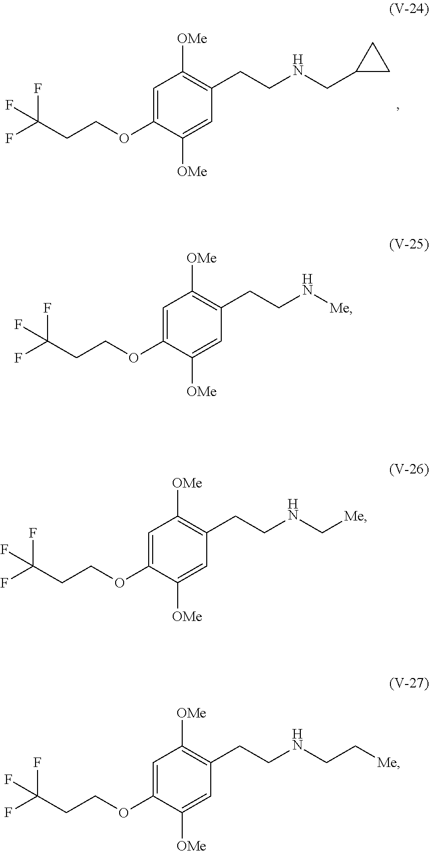 [SMILES: COc1cc(OCCC(F)(F)F)c(OC)cc1CCNCC1CC1 ; CNCCc1cc(OC)c(OCCC(F)(F)F)cc1OC ; CCNCCc1cc(OC)c(OCCC(F)(F)F)cc1OC ; CCCNCCc1cc(OC)c(OCCC(F)(F)F)cc1OC]

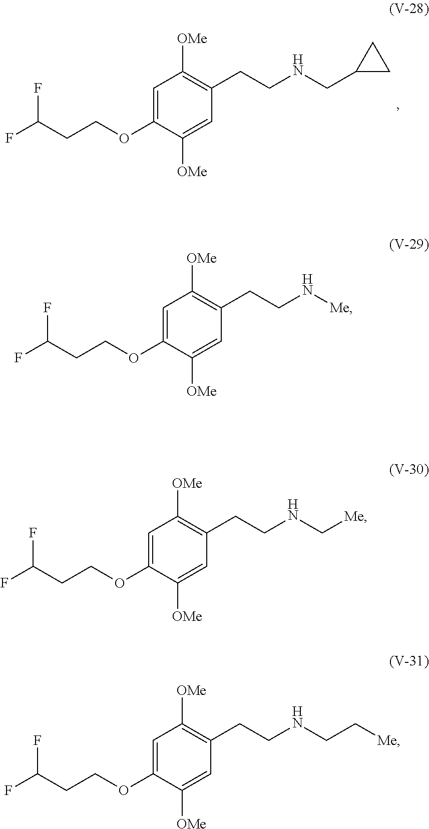 [SMILES: COc1cc(OCCC(F)F)c(OC)cc1CCNCC1CC1 ; CNCCc1cc(OC)c(OCCC(F)F)cc1OC ; CCNCCc1cc(OC)c(OCCC(F)F)cc1OC ; CCCNCCc1cc(OC)c(OCCC(F)F)cc1OC]

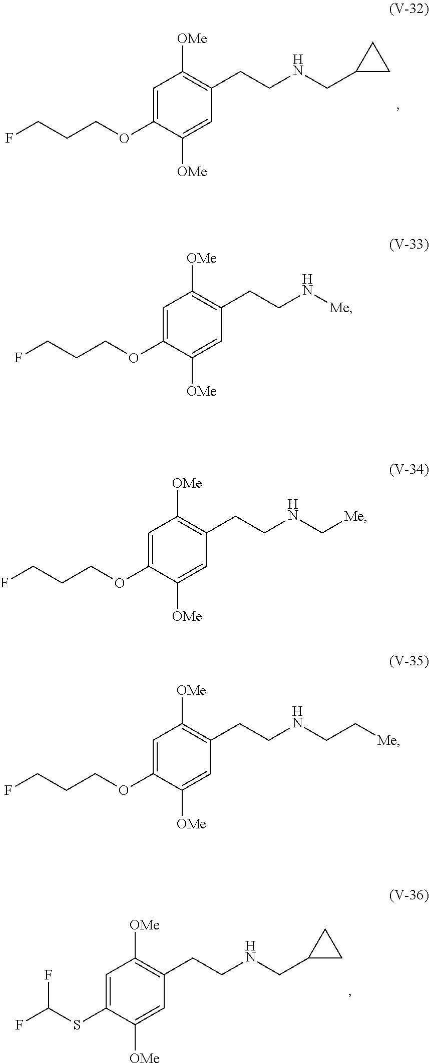 [SMILES: COc1cc(OCCCF)c(OC)cc1CCNCC1CC1 ; CNCCc1cc(OC)c(OCCCF)cc1OC ; CCNCCc1cc(OC)c(OCCCF)cc1OC ; CCCNCCc1cc(OC)c(OCCCF)cc1OC ; COc1cc(SC(F)F)c(OC)cc1CCNCC1CC1]

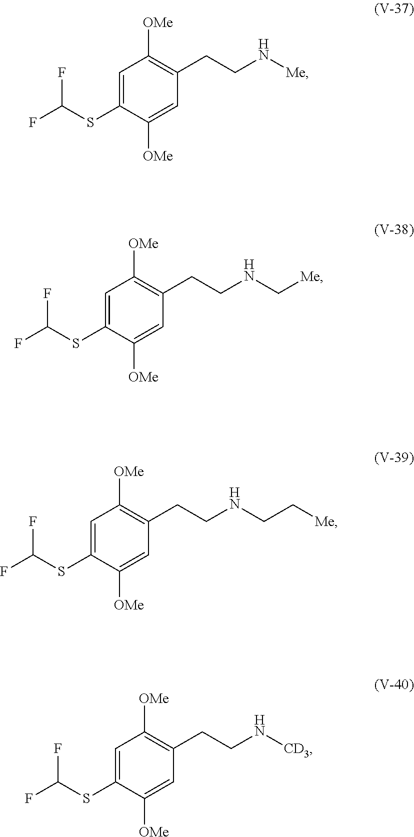 [SMILES: CNCCc1cc(OC)c(SC(F)F)cc1OC ; CCNCCc1cc(OC)c(SC(F)F)cc1OC ; CCCNCCc1cc(OC)c(SC(F)F)cc1OC ; [2H]C([2H])([2H])NCCc1cc(OC)c(SC(F)F)cc1OC]

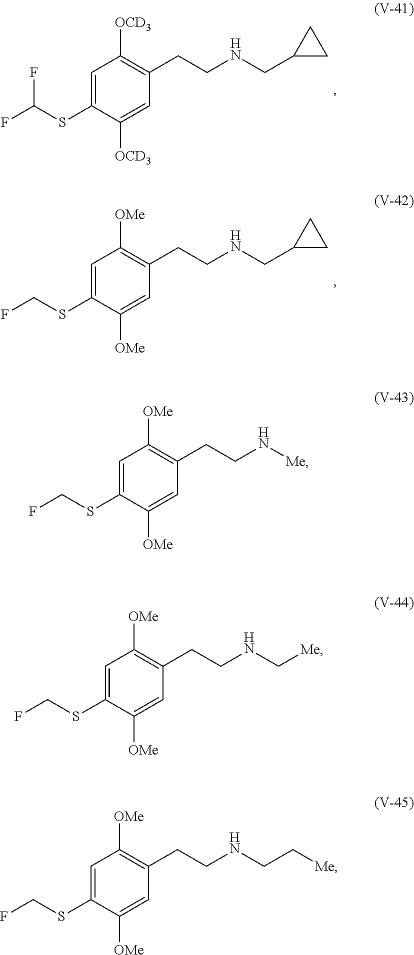

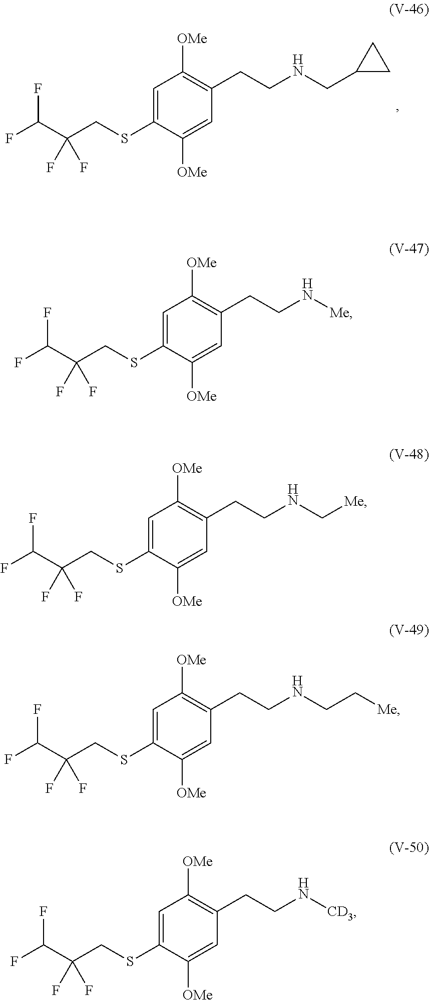 [SMILES: COc1cc(SCC(F)(F)C(F)F)c(OC)cc1CCNCC1CC1 ; CNCCc1cc(OC)c(SCC(F)(F)C(F)F)cc1OC ; CCNCCc1cc(OC)c(SCC(F)(F)C(F)F)cc1OC ; CCCNCCc1cc(OC)c(SCC(F)(F)C(F)F)cc1OC ; [2H]C([2H])([2H])NCCc1cc(OC)c(SCC(F)(F)C(F)F)cc1OC]

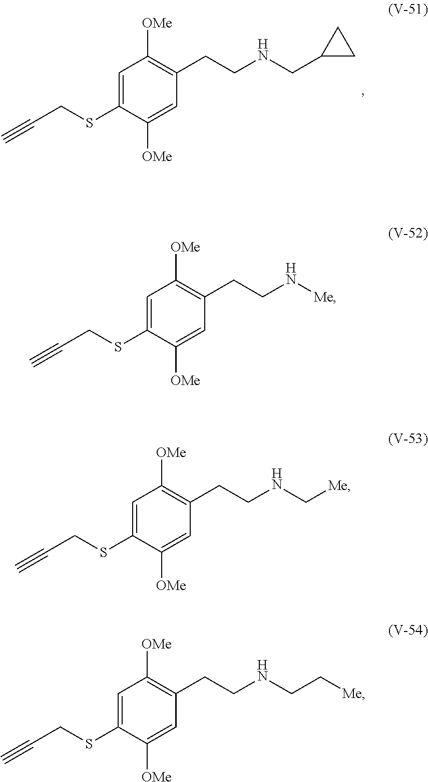 [SMILES: C#CCSc1cc(OC)c(CCNCC2CC2)cc1OC ; C#CCSc1cc(OC)c(CCNC)cc1OC ; C#CCSc1cc(OC)c(CCNCC)cc1OC ; C#CCSc1cc(OC)c(CCNCCC)cc1OC]

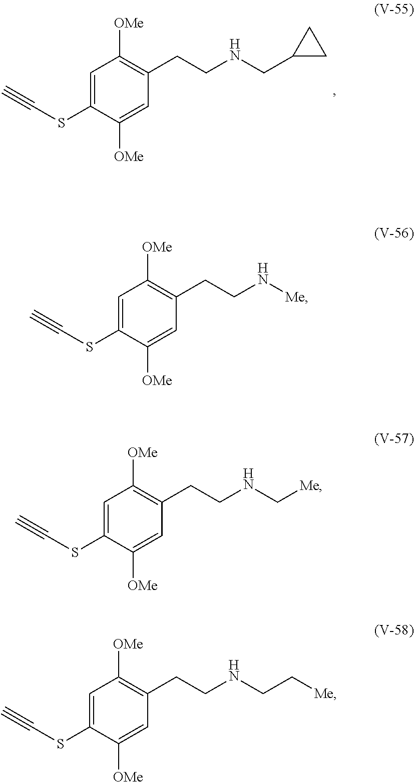 [SMILES: C#CSc1cc(OC)c(CCNCC2CC2)cc1OC ; C#CSc1cc(OC)c(CCNC)cc1OC ; C#CSc1cc(OC)c(CCNCC)cc1OC ; C#CSc1cc(OC)c(CCNCCC)cc1OC]

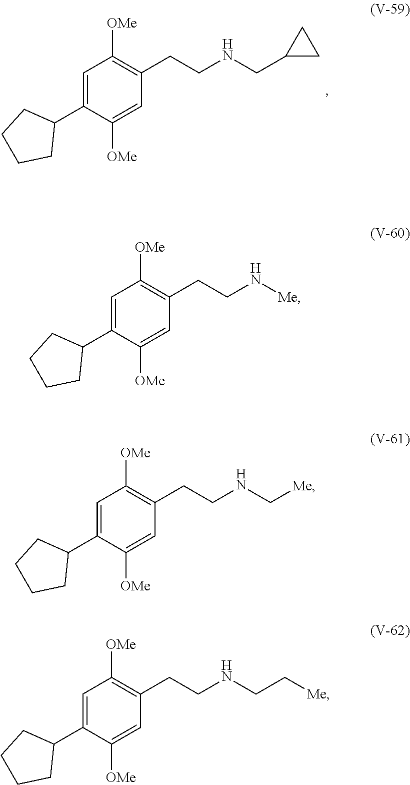 [SMILES: COc1cc(C2CCCC2)c(OC)cc1CCNCC1CC1 ; CNCCc1cc(OC)c(C2CCCC2)cc1OC ; CCNCCc1cc(OC)c(C2CCCC2)cc1OC ; CCCNCCc1cc(OC)c(C2CCCC2)cc1OC]

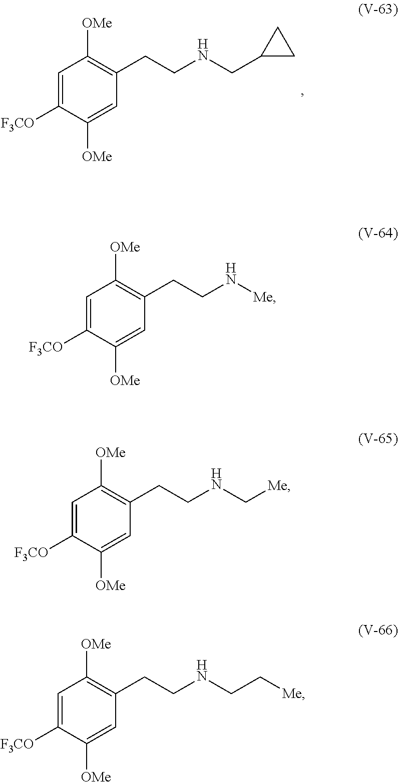 [SMILES: COc1cc(OC(F)(F)F)c(OC)cc1CCNCC1CC1 ; CNCCc1cc(OC)c(OC(F)(F)F)cc1OC ; CCNCCc1cc(OC)c(OC(F)(F)F)cc1OC ; CCCNCCc1cc(OC)c(OC(F)(F)F)cc1OC]

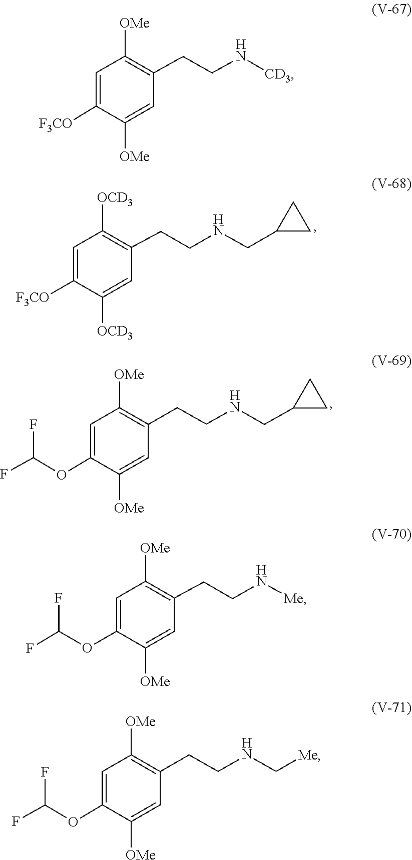

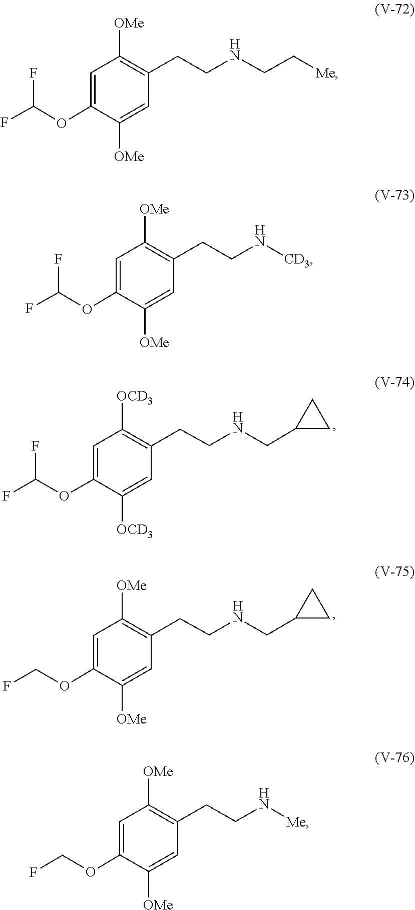

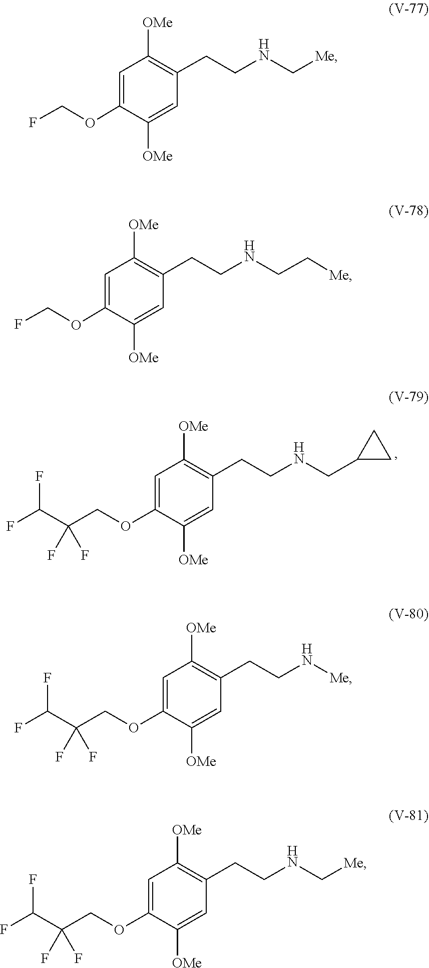 [SMILES: CCNCCc1cc(OC)c(OCF)cc1OC ; CCCNCCc1cc(OC)c(OCF)cc1OC ; COc1cc(OCC(F)(F)C(F)F)c(OC)cc1CCNCC1CC1 ; CNCCc1cc(OC)c(OCC(F)(F)C(F)F)cc1OC ; CCNCCc1cc(OC)c(OCC(F)(F)C(F)F)cc1OC]

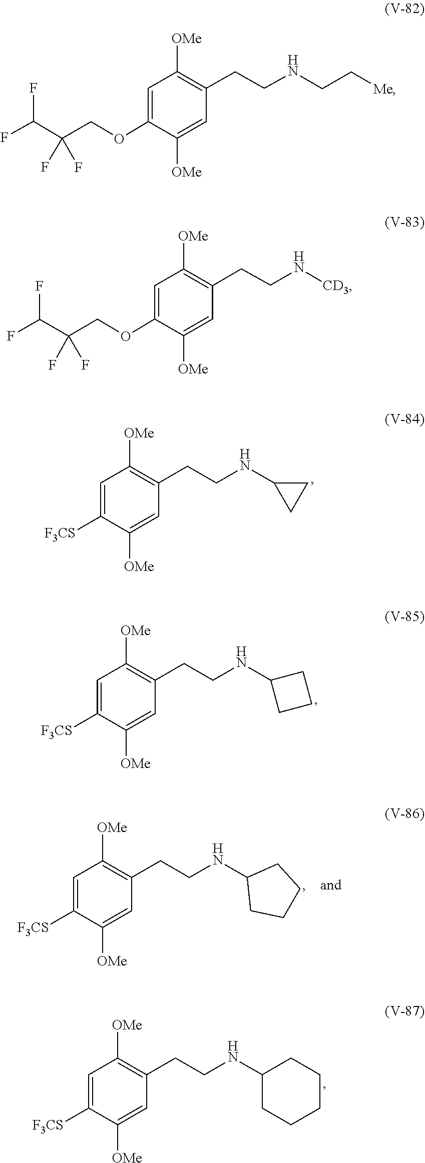

or a pharmaceutically acceptable salt, solvate, or prodrug thereof.

The compound number, IUPAC name, and substituent listing for several of the above-identified compounds are provided in Table 1.

TABLE 1
Exemplary compounds of Formula (I)

| | | Formula (I) | | | | | |
|---|---|---|---|---|---|---|---|
| Compound number and name | | X¹, X² | Y¹, Y² | R³ | R⁴ | R^{a}, R^{a} | R⁶, R⁷ |
| I-1 | 2-(2,5-dimethoxy-4- | H, H | H, H | H | —SCF₃ | —Me, | H, H |
| | ((trifluoromethyl)thio)phenyl)ethan-1- | | | | | —Me | |
| | amine | | | | | | |
| I-2 | 2-(2,5-dimethoxy-4-((3,3,3- | H, H | H, H | H | —SCH₂CH₂CF₃ | —Me, | H, H |
| | trifluoropropyl)thio)phenyl)ethan-1- | | | | | —Me | |
| | amine | | | | | | |
| I-3 | 2-(4-((3,3-difluoropropyl)thio)-2,5- | H, H | H, H | H | —SCH₂CH₂CF₂H | —Me, | H, H |
| | dimethoxyphenyl)ethan-1-amine | | | | | —Me | |
| I-4 | 2-(4-((3-fluoropropyl)thio)-2,5- | H, H | H, H | H | —SCH₂CH₂CFH₂ | —Me, | H, H |
| | dimethoxyphenyl)ethan-1-amine | | | | | —Me | |
| I-5 | 2-(2,5-dimethoxy-4-(3,3,3- | H, H | H, H | H | —OCH₂CH₂CF₃ | —Me, | H, H |
| | trifluoropropoxy)phenyl)ethan-1- | | | | | —Me | |
| | amine | | | | | | |
| I-6 | 2-(4-(3,3-difluoropropoxy)-2,5- | H, H | H, H | H | —OCH₂CH₂CF₂H | —Me, | H, H |
| | dimethoxyphenyl)ethan-1-amine | | | | | —Me | |
| I-7 | 2-(4-(3-fluoropropoxy)-2,5- | H, H | H, H | H | —OCH₂CH₂CFH₂ | —Me, | H, H |
| | dimethoxyphenyl)ethan-1-amine | | | | | —Me | |
| I-8 | 2-(4-((difluoromethyl)thio)-2,5- | H, H | H, H | H | —SCF₂H | —Me, | H, H |
| | dimethoxyphenyl)ethan-1-amine | | | | | —Me | |
| I-9 | 2-(4-((fluoromethyl)thio)-2,5- | H, H | H, H | H | —SCFH₂ | —Me, | H, H |
| | dimethoxyphenyl)ethan-1-amine | | | | | —Me | |
| I-10 | 2-(2,5-dimethoxy-4-((2,2,3,3- | H, H | H, H | H | —SCH₂CF₂CF₂H | —Me, | H, H |
| | tetrafluoropropyl)thio)phenyl)ethan-1- | | | | | —Me | |
| | amine | | | | | | |
| I-11 | 2-(2,5-dimethoxy-4-(prop-2-yn-1- | H, H | H, H | H | —SCH₂C≡CH | —Me, | H, H |
| | ylthio)phenyl)ethan-1-amine | | | | | —Me | |
| I-12 | 2-(4-(ethynylthio)-2,5- | H, H | H, H | H | —SC≡CH | —Me, | H, H |
| | dimethoxyphenyl)ethan-1-amine | | | | | —Me | |
| I-13 | 2-(4-cyclopentyl-2,5- | H, H | H, H | H | —C₅H₉ | —Me, | H, H |
| | dimethoxyphenyl)ethan-1-amine | | | | | —Me | |
| II-1 | 2-(2,5-bis(methoxy-d3)-4- | H, H | H, H | H | —SMe | —CD₃, | H, H |
| | (methylthio)phenyl)ethan-1-amine | | | | | —CD₃ | |
| II-2 | 2-(2,5-bis(methoxy-d3)-4- | H, H | H, H | H | —Me | —CD₃, | H, H |
| | methylphenyl)ethan-1-amine | | | | | —CD₃ | |
| II-3 | 2-(4-(tert-butyl)-2,5-bis(methoxy- | H, H | H, H | H | -t-Bu | —CD₃, | H, H |
| | d3)phenyl)ethan-1-amine | | | | | —CD₃ | |
| II-4 | 2-(4-cyclopentyl-2,5-bis(methoxy- | H, H | H, H | H | —C₅H₉ | —CD₃, | H, H |
| | d3)phenyl)ethan-1-amine | | | | | —CD₃ | |
| II-5 | 2-(2,5-bis(methoxy-d3)-4- | D, D | H, H | H | —OCF₃ | —CD₃, | H, H |
| | (trifluoromethoxy)phenyl)ethan-1,1- | | | | | —CD₃ | |
| | d2-1-amine | | | | | | |
| II-6 | 2-(2,4,5-tris(methoxy- | D, D | H, H | H | —OCD₃ | —CD₃, | H, H |
| | d3)phenyl)ethan-1,1-d2-1-amine | | | | | —CD₃ | |
| II-7 | 2-(4-bromo-2,5-bis(methoxy- | H, H | H, H | H | —Br | —CD₃, | H, H |
| | d3)phenyl)ethan-1-amine | | | | | —CD₃ | |
| II-8 | 2-(4-bromo-2,5-bis(methoxy- | D, D | H, H | H | —Br | —CD₃, | H, H |
| | d3)phenyl)ethan-1,1-d2-1-amine | | | | | —CD₃ | |
| II-9 | 2-(4-iodo-2,5-bis(methoxy- | H, H | H, H | H | —I | —CD₃, | H, H |
| | d3)phenyl)ethan-1-amine | | | | | —CD₃ | |
| II-10 | 2-(2,5-bis(methoxy-d3)-4- | H, H | H, H | H | —SCF₃ | —CD₃, | H, H |
| | ((trifluoromethyl)thio)phenyl)ethan-1- | | | | | —CD₃ | |
| | amine | | | | | | |
| II-11 | 2-(2,5-bis(methoxy-d3)-4- | D, D | H, H | H | —S—n—Pr | —CD₃, | H, H |
| | (propylthio)phenyl)ethan-1,1-d2-1- | | | | | —CD₃ | |
| | amine | | | | | | |
| II-12 | 2-(2,5-bis(methoxy-d3)-4- | D, D | D, D | H | —OCF₃ | —CD₃, | H, H |
| | (trifluoromethoxy)phenyl)ethan- | | | | | —CD₃ | |
| | 1,1,2,2-d4-1-amine | | | | | | |
| II-13 | 2-(2,4,5-tris(methoxy- | D, D | D, D | H | —OCD₃ | —CD₃, | H, H |
| | d3)phenyl)ethan-1,1,2,2-d4-1-amine | | | | | —CD₃ | |
| II-14 | 2-(4-bromo-2,5-bis(methoxy- | D, D | D, D | H | —Br | —CD₃, | H, H |
| | d3)phenyl)ethan-1,1,2,2-d4-1-amine | | | | | —CD₃ | |
| II-15 | 2-(2,5-bis(methoxy-d3)-4- | D, D | D, D | H | —S—n—Pr | —CD₃, | H, H |
| | (propylthio)phenyl)ethan-1,1,2,2-d4- | | | | | —CD₃ | |
| | 1-amine | | | | | | |
| II-16 | 2-(2,5-bis(methoxy-d3)-4-((3,3,3- | H, H | H, H | H | —SCH₂CH₂CF₃ | —CD₃, | H, H |
| | trifluoropropyl)thio)phenyl)ethan-1- | | | | | —CD₃ | |
| | amine | | | | | | |
| II-17 | 2-(4-((3,3-difluoropropyl)thio)-2,5- | H, H | H, H | H | —SCH₂CH₂CF₂H | —CD₃, | H, H |
| | bis(methoxy-d3)phenyl)ethan-1- | | | | | —CD₃ | |
| | amine | | | | | | |
| II-18 | 2-(4-((3-fluoropropyl)thio)-2,5- | H, H | H, H | H | —SCH₂CH₂CFH₂ | —CD₃, | H, H |
| | bis(methoxy-d3)phenyl)ethan-1- | | | | | —CD₃ | |
| | amine | | | | | | |
| II-19 | 2-(2,5-bis(methoxy-d3)-4-(3,3,3- | H, H | H, H | H | —OCH₂CH₂CF₃ | —CD₃, | H, H |
| | trifluoropropoxy)phenyl)ethan-1- | | | | | —CD₃ | |
| | amine | | | | | | |
| II-20 | 2-(4-(3,3-difluoropropoxy)-2,5- | H, H | H, H | H | —OCH₂CH₂CF₂H | —CD₃, | H, H |
| | bis(methoxy-d3)phenyl)ethan-1- | | | | | —CD₃ | |
| | amine | | | | | | |
| II-21 | 2-(4-(3-fluoropropoxy)-2,5- | H, H | H, H | H | —OCH₂CH₂CFH₂ | —CD₃, | H, H |
| | bis(methoxy-d3)phenyl)ethan-1- | | | | | —CD₃ | |
| | amine | | | | | | |
| II-22 | 2-(4-((difluoromethyl)thio)-2,5- | H, H | H, H | H | —SCF₂H | —CD₃, | H, H |
| | bis(methoxy-d3)phenyl)ethan-1- | | | | | —CD₃ | |
| | amine | | | | | | |
| II-23 | 2-(2,5-bis(methoxy-d3)-4- | H, H | H, H | H | —CF₃ | —CD₃, | H, H |
| | (trifluoromethyl)phenyl)ethan-1-amine | | | | | —CD₃ | |
| III-1 | 2-(2,5-dimethoxy-4-(methyl- | H, H | H, H | H | —CD₃ | —Me, | H, H |
| | d3)phenyl)ethan-1-amine | | | | | —Me | |
| III-2 | 2-(2,5-dimethoxy-4-(2-(methyl- | H, H | H, H | H | —C(CD₃)₃ | —Me, | H, H |
| | d3)propan-2-yl-1,1,1,3,3,3- | | | | | —Me | |
| | d6)phenyl)ethan-1-amine | | | | | | |
| IV-1 | 2-(2,5-dimethoxy-4- | D, D | H, H | H | —SMe | —Me, | H, H |
| | (methylthio)phenyl)ethan-1,1-d2-1- | | | | | —Me | |
| | amine | | | | | | |
| IV-2 | 2-(2,5-dimethoxy-4- | D, D | H, H | H | —S—n—Pr | —Me, | H, H |
| | (propylthio)phenyl)ethan-1,1-d2-1- | | | | | —Me | |
| | amine | | | | | | |
| IV-3 | 2-(2,5-dimethoxy-4- | D, D | H, H | H | —CF₃ | —Me, | H, H |
| | (trifluoromethyl)phenyl)ethan-1,1-d2- | | | | | —Me | |
| | 1-amine | | | | | | |
| IV-4 | 2-(2,4,5-trimethoxyphenyl)ethan-1,1- | D, D | H, H | H | —OMe | —Me, | H, H |
| | d2-1-amine | | | | | —Me | |
| IV-5 | 2-(4-bromo-2,5- | D, D | H, H | H | —Br | —Me, | H, H |
| | dimethoxyphenyl)ethan-1,1-d2-1- | | | | | —Me | |
| | amine | | | | | | |
| IV-6 | 2-(2,5-dimethoxy-4- | D, D | H, H | H | —SCF₃ | —Me, | H, H |
| | ((trifluoromethyl)thio)phenyl)ethan- | | | | | —Me | |
| | 1,1-d2-1-amine | | | | | | |
| IV-7 | 2-(2,5-dimethoxy-4- | D, D | H, H | H | —OCF₃ | —Me, | H, H |
| | (trifluoromethoxy)phenyl)ethan-1,1- | | | | | —Me | |
| | d2-1-amine | | | | | | |
| IV-8 | 2-(2,5-dimethoxy-4- | D, D | D, D | H | —SMe | —Me, | H, H |
| | (methylthio)phenyl)ethan-1,1,2,2-d4- | | | | | —Me | |
| | 1-amine | | | | | | |
| IV-9 | 2-(2,5-dimethoxy-4- | D, D | D, D | H | —S—n—Pr | —Me, | H, H |
| | (propylthio)phenyl)ethan-1,1,2,2-d4-1- | | | | | —Me | |
| | amine | | | | | | |
| IV-10 | 2-(2,5-dimethoxy-4- | D, D | D, D | H | —CF₃ | —Me, | H, H |
| | (trifluoromethyl)phenyl)ethan-1,1,2,2- | | | | | —Me | |
| | d4-1-amine | | | | | | |
| IV-11 | 2-(2,4,5-trimethoxyphenyl)ethan- | D, D | D, D | H | —OMe | —Me, | H, H |
| | 1,1,2,2-d4-1-amine | | | | | —Me | |
| IV-12 | 2-(4-bromo-2,5- | D, D | D, D | H | —Br | —Me, | H, H |
| | dimethoxyphenyl)ethan-1,1,2,2-d4-1- | | | | | —Me | |
| | amine | | | | | | |
| IV-13 | 2-(2,5-dimethoxy-4- | D, D | D, D | H | —SCF₃ | —Me, | H, H |
| | ((trifluoromethyl)thio)phenyl)ethan- | | | | | —Me | |
| | 1,1,2,2-d4-1-amine | | | | | | |
| IV-14 | 2-(2,5-dimethoxy-4- | D, D | D, D | H | —OCF₃ | —Me, | H, H |
| | (trifluoromethoxy)phenyl)ethan-1,1,2,2- | | | | | —Me | |
| | d4-1-amine | | | | | | |
| IV-15 | 2-(4-((difluoromethyl)thio)-2,5- | D, D | H, H | H | —SCF₂H | —Me, | H, H |
| | dimethoxyphenyl)ethan-1,1-d2-1-amine | | | | | —Me | |
| IV-16 | 2-(4-((difluoromethyl)thio)-2,5- | D, D | D, D | H | —SCF₂H | —Me, | H, H |
| | dimethoxyphenyl)ethan-1,1,2,2-d4-1- | | | | | —Me | |
| | amine | | | | | | |
| V-1 | N-(cyclopropylmethyl)-2-(2,5-dimethoxy- | H, H | H, H | H | —SCF₃ | —Me, | —H, |
| | 4-((trifluoromethyl)thio)phenyl)ethan-1- | | | | | —Me | —CH₂C₃H₅ |
| | amine | | | | | | |
| V-2 | 2-(2,5-dimethoxy-4- | H, H | H, H | H | —SCF₃ | —Me, | —H, |
| | ((trifluoromethyl)thio)phenyl)-N- | | | | | —Me | —Me |
| | methylethan-1-amine | | | | | | |
| V-3 | 2-(2,5-dimethoxy-4- | H, H | H, H | H | —SCF₃ | —Me, | —H, |
| | ((trifluoromethyl)thio)phenyl)-N- | | | | | —Me | —Et |
| | ethylethan-1-amine | | | | | | |
| V-4 | N-(2,5-dimethoxy-4- | H, H | H, H | H | —SCF₃ | —Me, | —H, |
| | ((trifluoromethyl)thio)phenethyl)propan- | | | | | —Me | -n-Pr |
| | 1-amine | | | | | | |
| V-5 | 2-(2,5-dimethoxy-4- | H, H | H, H | H | —SCF₃ | —Me, | —H, |
| | ((trifluoromethyl)thio)phenyl)-N-(methyl- | | | | | —Me | —CD₃ |
| | d3)ethan-1-amine | | | | | | |
| V-6 | 2-(2,5-bis(methoxy-d3)-4- | H, H | H, H | H | —SCF₃ | —CD₃, | —H, |
| | ((trifluoromethyl)thio)phenyl)-N- | | | | | —CD₃ | —CH₂C₃H₅ |
| | (cyclopropylmethyl)ethan-1-amine | | | | | | |
| V-7 | 2-(2,5-bis(methoxy-d3)-4- | D, D | H, H | H | —SCF₃ | —CD₃, | —H, |
| | ((trifluoromethyl)thio)phenyl)-N- | | | | | —CD₃ | —CH₂C₃H₅ |
| | (cyclopropylmethyl)ethan-1,1-d2-1-amine | | | | | | |
| V-8 | 2-(2,5-bis(methoxy-d3)-4- | D, D | D, D | H | —SCF₃ | —CD₃, | —H, |
| | ((trifluoromethyl)thio)phenyl)-N- | | | | | —CD₃ | —CH₂C₃H₅ |
| | (cyclopropylmethyl)ethan-1,1,2,2-d4-1- | | | | | | |
| | amine | | | | | | |
| V-36 | N-(cyclopropylmethyl)-2-(4- | H, H | H, H | H | —SCF₂H | —Me, | —H, |
| | ((difluoromethyl)thio)-2,5- | | | | | —Me | —CH₂C₃H₅ |
| | dimethoxyphenyl)ethan-1-amine | | | | | | |

In some embodiments, where R⁴ is —OR^{b} or —SR^{b}, R^{b} is not a substituted C₂ alkyl group, e.g., a C₂ fluoroalkyl group. In some embodiments, when X¹, X², Y¹, Y², R³, R⁶, and R⁷ are each hydrogen and each R^{a} is methyl, R⁴ is not —SCH₂CH₂CFH₂, —SCH₂C≡CH₁—SCFH₂, —SCF₂H, or —SCF₃. In some embodiments, R⁴ is not —SCH₂CH₂CFH₂, for example when X¹, X², Y¹, Y², R³, R⁶, and R⁷ are each hydrogen and each R^{a} is methyl (e.g., in some embodiments the compound is not compound I-4). In some embodiments, R⁴ is not —SCH₂C≡CH, for example when X¹, X², Y¹, Y², R³, R⁶, and R⁷ are each hydrogen and each R^{a} is methyl (e.g., in some embodiments the compound is not compound I-11). In some embodiments, R⁴ is not —SCFH₂, for example when X¹, X², Y¹, Y², R³, R⁶, and R⁷ are each hydrogen and each R^{a} is methyl (e.g., in some embodiments the compound is not compound I-9). In some embodiments, R⁴ is not —SCF₂H, for example when X¹, X², Y¹, Y², R³, R⁶, and R⁷ are each hydrogen and each R^{a} is methyl (e.g., in some embodiments the compound is not compound I-8). In some embodiments, R⁴ is not —SCF₃, for example when X¹, X², Y¹, Y², R³, R⁶, and R⁷ are each hydrogen and each R^{a} is methyl (e.g., in some embodiments the compound is not compound I-1).

### Formula (VI)

Disclosed herein is a compound according to Formula (VI):

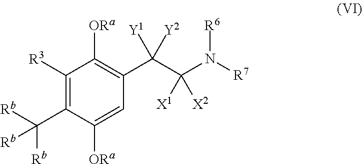

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R³ is hydrogen or deuterium;
R⁶ and R⁷ are independently hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl; or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
each R^{b} is independently a substituted or unsubstituted C₁-C₂ alkyl; or alternatively two R^{b}s together with the carbon atom attached thereto are optionally joined to form a substituted or unsubstituted cycloalkyl.

X¹ and X² may be the same, or different. In some embodiments, X¹ and X² are the same. In some embodiments, X¹ and X² are hydrogen. In some embodiments, X¹ and X² are deuterium.

Y¹ and Y² may be the same, or different. In some embodiments, Y¹ and Y² are the same. In some embodiments, Y¹ and Y² are hydrogen. In some embodiments, Y¹ and Y² are deuterium.

In some embodiments, R³ is deuterium. In some embodiments, R³ is hydrogen.

R⁶ and R⁷ may be the same, or different. In some embodiments, R⁶ and R⁷ are the same. For example, in some embodiments, both R⁶ and R⁷ are hydrogen. In some embodiments, R⁶ and R⁷ are different. For example, in some embodiments, R⁶ is hydrogen, and R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl.

In some embodiments, R⁶ and/or R⁷ is an unsubstituted C₁-C₆ alkyl, for example, an unsubstituted C₁ alkyl, an unsubstituted C₂ alkyl, an unsubstituted C₃ alkyl, an unsubstituted C₄ alkyl, an unsubstituted C₅ alkyl, or an unsubstituted C₆ alkyl. In some embodiments, R⁶ and/or R⁷ is an unsubstituted linear C₂-C₆ alkyl. In some embodiments, R⁶ and/or R⁷ is an unsubstituted branched C₃-C₁₀ alkyl. Examples of an unsubstituted C₁-C₁₀ alkyl include, but are not limited to, methyl, ethyl, n-propyl, isopropyl, butyl, isobutyl, t-butyl, n-pentyl, isopentyl, n-hexyl, and isohexyl.

In some embodiments, R⁶ and/or R⁷ is a substituted C₁-C₆ alkyl, e.g., a substituted C₁ alkyl, a substituted C₂ alkyl, a substituted C₃ alkyl, a substituted C₄ alkyl, a substituted C₅ alkyl, or a substituted C₆ alkyl. The alkyl group may contain one, or more than one, substituent. The alkyl group may be substituted with any one or more substituents recited herein, examples of those substituents may include, but are not limited to, deuterium, halogen (e.g., fluorine), polar substituents such as hydroxyl, oxo, unsubstituted alkoxy, substituted alkoxy (e.g., polyether groups), unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. In some embodiments, R⁶ and/or R⁷ is a C₁-C₆ alkyl substituted with one or more deuterium atoms, examples of which include, but are not limited to, —CDH₂, —CD₂H, —CD₃, —CD₂CD₃, and —CD₂CD₂CD₃. In some embodiments, R⁶ and/or R⁷ is a C₁-C₆ alkyl substituted with one or more fluorine atoms, i.e., is a fluoroalkyl group. Examples of fluoroalkyl groups include, but are not limited to, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, —CH₂CH₂CH₂CF₃, —CH₂CF₂CHF₂, —CH₂CF₂CF₃, —CH(CF₃)₂, and —CH(CH₃)CF₃.

In some embodiments, R⁶ and/or R⁷ is a C₁-C₆ alkyl substituted with one or more deuterium atoms and one or more fluorine atoms, examples of which include, but are not limited to, —CD₂CH₂F, —CD₂CHF₂, —CD₂CF₃, —CD₂CH₂CH₂F, —CD₂CH₂CHF₂, —CD₂CH₂CF₃, —CD₂CD₂CH₂, —CD₂CD₂CHF₂, —CD₂CD₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃.

In some embodiments, R⁶ and/or R⁷ is a C₁-C₆ alkyl substituted with a substituted or unsubstituted cycloalkyl. The C₁-C₆ alkyl may be substituted with, e.g., a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, the C₁-C₆ alkyl is substituted with an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, the C₁-C₆ alkyl is substituted with a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent. In some embodiments, R⁶ and/or R⁷ is a C₁ alkyl substituted with a substituted or unsubstituted cycloalkyl, with particular mention being made to cyclopropylmethyl (—CH₂C₃H₅).

In some embodiments, R⁶ and/or R⁷ is a substituted or unsubstituted alkenyl, e.g., a substituted or unsubstituted allyl, butenyl, crotyl, etc.

In some embodiments, R⁶ and/or R⁷ is a substituted or unsubstituted alkynyl, e.g., a substituted or unsubstituted propargyl.

In some embodiments, R⁶ and/or R⁷ is a substituted or unsubstituted cycloalkyl, for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, R⁶ and/or R⁷ is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R⁶ and/or R⁷ is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁶ and/or R⁷ is a substituted or unsubstituted heterocycloalkyl. In some embodiments, the unsubstituted or substituted heterocycloalkyl group may be a 3-membered ring, a 4-membered ring, a 5-membered ring, a 6-membered ring, a 7-membered ring, or an 8-membered ring. In some embodiments, R⁶ and/or R⁷ is an unsubstituted heterocycloalkyl, such as those set forth herein, examples of which include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, tetrahydrofuran, tetrahydropyran, and 1,3-dioxolane. In some embodiments, R⁶ and/or R⁷ is a substituted heterocycloalkyl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), oxo, and hydroxyl. The heterocycloalkyl group may contain one, or more than one, substituent.

In some embodiments, R⁶ and/or R⁷ is a substituted or unsubstituted aryl. In some embodiments, R⁶ and/or R⁷ is an unsubstituted aryl, examples of which include, but are not limited to, phenyl and naphthyl. In some embodiments, R⁶ and/or R⁷ is a substituted aryl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), hydroxyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The aryl group may contain one, or more than one, substituent.

In some embodiments, R⁶ and/or R⁷ is a substituted or unsubstituted heteroaryl. In some embodiments, R⁶ and/or R⁷ is an unsubstituted heteroaryl, examples of which include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothiophenyl, thiophenyl, benzimidazolyl, quinolinyl, isoquinolinyl, quinazolinyl, quinoxalinyl, and pyrazolyl. In some embodiments, R⁶ and/or R⁷ is a substituted heteroaryl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), hydroxyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The heteroaryl group may contain one, or more than one, substituent.

In some embodiments, R⁶ is hydrogen, and R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl. In some embodiments, R⁶ is hydrogen, and R⁷ is a substituted or unsubstituted C₁-C₆ alkyl. In some embodiments, R⁶ is hydrogen, and R⁷ is an unsubstituted C₁-C₆ alkyl, a C₁-C₆ alkyl substituted with one or more deuterium atoms, a C₁-C₆ alkyl substituted with one or more fluorine atoms, or a C₁-C₆ alkyl substituted with a substituted or unsubstituted cycloalkyl. For example, in some embodiments, R⁶ is hydrogen, and R⁷ is methyl, ethyl, propyl, —CD₃, or cyclopropylmethyl (—CH₂C₃H₅).

In some embodiments, R⁶ and R⁷ together with the nitrogen atom attached thereto are joined to form a substituted or unsubstituted heterocycloalkyl. In some embodiments, R⁶ and R⁷ together with the nitrogen atom attached thereto are joined to form an unsubstituted heterocycloalkyl. The unsubstituted heterocycloalkyl group may be, e.g., a 3-membered ring, a 4-membered ring, 5-membered ring, a 6-membered ring, a 7-membered ring, an 8-membered ring, etc., which may be optionally fused to other ring(s). The unsubstituted heterocycloalkyl group contains a minimum of one nitrogen ring atom (the nitrogen atom intervening R⁶ and R⁷), and may optionally contain at least one additional hetero-ring atom, which may be one or more of nitrogen, sulfur, or oxygen, for a total of 1, 2, 3, or 4 hetero-ring atoms (at least one of which is a nitrogen ring atom). Examples of unsubstituted heterocycloalkyl groups formed from joining R⁶ and R⁷ together with the nitrogen atom attached thereto include, but are not limited to,

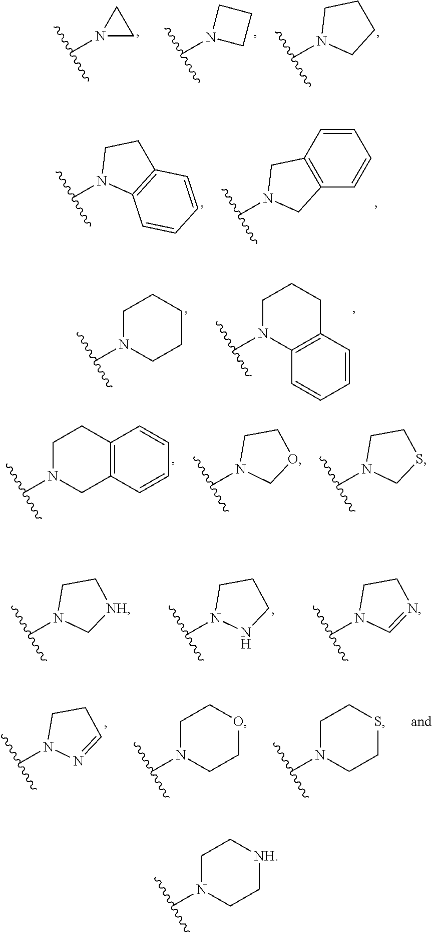

In some embodiments, R⁶ and R⁷ together with the nitrogen atom attached thereto are joined to form a substituted heterocycloalkyl. The substituted heterocycloalkyl group may be, e.g., a 3-membered ring, a 4-membered ring, a 5-membered ring, a 6-membered ring, a 7-membered ring, an 8-membered ring, etc., which may be optionally fused to other ring(s). The substituted heterocycloalkyl group contains a minimum of one nitrogen ring atom (the nitrogen atom intervening R⁶ and R⁷), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the substituted heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, 1,2,3,4-tetrahydroisoquinoline, thiazolidine, morpholine, or thiomorpholine, which is substituted with at least one substituent. The substituent(s) may be any recited herein, including, but not limited to, deuterium, halogen (e.g., fluorine), polar substituents such as hydroxyl, oxo, unsubstituted alkoxy, substituted alkoxy (e.g., polyether groups), unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The substituted heterocycloalkyl formed from joining R⁶ and R⁷ together with the nitrogen atom attached thereto contains a heterocycloalkyl group substituted with one, two, three, four, or more substituents. The substituent may be located on a carbon ring atom or on a hetero-ring atom.

Examples of substituted heterocycloalkyl groups formed from joining R⁶ and R⁷ together with the nitrogen atom attached thereto include, but are not limited to,

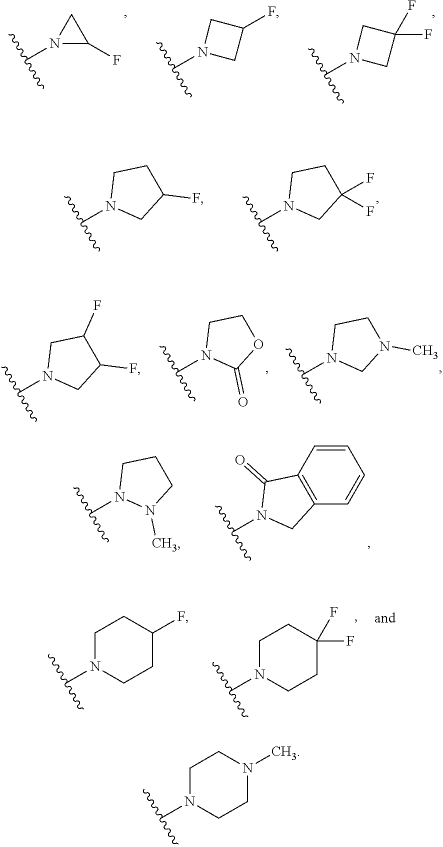

Each R^{a} may be the same, or different. In some embodiments, each R^{a} is the same. Each R^{a} may be, independently, a substituted or unsubstituted C₁-C₆ alkyl, preferably a substituted or unsubstituted C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, each R^{a} is different, e.g., one R^{a} is —CH₃, while another is —CD₃.

Each R^{b} may be the same, or different. In some embodiments, each R^{b} is the same. Each R^{b} may be, independently, a substituted or unsubstituted C₁-C₂ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{b} is selected from the group consisting of —CDH₂, —CD₂H, —CD₃, and —CH₃. In some embodiments, each R^{b} is —CH₃. In some embodiments, each R^{b} is —CD₃. In some embodiments, at least one R^{b} is different from the others, e.g., one R^{b} is —CH₂CH₃, while the other two are —CH₃. In some embodiments, one R^{b} is —CF₃, while the other two are —CH₃. In some embodiments, one R^{b} is —CH₃, while the other two are —CF₃.

In some embodiments, two R^{b}s together with the carbon atom attached thereto are joined to form a substituted or unsubstituted cycloalkyl group. In some embodiments, the substituted or unsubstituted cycloalkyl group formed from joining two R^{b}s, together with the carbon atom attached thereto, may be for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, two R^{b}s, together with the carbon atom attached thereto, are joined to form an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, two R^{b}s, together with the carbon atom attached thereto, are joined to form a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent. When two R^{b}s together with the carbon atom attached thereto are joined to form a substituted or unsubstituted cycloalkyl group, the remaining R^{b} is selected as set forth above, e.g., —CH₃, —CDH₂, —CD₂H, —CD₃, —CFH₂, —CF₂H, or —CF₃.

In some embodiments, Y¹ and Y² are each hydrogen or each deuterium; R³ is hydrogen; X¹ and X² are each hydrogen or each deuterium; each R^{a} is —CH₃ or —CD₃; and each R^{b} is —CD₃, or —CH₃.

In some embodiments, the compound, e.g., the compound of Formula (VI) is selected from the group consisting of:

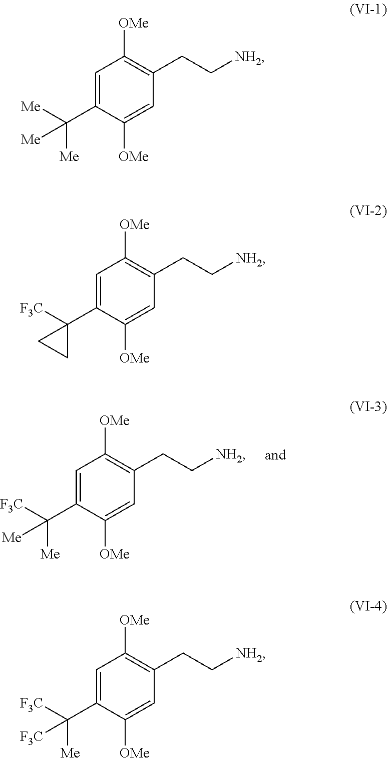 [SMILES: COc1cc(C(C)(C)C)c(OC)cc1CCN ; COc1cc(C2(C(F)(F)F)CC2)c(OC)cc1CCN ; COc1cc(C(C)(C)C(F)(F)F)c(OC)cc1CCN ; COc1cc(C(C)(C(F)(F)F)C(F)(F)F)c(OC)cc1CCN]

or a pharmaceutically acceptable salt, solvate, or prodrug thereof.

The compounds of Formula (VI) containing a quaternary carbon atom attached to the 4-position of the phenyl ring, in particular a lipophilic tertiary alkyl group in the 4-position of the phenyl ring (e.g., a t-butyl group) have been found to possess a surprisingly strong binding affinity to 5-HT₂ receptors such as 5-HT_{2A}, especially when compared to compounds lacking a quaternary carbon at the same position, such as the case in cyclopentyl groups. In addition to their excellent pharmacodynamic properties, the compounds of Formula (VI), bearing a quaternary carbon atom attached to the 4-position of the phenyl ring, may also provide advantageous pharmacokinetics by slowing or shunting metabolism at this position, for improved bioavailability, exposure, and brain penetrability.

### Formula (VII)

Disclosed herein is a compound according to Formula (VII):

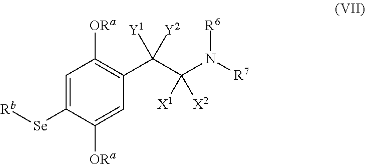

or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁶ is hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl.

X¹ and X² may be the same, or different. In some embodiments, X¹ and X² are the same. In some embodiments, X¹ and X² are hydrogen. In some embodiments, X¹ and X² are deuterium.

Y¹ and Y² may be the same, or different. In some embodiments, Y¹ and Y² are the same. In some embodiments, Y¹ and Y² are hydrogen. In some embodiments, Y¹ and Y² are deuterium.

In some embodiments, R^{b} is a substituted or unsubstituted C₁-C₆ alkyl. In some embodiments, R^{b} is an unsubstituted C₁-C₆ alkyl, examples of which include, but are not limited to, methyl, ethyl, n-propyl, isopropyl, n-butyl, isobutyl, sec-butyl, t-butyl, n-pentyl, neopentyl, and hexyl. In some embodiments, R^{b} is a substituted C₁-C₆ alkyl. Preferred substituents may include, but are not limited to, deuterium, halogen (e.g., fluorine), cyano, polar substituents such as hydroxyl or polyether substituents, etc. The C₁-C₆ alkyl group may contain one, or more than one, substituent. In some embodiments, R^{b} is a substituted C₁ alkyl group, examples of which may include, but are not limited to, —CDH₂, —CD₂H, —CD₃, —CFH₂, —CF₂H, —CF₃, and —CH₂C≡N. In some embodiments, R^{b} is a substituted C₂ alkyl group, examples of which may include, but are not limited to, —CDHCDH₂, —CDHCD₂H, —CD₂CD₃, —CH₂CFH₂, —CH₂CF₂H, —CH₂CF₃, and —CH₂CH₂C≡N. In some embodiments, R^{b} is not a substituted C₂ alkyl group such as a C₂ fluoroalkyl group. For example, in some embodiments when R⁴ is —OR^{b} or —SR^{b}, R^{b} is not —CH₂CFH₂, —CH₂CF₂H, or —CH₂CF₃. In some embodiments, R^{b} is a substituted C₃ alkyl group, examples of which may include, but are not limited to —CH₂CH₂CF₃, —CH₂CH₂CF₂H, —CH₂CH₂CFH₂, —CH₂CF₂CF₂H, and —CH₂CH₂CH₂C≡N. In some embodiments, R^{b} is not —CH₂CH₂CFH₂.

In some embodiments, R^{b} is a substituted or unsubstituted alkenyl, e.g., a substituted or unsubstituted allyl, butenyl, crotyl, etc.

In some embodiments, R^{b} is a substituted or unsubstituted alkynyl, e.g., a substituted or unsubstituted acetylenyl, propargyl, homopropargyl, etc. In some embodiments, R^{b} is an unsubstituted alkynyl. In some embodiments, R^{b} is an unsubstituted acetylenyl (—C≡CH). In some embodiments, R^{b} is an unsubstituted propargyl (—CH₂C≡CH). In some embodiments, R^{b} is —CH₂CH₂C≡CH. In some embodiments, R^{b} is —CH₂CH₂CH₂C≡CH. In some embodiments, R^{b} is —CH₂CH₂CH₂CH₂C≡CH. In some embodiments, R^{b} is a substituted alkynyl. In some embodiments, R^{b} is a substituted propargyl (e.g., —CF₂C≡CH). In some embodiments, R^{b} is —CF₂CH₂C≡CH. In some embodiments, R^{b} is —CF₂CH₂CH₂C≡CH. In some embodiments, R^{b} is —CF₂CH₂CH₂CH₂C≡CH.

In some embodiments, R^{b} is a substituted or unsubstituted cycloalkyl, for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, R^{b} is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R^{b} is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R^{b} is —C≡CH, —CH₂C≡N, —CH₂C≡CH, —CF₂C≡CH, —CH₂CH₂C≡CH, —CF₂CH₂C≡CH, —CH₂CH₂CH₂C≡CH, —CF₂CH₂CH₂C≡CH, —CH₂CH₂CH₂CH₂C≡CH, and —CF₂CH₂CH₂CH₂C≡CH. R⁶ and R⁷ may be the same, or different. In some embodiments, R⁶ and R⁷ are the same. In some embodiments, R⁶ and R⁷ are different. For example, in some embodiments, R⁶ is hydrogen, and R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl.

In some embodiments, R⁶ and/or R⁷ is an unsubstituted C₁-C₆ alkyl, for example, an unsubstituted C₁ alkyl, an unsubstituted C₂ alkyl, an unsubstituted C₃ alkyl, an unsubstituted C₄ alkyl, an unsubstituted C₅ alkyl, or an unsubstituted C₆ alkyl. In some embodiments, R⁶ and/or R⁷ is an unsubstituted linear C₂-C₆ alkyl. In some embodiments, R⁶ and/or R⁷ is an unsubstituted branched C₃-C₁₀ alkyl. Examples of an unsubstituted C₁-C₁₀ alkyl include, but are not limited to, methyl, ethyl, n-propyl, isopropyl, butyl, isobutyl, t-butyl, n-pentyl, isopentyl, n-hexyl, and isohexyl.

In some embodiments, R⁶ and/or R⁷ is a substituted C₁-C₆ alkyl, e.g., a substituted C₁ alkyl, a substituted C₂ alkyl, a substituted C₃ alkyl, a substituted C₄ alkyl, a substituted C₅ alkyl, or a substituted C₆ alkyl. The alkyl group may contain one, or more than one, substituent. The alkyl group may be substituted with any one or more substituents recited herein, examples of those substituents may include, but are not limited to, deuterium, halogen (e.g., fluorine), polar substituents such as hydroxyl, oxo, unsubstituted alkoxy, substituted alkoxy (e.g., polyether groups), unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. In some embodiments, R⁶ and/or R⁷ is a C₁-C₆ alkyl substituted with one or more deuterium atoms, examples of which include, but are not limited to, —CDH₂, —CD₂H, —CD₃, —CD₂CD₃, and —CD₂CD₂CD₃. In some embodiments, R⁶ and/or R⁷ is a C₁-C₆ alkyl substituted with one or more fluorine atoms, i.e., is a fluoroalkyl group. Examples of fluoroalkyl groups include, but are not limited to, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂CH₂CH₂CH₂F, —CH₂CH₂CH₂CHF₂, —CH₂CH₂CH₂CF₃, —CH₂CF₂CHF₂, —CH₂CF₂CF₃, —CH(CF₃)₂, and —CH(CH₃)CF₃.

In some embodiments, R⁶ and/or R⁷ is a C₁-C₆ alkyl substituted with one or more deuterium atoms and one or more fluorine atoms, examples of which include, but are not limited to, —CD₂CH₂F, —CD₂CHF₂, —CD₂CF₃, —CD₂CH₂CH₂F, —CD₂CH₂CHF₂, —CD₂CH₂CF₃, —CD₂CD₂CH₂, —CD₂CD₂CHF₂, —CD₂CD₂CF₃, —CD₂CH₂CH₂CH₂F, —CD₂CH₂CH₂CHF₂, —CD₂CH₂CH₂CF₃, —CD₂CD₂CH₂CH₂F, —CD₂CD₂CH₂CHF₂, —CD₂CD₂CH₂CF₃, —CD₂CD₂CD₂CH₂F, —CD₂CD₂CD₂CHF₂, and —CD₂CD₂CD₂CF₃.

In some embodiments, R⁶ and/or R⁷ is a C₁-C₆ alkyl substituted with a substituted or unsubstituted cycloalkyl. The C₁-C₆ alkyl may be substituted with, e.g., a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, the C₁-C₆ alkyl is substituted with an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, the C₁-C₆ alkyl is substituted with a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent. In some embodiments, R⁶ and/or R⁷ is a C₁ alkyl substituted with a substituted or unsubstituted cycloalkyl, with particular mention being made to cyclopropylmethyl (—CH₂C₃H₅).

In some embodiments, R⁶ and/or R⁷ is a substituted or unsubstituted alkenyl, e.g., a substituted or unsubstituted allyl, butenyl, crotyl, etc.

In some embodiments, R⁶ and/or R⁷ is a substituted or unsubstituted alkynyl, e.g., a substituted or unsubstituted propargyl.

In some embodiments, R⁶ and/or R⁷ is a substituted or unsubstituted cycloalkyl, for example a substituted or unsubstituted C₃-C₁₀ cycloalkyl, or a substituted or unsubstituted C₄-C₈ cycloalkyl, or a substituted or unsubstituted C₅-C₆ cycloalkyl. In some embodiments, R⁶ and/or R⁷ is an unsubstituted cycloalkyl (e.g., an unsubstituted C₃-C₁₀ cycloalkyl), examples of which may include, but are not limited to, adamantyl, cyclopropyl, cyclobutyl, cyclopentyl, cyclohexyl, cycloheptyl, and cyclooctyl. In some embodiments, R⁶ and/or R⁷ is a substituted cycloalkyl (e.g., a substituted C₃-C₁₀ cycloalkyl). The cycloalkyl group may be substituted with any one or more substituents recited herein, examples of those substituents may include, but are not limited to, deuterium, unsubstituted alkyl, substituted alkyl, unsubstituted alkoxy, substituted alkoxy (e.g., polyether groups), halogen (e.g., fluorine), hydroxyl, oxo, unsubstituted heterocycloalkyl, substituted heterocycloalkyl, unsubstituted aryl, substituted aryl, unsubstituted heteroaryl, and substituted heteroaryl. The cycloalkyl group may contain one, or more than one, substituent.

In some embodiments, R⁶ and/or R⁷ is a substituted or unsubstituted heterocycloalkyl. In some embodiments, the unsubstituted or substituted heterocycloalkyl group may be a 3-membered ring, a 4-membered ring, a 5-membered ring, a 6-membered ring, a 7-membered ring, or an 8-membered ring. In some embodiments, R⁶ and/or R⁷ is an unsubstituted heterocycloalkyl, such as those set forth herein, examples of which include, but are not limited to, aziridine, azetidine, pyrrolidine, isoindole, indole, dihydroindole, indazole, purine, carbazole, carboline, imidazolidine, imidazoline, piperidine, piperazine, indoline, phthalimide, 1,2,3,4-tetrahydroisoquinoline, thiazolidine, morpholine, thiomorpholine, tetrahydrofuran, tetrahydropyran, and 1,3-dioxolane. In some embodiments, R⁶ and/or R⁷ is a substituted heterocycloalkyl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), oxo, and hydroxyl. The heterocycloalkyl group may contain one, or more than one, substituent.

In some embodiments, R⁶ and/or R⁷ is a substituted or unsubstituted aryl. In some embodiments, R⁶ and/or R⁷ is an unsubstituted aryl, examples of which include, but are not limited to, phenyl and naphthyl. In some embodiments, R⁶ and/or R⁷ is a substituted aryl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), hydroxyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The aryl group may contain one, or more than one, substituent.

In some embodiments, R⁶ and/or R⁷ is a substituted or unsubstituted heteroaryl. In some embodiments, R⁶ and/or R⁷ is an unsubstituted heteroaryl, examples of which include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothiophenyl, thiophenyl, benzimidazolyl, quinolinyl, isoquinolinyl, quinazolinyl, quinoxalinyl, and pyrazolyl. In some embodiments, R⁶ and/or R⁷ is a substituted heteroaryl. The substituent(s) may be any recited herein, including, but not limited to, deuterium, unsubstituted alkyl, substituted alkyl, unsubstituted alkoxy, substituted alkoxy (e.g., polyether groups), halogen (e.g., fluorine), hydroxyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The heteroaryl group may contain one, or more than one, substituent.

In some embodiments, R⁶ is hydrogen, and R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl. In some embodiments, R⁶ is hydrogen, and R⁷ is a substituted or unsubstituted C₁-C₆ alkyl. In some embodiments, R⁶ is hydrogen, and R⁷ is an unsubstituted C₁-C₆ alkyl, a C₁-C₆ alkyl substituted with one or more deuterium atoms, a C₁-C₆ alkyl substituted with one or more fluorine atoms, or a C₁-C₆ alkyl substituted with a substituted or unsubstituted cycloalkyl. For example, in some embodiments, R⁶ is hydrogen, and R⁷ is methyl, ethyl, propyl, —CD₃, or cyclopropylmethyl (—CH₂C₃H₅).

In some embodiments, R⁶ and R⁷ together with the nitrogen atom attached thereto are joined to form a substituted or unsubstituted heterocycloalkyl. In some embodiments, R⁶ and R⁷ together with the nitrogen atom attached thereto are joined to form an unsubstituted heterocycloalkyl. The unsubstituted heterocycloalkyl group may be, e.g., a 3-membered ring, a 4-membered ring, 5-membered ring, a 6-membered ring, a 7-membered ring, an 8-membered ring, etc., which may be optionally fused to other ring(s). The unsubstituted heterocycloalkyl group contains a minimum of one nitrogen ring atom (the nitrogen atom intervening R⁶ and R⁷), and may optionally contain at least one additional hetero-ring atom, which may be one or more of nitrogen, sulfur, or oxygen, for a total of 1, 2, 3, or 4 hetero-ring atoms (at least one of which is a nitrogen ring atom). Examples of unsubstituted heterocycloalkyl groups formed from joining R⁶ and R⁷ together with the nitrogen atom attached thereto include, but are not limited to,

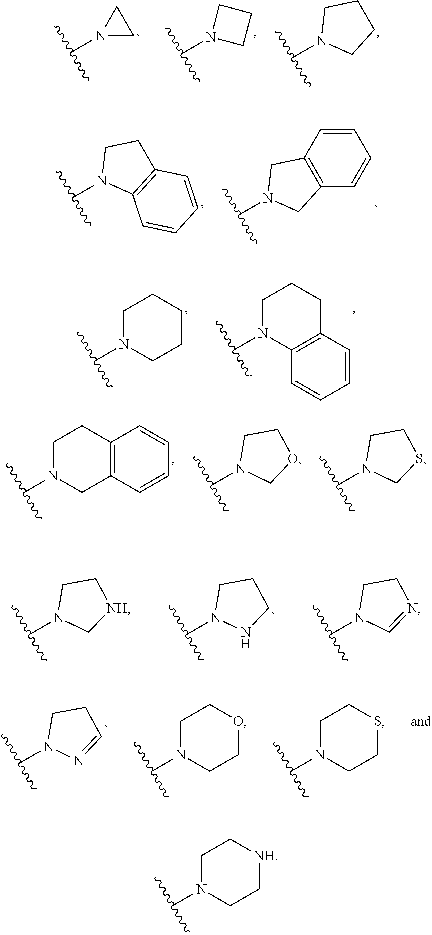

In some embodiments, R⁶ and R⁷ together with the nitrogen atom attached thereto are joined to form a substituted heterocycloalkyl. The substituted heterocycloalkyl group may be, e.g., a 3-membered ring, a 4-membered ring, a 5-membered ring, a 6-membered ring, a 7-membered ring, an 8-membered ring, etc., which may be optionally fused to other ring(s). The substituted heterocycloalkyl group contains a minimum of one nitrogen ring atom (the nitrogen atom intervening R⁶ and R⁷), and may optionally contain additional hetero-ring atoms (e.g., nitrogen, sulfur, or oxygen) for a total of 1, 2, 3, or 4 hetero-ring atoms (at least one of which is a nitrogen ring atom). Examples of the substituted heterocycloalkyl group include, but are not limited to, aziridine, azetidine, pyrrolidine, isoindole, indole, dihydroindole, indazole, purine, carbazole, carboline, imidazolidine, imidazoline, piperidine, piperazine, indoline, 1,2,3,4-tetrahydroisoquinoline, thiazolidine, morpholine, or thiomorpholine, which is substituted with at least one substituent. The substituent(s) may be any recited herein, including, but not limited to, deuterium, halogen (e.g., fluorine), polar substituents such as hydroxyl, oxo, unsubstituted alkoxy, substituted alkoxy (e.g., polyether groups), unsubstituted alkyl, substituted alkyl, unsubstituted alkenyl, substituted alkenyl, unsubstituted alkynyl, substituted alkynyl, unsubstituted cycloalkyl, substituted cycloalkyl, unsubstituted heterocycloalkyl, substituted heterocycloalkyl, unsubstituted aryl, substituted aryl, unsubstituted heteroaryl, and substituted heteroaryl. The substituted heterocycloalkyl formed from joining R⁶ and R⁷ together with the nitrogen atom attached thereto contains a heterocycloalkyl group substituted with one, two, three, four, or more substituents. The substituent may be located on a carbon ring atom or on a hetero-ring atom.

Examples of substituted heterocycloalkyl groups formed from joining R⁶ and R⁷ together with the nitrogen atom attached thereto include, but are not limited to,

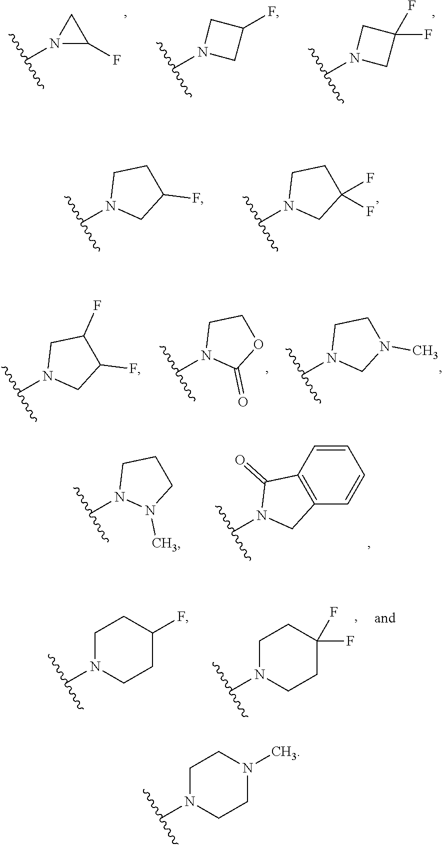

Each R^{a} may be the same, or different. In some embodiments, each R^{a} is the same. Each R^{a} may be, independently, a substituted or unsubstituted C₁-C₆ alkyl, preferably a substituted or unsubstituted C₁-C₃ alkyl, preferably a substituted or unsubstituted C₁ alkyl, examples of which include, but are not limited to, —CH₃, —CDH₂, —CD₂H, —CD₃, —CFH₂, —CF₂H, —CF₃. In some embodiments, each R^{a} is —CH₃. In some embodiments, each R^{a} is —CD₃. In some embodiments, each R^{a} is different, e.g., one R^{a} is —CH₃, while another is —CD₃.

In some embodiments, the compound, e.g., the compound of Formula (VII) is selected from the group consisting of

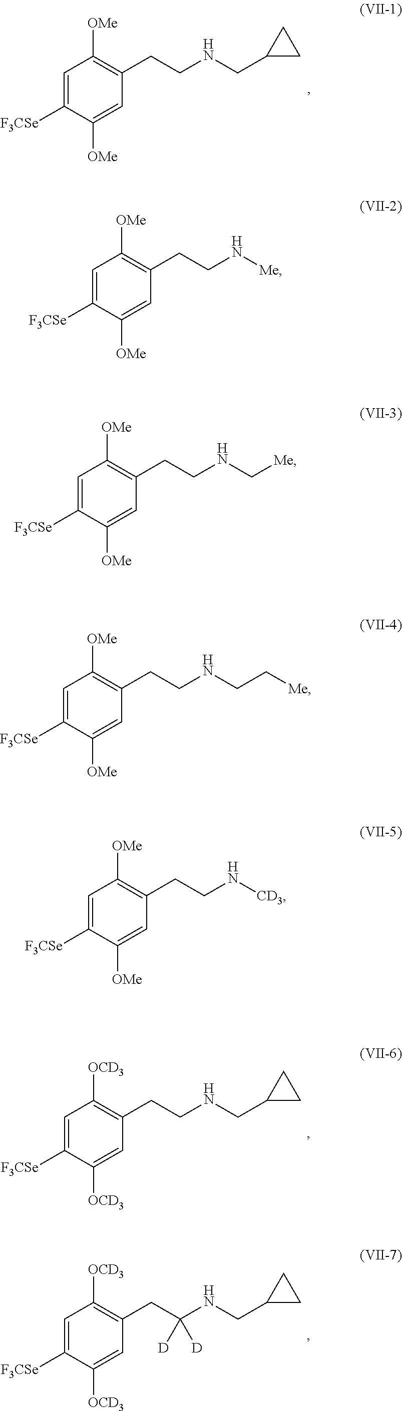

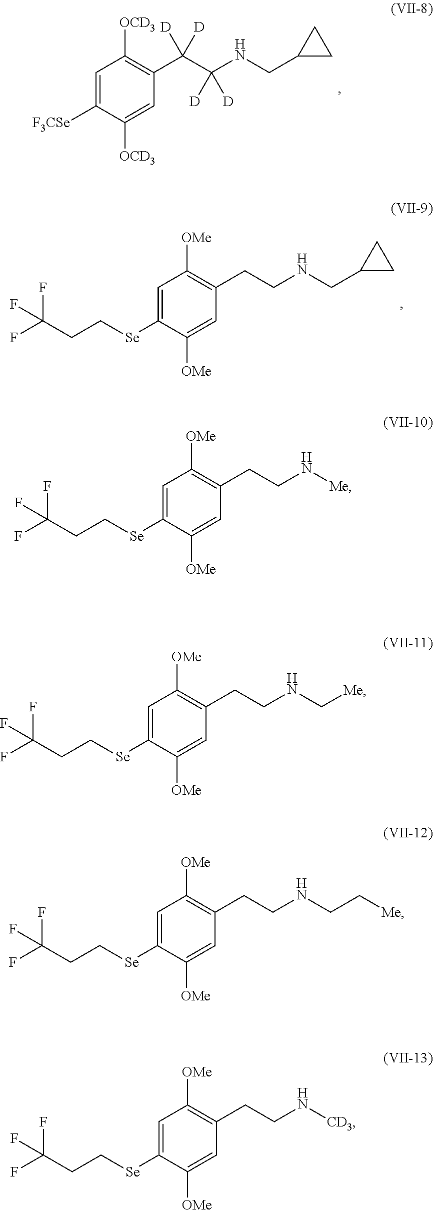

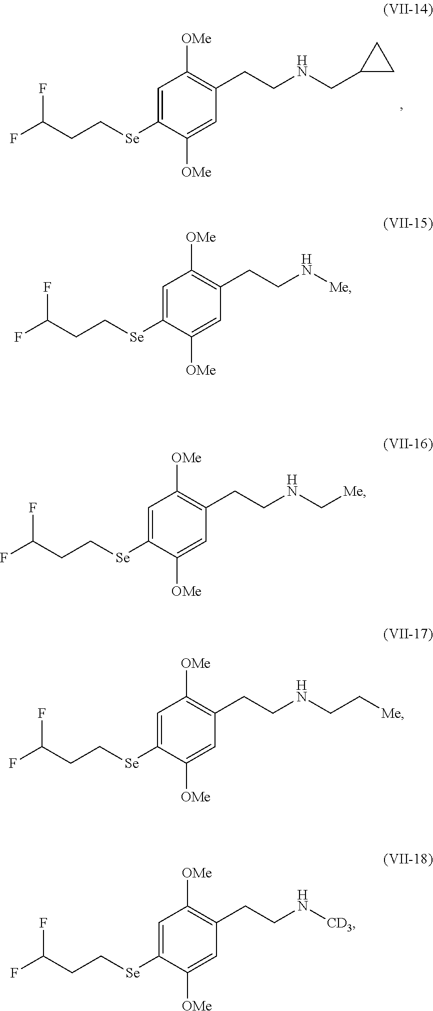 [SMILES: COc1cc([Se]CCC(F)F)c(OC)cc1CCNCC1CC1 ; CNCCc1cc(OC)c([Se]CCC(F)F)cc1OC ; CCNCCc1cc(OC)c([Se]CCC(F)F)cc1OC ; CCCNCCc1cc(OC)c([Se]CCC(F)F)cc1OC ; [2H]C([2H])([2H])NCCc1cc(OC)c([Se]CCC(F)F)cc1OC]

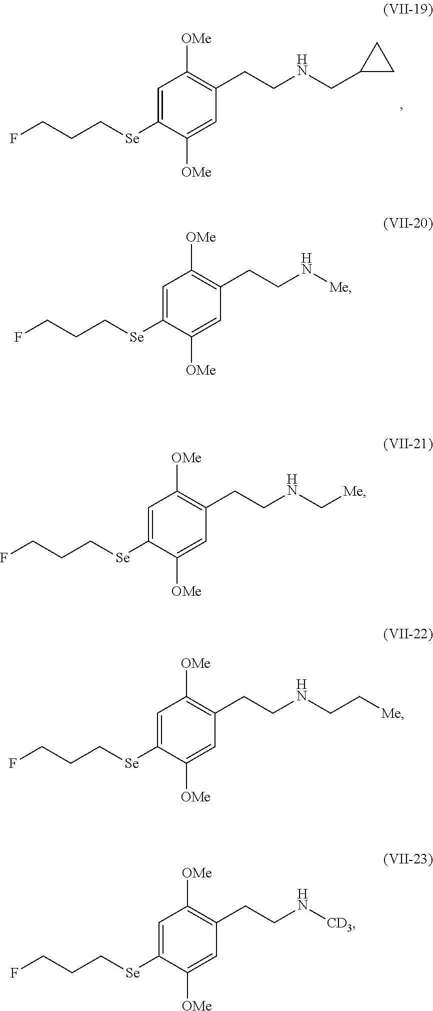 [SMILES: COc1cc([Se]CCCF)c(OC)cc1CCNCC1CC1 ; CNCCc1cc(OC)c([Se]CCCF)cc1OC ; CCNCCc1cc(OC)c([Se]CCCF)cc1OC ; CCCNCCc1cc(OC)c([Se]CCCF)cc1OC ; [2H]C([2H])([2H])NCCc1cc(OC)c([Se]CCCF)cc1OC]

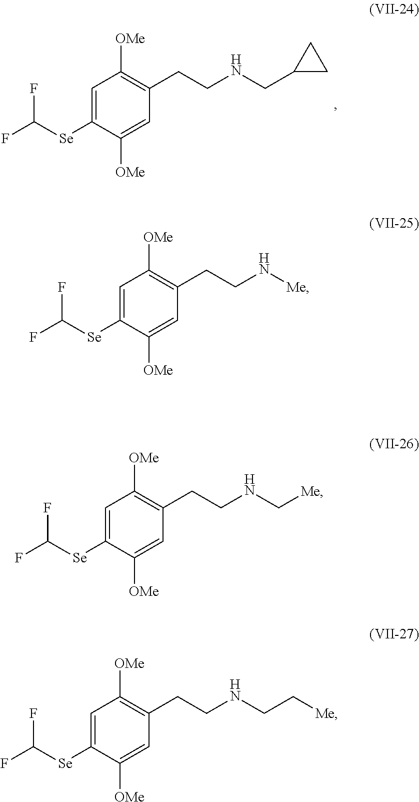 [SMILES: COc1cc([Se]C(F)F)c(OC)cc1CCNCC1CC1 ; CNCCc1cc(OC)c([Se]C(F)F)cc1OC ; CCNCCc1cc(OC)c([Se]C(F)F)cc1OC ; CCCNCCc1cc(OC)c([Se]C(F)F)cc1OC]

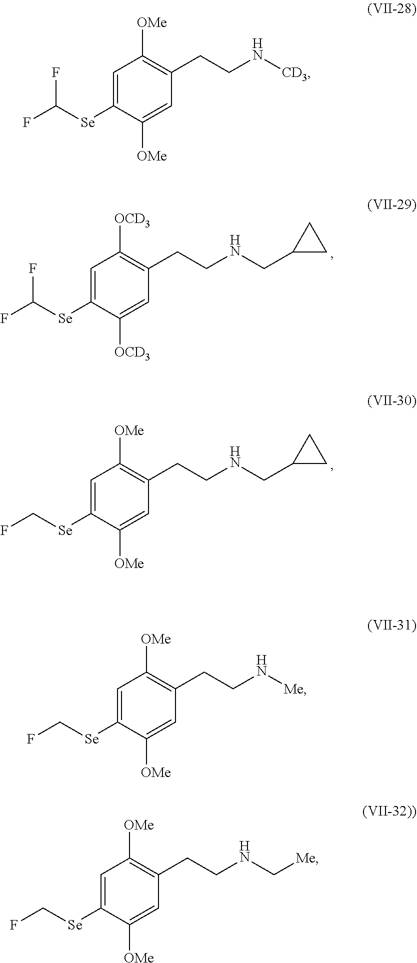

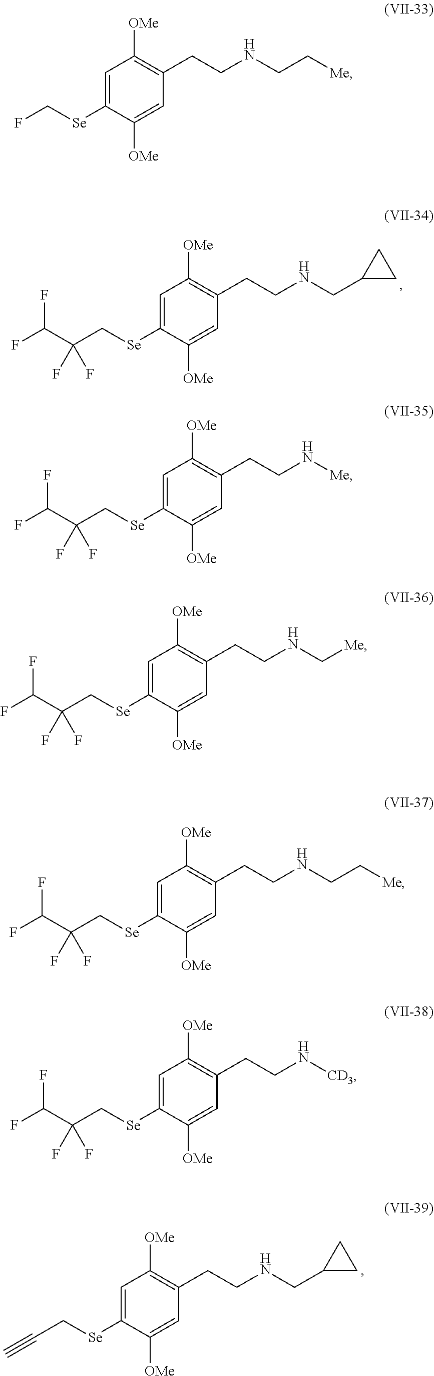

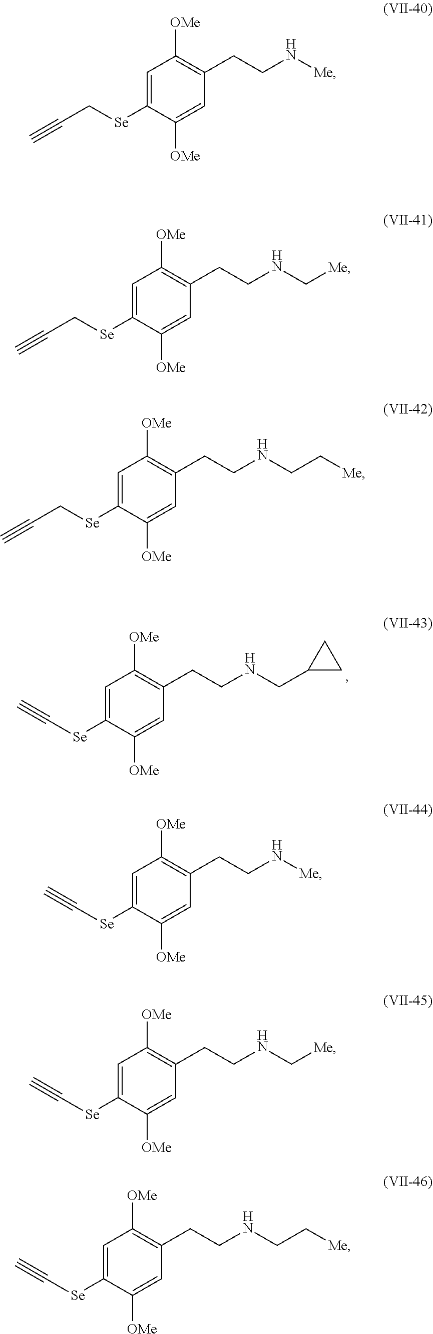

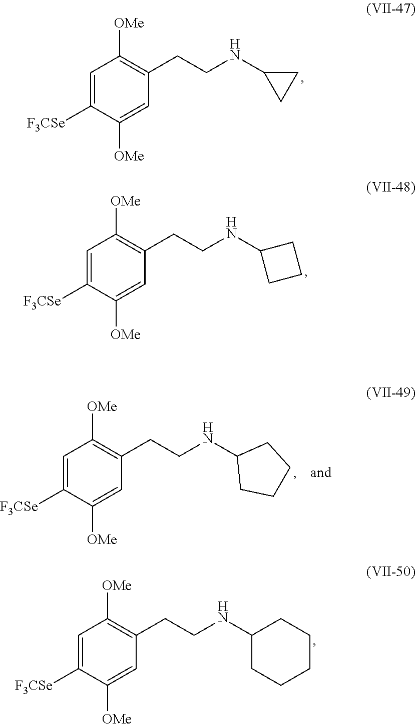

or a pharmaceutically acceptable salt, solvate, or prodrug thereof.

The compounds of Formula (I) through (VII) may contain a stereogenic center. In such cases, the compounds may exist as different stereoisomeric forms, even though Formula (I) through (VII) are drawn without reference to stereochemistry. Accordingly, the present disclosure includes all possible stereoisomers and includes not only racemic compounds but the individual enantiomers (enantiomerically pure compounds) and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

In some embodiments, the compounds described herein, e.g., compounds of Formula (I) through (VII), are racemic. In some embodiments, the compounds described herein, e.g., compounds of Formula (I) through (VII), are enantiomerically pure.

In some embodiments, the compound is an agonist of a serotonin 5-HT₂ receptor. In some embodiments, the compound is an agonist of a serotonin 5-HT_{2A} receptor. In some embodiments, the compound is an agonist of a serotonin 5-HT_{1A} receptor. In some embodiments, the compound is an agonist of a serotonin 5-HT_{2C} receptor. In some embodiments, administration of the compound elicits psychedelic effects. In some embodiments, administration of the compound does not elicit psychedelic effects (e.g., at dosages that would classically produce psychedelic effects).

Also disclosed herein is a pharmaceutically acceptable salt of the compounds of the present disclosure, e.g., a compound of Formula (I) through (VII). The acid used to form the pharmaceutically acceptable salt of the compound of Formula (I) through (VII) may be a monoacid, a diacid, a triacid, a tetraacid, or may contain a higher number of acid groups. The acid groups may be, e.g., a carboxylic acid, a sulfonic acid, a phosphonic acid, or other acidic moieties containing at least one replaceable hydrogen atom. Examples of acids for use in the preparation of the pharmaceutically acceptable (acid addition) salts disclosed herein include, but are not limited to, acetic acid, 2,2-dichloroacetic acid, phenylacetic acid, acylated amino acids, alginic acid, ascorbic acid, L-aspartic acid, sulfonic acids (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.), benzoic acids (e.g., benzoic acid, 4-acetamidobenzoic acid, 2-acetoxybenzoic acid, salicylic acid, 4-amino-salicylic acid, gentisic acid, etc.), boric acid, (+)-camphoric acid, cinnamic acid, citric acid, cyclamic acid, cyclohexanesulfamic acid, dodecylsulfuric acid, formic acid, fumaric acid, galactaric acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (−)-D-lactic acid, (±)-DL-lactic acid, lactobionic acid, maleic acid, malic acid, (−)-L-malic acid, (+)-D-malic acid, hydroxymaleic acid, malonic acid, (±)-DL-mandelic acid, isethionic acid, 1-hydroxy-2-naphthoic acid, nicotinic acid, nitric acid, orotic acid, oxalic acid, pamoic acid, perchloric acid, phosphoric acid, L-pyroglutamic acid, saccharic acid, succinic acid, sulfuric acid, sulfamic acid, tannic acid, tartaric acids (e.g., DL-tartaric acid, (+)-L-tartaric acid, (−)-D-tartaric acid), thiocyanic acid, propionic acid, valeric acid, and fatty acids (including fatty mono- and di-acids, e.g., adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc.).

In some embodiments, the pharmaceutically acceptable salt of the compound of Formula (I) through (VII) is a benzenesulfonate salt, a tartrate salt, a hemi-fumarate salt, an acetate salt, a citrate salt, a malonate salt, a fumarate salt, a succinate salt, an oxalate salt, a benzoate salt, a salicylate salt, an ascorbate salt, a hydrochloride salt, a maleate salt, a malate salt, a methanesulfonate salt, a toluenesulfonate salt, a glucuronate salt, or a glutarate salt of the compound of Formula (I) through (VII). In some embodiments, the pharmaceutically acceptable salt of the compound of Formula (I) through (VII) is a salt formed from a sulfonic acid (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.). In some embodiments, the pharmaceutically acceptable salt of the compound of Formula (I) through (VII) is a salt formed from a benzoic acid (e.g., benzoic acid, 4-acetamidobenzoic acid, 2-acetoxybenzoic acid, salicylic acid, 4-amino-salicylic acid, etc.).

In some embodiments, the pharmaceutically acceptable salt of the compound of Formula (I) through (VII) is a fatty acid salt. The fatty acid used to make the fatty acid salt of the compound of Formula (I) through (VII) may be a fatty monoacid or a fatty diacid, and may contain a fatty hydrocarbon portion made up of hydrogen and anywhere from 4, from 6, from 8, from 10, from 12, from 14, from 16, and up to 26, up to 24, up to 22, up to 20, up to 18 carbon atoms, which may be fully saturated or partially unsaturated. In some embodiments, the pharmaceutically acceptable salt of the compound of Formula (I) through (VII) is an adipate salt, a laurate salt, a linoleate salt, a myristate salt, a caprate salt, a stearate salt, an oleate salt, a caprylate salt, a palmitate salt, a sebacate salt, an undecylenate salt, or a caproate salt of the compound of Formula (I) through (VII).

Methods for preparing pharmaceutically acceptable salt forms of pharmaceutical compounds are known by those of ordinary skill in the art. In some embodiments, the method includes:
(a) suspending the compound of Formula (I) through (VII) in a solvent or mixture of solvents;
(b) contacting an acid with the compound of Formula (I) through (VII) to provide a mixture;
(c) optionally heating the mixture;
(d) optionally cooling the mixture; and
(e) isolating the salt.

Various solvents may be used in the disclosed methods, including one or more protic solvents, one or more aprotic solvents, or mixtures thereof. In some embodiments, the solvent(s) used in the method of preparing the salt is/are a protic solvent(s). In some embodiments, the solvent used in the method of preparing the salt is selected from the group consisting of methanol, ethanol, propanol, isopropanol (IPA), butanol, 2-butanol, acetone, butanone, dioxanes (1,4-dioxane), water, tetrahydrofuran (THF), acetonitrile (MeCN), ether solvents (e.g., t-butylmethyl ether (TBME)), hexane, heptane, octane, and combinations thereof. In some embodiments, the solvent is ethanol. In some embodiments, the solvent is 1,4-dioxane. In some embodiments, the solvent is acetonitrile. In some embodiments, the solvent is tetrahydrofuran.

Suitable acids for use in the preparation of pharmaceutically acceptable acid addition salts may include those described heretofore. The acid may be an inorganic acid such as hydrochloric acid, or an organic acid, with organic acids being preferred. In some embodiments, the acid is an organic acid selected from the group consisting of ascorbic acid, citric acid, fumaric acid, maleic acid, malonic acid, (−)-L-malic acid, (+)-L-tartaric acid, methanesulfonic acid, benzenesulfonic acid, toluenesulfonic acid, benzoic acid, salicylic acid, succinic acid, oxalic acid, D-glucuronic acid, glutaric acid salt, and acetic acid. In some embodiments, the acid is an organic acid selected from the group consisting of benzenesulfonic acid, (+)-L-tartaric acid, fumaric acid, acetic acid, citric acid, malonic acid, succinic acid, oxalic acid, benzoic acid, and salicylic acid. In some embodiments, the acid is a fatty acid, such as adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, caproic acid, etc., with particular mention being made to adipic (hexandioic) acid, lauric (dodecanoic) acid, linoleic acid, myristic (tetradecanoic) acid, capric (decanoic) acid, stearic (octadecanoic) acid, oleic acid, and caprylic (octanoic) acid.

In some embodiments, a stoichiometric (or superstoichiometric) quantity of the acid is contacted with the compound of Formula (I) through (VII). In some embodiments, a sub-stoichiometric (e.g., 0.5 molar equivalents) quantity of the acid is contacted with the compound of Formula (I) through (VII). The use of sub-stoichiometric quantities of the acid may be desirable when, for example, the acid contains at least two acidic protons (e.g., two or more carboxylic acid groups) and the target salt is a hemi-acid salt.

In some embodiments, the mixture is heated, e.g., refluxed, prior to cooling.

In some embodiments, the mixture is cooled and the salt is precipitated out of the solution. In some embodiments, the salt is precipitated out of solution in crystalline form. In some embodiments, the salt is precipitated out of solution in amorphous form.

Isolation of the salt may be performed by various well-known isolation techniques, such as filtration, decantation, and the like. In some embodiments, the isolating step includes filtering the mixture.

After isolation, additional crystallization and/or recrystallization steps may also optionally be performed, if desired, for example to increase purity, crystallinity, etc.

In some embodiments, compounds of the present disclosure, e.g., a compound of Formula (I) through (VII), or a pharmaceutically acceptable salt, stereoisomer, or prodrug thereof, is in the form of a solvate. Examples of solvate forms include, but are not limited to, hydrates, methanolates, ethanolates, isopropanolates, etc., with hydrates and ethanolates being preferred. The solvate may be formed from stoichiometric or nonstoichiometric quantities of solvent molecules. Solvates of the compounds herein may be in the form of isolable solvates. In one non-limiting example, as a hydrate, the compound may be a monohydrate, a dihydrate, etc. Solvates of the compounds herein also include solution-phase forms. Thus, in some embodiments, the present disclosure provides solution-phase compositions of the compounds of the present disclosure, or any pharmaceutically acceptable salts thereof, which are in solvated form, preferably fully solvated form.

In some embodiments, the compound of the present disclosure, e.g., a compound of Formula (I) through (VII), or a pharmaceutically acceptable salt, solvate, stereoisomer, or prodrug thereof, is provided in crystalline form, e.g., as determined by XRPD. Accordingly, pharmaceutical compositions may be prepared from a compound of Formula (I) through (VII), in crystalline form including in one or more polymorphic forms, and may be used for treatment as set forth herein. Crystalline forms may be advantageous in terms of stability and providing well-defined physical properties, which is desirable for pharmaceutical preparation and administration.

In some embodiments, the compound of the present disclosure, e.g., a compound of Formula (I) through (VII), or a pharmaceutically acceptable salt, solvate, stereoisomer, or prodrug thereof, is provided in amorphous form, e.g., as determined by XRPD. Accordingly, pharmaceutical compositions may be prepared from a compound of Formula (I) through (VII), or a pharmaceutically acceptable salt, solvate, stereoisomer, or prodrug thereof, in one or more amorphic forms, and may be used for treatment as set forth herein. Amorphous forms typically possess higher aqueous solubility and rates of dissolution compared to their crystalline counterparts, and thus may be well suited for quick acting dosage forms adapted to rapidly release the active agent, such as for orodispersible dosage forms (ODxs), immediate release (IR) dosage forms, and the like.

Compounds of the present disclosure, e.g., a compound of Formula (I) through (VII), or a pharmaceutically acceptable salt, solvate, stereoisomer, or prodrug thereof, may generally be prepared according to, or analogous to, the synthetic routes exemplified herein. Other synthetic routes may also be used according to techniques and procedures known to those of ordinary skill in the art.

### Therapeutic Applications and Methods

Also disclosed herein is a method of treating a subject with a disease or disorder comprising administering to the subject a therapeutically effective amount of a compound as disclosed herein (e.g., a compound of Formula (I) through (VII), or a pharmaceutically acceptable salt, solvate, stereoisomer, or prodrug thereof). In some embodiments, the disease or disorder is associated with a serotonin 5-HT₂ receptor.

The dosage and frequency (single or multiple doses) of the compounds administered herein can vary depending upon a variety of factors, including, but not limited to, the compound to be administered; the disease/condition being treated; route of administration; size, age, sex, health, body weight, body mass index, and diet of the subject; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response to the treatment and adjusting the dosage upwards (e.g., up-titration) or downwards (e.g., down-titration).

Dosages may be varied depending upon the requirements of the subject and the active ingredient (e.g., a compound of Formula (I) through (VII), or a pharmaceutically acceptable salt, solvate, stereoisomer, or prodrug thereof) being employed. The dose administered to a subject, in the context of the pharmaceutical compositions presented herein, should be sufficient to affect a beneficial therapeutic response in the subject over time. The size of the dose also will be determined by the existence, nature, and extent of any adverse side effects. Generally, treatment is initiated with smaller dosages, which are less than the optimum dose of the active ingredient. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Routes of administration may include oral routes (e.g., enteral/gastric delivery, intraoral administration such buccal, lingual, and sublingual routes), parenteral routes (e.g., intravenous, intradermal, intraarterial, intraperitoneal, intrathecal, intraventricular, intraurethral, intrasternal, intracranial, intramuscular, intrasynovial, and subcutaneous administration), topical routes (e.g., conjuctival, intracorneal, intraocular, ophthalmic, auricular, transdermal, nasal (e.g., intranasal), vaginal, uretheral, respiratory, and rectal administration), inhalation, or others sufficient to affect a beneficial therapeutic response.

Administration may follow a continuous administration schedule, or an intermittent administration schedule. The administration schedule may be varied depending on the active ingredient(s) employed, the condition being treated, the administration route, etc. For example, administration of a compound of Formula (I) through (VII), or a pharmaceutically acceptable salt, solvate, stereoisomer, or prodrug thereof, may be performed once a day (QD), or in divided dosages throughout the day, such as 2-times a day (BID), 3-times a day (TID), 4-times a day (QID), or more. In some embodiments administration may be performed nightly (QHS). In some embodiments, administration is performed as needed (PRN). Administration may also be performed on a weekly basis, e.g., once a week, twice a week, three times a week, four times a week, every other week, every two weeks, etc., or less. The administration schedule may also designate a defined number of treatments per treatment course, for example, the compound of Formula (I) through (VII), or a pharmaceutically acceptable salt, solvate, stereoisomer, or prodrug thereof, may be administered 1 time, 2 times, 3 times, 4 times, 5 times, 6 times, 7 times, or 8 times per treatment course. Other administration schedules may also be deemed appropriate using sound medical judgement.

The dosing can be continuous (7 days of administration in a week) or intermittent, for example, depending on the pharmacokinetics and a particular subject's clearance/accumulation of the drug. If intermittently, the schedule may be, for example, 4 days of administration and 3 days off (rest days) in a week or any other intermittent dosing schedule deemed appropriate using sound medical judgement. For example, intermittent dosing may involve administration of a single dose within a treatment course. The dosing whether continuous or intermittent is continued for a particular treatment course, typically at least a 28-day cycle (1 month), which can be repeated with or without a drug holiday. Longer or shorter courses can also be used such as 14 days, 18 days, 21 days, 24 days, 35 days, 42 days, 48 days, or longer, or any range therebetween. The course may be repeated without a drug holiday or with a drug holiday depending upon the subject. Other schedules are possible depending upon the presence or absence of adverse events, response to the treatment, patient convenience, and the like.

Utilizing the teachings provided herein, an effective prophylactic or therapeutic treatment regimen can be planned that does not cause substantial toxicity or adverse side effects (e.g., caused by sedative or psychotomimetic toxic spikes in plasma concentration of any of the compounds Formula (I) through (VII)), and yet is entirely effective to treat the clinical symptoms demonstrated by the particular patient. This planning should involve the careful choice of active ingredient by considering factors such as compound potency, relative bioavailability, patient body weight, presence and severity of adverse side effects, preferred mode of administration, and the toxicity profile of the selected agent.

A therapeutically effective dose of the compounds of the present disclosure may vary depending on the variety of factors described above, but is typically that which provides the compound of Formula (I) through (VII) in an amount of about 0.00001 mg to about 10 mg per kilogram body weight of the subject, or any range in between, e.g., about 0.00001 mg/kg, about 0.00005 mg/kg, about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg of the compound of Formula (I) through (VII) (on an active basis).

The compounds of the present disclosure (e.g., a compound of Formula (I) through (VII), or a pharmaceutically acceptable salt, solvate, stereoisomer, or prodrug thereof) may be administered at a psychedelic dose. Psychedelic dosing, by mouth or otherwise, may in some embodiments range from about 0.3 mg/kg, about 0.35 mg/kg, about 0.4 mg/kg, about 0.45 mg/kg, about 0.5 mg/kg, and up to about 5 mg/kg, about 4 mg/kg, about 3 mg/kg, about 2 mg/kg, about 1 mg/kg, about 0.95 mg/kg, about 0.9 mg/kg, about 0.85 mg/kg, about 0.8 mg/kg, about 0.75 mg/kg, about 0.7 mg/kg, about 0.65 mg/kg, about 0.6 mg/kg, about 0.55 mg/kg of the compound of Formula (I) through (VII) (on an active basis). Higher dosing may also be used in some embodiments, as described above. In some embodiments, psychedelic doses are administered once by mouth or otherwise, with the possibility of repeat doses at least one week apart. In some instances, no more than 5 doses are given in any one course of treatment. Courses can be repeated as necessary, with or without a drug holiday. Such acute treatment regimens may be accompanied by psychotherapy, before, during, and/or after the psychedelic dose. These treatments are appropriate for a variety of mental health disorders disclosed herein, examples of which include, but are not limited to, major depressive disorder (MDD), therapy resistant depression (TRD), anxiety disorders, and substance use disorders (e.g., alcohol use disorder, opioid use disorder, amphetamine use disorder, nicotine use disorder, smoking, and cocaine use disorder).

The compounds of the present disclosure (e.g., a compound of Formula (I) through (VII), or a pharmaceutically acceptable salt, solvate, stereoisomer, or prodrug thereof), may be administered at sub-psychoactive (yet still potentially serotonergic concentrations) concentrations to achieve durable therapeutic benefits, with decreased toxicity, and may thus be suitable for microdosing. Sub-psychedelic dosing, by mouth or otherwise, may in some embodiments range from about 0.00001 mg/kg, about 0.00005 mg/kg, about 0.0001 mg/kg, about 0.0005 mg/kg, about 0.001 mg/kg, about 0.005 mg/kg, about 0.006 mg/kg, about 0.008 mg/kg, about 0.009 mg/kg, about 0.01 mg/kg, and up to about 0.3 mg/kg, about 0.25 mg/kg, about 0.2 mg/kg, about 0.15 mg/kg, about 0.1 mg/kg, about 0.083 mg/kg, about 0.08 mg/kg, about 0.075 mg/kg, about 0.07 mg/kg, about 0.06 mg/kg, about 0.05 mg/kg, about 0.04 mg/kg, about 0.03 mg/kg, about 0.02 mg/kg of the compound of Formula (I) through (VII) (on an active basis). Typically, sub-psychedelic doses are administered orally up to every day, for a treatment course (e.g., 1 month). However, there is no limitation on the number of doses at sub-psychedelic doses-dosing can be less frequent or more frequent as deemed appropriate. Courses can be repeated as necessary, with or without a drug holiday.

Sub-psychedelic dosing can also be carried out, for example, by transdermal delivery, subcutaneous administration, etc., via modified, controlled, slow, or extended release dosage forms, including, but not limited to, depot dosage forms, implants, patches, and pumps, which can be optionally remotely controlled. Here, doses would achieve similar blood levels as low oral dosing, but would nevertheless be sub-psychedelic.

Sub-psychedelic doses can be used, e.g., for the chronic treatment or maintenance of a variety of diseases or disorders disclosed herein, examples of which include, but are not limited to, depression (e.g., MDD), inflammation, pain, and neuroinflammation.

The compounds of the present disclosure (e.g., a compound of Formula (I) through (VII), or a pharmaceutically acceptable salt, solvate, stereoisomer, or prodrug thereof), may be used for a maintenance regimen. As used herein, a “maintenance regimen” generally refers to the administration of the compounds of the present disclosure (e.g., a compound of Formula (I) through (VII), or a pharmaceutically acceptable salt, solvate, stereoisomer, or prodrug thereof) following achievement of a target dose, e.g., following completion of an up-titration regimen, and/or following a positive clinical response, e.g., improvement of the patient's condition, either to the same drug or to a different drug. In some embodiments, the patient is administered a first drug for a therapeutic regimen and a second drug for a maintenance regimen, wherein the first and second drugs are different. For example, the patient may be administered a therapeutic regimen of a first drug which is not a compound of the present disclosure (e.g., the first drug is a serotonergic psychedelic such as LSD, psilocybin, MDMA, dimethyltryptamine, etc., or a non-psychedelic drug), followed by a compound of the present disclosure (as the second drug) in a maintenance regimen. In another example, a different compound of the present disclosure is used for the therapeutic regimen (first drug) than is used for the maintenance regimen (second drug). In some embodiments, the patient is administered the same compound of the present disclosure for both a therapeutic regimen and a maintenance regimen. In any case, the maintenance dose of the compounds of the present disclosure may be used to ‘maintain’ the therapeutic response and/or to prevent occurrences of relapse. When the same compound of the present disclosure is used for both the original therapeutic regimen and for the maintenance regimen, the maintenance dose of the compound may be at or below the therapeutic dose. In some embodiments, the maintenance dose is a psychedelic dose. In some embodiments, the maintenance dose is a sub-psychedelic dose. Generally, dosing is carried out daily or intermittently for the maintenance regimen, however, maintenance regimens can also be carried out continuously, for example, over several days, weeks, months, or years. Moreover, the maintenance dose may be given to a patient over a long period of time, even chronically.

The subjects treated herein may have a disease or disorder associated with a serotonin 5-HT₂ receptor.

In some embodiments, the disease or disorder is a neuropsychiatric disease or disorder or an inflammatory disease or disorder. In some embodiments, the neuropsychiatric disease or disorder is not schizophrenia or cognitive deficits in schizophrenia.

In some embodiments, the disease or disorder is a central nervous system (CNS) disorder, including, but not limited to, major depressive disorder (MDD), treatment-resistant depression (TRD), post-traumatic stress disorder (PTSD), bipolar and related disorders (including, but not limited to, bipolar I disorder, bipolar II disorder, cyclothymic disorder), obsessive-compulsive disorder (OCD), generalized anxiety disorder (GAD), social anxiety disorder, substance use disorders (including, but not limited to, alcohol use disorder, opioid use disorder, amphetamine use disorder, nicotine use disorder, smoking, and cocaine use disorder), eating disorders (including, but not limited to anorexia nervosa, bulimia nervosa, binge-eating disorder, etc.), Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, suicidal ideation, suicidal behavior, major depressive disorder with suicidal ideation or suicidal behavior, melancholic depression, atypical depression, dysthymia, non-suicidal self-injury disorder (NSSID), chronic fatigue syndrome, Lyme disease, gambling disorder, paraphilic disorders (including, but not limited to, pedophilic disorder, exhibitionistic disorder, voyeuristic disorder, fetishistic disorder, sexual masochism or sadism disorder, and transvestic disorder, etc.), sexual dysfunction (e.g., low libido, hypoactive sexual desire disorder (HSDD), etc.), peripheral neuropathy, and obesity.

In some embodiments, the methods provided herein are used to treat a subject with a depressive disorder. As used herein, the terms “depressive disorder” or “depression” refers to a group of disorders characterized by low mood that can affect a person's thoughts, behavior, feelings, and sense of well-being lasting for a period of time. In some embodiments, the depressive disorder disrupts the physical and psychological functions of a person. In some embodiments, the depressive disorder causes a physical symptom such as weight loss, aches or pains, headaches, cramps, or digestive problems. In some embodiments, the depressive disorder causes a psychological symptom such as persistent sadness, anxiety, feelings of hopelessness and irritability, feelings of guilt, worthlessness, or helplessness, loss of interest or pleasure in hobbies and activities, difficulty concentrating, remembering, or making decisions. In some embodiments, the depressive disorder is major depressive disorder (MDD), atypical depression, bipolar disorder, catatonic depression, depressive disorder due to a medical condition, postpartum depression, premenstrual dysphoric disorder, seasonal affective disorder, or treatment-resistant depression (TRD).

In some embodiments, the disease or disorder is major depressive disorder (MDD). As used herein, the term “major depressive disorder” refers to a condition characterized by a time period of low mood that is present across most situations. Major depressive disorder is often accompanied by low self-esteem, loss of interest in normally enjoyable activities, low energy, and pain without a clear cause. In some instances, major depressive order is characterized by symptoms of depression lasting at least two weeks. In some instances, an individual experiences periods of depression separated by years. In some instances, an individual experiences symptoms of depression that are nearly always present. Major depressive disorder can negatively affect a person's personal, work, or school life, as well as sleeping, eating habits, and general health. Approximately 2-7% of adults with major depressive disorder commit suicide, and up to 60% of people who commit suicide had major depressive disorder or another related mood disorder. Dysthymia is a subtype of major depressive disorder consisting of the same cognitive and physical problems as major depressive disorder with less severe but longer-lasting symptoms. Exemplary symptoms of a major depressive disorder include, but are not limited to, feelings of sadness, tearfulness, emptiness or hopelessness, angry outbursts, irritability or frustration, even over small matters, loss of interest or pleasure in most or all normal activities, sleep disturbances, including insomnia or sleeping too much, tiredness and lack of energy, reduced appetite, weight loss or gain, anxiety, agitation or restlessness, slowed thinking, speaking, or body movements, feelings of worthlessness or guilt, fixating on past failures or self-blame, trouble thinking, concentrating, making decisions, and remembering things, frequent thoughts of death, suicidal thoughts, suicide attempts, or suicide, and unexplained physical problems, such as back pain or headaches.

As used herein, the term “atypical depression” refers to a condition wherein an individual shows signs of mood reactivity (i.e., mood brightens in response to actual or potential positive events), significant weight gain, increase in appetite, hypersomnia, heavy, leaden feelings in arms or legs, and/or long-standing pattern of interpersonal rejection sensitivity that results in significant social or occupational impairment. Exemplary symptoms of atypical depression include, but are not limited to, daily sadness or depressed mood, loss of enjoyment in things that were once pleasurable, major changes in weight (gain or loss) or appetite, insomnia or excessive sleep almost every day, a state of physical restlessness or being rundown that is noticeable by others, daily fatigue or loss of energy, feelings of hopelessness, worthlessness, or excessive guilt almost every day, problems with concentration or making decisions almost every day, recurring thoughts of death or suicide, suicide plan, or suicide attempt.

As used herein, the term “bipolar disorder” refers to a condition that causes an individual to experience unusual shifts in mood, energy, activity levels, and the ability to carry out day-to day tasks. Individuals with bipolar disorder experience periods of unusually intense emotion, changes in sleep patterns and activity levels, and unusual behaviors. These distinct periods are called “mood episodes.” Mood episodes are drastically different from the moods and behaviors that are typical for the person. Exemplary symptoms of mania, excessive behavior, include, but are not limited to, abnormally upbeat, jumpy, or wired behavior; increased activity, energy, or agitation, exaggerated sense of well-being and self-confidence, decreased need for sleep, unusual talkativeness, racing thoughts, distractibility, and poor decision-making—for example, going on buying sprees, taking sexual risks, or making foolish investments. Exemplary symptoms of depressive episodes or low mood, include, but are not limited to, depressed mood, such as feelings of sadness, emptiness, hopelessness, or tearfulness; marked loss of interest or feeling no pleasure in all-or almost all-activities, significant weight loss, weight gain, or decrease or increase in appetite, insomnia or hypersomnia (excessive sleeping or excessive sleepiness), restlessness or slowed behavior, fatigue or loss of energy, feelings of worthlessness or excessive or inappropriate guilt, decreased ability to think or concentrate, or indecisiveness, and thinking about, planning or attempting suicide. Bipolar disorder includes bipolar I disorder, bipolar II disorder, and cyclothymic disorder. Bipolar I disorder is defined by manic episodes that last at least 7 days or by severe manic symptoms that require hospitalization. A subject with bipolar I disorder may also experience depressive episodes typically lasting at least 2 weeks. Episodes of depression with mixed features, i.e., depressive and manic symptoms at the same time, are also possible. Bipolar II disorder is characterized by a pattern of depressive and hypomanic episodes, but not severe manic episodes typical of bipolar I disorder. Cyclothymic disorder (also referred to as cyclothymia) is characterized by periods of hypomanic symptoms (elevated mood and euphoria) and depressive symptoms lasting over a period of at least 2 years. The mood fluctuations are not sufficient in number, severity, or duration to meet the full criteria for a hypomanic or depressive episode.

As used herein, the term “catatonic depression” refers to a condition causing an individual to remain speechless and motionless for an extended period. Exemplary symptoms of catatonic depression include, but are not limited to, feelings of sadness, which can occur daily, a loss of interest in most activities, sudden weight gain or loss, a change in appetite, trouble falling asleep, trouble getting out of bed, feelings of restlessness, irritability, feelings of worthlessness, feelings of guilt, fatigue, difficulty concentrating, difficulty thinking, difficulty making decisions, thoughts of suicide or death, and/or a suicide attempt.

As used herein, the term “depressive disorder due to a medical condition” refers to a condition wherein an individual experiences depressive symptoms caused by another illness. Examples of medical conditions known to cause a depressive disorder include, but are not limited to, HIV/AIDS, diabetes, arthritis, strokes, brain disorders such as Parkinson's disease, Huntington's disease, multiple sclerosis, and Alzheimer's disease, metabolic conditions (e.g., vitamin B12 deficiency), autoimmune conditions (e.g., lupus and rheumatoid arthritis), viral or other infections (hepatitis, mononucleosis, herpes), back pain, and cancer (e.g., pancreatic cancer).

As used herein, the term “postpartum depression” refers to a condition as the result of childbirth and hormonal changes, psychological adjustment to parenthood, and/or fatigue. Postpartum depression is often associated with women, but men can also suffer from postpartum depression as well. Exemplary symptoms of postpartum depression include, but are not limited to, feelings of sadness, hopeless, emptiness, or overwhelmed; crying more often than usual or for no apparent reason; worrying or feeling overly anxious; feeling moody, irritable, or restless; oversleeping, or being unable to sleep even when the baby is asleep; having trouble concentrating, remembering details, and making decisions; experiencing anger or rage; losing interest in activities that are usually enjoyable; suffering from physical aches and pains, including frequent headaches, stomach problems, and muscle pain; eating too little or too much; withdrawing from or avoiding friends and family; having trouble bonding or forming an emotional attachment with the baby; persistently doubting his or ability to care for the baby; and thinking about harming themselves or the baby.

As used herein, the term “premenstrual dysphoric disorder” refers to a condition wherein an individual expresses mood lability, irritability, dysphoria, and anxiety symptoms that occur repeatedly during the premenstrual phase of the cycle and remit around the onset of menses or shortly thereafter. Exemplary symptoms of premenstrual dysphoric disorder includes, but are not limited to, lability (e.g., mood swings), irritability or anger, depressed mood, anxiety and tension, decreased interest in usual activities, difficulty in concentration, lethargy and lack of energy, change in appetite (e.g., overeating or specific food cravings), hypersomnia or insomnia, feeling overwhelmed or out of control, physical symptoms (e.g., breast tenderness or swelling, joint or muscle pain, a sensation of ‘bloating’ and weight gain), self-deprecating thoughts, feelings of being keyed up or on edge, decreased interest in usual activities (e.g., work, school, friends, hobbies), subjective difficulty in concentration, and easy fatigability.

As used herein, the term “seasonal affective disorder” refers to a condition wherein an individual experiences mood changes based on the time of the year. In some instances, an individual experiences low mood, low energy, or other depressive symptoms during the fall and/or winter season. In some instances, an individual experiences low mood, low energy, or other depressive symptoms during the spring and/or summer season. Exemplary symptoms of seasonal affective disorder include, but are not limited to, feeling depressed most of the day or nearly every day, losing interest in activities once found enjoyable, having low energy, having problems with sleeping, experiencing changes in appetite or weight, feeling sluggish or agitated, having difficulty concentrating, feeling hopeless, worthless, or guilty, and having frequent thoughts of death or suicide.

In some embodiments, a depressive disorder comprises a medical diagnosis based on the criteria and classification from Diagnostic and Statistical Manual of Mental Disorders, 5th Ed. In some embodiments, a depressive disorder comprises a medical diagnosis based on an independent medical evaluation.

In some embodiments, the methods described herein are provided to a subject with depression that is resistant to treatment. In some embodiments, the subject has been diagnosed with treatment-resistant depression (TRD). The term “treatment-resistant depression” refers to a kind of depression that does not respond or is resistant to at least one or more treatment attempts of adequate dose and duration. In some embodiments, the subject with treatment-resistant depression has failed to respond to 1 treatment attempt, 2 treatment attempts, 3 treatment attempts, 4 treatment attempts, 5 treatment attempts, or more. In some embodiments, the subject with treatment-resistant depression has been diagnosed with major depressive disorder and has failed to respond to 3 or more treatment attempts. In some embodiments, the subject with treatment resistant depression has been diagnosed with bipolar disorder and has failed to respond to 1 treatment attempt.

In some embodiments, the methods provided herein reduce at least one sign or symptom of a depressive disorder. In some embodiments, the methods provided herein reduce at least one sign or symptom of a depressive disorder by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the disease or disorder is an anxiety disorder. As used herein, the term “anxiety disorder” refers to a state of apprehension, uncertainty, and/or fear resulting from the anticipation of an event and/or situation. Anxiety disorders cause physiological and psychological signs or symptoms. Non-limiting examples of physiological symptoms include muscle tension, heart palpitations, sweating, dizziness, shortness of breath, tachycardia, tremor, fatigue, worry, irritability, and disturbed sleep. Non-limiting examples of psychological symptoms include fear of dying, fear of embarrassment or humiliation, fear of an event occurring, etc. Anxiety disorders also impair a subject's cognition, information processing, stress levels, and immune response. In some embodiments, the methods disclosed herein treat chronic anxiety disorders. As used herein, a “chronic” anxiety disorder is recurring. Examples of anxiety disorders include, but are not limited to, generalized anxiety disorder (GAD), social anxiety disorder, panic disorder, panic attack, a phobia-related disorder (e.g., phobias related to flying, heights, specific animals such as spiders/dogs/snakes, receiving injections, blood, etc., agoraphobia), separation anxiety disorder, selective mutism, anxiety due to a medical condition, post-traumatic stress disorder (PTSD), obsessive-compulsive disorder (OCD), substance-induced anxiety disorder, etc.

In some embodiments, the subject in need thereof develops an anxiety disorder after experiencing the effects of a disease. The effects of a disease include diagnosis of an individual with said disease, diagnosis of an individual's loved ones with said disease, social isolation due to said disease, quarantine from said disease, or social distancing as a result of said disease. In some embodiments, an individual is quarantined to prevent the spread of the disease. In some embodiments, the disease is COVID-19, SARS, or MERS. In some embodiments, a subject develops an anxiety disorder after job loss, loss of housing, or fear of not finding employment.

In some embodiments, the disease or disorder is generalized anxiety disorder (GAD). Generalized anxiety disorder is characterized by excessive anxiety and worry, fatigue, restlessness, increased muscle aches or soreness, impaired concentration, irritability, and/or difficulty sleeping. In some embodiments, a subject with generalized anxiety disorder does not have associated panic attacks. In some embodiments, the subject has generalized anxiety disorder with depression. In some embodiments, the methods herein are provided to a subject with generalized anxiety disorder also having symptoms of depression. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is social anxiety disorder. As used herein, “social anxiety disorder” is a marked fear or anxiety about one or more social situations in which the individual is exposed to possible scrutiny by others. Non-limiting examples of situations which induce social anxiety include social interactions (e.g., having a conversation, meeting unfamiliar people), being observed (e.g., eating or drinking), and performing in front of others (e.g., giving a speech). In some embodiments, the social anxiety disorder is restricted to speaking or performing in public. In some embodiments, treating according to the methods of the disclosure reduces or ameliorates a symptom of social anxiety disorder. In some embodiments, after treating the symptom is reduced compared to prior to treating by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the disease or disorder is a compulsive disorder, such as obsessive-compulsive disorder (OCD), body-focused repetitive behavior, hoarding disorder, gambling disorder, compulsive buying, compulsive internet use, compulsive video gaming, compulsive sexual behavior, compulsive eating, compulsive exercise, body dysmorphic disorder, hoarding disorder, dermatillomania, trichotillomania, excoriation, substance-induced obsessive compulsive and related disorder, or an obsessive-compulsive disorder due to another medical condition, etc., or a combination thereof. In some embodiments, the disease or disorder is obsessive-compulsive disorder (OCD).

In some embodiments, at least one sign or symptom of an anxiety disorder is improved following the administration of a compound as disclosed herein. In some embodiments, a sign or symptom of an anxiety disorder is measured according to a diary assessment, an assessment by a clinician or caregiver, or a clinical scale. In some embodiments, treatment causes a demonstrated improvement in one or more of the following: State-Trait Anxiety Inventory (STAI), Beck Anxiety Inventory (BAI), Hospital Anxiety and Depression Scale (HADS), Generalized Anxiety Disorder questionnaire-IV (GADQ-IV), Hamilton Anxiety Rating Scale (HARS), Leibowitz Social Anxiety Scale (LSAS), Overall Anxiety Severity and Impairment Scale (OASIS), Hospital Anxiety and Depression Scale (HADS), Patient Health Questionnaire 4 (PHQ-4), Social Phobia Inventory (SPIN), Brief Trauma Questionnaire (BTQ), Combat Exposure Scale (CES), Mississippi Scale for Combat-Related PTSD (M-PTSD), Posttraumatic Maladaptive Beliefs Scale (PMBS), Perceived Threat Scale (DRRI-2 Section: G), PTSD Symptom Scale-Interview for DSM-5 (PSS-I-5), Structured Interview for PTSD (SI-PTSD), Davidson Trauma Scale (DTS), Impact of Event Scale-Revised (IES-R), Posttraumatic Diagnostic Scale (PDS-5), Potential Stressful Events Interview (PSEI), Stressful Life Events Screening Questionnaire (SLESQ), Spielberger's Trait and Anxiety, Generalized Anxiety Dis-order 7-Item Scale, The Psychiatric Institute Trichotillomania Scale (PITS), The MGH Hairpulling Scale (MGH-HPS), The NIMH Trichotillomania Severity Scale (NIMH-TSS), The NIMH Trichotillomania Impairment Scale (NIMH-TIS), The Clinical Global Impression (CGI), the Brief Social Phobia Scale (BSPS), The Panic Attack Questionnaire (PAQ), Panic Disorder Severity Scale, Florida Obsessive-Compulsive Inventory (FOCI), The Leyton Obsessional Inventory Survey Form, The Vancouver Obsessional Compulsive Inventory (VOCI), The Schedule of Compulsions, Obsessions, and Pathological Impulses (SCOPI), Padua Inventory-Revised (PI-R), Quality of Life (QoL), The Clinical Global Improvement (CGI) scale, The Yale-Brown Obsessive-Compulsive Scale (Y-BOCS), The Yale-Brown Obsessive-Compulsive Scale Second Edition (Y-BOCS-II), The Dimensional Yale-Brown Obsessive-Compulsive Scale (DY-BOCS), The National Institute of Mental Health-Global Obsessive-Compulsive Scale (NIMH-GOCS), The Yale-Brown Obsessive-Compulsive Scale Self-Report (Y-BOCS-SR), The Obsessive-Compulsive Inventory-Re-vised (OCI-R), and the Dimensional Obsessive-Compulsive Scale (DOCS), or a combination thereof. In some embodiments, treating according to the methods of the disclosure results in an improvement in an anxiety disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is attention deficit disorder (ADD). ADD is most commonly diagnosed in children under the age of 16 who have 6 or more symptoms of inattention (5 or more for older teenagers) for at least 6 consecutive months, but no signs of hyperactivity/impulsivity. The symptoms of inattention include, but are not limited to, trouble paying attention, avoids long mental tasks such as homework, trouble staying on task, disorganized or forgetful, doesn't appear to listen when spoken to, doesn't pay close attention to details. Loses things often, makes careless mistakes, and struggles to follow through with instructions. In some embodiments, the disease or disorder is attention deficit hyperactivity disorder (ADHD). ADHD is marked by an ongoing pattern of inattention and/or hyperactivity-impulsivity. Hyperactivity-impulsivity symptoms may often include, but are not limited to, fidgeting or squirming while seated, leaving their seats in situations where staying seated is expected, running, dashing, or climbing around at inappropriate times, being unable to engage in hobbies quietly, being constantly in motion, talking excessively, answering questions before they are fully asked, having difficulty waiting for one's turn, and interrupting or intruding on others during conversations or activities.

In some embodiments, the disease or disorder is a headache disorder. As used herein, the term “headache disorder” refers to a disorder characterized by recurrent headaches. Headache disorders include migraine, tension-type headache, cluster headache, and chronic daily headache syndrome.

In some embodiments, a method of treating cluster headaches in a subject in need thereof is disclosed herein. In some embodiments, at least one sign or symptom of cluster headache is improved following treatment. In some embodiments, the sign or symptom of cluster headache is measured according to a diary assessment, a physical or psychological assessment by clinician, an imaging test, or a neurological examination. Cluster headache is a primary headache disorder and belongs to the trigeminal autonomic cephalalgias. The definition of cluster headaches is a unilateral headache with at least one autonomic symptom ipsilateral to the headache. Attacks are characterized by severe unilateral pain predominantly in the first division of the trigeminal nerve-the fifth cranial nerve whose primary function is to provide sensory and motor innervation to the face. Attacks are also associated with prominent unilateral cranial autonomic symptoms and subjects often experience agitation and restlessness during attacks. In some embodiments, a subject with cluster headaches also experiences nausea and/or vomiting. In some embodiments, a subject with cluster headaches experiences unilateral pain, excessive tearing, facial flushing, a droopy eyelid, a constricted pupil, eye redness, swelling under or around one or both eyes, sensitivity to light, nausea, agitation, and restlessness.

In some embodiments, a method of treating migraines in a subject in need thereof is disclosed herein. A migraine is a moderate to severe headache that affects one half or both sides of the head, is pulsating in nature, and last from 2 to 72 hours. Symptoms of migraine include headache, nausea, sensitivity to light, sensitivity to sound, sensitivity to smell, dizziness, difficulty speaking, vertigo, vomiting, seizure, distorted vision, fatigue, or loss of appetite. Some subjects also experience a prodromal phase, occurring hours or days before the headache, and/or a postdromal phase following headache resolution. Prodromal and postdromal symptoms include hyperactivity, hypoactivity, depression, cravings for particular foods, repetitive yawning, fatigue and neck stiffness and/or pain. In some embodiments, the migraine is a migraine without aura, a migraine with aura, a chronic migraine, an abdominal migraine, a basilar migraine, a menstrual migraine, an ophthalmoplegic migraine, an ocular migraine, an ophthalmic migraine, or a hemiplegic migraine. In some embodiments, the migraine is a migraine without aura. A migraine without aura involves a migraine headache that is not accompanied by a headache. In some embodiments, the migraine is a migraine with aura. A migraine with aura is primarily characterized by the transient focal neurological symptoms that usually precede or sometimes accompany the headache. Less commonly, an aura can occur without a headache, or with a non-migraine headache. In some embodiments, the migraine is a hemiplegic migraine. A hemiplegic migraine is a migraine with aura and accompanying motor weakness. In some embodiments, the hemiplegic migraine is a familial hemiplegic migraine or a sporadic hemiplegic migraine. In some embodiments, the migraine is a basilar migraine. A subject with a basilar migraine has a migraine headache and an aura accompanied by difficulty speaking, world spinning, ringing in ears, or a number of other brainstem-related symptoms, not including motor weakness. In some embodiments, the migraine is a menstrual migraine. A menstrual migraine occurs just before and during menstruation. In some embodiments, the subject has an abdominal migraine. Abdominal migraines are often experienced by children. Abdominal migraines are not headaches, but instead stomach aches. In some embodiments, a subject with abdominal migraines develops migraine headaches. In some embodiments, the subject has an ophthalmic migraine also called an “ocular migraine.” Subjects with ocular migraines experience vision or blindness in one eye for a short time with or after a migraine headache. In some embodiments, a subject has an ophthalmoplegic migraine. Ophthalmoplegic migraines are recurrent attacks of migraine headaches associated with paresis of one or more ocular cranial nerves. In some embodiments, the subject in need of treatment experiences chronic migraines. As defined herein, a subject with chronic migraines has more than fifteen headache days per month. In some embodiments, the subject in need of treatment experiences episodic migraines. As defined herein, a subject with episodic migraines has less than fifteen headache days per month.

In some embodiments, a method of treating chronic daily headache syndrome (CDHS) in a subject in need thereof is disclosed herein. A subject with CDHS has a headache for more than four hours on more than 15 days per month. Some subjects experience these headaches for a period of six months or longer. CHDS affects 4% of the general population. Chronic migraine, chronic tension-type headaches, new daily persistent headache, and medication overuse headaches account for the vast majority of chronic daily headaches.

In some embodiments, after treating according to the methods of the disclosure, the frequency of headaches and/or related symptoms decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, after treating according to the methods of the disclosure, the length of a headache attack decreases by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, compared to prior to said treating.

In some embodiments, at least one sign or symptom of headache disorder is improved following administration of a compound disclosed herein. In some embodiments, a sign or symptom of a headache disorder is measured according to a diary assessment, an assessment by a clinician or caregiver, or a clinical scale. In some embodiments, treatment of the present disclosure causes a demonstrated improvement in one or more of the following: the Visual Analog Scale, Numeric Rating Scale, the Short Form Health Survey, Profile of Mood States, the Pittsburgh Sleep Quality Index, the Major Depression Inventory, the Perceived Stress Scale, the 5-Level EuroQoL-5D, the Headache Impact Test; the ID-migraine; the 3-item screener; the Minnesota Multiphasic Personality Inventory; the Hospital Anxiety and Depression Scale (HADS), the 50 Beck Depression Inventory (BDI; both the original BD151 and the second edition, BDI-1152), the 9-item Patient Health Questionnaire (PHQ-9), the Migraine Disability Assessment Questionnaire (MI-DAS), the Migraine-Specific Quality of Life Questionnaire version 2.1 (MSQ v2.1), the European Quality of Life-5 Dimensions (EQ-5D), the Short-form 36 (SF-36), or a combination thereof. In some embodiments, treating according to the methods of the disclosure results in an improvement in a headache disorder compared to pre-treatment of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art. In some embodiments, the sign or symptom of the headache disorder is measured according to a diary assessment, a physical or psychological assessment by clinician, an imaging test, an electroencephalogram, a blood test, a neurological examination, or combination thereof. In some embodiments, the blood test evaluates blood chemistry and/or vitamins.

In some embodiments, the disease or disorder is a substance use disorder. Substance addictions which can be treated using the methods herein include addictions to addictive substances/agents such as recreational drugs and addictive medications. Examples of addictive substances/agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine. In some embodiments, the disease or disorder is alcohol use disorder (AUD). In some embodiments, the disease or disorder is nicotine use (e.g., smoking) disorder, and the therapy is used for e.g., smoking cessation.

In some embodiments, the disclosure provides for the management of sexual dysfunction, which may include, but is not limited to, sexual desire disorders, for example, decreased libido; sexual arousal disorders, for example, those causing lack of desire, lack of arousal, pain during intercourse, and orgasm disorders such as anorgasmia; and erectile dysfunction; particularly sexual dysfunction disorders stemming from psychological factors.

In some embodiments, the disease or disorder is an eating disorder. As used herein, the term “eating disorder” refers to any of a range of psychological disorders characterized by abnormal or disturbed eating habits. Non-limiting examples of eating disorders include pica, anorexia nervosa, bulimia nervosa, rumination disorder, avoidant/restrictive food intake disorder, binge-eating disorder, other specified feeding or eating disorder, unspecified feeding or eating disorder, or combinations thereof. In some embodiments, the eating disorder is pica, anorexia nervosa, bulimia nervosa, rumination disorder, avoidant/restrictive food intake disorder, binge-eating disorder, or combinations thereof. In some embodiments, the methods disclosed herein treat chronic eating disorders. As used herein, a “chronic” eating disorder is recurring. In some embodiments, at least one sign or symptom of an eating disorder is improved following administration of a compound disclosed herein. In some embodiments, a sign or symptom of an eating disorder is measured according to a diary assessment, an assessment by a clinician or caregiver, or a clinical scale. Non-limiting examples of clinical scales, diary assessments, and assessments by a clinician or caregiver include: the Mini International Neuropsychiatric Interview (MINI), the McLean Screening Instrument for Borderline Personality Disorder (MSI-BPD), the Eating Disorder Examination (EDE), the Eating Disorder Questionnaire (EDE-Q), the Eating Disorder Examination Questionnaire Short Form (EDE-QS), the Physical Appearance State and Trait Anxiety Scale-State and Trait version (PASTAS), Spielberger State-Trait Anxiety Inventory (STAI), Eating Disorder Readiness Ruler (ED-RR), Visual Analogue Rating Scales (VAS), the Montgomery-Asberg Depression Rating Scale (MADRS), Yale-Brown Cornell Eating Disorder Scale (YBC-EDS), Yale-Brown Cornell Eating Disorder Scale Self Report (YBC-EDS-SRQ), the Body Image State Scale (BISS), Clinical impairment assessment (CIA) questionnaire, the Eating Disorder Inventory (EDI) (e.g. version 3: EDI-3), the Five Dimension Altered States of Consciousness Questionnaire (5D-ASC), the Columbia-Suicide Severity Rating Scale (C—SSRS), the Life Changes Inventory (LCI), and combinations thereof. In some embodiments, treating according to the methods of the disclosure results in an improvement in an eating disorder compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of the diary assessments, assessments by a clinical or caregiver, or clinical scales, described herein or known in the art.

In some embodiments, the disease or disorder is multiple sclerosis (MS). MS is a chronic, inflammatory disease of unknown etiology that involves an immune-mediated attack on the central nervous system. Myelin and the oligodendrocytes that form myelin appear to be the primary targets of the inflammatory attack, although the axons themselves are also damaged. MS disease activity can be monitored by cranial scans, including magnetic resonance imaging (MRI) of the brain, accumulation of disability, as well as rate and severity of relapses. The diagnosis of clinically definite MS as determined by the Poser criteria requires at least two neurological events suggesting demyelination in the CNS separated in time and in location. Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination. In some embodiments, the multiple sclerosis is relapsing multiple sclerosis. In some embodiments, the relapsing multiple sclerosis is relapsing-remitting multiple sclerosis. In some embodiments, the methods herein reduce a symptom of multiple sclerosis in the subject. In some embodiments, the symptom is a MRI-monitored multiple sclerosis disease activity, relapse rate, accumulation of physical disability, frequency of relapses, decreased tune to confirmed disease progression, decreased time to confirmed relapse, frequency of clinical exacerbation, brain atrophy, neuronal dysfunction, neuronal injury, neuronal degeneration, neuronal apoptosis, risk for confirmed progression, deterioration of visual function, fatigue, impaired mobility, cognitive impairment, reduction of brain volume, abnormalities observed in whole Brain MTR histogram, deterioration in general health status, functional status, quality of life, and/or symptom severity on work. In some embodiments, the methods herein decrease or inhibit reduction of brain volume. In some embodiments, brain volume is measured by percent brain volume change (PBVC). In some embodiments, the methods herein increase time to confirmed disease progression. In some embodiments, time to confirmed disease progression is increased by at least 10%, at least 20%, at least 30%, at least 40%, at least 50%, for example at least 20-60%. In some embodiments, the methods herein decrease abnormalities observed in whole Brain MTR histogram. In some embodiments, the accumulation of physical disability is measured by Kurtzke Expanded Disability Status Scale (EDSS) score. In some embodiments, the accumulation of physical disability is assessed by the time to confirmed disease progression as measured by Kurtzke Expanded Disability Status Scale (EDSS) score.

In some embodiments, the disease or disorder is a disease or disorder characterized by, or otherwise associated with, neuroinflammation. Compounds and compositions of the present disclosure may provide cognitive benefits to subject's suffering from neurological and neurodegenerative diseases such as Alzheimer's disease and other dementia subtypes, Parkinson's disease, amyotrophc lateral sclerosis (ALS), and others where neuroinflammation is a hallmark of disease pathophysiology and progression. For example, emerging psychedelic research/clinical evidence indicates that psychedelics may be useful as disease-modifying treatments in subjects suffering from neurodegenerative diseases such as Alzheimer's disease and other forms of dementia. See Vann Jones, S. A. and O'Kelly, A. “Psychedelics as a Treatment for Alzheimer's Disease Dementia” Front. Synaptic Neurosci., 21, August 2020; Kozlowska, U., Nichols, C., Wiatr, K., and Figiel, M. (2021), “From psychiatry to neurology: Psychedelics as prospective therapeutics for neurodegenerative disorders” Journal of Neurochemistry, 00, 1-20; Garcia-Romeu, A., Darcy, S., Jackson, H., White, T., Rosenberg, P. (2021), “Psychedelics as Novel Therapeutics in Alzheimer's Disease: Rationale and Potential Mechanisms” In: Current Topics in Behavioral Neurosciences. Springer, Berlin, Heidelberg. For example, psychedelics are thought to stimulate neurogenesis, provoke neuroplastic changes, and to reduce neuroinflammation. Thus, in some embodiments, the compounds of the present disclosure (e.g., a compound of Formula (I) through (VII)) are used for the treatment of neurological and neurodegenerative disorders such as Alzheimer's disease, dementia subtypes, Parkinson's disease, and amyotrophc lateral sclerosis (ALS), where neuroinflammation is associated with disease pathogenesis. In some embodiments, the compounds of the present disclosure are used for the treatment of Alzheimer's disease. In some embodiments, the compounds of the present disclosure are used for the treatment of dementia. In some embodiments, the compounds of the present disclosure are used for the treatment of Parkinson's disease. In some embodiments, the compounds of the present disclosure are used for the treatment of amyotrophc lateral sclerosis (ALS). As described above, such treatment may stimulate neurogenesis, provoke neuroplastic changes, and/or provide neuroinflammatory benefits (e.g., reduced neuroinflammation compared to prior to the beginning of treatment), and as a result, may slow or prevent disease progression, slow or reverse brain atrophy, and reduce symptoms associated therewith (e.g., memory loss in the case of Alzheimer's and related dementia disorders). While not limited thereto, pharmaceutical compositions adapted for oral and/or extended-release dosing are appropriate for such treatment methods, with sub-psychedelic dosing being preferred. In some embodiments, treating according to the methods of the disclosure results in an improvement in cognition in subject's suffering from a neurological or neurodegenerative disease compared to pre-treatment of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, according to any one of a diary assessments, assessments by a clinical or caregiver, or clinical scales, described herein or known in the art.

Further, many of the behavioral issues associated with chronic and/or life-threatening illnesses, including neurodegenerative disorders such as Alzheimer's disease, may benefit from treatment with the compounds disclosed herein. Indeed, depression, anxiety, or stress can be common among patients who have chronic and/or life-threatening illnesses such as Alzheimer's disease, autoimmune diseases (e.g., systemic lupus erythematosus, rheumatoid arthritis, and psoriasis), cancer, coronary heart disease, diabetes, epilepsy, HIV/AIDS, hypothyroidism, multiple sclerosis, Parkinson's disease, and stroke. For example, depression is common in Alzheimer's disease as a consequence of the disease, as well as being a risk factor for the disease itself. Symptoms of depression, anxiety, or stress can occur after diagnosis with the disease or illness. Patients that have depression, anxiety, or stress concurrent with another medical disease or illness can have more severe symptoms of both illnesses and symptoms of depression, anxiety, or stress can continue even as a patient's physical health improves. Compounds described herein can be used to treat depression, anxiety, and/or stress associated with a chronic or life-threatening disease or illness.

Accordingly, in some embodiments, the methods herein are used to treat symptoms, e.g., depression, anxiety, and/or stress, associated with a chronic and/or life-threatening disease or disorder, including neurological and neurodegenerative diseases. In some embodiments, the methods provided herein reduce at least one sign or symptom of a neurological and/or neurodegenerative disease. In some embodiments, the methods provided herein reduce at least one sign or symptom of a neurological and/or neurodegenerative disease (e.g., depression, anxiety, and/or stress) by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment, e.g., according to any one of the diary assessments, assessments by a clinical or caregiver, or clinical scales, described herein or known in the art.

In some embodiments, the disease or disorder is Alzheimer's disease. In some embodiments, the methods herein are used for the treatment of depression, anxiety, and/or stress associated with Alzheimer's disease. In some embodiments, the disease or disorder is Parkinson's disease. In some embodiments, the methods herein are used for the treatment of depression, anxiety, and/or stress associated with Parkinson's disease. In some embodiments, the disease or disorder is amyotrophc lateral sclerosis (ALS). In some embodiments, the methods herein are used for the treatment of depression, anxiety, and/or stress associated with amyotrophc lateral sclerosis (ALS). In some embodiments, the disease or disorder is cancer related depression and anxiety. As discussed above, oral and/or extended-release dosing is appropriate for such applications, particularly when blood concentrations of active ingredient (e.g., a compound of Formula (I) through (VII)) are kept below the psychedelic threshold.

In some embodiments, the compounds and compositions disclosed herein are used for treatment of brain injury, including traumatic brain injury (TBI). TBI is an injury to the brain caused by an external force, and can be classified based on severity, ranging from mild traumatic brain injury (mTBI/concussion) to severe traumatic brain injury. TBI can also be categorized by mechanism, as either a closed or penetrating head injury, or other features such as whether it is occurring in a specific location or over a widespread area. TBI can result in physical, cognitive, social, emotional and behavioral symptoms, which may be treated herein. Some of the imaging techniques used for diagnosis and recovery markers include computed tomography (CT) and magnetic resonance imaging (MRIs).

In some embodiments, the disease or disorder is a neurological and developmental disorder such as autism spectrum disorder, including Asperger's syndrome. For example, Asperger's syndrome is a subtype of autism spectrum disorder that is treatable with anxiety drugs. Subjects with autism spectrum disorder may present with various signs and symptoms, including, but not limited to, a preference for non-social stimuli, aberrant non-verbal social behaviors, decreased attention to social stimuli, irritability, anxiety (e.g., generalized anxiety and social anxiety in particular), and depression. In some embodiments, the autism spectrum disorder comprises a medical diagnosis based on the criteria and classification from Diagnostic and Statistical Manual of Mental Disorders, 5th Ed (DSM-5). Current evidence supports the use of psychedelics for ameliorating behavior atypicalities of autism spectrum disorder, including reduced social behavior, anxiety, and depression (see Markopoulos A, Inserra A, De Gregorio D, Gobbi G. Evaluating the Potential Use of Serotonergic Psychedelics in Autism Spectrum Disorder. Front Pharmacol. 2022; 12:749068). The signs and symptoms of autism spectrum disorder may be treated with the methods herein.

In some embodiments, the disease or disorder is a genetic condition that causes learning disabilities and cognitive impairment. An example of such a genetic condition is fragile X syndrome, caused by changes in the gene Fragile X Messenger Ribonucleoprotein 1 (FMR1), which can cause mild to moderate intellectual disabilities in most males and about one-third of affected females. Fragile X syndrome and autism spectrum disorder are closely associated because the FMR1 gene is a leading genetic cause of autism spectrum disorder (see Markopoulos A, Inserra A, De Gregorio D, Gobbi G. Evaluating the Potential Use of Serotonergic Psychedelics in Autism Spectrum Disorder. Front Pharmacol. 2022; 12:749068). Subjects with fragile X syndrome may display anxiety, hyperactive behavior (e.g., fidgeting and impulsive actions), attention deficit disorder, mood and aggression abnormalities, poor recognition memory, and/or features of autism spectrum disorder, and these signs and symptoms may be treated with the methods herein. Clinical trials with psychedelics for the treatment of fragile X syndrome and autism spectrum disorder are currently ongoing (ClinicalTrials.gov, number NCT04869930).

In some embodiments, the disease or disorder is mental distress, e.g., mental distress in frontline healthcare workers.

In some embodiments, the compounds and compositions disclosed herein are used for treatment of tic disorders, including Tourette's Syndrome, which is also variously referred to as Tourette Syndrome, Tourette's Disorder, Gilles de la Tourette syndrome (GTS), or simply Tourette's or TS. The tic disorder may also be a pediatric autoimmune disorder associated with streptococcal infection (PANDAS), a transient tic disorder, a chronic tic disorder, or a tic disorder not otherwise specified (NOS). Tic disorders are defined in the Diagnostic and Statistical Manual of Mental Disorders (DSM) based on type (motor or phonic) and duration of tics (sudden, rapid, nonrhythmic movements), or similarly by the World Health Organization (ICD-10 codes). Tics are involuntary or semi-voluntary, sudden, brief, intermittent, repetitive movements (motor) or sounds (phonic) that are classified as simple or complex. Simple tics, for example, eye blinking or facial grimacing, are relatively easy to camouflage and may go largely unnoticed. Complex tics, such as body contortions, self-injurious behavior, obscene gestures, or shouting of socially inappropriate word or phrases, can appear to be purposeful actions and are particularly distressing. Transient tic disorders are generally characterized by multiple motor and/or phonic tics that occur for at least four weeks but less than 12 months. Chronic tic disorders are generally characterized by either single or multiple motor or phonic tics, but not both, which are present for more than a year. Tourette's Syndrome is a neurodevelopment disorder diagnosed when both motor and phonic tics are present (although not necessarily concurrently) for more than one year. Thus, Tourette's syndrome (TS) is a chronic neuropsychiatric disorder characterized by the presence of fluctuating motor and phonic tics. The typical age of onset is between five and seven years. Affected children may become the target of teasing by peers, which in turn can result in low self-esteem, social isolation, poor school performance, depression and anxiety. In addition to causing social embarrassment, sudden, forceful tics can be painful, and violent head and neck tics have been reported to cause secondary neurologic deficits, such as compressive cervical myelopathy. Tourette's Syndrome patients are also at increased risk for obsessive-compulsive disorder (OCD), depression, and attention-deficit-hyperactivity disorder (ADHD). Tic disorder NOS is diagnosed when tics are present but do not meet the criteria for any specific tic disorder. The present compounds and compositions can also be administered for the treatment of tics induced as a side effect of a medication; tics associated with autism; and Tourettism (the presence of Tourette-like symptoms in the absence of Tourette's Syndrome (e.g., as a result of another disease or condition, such as a sporadic, genetic, or neurodegenerative disorder)).

In some embodiments, the disclosure provides for the management of different kinds of pain, including but not limited to cancer pain, e.g., refractory cancer pain; neuropathic pain; postoperative pain; opioid-induced hyperalgesia and opioid-related tolerance; neurologic pain; postoperative/post-surgical pain; complex regional pain syndrome (CRPS); shock; limb amputation; severe chemical or thermal burn injury; sprains, ligament tears, fractures, wounds and other tissue injuries; dental surgery, procedures and maladies; labor and delivery; during physical therapy; radiation poisoning; acquired immunodeficiency syndrome (AIDS); epidural (or peridural) fibrosis; orthopedic pain; back pain; failed back surgery and failed laminectomy; sciatica; painful sickle cell crisis; arthritis; autoimmune disease; intractable bladder pain; pain associated with certain viruses, e.g., shingles pain or herpes pain; acute nausea, e.g., pain that may be causing the nausea or the abdominal pain that frequently accompanies sever nausea; migraine, e.g., with aura; and other conditions including depression (e.g., acute depression or chronic depression), depression along with pain, alcohol dependence, acute agitation, refractory asthma, acute asthma (e.g., unrelated pain conditions can induce asthma), epilepsy, acute brain injury and stroke, Alzheimer's disease and other disorders. The pain may be persistent or chronic pain that lasts for weeks to years, in some cases even though the injury or illness that caused the pain has healed or gone away, and in some cases despite previous medication and/or treatment. In addition, the disclosure includes the treatment/management of any combination of these types of pain or conditions.

In some embodiments, the pain treated/managed is acute breakthrough pain or pain related to wind-up that can occur in a chronic pain condition. In some embodiments, the pain treated/managed is cancer pain, e.g., refractory cancer pain. In some embodiments, the pain treated/managed is post-surgical pain. In some embodiments, the pain treated/managed is orthopedic pain. In some embodiments, the pain treated/managed is back pain. In some embodiments, the pain treated/managed is neuropathic pain. In some embodiments, the pain treated/managed is dental pain. In some embodiments, the condition treated/managed is depression. In some embodiments, the pain treated/managed is chronic pain in opioid-tolerant patients.

In some embodiments, the disease or disorder is arthritis. Types of arthritis include osteoarthritis, rheumatoid arthritis, childhood arthritis, fibromyalgia, gout, and lupus. In some embodiments, the disease or disorder is osteoarthritis. In some embodiments, the disease or disorder is rheumatoid arthritis. In some embodiments, the disease or disorder is childhood arthritis. In some embodiments, the disease or disorder is gout. In some embodiments, the disease or disorder is lupus. In some embodiments, the disease or disorder is fibromyalgia. Fibromyalgia is a disorder characterized by widespread musculoskeletal pain accompanied by fatigue, sleep, memory and mood issues. Fibromyalgia is believed to amplify painful sensations by affecting brain and spinal cord processes involving painful and nonpainful signaling. Symptoms often begin after an event, such as physical trauma, surgery, infection or significant psychological stress. In other cases, symptoms gradually accumulate over time with no single triggering event. Women are more likely to develop fibromyalgia than are men. Many people who have fibromyalgia also have tension headaches, temporomandibular joint (TMJ) disorders, irritable bowel syndrome, anxiety and depression.

In some embodiments, the disease or disorder is inflammatory bowel disease (IBD). IBD is a term for two conditions, Crohn's disease and ulcerative colitis, that are characterized by chronic inflammation of the gastrointestinal (GI) tract, with such prolonged inflammation resulting in damage to the GI tract. Subjects suffering from IBD may experience persistent diarrhea, abdominal pain, rectal bleeding/bloody stools, weight loss, and fatigue. IBD may be diagnosed, and treatment may be monitored, using one or more of endoscopy, colonoscopy, contrast radiography, MRI, computed tomography (CT), stool samples, and blood tests, known by those of ordinary skill in the art.

In some embodiments, the disease or disorder includes conditions of the autonomic nervous system (ANS). In these embodiments, it may be preferable to use compounds of the present disclosure which are peripherally-restricted.

In some embodiments, the disease or disorder includes pulmonary disorders including asthma and chronic obstructive pulmonary disorder (COPD).

In some embodiments, the disease or disorder includes cardiovascular disorders including atherosclerosis.

In some embodiments, the disease or disorder is a sleep disorder such as narcolepsy, insomnia, nightmare disorder, sleep apnea, central sleep apnea, obstructive sleep apnea, hypopnea, sleep-related hypoventilation, restless legs syndrome, and jet lag. In some embodiments, the disease or disorder is narcolepsy.

In some embodiments, the disclosure relates to a method of treating a disease or condition by modulating N-methyl-D-aspartic acid (NMDA) activity, where the method comprises administering an effective amount of any of the compounds described herein (e.g., any of the compounds described herein (e.g., a compound of Formula (I) through (VII), or a pharmaceutically acceptable salt, solvate, stereoisomer, or prodrug thereof) to a subject in need thereof. In some embodiments, the disease or condition is selected from: levodopa-induced dyskinesia; dementia (e.g., Alzheimer's dementia), tinnitus, treatment resistant depression (TRD), major depressive disorder, neuropathic pain, agitation resulting from or associated with Alzheimer's disease, pseudobulbar effect, autism, Bulbar function, generalized anxiety disorder, Alzheimer's disease, schizophrenia, diabetic neuropathy, acute pain, depression, bipolar depression, suicidality, neuropathic pain, or post-traumatic stress disorder (PTSD). In some embodiments, the disease or condition is a psychiatric or mental disorder (e.g., schizophrenia, mood disorder, substance induced psychosis, major depressive disorder (MDD), bipolar disorder, bipolar depression (BDep), post-traumatic stress disorder (PTSD), suicidal ideation, anxiety, obsessive compulsive disorder (OCD), and treatment-resistant depression (TRD)). In other embodiments, the disease or condition is a neurological disorder (e.g., Huntington's disease (HD), Alzheimer's disease (AD), or systemic lupus erythematosus (SLE)).

In some embodiments, the disclosure relates to a method of treating an ocular disease, such as uveitis, corneal disease, iritis, iridocyclitis, glaucoma, and cataracts, by administering ophthalmically a therapeutically effective amount of any of the compounds described herein (e.g., any of the compounds described herein (e.g., a compound of Formula (I) through (VII)) to a subject in need thereof. For example, compounds herein may be administered in the form of solutions, suspensions, ointments, emulsions, gel-forming solutions, powders for solutions, gels, ocular inserts, and implants. In some embodiments, the compounds are administered in the form of an eye drop formulation.

In some embodiments, the disclosure provides a method of treating a subject with any of the compounds described herein (e.g., a compound of Formula (I) through (VII), or a pharmaceutically acceptable salt, solvate, stereoisomer, or prodrug thereof), comprising the step of administering to a subject an orally administered tablet composition, e.g., matrix composition, of the disclosure comprising any of the compounds described herein (e.g., a compound of Formula (I) through (VII), or a pharmaceutically acceptable salt, solvate, stereoisomer, or prodrug thereof), such that the subject is treated.

The administering physician can provide a method of treatment that is prophylactic or therapeutic by adjusting the amount and timing of any of the compounds described herein (e.g., a compound of Formula (I) through (VII), or a pharmaceutically acceptable salt, solvate, stereoisomer, or prodrug thereof), on the basis of observations of one or more symptoms of the disorder or condition being treated. In some embodiments, the subject is a mammal. In some embodiments, the mammal is a human.

In some embodiments, the disclosure provides a method of continuous oral administration of any of the compounds described herein (e.g., a compound of Formula (I) through (VII), or a pharmaceutically acceptable salt, solvate, stereoisomer, or prodrug thereof). Any of the compounds described herein (e.g., a compound of Formula (I) through (VII), or a pharmaceutically acceptable salt, solvate, stereoisomer, or prodrug thereof), may be formulated into a dosage form such as a tablet composition, e.g., single-layer tablet, that provides a steady release of a therapeutically effective concentration of the compound over a complete release period without neurologically toxic spikes, e.g., no sedative or psychotomimetic toxic spikes in plasma concentration of any of the compounds described herein (e.g., a compound of Formula (I) through (VII), or a pharmaceutically acceptable salt, solvate, stereoisomer, or prodrug thereof).

Compounds of the present disclosure possess advantageous metabolic degradation profiles which prevent high drug concentrations observed acutely after administration, while also enhancing brain levels of the active compound, so that in some embodiments the therapeutic doses may be reduced. As a result, the compounds may be suitable for microdosing to achieve durable therapeutic benefits, with decreased toxicity, e.g., toxicity associated with activation of 5-HT_{2B} receptors associated with valvular heart disease (Rothman, R. B., and Baumann, M. H., 2009, Serotonergic drugs and valvular heart disease, Expert Opin Drug Saf 8, 317-329).

Treatment methods involving administration of a compound of the present disclosure may induce transcriptional changes, e.g., in the frontal cortex, which underlie the beneficial clinical effects. In some embodiments, the methods disclosed herein result in an increased expression of one or more of brain-derived neurotrophic factor (bdnf), homer protein homolog 1 (homer1), Early growth response protein 2 (Egr2 or krox20), and cFos mRNA in the subject's brain, e.g., frontal cortex, as compared to expression levels prior to treatment. In some embodiments, the methods disclosed herein result in an increased expression of bdnf mRNA in the subject's brain, e.g., frontal cortex, as compared to expression levels prior to treatment. The neurotrophin bdnf regulates the survival and growth of neurons, and thus in some embodiments, the treatment methods of the present disclosure increase the levels of bdnf, resulting in positive changes in the subject's synaptic efficiency and plasticity. In some embodiments, the methods disclosed herein result in an increased expression of homer1 mRNA in the subject's brain, e.g., frontal cortex, as compared to expression levels prior to treatment. Increased homer1 expression is associated with the formation of postsynaptic scaffolds that cluster and colocalize NMDAR and mGluR, and thus in some embodiments, the treatment methods of the present disclosure increase the levels of homer1, resulting in improved neuroplasticity such as through regulation of glutamatergic synapses and spine morphogenesis. In some embodiments, the methods disclosed herein result in an increased expression of Egr2 mRNA in the subject's brain, e.g., frontal cortex, as compared to expression levels prior to treatment. Egr2 is a growth factor that has been proposed to be a marker for psychedelic activation of 5-HT_{2A} receptors (Gonzalez-Maeso et al., Transcriptome fingerprints distinguish hallucinogenic and nonhallucinogenic 5-hydroxytryptamine 2A receptor agonist effects in mouse somatosensory cortex. J Neurosci. 2003; 23(26), 8836-43), and thus in some embodiments, the treatment methods of the present disclosure increase the levels of Egr2, resulting in neuroplastic and neuroprotective outcomes. In some embodiments, the methods disclosed herein result in an increased expression of cFos mRNA in the subject's brain, e.g., frontal cortex, as compared to expression levels prior to treatment. cFos is an immediate early gene which is associated with neuronal activity, and thus in some embodiments, the treatment methods of the present disclosure increase the levels of cFos, resulting in increased neuronal firing.

In some embodiments, the compounds/compositions of the disclosure may be used as a standalone therapy. In some embodiments, the compounds/compositions of the disclosure may be used as an adjuvant/combination therapy. In some embodiments, the subject with a disorder is administered the compound/composition of the present disclosure and at least one additional therapy and/or therapeutic. In some embodiments, administration of an additional therapy and/or therapeutic is prior to administration of the compound/composition of the present disclosure. In some embodiments, administration of an additional therapy and/or therapeutic is after administration of the compound/composition of the present disclosure. In some embodiments, administration of an additional therapy and/or therapeutic is concurrent with administration of the compound/composition of the present disclosure. In some embodiments, the additional therapy is an antidepressant, an anticonvulsant, lisdexamfetamine dimesylate, an antipsychotic, an anxiolytic, an anti-inflammatory drug, a benzodiazepine, an analgesic drug, a cardiovascular drug, an opioid antagonist, or combinations thereof.

In some embodiments, the additional therapy is a benzodiazepine. In some embodiments, the benzodiazepine is diazepam or alprazolam.

In some embodiments, the additional therapy is a N-methyl-D-aspartate (NMDA) receptor antagonist. In some embodiments, the NMDA receptor antagonist is ketamine. In some embodiments, the NMDA receptor antagonist is nitrous oxide.

In some embodiments, the additional therapy is an antidepressant. In some embodiments, an antidepressant indirectly affects a neurotransmitter receptor, e.g., via interactions affecting the reactivity of other molecules at a neurotransmitter receptor. In some embodiments, an antidepressant is an agonist. In some embodiments, an antidepressant is an antagonist. In some embodiments, an antidepressant acts (either directly or indirectly) at more than one type of neurotransmitter receptor. In some embodiments, an antidepressant is chosen from buproprion, citalopram, duloxetine, escitalopram, fluoxetine, fluvoxamine, milnacipran, mirtazapine, paroxetine, reboxetine, sertraline, and venlafaxine.

In some embodiments, the antidepressant is a tricyclic antidepressant (“TCA”), selective serotonin reuptake inhibitor (“SSRI”), serotonin and noradrenaline reuptake inhibitor (“SNRI”), dopamine reuptake inhibitor (“DRI”), noradrenaline reuptake Monoamine oxidase inhibitor (“MAOI”), including inhibitor (“NRU”), dopamine, serotonin and noradrenaline reuptake inhibitor (“DSNRI”), a reversible inhibitor of monoamine oxidase type A (RIMA), or combination thereof. In some embodiments, the antidepressant is a TCA. In some embodiments, the TCA is imipramine or clomipramine. In some embodiments, the antidepressant is an SRI. In some embodiments, the SSRI is escitalopram, paroxetine, sertraline, fluvoxamine, fluoxetine, or combinations thereof. In some embodiments, the SNRI is venlafaxine. In some embodiments, the additional therapy is pregabalin.

In some embodiments, the additional therapeutic is an anticonvulsant. In some embodiments, the anticonvulsant is gabapentin, carbamazepine, ethosuximide, lamotrigin, felbamate, topiramate, zonisamide, tiagabine, oxcarbazepine, levetiracetam, divalproex sodium, phenytoin, fosphenytoin. In some embodiments, the anticonvulsant is topiramate.

In some embodiments, the additional therapeutic is an antipsychotic. In some embodiments, the antipsychotic is a phenothiazine, butryophenone, thioxanthene, clozapine, risperidone, olanzapine, or sertindole, quetiapine, aripiprazole, zotepine, perospirone, a neurokinin-3 antagonist, such as osanetant and talnetant, rimonabant, or a combination thereof.

In some embodiments, the additional therapeutic is an anti-inflammatory drug. In some embodiments, the anti-inflammatory drug is a nonsteroidal anti-inflammatory drugs (NSAIDS), steroid, acetaminophen (COX-3 inhibitors), 5-lipoxygenase inhibitor, leukotriene receptor antagonist, leukotriene A4 hydrolase inhibitor, angiotensin converting enzyme antagonist, beta blocker, antihistaminic, histamine 2 receptor antagonist, phosphodiesterase-4 antagonist, cytokine antagonist, CD44 antagonist, antineoplastic agent, 3-hydroxy-3-methylglutaryl coenzyme A inhibitor (statins), estrogen, androgen, antiplatelet agent, antidepressant, Helicobacter pylori inhibitors, proton pump inhibitor, thiazolidinedione, dual-action compounds, or combination thereof.

In some embodiments, the additional therapeutic is an anti-anxiolytic. In some embodiments, an anxiolytic is chosen from alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, or triazolam.

In some embodiments, the pharmaceutical compositions of the disclosure are administered in combination with an opioid, which may be used for example to reduce pain. In some embodiments, the pharmaceutical compositions of the present disclosure serve the purpose of an opioid-sparing medication, i.e., to reduce the amount of opioids necessary to treat a patient.

In some embodiments, the additional therapy is an opioid antagonist. Non-limiting examples of opioid antagonists include naloxone, naltrexone, nalmefene, nalorphine, nalrphine dinicotinate, levallrphan, samidorphan, nalodeine, alvimopan, methylnaltrexone, naloxegol, 6-naltrexol, axelopran, bevenopran, methylsamidorphan, naldemedine, buprenorphine, decozine, butorphanol, levorphanol, nalbuphine, pentazocine, and phenazocine.

In some embodiments, the additional therapy is a cardiovascular drug. Non-limiting examples of cardiovascular drugs include digoxin or (3β,5β,12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribohexopyranosyl) oxy]-12,14-dihydroxy-card-20(22)-enolide, lisinopril, captopril, ramipril, trandolapril, benazepril, cilazapril, enalapril, moexipril, perindopril, quinapril, fludrocortisone, enalaprilate, quinapril, perindopril, apixaban, dabigatran, edoxaban, heparin, rivaroxaban, warfarin, aspirin, clopidogrel, dipyridamole, prasugrel, ticagrelor, azilsartan, candesartan, eprosartan, irbesartan, losartan, olmesartan, telmisartan, valsartanscaubitril, acebutolol, atenolol, betaxolol, bisoprolol, metoprolol, nadolol, propranolol, sotalol, amlodipine, diltiazem, felodipine, nifedipine, nimodipine, nisolidipine, verapamil, statins, nicotinic acids, diuretics, vasodilators, and combinations thereof.

In some embodiments, the subject is administered at least one therapy. Non-limiting examples of therapies include transcranial magnetic stimulation, cognitive behavioral therapy, interpersonal psychotherapy, dialectical behavior therapy, mindfulness techniques, or acceptance, commitment therapy, or combinations thereof.

### Pharmaceutical Compositions

Also disclosed herein is a pharmaceutical composition comprising a compound as disclosed herein (e.g., a compound of Formula (I) through (VII), or a pharmaceutically acceptable salt, solvate, stereoisomer, or prodrug thereof) and a pharmaceutically acceptable excipient. The pharmaceutical compositions may contain one, or more than one, compound of the present disclosure.

The pharmaceutical composition may comprise a single compound of Formula (I) through (VII) or a mixture of compounds of Formula (I) through (VII). The pharmaceutical composition may be formed from an isotopologue mixture of the disclosed compounds. In some embodiments, a subject compound of Formula (I) through (VII) may be present in the pharmaceutical composition at a purity of at least 20% by weight, at least 30% by weight, at least 40% by weight, at least 50% by weight, at least 60% by weight, at least 70% by weight, at least 80% by weight, at least 90% by weight, at least 95% by weight, at least 99% by weight, based on a total weight of isotopologues of the compound of Formula (I) through (VII) present in the pharmaceutical composition. In some embodiments, the composition comprises a subject compound of Formula (I) through (VII), and is substantially free of other isotopologues of the subject compound, in either free base or salt form, e.g., the composition has less than 20, 15, 10, 9, 8, 7, 6, 5, 4, 3, 2 or 1 or 0.5 mole percent of other isotopologues of the subject compound.

In some embodiments, any position in the compound having deuterium has a minimum deuterium incorporation that is greater than that found naturally occurring in hydrogen (about 0.016 atom %). In some embodiments, any position in the compound having deuterium has a minimum deuterium incorporation of at least 10 atom %, at least 20 atom %, at least 25 atom %, at least 30 atom %, at least 40 atom %, at least 45 atom %, at least 50 atom %, at least 60 atom %, at least 70 atom %, at least 80 atom %, at least 90 atom %, at least 95 atom %, at least 99 atom % at the site of deuteration. In preferred embodiments, the composition is substantially free of other isotopologues of the compound, e.g., the composition has less than 20, 15, 10, 9, 8, 7, 6, 5, 4, 3, 2 or 1 or 0.5 mole percent of other isotopologues of the compound.

In some embodiments, the compound of Formula (I) through (VII), or a pharmaceutically acceptable salt, solvate, stereoisomer, or prodrug thereof, is chemically pure, for example has a chemical purity of greater than 90%, 92%, 94%, 96%, 97%, 98%, or 99% by liquid chromatography (e.g., UPLC or HPLC). In some embodiments, the compound of Formula (I) through (VII), or a pharmaceutically acceptable salt, solvate, stereoisomer, or prodrug thereof, has no single impurity of greater than 1%, greater than 0.5%, greater than 0.4%, greater than 0.3%, or greater than 0.2%, measured by liquid chromatography (e.g., UPLC or HPLC). In some embodiments, the compound of Formula (I) through (VII), or a pharmaceutically acceptable salt, solvate, stereoisomer, or prodrug thereof, has a chemical purity of greater than 97 area %, greater than 98 area %, or greater than 99 area % by liquid chromatography (e.g., UPLC or HPLC). In some embodiments, the compound of Formula (I) through (VII), or a pharmaceutically acceptable salt, solvate, stereoisomer, or prodrug thereof, has no single impurity greater than 1 area %, greater than 0.5 area %, greater than 0.4 area %, greater than 0.3 area %, or greater than 0.2 area % as measured by liquid chromatography (e.g., UPLC or HPLC).

The pharmaceutical composition may be formulated with an enantiomerically pure compound of the present disclosure, e.g., a compound of Formula (I) through (VII), or a racemic mixture of the compounds. As described herein, a racemic compound of Formula (I) through (VII) may contain about 50% of the R- and S-stereoisomers based on a molar ratio (about 48 to about 52 mol %, or about a 1:1 ratio)) of one of the isomers. In some embodiments, a composition, medicament, or method of treatment may involve combining separately produced compounds of the R- and S-stereoisomers in an approximately equal molar ratio (about 48 to 52%). In some embodiments, a medicament or pharmaceutical composition may contain a mixture of separate compounds of the R- and S-stereoisomers in different ratios. In some embodiments, the pharmaceutical composition contains an excess (greater than 50%) of the R-enantiomer. Suitable molar ratios of R/S may be from about 1.5:1, 2:1, 3:1, 4:1, 5:1, 10:1, or higher. In some embodiments, a pharmaceutical composition may contain an excess of the S-enantiomer, with the ratios provided for R/S reversed. Other suitable amounts of R/S may be selected. For example, the R-enantiomer may be present in amounts of at least about 55% to 100%, or at least 65%, at least 75%, at least 80%, at least 85%, at least 90%, about 95%, about 98%, or 100%. In other embodiments, the S-enantiomer may be present in a higher percentage, e.g., in amounts of at least about 55% to 100%, or at least 65%, at least 75%, at least 80%, at least 85%, at least 90%, about 95%, about 98%, or 100%. Ratios between all these exemplary embodiments as well as greater than and less than them while still within the disclosure, all are included. Compositions may contain a mixture of the racemate and a separate compound of Formula (I) through (VII), in free base and/or in salt form.

The pharmaceutical composition may be formulated with one or more polymorphs of the compound of Formula (I) through (VII), or a pharmaceutically acceptable salt, solvate, stereoisomer, or prodrug thereof, including crystalline and/or amorphous polymorphs of the compounds or salts thereof.

A pharmaceutical composition can be in unit dosage form. In such form, the pharmaceutical composition is subdivided into unit doses containing appropriate quantities of the active ingredient. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. Pharmaceutical compositions may be generally provided herein which comprise about 0.001 to about 1000 mg, about 1 to about 500 mg, about 2 to about 100 mg, about 0.001 mg, about 0.01 mg, about 0.1 mg, about 1 mg, about 2 mg, about 3 mg, about 5 mg, about 10 mg, about 20 mg, about 30 mg, about 40 mg, about 50 mg, about 75 mg, about 100 mg, about 150 mg, about 200 mg, about 300 mg, about 400 mg, about 500 mg of one or more compounds as disclosed herein (on active basis). The quantity of compound(s) (e.g., compound(s) of Formula (I) through (VII)) (on active basis) in a unit dose preparation may be varied or adjusted within the above ranges as deemed appropriate using sound medical judgment, according to the particular application, administration route, potency of the active ingredient, etc. The composition can, if desired, also contain other compatible therapeutic agents/active ingredients.

In some embodiments, the pharmaceutical composition comprises at least 0.1% by weight, at least 0.5% by weight, at least 1% by weight, at least 5% by weight, at least 10% by weight, at least 15% by weight, at least 20% by weight, at least 25% by weight, at least 30% by weight, at least 35% by weight, at least 40% by weight, at least 45% by weight, at least 50% by weight, and up to 99.9% by weight, up to 99.5% by weight, up to 99% by weight, up to 98% by weight, up to 97% by weight, up to 95% by weight, up to 90% by weight, up to 85% by weight, up to 80% by weight, up to 75% by weight, up to 70% by weight, up to 65% by weight, up to 60% by weight, up to 55% by weight of the compound of Formula (I) through (VII), based on a total weight of the pharmaceutical composition.

The term “excipient” refers to a diluent, adjuvant, vehicle, or carrier with which a compound of the present disclosure is formulated for administration to a mammal. “Pharmaceutically acceptable excipients” may be those diluents, adjuvants, vehicles, or carriers approved by a regulatory agency of the Federal or a state government or listed in the U.S. Pharmacopeia or other generally recognized pharmacopeia for use in mammals, such as humans. Such pharmaceutically acceptable excipients can be solids, semi-solids, or liquids. Examples of solid or semi-solid pharmaceutically acceptable excipients include, but are not limited to, magnesium carbonate, magnesium stearate, talc, keratin, sugar, lactose, pectin, dextrin, fructose, starch, starch paste, gum acacia, gelatin, tragacanth, methylcellulose, sodium carboxymethylcellulose, a low melting wax, cocoa butter, colloidal silica, urea, and the like. Examples of liquid pharmaceutically acceptable excipients include, but are not limited to, water, saline, and oils, including those of petroleum, animal, vegetable or synthetic origin, such as peanut oil, soybean oil, mineral oil, sesame oil and the like. In addition, auxiliary, stabilizing, solubilizing, disintegrating, thickening, lubricating, flavoring, buffering, coloring agents, sweetening agents, and other pharmaceutical additives may be included in the disclosed compositions, for example those set forth hereinafter.

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the compounds may be given continuously or temporarily suspended for a certain length of time (i.e., a “drug holiday”).

Once improvement of the patient's conditions has occurred, a maintenance dose is administered if desired or necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. Administration of the subject compounds may be systemic or local. In some instances, the pharmaceutical compositions are formulated for administration in accordance with routine procedures as a pharmaceutical composition adapted for oral, intravenous, intradermal, transdermal, or inhalation administration, or other routes of administration as set forth herein, to humans. Examples of suitable pharmaceutical excipients and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference. The choice of excipient will be determined in part by the particular compound, as well as by the particular method used to administer the composition. Accordingly, there is a wide variety of suitable formulations of the subject pharmaceutical compositions. Solid form preparations include powders, tablets, pills, capsules, cachets, suppositories, dispersible granules, and the like. A solid excipient may be one or more substance that may also act as diluents, flavoring agents, binders, preservatives, disintegrating agents, or an encapsulating material. Preparations include the formulation of the active compound with encapsulating material as a carrier providing a capsule in which the active component with or without other carriers, is surrounded by a carrier, which is thus in association with it. In powders, the excipient may be a finely divided solid in a mixture with the finely divided active ingredient. In tablets, the active ingredient may be mixed with the excipient(s) having the necessary binding properties in suitable proportions and compacted in the shape and size desired. Liquid form preparations include solutions and emulsions, for example, water, water/propylene glycol solutions, or organic solvents. For parenteral injection, liquid preparations can be formulated in solution in aqueous polyethylene glycol solution. When administered to a mammal, the compounds and compositions of the present disclosure and pharmaceutically acceptable excipients may be sterile. In some instances, an aqueous medium is employed as a vehicle e.g., when the subject compound is administered intravenously, intradermally, or via inhalation, such as water, saline solutions, and aqueous dextrose and glycerol solutions.

In some embodiments, administration to a mammal will result in systemic release of a compound of the present disclosure (for example, into the bloodstream). Routes of administration may include oral routes (e.g., enteral/gastric delivery, intraoral administration such buccal, lingual, and sublingual routes); topical administration (e.g., conjuctival, intracorneal, intraocular, ophthalmic, auricular, transdermal, nasal (e.g., intranasal), vaginal, uretheral, respiratory, and rectal administration); administration by inhalation via, for example a nebulizer or inhaler; and parenteral routes (e.g., intravenous, intradermal, intraarterial, intraperitoneal, intrathecal, intraventricular, intraurethral, intrasternal, intracranial, intramuscular, intrasynovial, and subcutaneous administration, including those routes using an automatic injection device). In some embodiments, the pharmaceutical composition herein is formulated for oral administration. In some embodiments, the pharmaceutical composition herein is formulated for administration via inhalation. In some embodiments, the pharmaceutical composition herein is formulated for administration via injection, e.g. intravenously, intramuscularly, intracutaneously, subcutaneously, intraduodenally, or intraperitoneally. In some embodiments, the pharmaceutical composition herein is formulated for topical administration, e.g., as a cream or in the form of a skin patch for transdermal administration. In some embodiments, the compounds described herein may be administered via an automatic injection device.

As described below, the pharmaceutical compositions of the present disclosure may be specially formulated for administration in solid, semi-solid, or liquid form, including those adapted for the following:
A. Oral administration, for example, drenches (aqueous or non-aqueous solutions or suspensions), tablets, pills, cachets, lozenges, films, or capsules, e.g., those targeted for buccal, sublingual, and systemic absorption, boluses, powders, granules, syrups, pastes for application to the tongue;
B. Parenteral administration, for example, by subcutaneous, intradermal, intramuscular, intravenous or epidural injection as, for example, a sterile solution or suspension, or sustained release formulation;
C. Topical application/transdermal administration, for example, as a cream, ointment, or a controlled release patch or spray applied to the skin, or orifices and/or mucosal surfaces such as intravaginally or intrarectally, for example, as a pessary, cream or foam;
D. Modified release dosage forms, including delayed-, extended-, prolonged-, sustained-, pulsatile-, controlled-, accelerated-, fast-, targeted-, programmed-release, and gastric retention dosage forms, such modified release dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126); and
E. Inhalation administration, for example as an aerosol, preferably a mist.

Tamper resistant dosage forms/packaging of any of the disclosed pharmaceutical compositions are contemplated.

### A. Oral Administration

The pharmaceutical compositions disclosed herein may be provided in solid, semisolid, or liquid dosage forms for oral administration. As used herein, oral administration includes gastric (enteral) delivery, for example whereby the medication is taken by mouth and swallowed, as well as intraoral administration such as through the mucosal linings of the oral cavity, e.g., buccal, lingual, and sublingual administration. Suitable oral dosage forms include, but are not limited to, tablets, capsules, caplets, pills, troches, lozenges, pastilles, cachets, gelcaps, caps, pellets, orodispersible dosage forms (e.g., orally disintegrating tablets), sublingual tablets, buccal tablets, medicated chewing gum, granules, bulk powders, effervescent or non-effervescent powders or granules, solutions, boluses, emulsions, suspensions, solutions, wafers, films, sprinkles, elixirs, and syrups. In addition to the active ingredient(s), the pharmaceutical compositions may contain one or more pharmaceutically acceptable excipients (e.g., carriers or vehicles), including, but not limited to, binders, fillers, diluents, disintegrants, wetting agents, lubricants, glidants, coloring agents, dye-migration inhibitors, sweetening agents, preservatives, antioxidants, lyoprotectants, stabilizing agents, solubilizing agents, complexing agents, and flavoring agents. In some embodiments, the pharmaceutical composition contains from about 1% to about 95% by weight, 5% to about 70% by weight, or from about 10% to about 60% by weight, or from about 20% to about 50% by weight, or from about 30% to about 40% by weight of the active ingredient (e.g., a compound of Formula (I) through (VII)).

In some embodiments, pharmaceutical compositions of the present disclosure may be in orodispersible dosage forms (ODxs), including orally disintegrating tablets (ODTs) (also sometimes referred to as fast disintegrating tablets, orodispersible tablets, or fast dispersible tablets) or orodispersible films (ODFs) (or wafers). Such dosage forms allow for pre-gastric absorption of the compounds herein, e.g., when administered intraorally through the mucosal linings of the oral cavity, e.g., buccal, lingual, and sublingual administration, for increased bioavailability and faster onset compared to oral administration through the gastrointestinal tract.

Orodispersible dosage forms can be prepared by different techniques, such as freeze drying (lyophilization), molding, spray drying, mass extrusion or compressing. Preferably, the orodispersible dosage forms are prepared by lyophilization. In some embodiments, the orodispersible dosage forms disintegrate in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the orodispersible dosage forms dissolve in less than about 90 seconds, in less than about 60 seconds, or in less than about 30 seconds after being received in the oral cavity. In some embodiments, the orodispersible dosage forms disperse in less than about 90 seconds, in less than about 60 seconds, in less than about 30 seconds, in less than about 20, in less than about 10 seconds, in less than about 5 seconds, or in less than about 2 seconds after being received in the oral cavity. In some embodiments, the pharmaceutical compositions are in the form of orodispersible dosage forms, such as oral disintegrating tablets (ODTs), having a disintegration time according to the United States Phamacopeia (USP) disintegration test <701> of not more than about 30 seconds, not more than about 20, not more than about 10 seconds, not more than about 5 seconds, not more than about 2 seconds. Orodispersible dosage forms having longer disintegration times according to the United States Phamacopeia (USP) disintegration test <701>, such as when adapted for extended release, for example 2 minutes, 3 minutes, 4 minutes, 5 minutes, 10 minutes, 15 minutes, 20 minutes, 25 minutes, 30 minutes, 45 minutes, 60 minutes, or any range therebetween, or longer, are also contemplated.

In some embodiments, the pharmaceutical compositions are in the form of lyophilized orodispersible dosage forms, such as lyopholized ODTs. In some embodiments, the lyophilized orodispersible dosage forms (e.g., lyophilized ODTs) are created by creating a porous matrix by subliming the water from pre-frozen aqueous formulation of the drug containing matrix-forming agents and other excipients such as those set forth herein, e.g., one or more lyoprotectants, preservatives, antioxidants, stabilizing agents, solubilizing agents, flavoring agents, etc. In some embodiments, the orodispersible dosage form comprises two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the dosage form (binder). In some embodiments, the second constituent is a matrix-supporting/disintegration-enhancing agent such as sucrose, lactose, mannitol, xylitol, microcrystalline cellulose, calcium diphosphate, and/or starch, which acts by cementing the porous framework, provided by the water-soluble polymer and accelerates the disintegration of the orodispersible dosage form. In some embodiments, the lyophilized orodispersible dosage form (e.g., lyophilized ODT) includes gelatin and mannitol. In some embodiments, the lyophilized orodispersible dosage form (e.g., lyophilized ODT) includes gelatin, mannitol, and one or more of a lyoprotectant, a preservative, an antioxidant, a stabilizing agent, a solubilizing agent, a flavoring agent, etc., with particular mention being made to citric acid. A non-limiting example of an ODT formulation is Zydis® orally dispersible tablets (available from Catalent). In some embodiments, the ODT formulation (e.g., Zydis® orally dispersible tablets) includes one or more water-soluble polymers, such as gelatin, one or more matrix materials, fillers, or diluents such as mannitol, a compound of Formula (I) through (VII), or a pharmaceutically acceptable salt, solvate, stereoisomer, or prodrug thereof, and optionally a lyoprotectant, a preservative, an antioxidant, a stabilizing agent, a solubilizing agent, and/or a flavoring agent. In some embodiments, the ODT formulation (e.g., Zydis® orally dispersible tablets) includes gelatin, mannitol, a compound of Formula (I) through (VII), or a pharmaceutically acceptable salt, solvate, stereoisomer, or prodrug thereof, and an organic acid, non-limiting examples of which are citric acid and/or tartaric acid, or any suitable organic acid set forth herein.

In some embodiments, the pharmaceutical composition is in the form of lyophilized orodispersible film (ODF) (or wafer). In some embodiments, the pharmaceutical compositions are in the form of lyophilized ODFs protected for the long-term storage by a specialty packaging excluding moisture, oxygen, and light. In some embodiments, the lyophilized ODFs are created by creating a porous matrix by subliming the water from pre-frozen aqueous formulation of the drug containing matrix-forming agents and other excipients such as those set forth herein, e.g., one or more of a lyoprotectant, a preservative, an antioxidant, a stabilizing agent, a solubilizing agent, a flavoring agent, etc. In some embodiments, the lyophilized ODF includes a thin water-soluble film matrix. In some embodiments, the ODFs comprise two component frameworks of a lyophilized matrix system that work together to ensure the development of a successful formulation. In some embodiments, the first component is a water-soluble polymer such as gelatin, dextran, alginate, and maltodextrin. This component maintains the shape and provides mechanical strength to the film/wafer (binder). In some embodiments, the second constituent is matrix-supporting/disintegration-enhancing agents such as sucrose and mannitol, which acts by cementing the porous framework, provided by the water-soluble polymer and accelerates the disintegration of the wafer. In some embodiments, the lyophilized ODFs include gelatin and mannitol. In some embodiments, the lyophilized ODFs include gelatin, mannitol, and one or more of a lyoprotectant, a preservative, an antioxidant, a stabilizing agent, a solubilizing agent, a flavoring agent, etc., with particular mention being made to citric acid.

In some embodiments, the ODF (or wafer) can comprise a monolayer, bilayer, or trilayer. In some embodiments, the monolayer ODF contains an active agent and one or more pharmaceutically acceptable excipients (e.g., carrier or excipients). In some embodiments, the bilayer ODF contains one or more excipients, such as a solubilizing agent, in a first layer and an active agent in the second layer. This configuration allows the active agent to be stored separately from the excipients and can increase the stability of the active agent and optionally increase the shelf life of the composition compared to the case where the excipients and the active agent were contained in a single layer. For tri-layer ODFs, each of the layers may be different or two of the layers, such as the upper and lower layers, may have substantially the same composition. In some embodiments, the lower and upper layers surround a core layer containing the active agent. In some embodiments, the lower and upper layers may contain one or more excipients, such as a solubilizing agent. In some embodiments, the lower and upper layers have the same composition. Alternatively, the lower and upper layers may contain different excipients or different amounts of the same excipient. The core layer typically contains the active agent, optionally with one or more excipients.

In some embodiments, in addition to the active ingredient(s), the pharmaceutical compositions in orodispersible dosage forms (ODxs) may contain one or more pharmaceutically acceptable excipients (e.g., carriers or vehicles). For example, in some embodiments, pharmaceutical compositions in orodispersible dosage forms include one or more of pharmaceutically acceptable a lyoprotectant, a preservative, an antioxidant, a stabilizing agent, a solubilizing agent, a flavoring agent, etc.

Examples of pharmaceutically acceptable lyoprotectants include, but are not limited to, disaccharides such as sucrose and trehalose, anionic polymers such as sulfobutylether-β-cyclodextrin (SBECD) and hyaluronic acid, and hydroxylated cyclodextrins.

Examples of pharmaceutically acceptable preservatives include, but are not limited to, glycerin, methyl and propylparaben, benzoic acid, sodium benzoate and alcohol.

Examples of pharmaceutically acceptable antioxidants, which may act to further enhance stability of the composition, include: (1) water soluble antioxidants, such as ascorbic acid, cysteine or salts thereof (cysteine hydrochloride), sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of pharmaceutically acceptable stabilizing agents include, but are not limited to, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, glycerol, methionine, monothioglycerol, ascorbic acid, citric acid, polysorbate, arginine, cyclodextrins, microcrystalline cellulose, modified celluloses (e.g., carboxymethylcellulose, sodium salt), sorbitol, and cellulose gel.

Examples of pharmaceutically acceptable solubilizing agents (or dissolution aids) include, but are not limited to, citric acid, hydroxypropylcellulose, hydroxypropylmethylcellulose, sodium stearyl fumarate, methacrylic acid copolymer LD, methylcellulose, sodium lauryl sulfate, polyoxyl 40 stearate, purified shellac, sodium dehydroacetate, fumaric acid, DL-malic acid, L-ascorbyl stearate, L-asparagine acid, adipic acid, aminoalkyl methacrylate copolymer E, propylene glycol alginate, casein, casein sodium, a carboxyvinyl polymer, carboxymethylethylcellulose, powdered agar, guar gum, succinic acid, copolyvidone, cellulose acetate phthalate, tartaric acid, dioctylsodium sulfosuccinate, zein, powdered skim milk, sorbitan trioleate, lactic acid, aluminum lactate, ascorbyl palmitate, hydroxyethylmethylcellulose, hydroxypropylmethylcelluloseacetate succinate, polyoxyethylene (105) polyoxypropylene (5) glycol, polyoxyethylene hydrogenated castor oil 60, polyoxyl 35 castor oil, poly(sodium 4-styrenesulfonate), polyvinylacetaldiethylamino acetate, polyvinyl alcohol, maleic acid, methacrylic acid copolymer S, lauromacrogol, sulfuric acid, aluminum sulfate, phosphoric acid, calcium dihydrogen phosphate, sodium dodecylbenzenesulfonate, a vinyl pyrrolidone-vinyl acetate copolymer, sodium lauroyl sarcosinate, acetyl tryptophan, sodium methyl sulfate, sodium ethyl sulfate, sodium butyl sulfate, sodium octyl sulfate, sodium decyl sulfate, sodium tetradecyl sulfate, sodium hexadecyl sulfate, and sodium octadecyl sulfate. Of these, in some embodiments, such as in ODT formulation, citric acid is preferred.

Flavoring agents include natural flavors extracted from plants, such as fruits, and synthetic blends of compounds which produce a pleasant taste sensation or taste masking effect. Examples of flavoring agents include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, glucose, wild orange peel, citric acid, tartaric acid, oil of wintergreen, oil of peppermint, methyl salicylate, oil of spearmint, oil of sassafras, oil of clove, cinnamon, anethole, menthol, thymol, eugenol, eucalyptol, lemon, lime, and lemon-lime.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

Pharmaceutical compositions adapted for oral administration, e.g., general tablets including single-layer tablets, compressed tablets, coated tablets, etc. may be formulated with various excipients such as those set forth herein. Examples of suitable excipients may include, but are not limited to, binders, fillers, diluents, disintegrants, wetting agents, lubricants, glidants, coloring agents, dye-migration inhibitors, sweetening agents, preservatives, antioxidants, stabilizing agents, solubilizing agents, and flavoring agents.

Binders or granulators impart cohesiveness to a tablet to ensure the tablet remains intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present, e.g., from about 10%, about 20%, about 30%, about 40%, about 50%, about 60%, about 70%, about 80%, about 90%, about 99% by weight in the pharmaceutical compositions disclosed herein, or any range therebetween.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain, e.g., from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; sodium stearyl fumarate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions disclosed herein may contain, e.g., about 0.1 to about 5% by weight of a lubricant.

Suitable glidants include colloidal silicon dioxide, CAB-O-SIL® (Cabot Co. of Boston, Mass.), and asbestos-free talc.

Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Sweetening agents include sucrose, lactose, mannitol, syrups, glycerin, and artificial sweeteners, such as saccharin and aspartame.

Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate.

Suspending and dispersing agents include sodium carboxymethylcellulose, pectin, tragacanth, Veegum, acacia, sodium carbomethylcellulose, hydroxypropyl methylcellulose, and polyvinylpyrolidone.

Preservatives include glycerin, methyl and propylparaben, benzoic add, sodium benzoate and alcohol.

Wetting agents include propylene glycol monostearate, sorbitan monooleate, diethylene glycol monolaurate, and polyoxyethylene lauryl ether.

Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many excipients (carriers, vehicles, etc.) may serve several functions, even within the same formulation. Particular mention is made to pharmaceutical compositions herein containing citric acid, which may play multiple roles as a stabilizing agent, as a solubilizing agent to provide fast dissolution of the active for rapid onset, etc., particularly for dosage forms adapted for rapid onset and a shorter duration of drug action, such as orodispersible dosage forms (e.g., ODTs and ODFs).

The tablet dosage forms may be prepared from the active ingredient in powdered, crystalline, or granular forms, alone or in combination with one or more excipients described herein, including binders, disintegrants, controlled-release polymers, lubricants, diluents, and/or colorants. Flavoring and sweetening agents are especially useful in the formation of chewable tablets and lozenges.

The pharmaceutical compositions herein may be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. In some embodiments, the enteric coatings protect the dosage form from the acidic environment of the stomach and maintain an extended-release profile. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets. In some embodiments, the solid oral dosage form (e.g., a single-layer tablet or caplet) is coated with one or more protective layers of inactive pharmaceutically acceptable excipients to form a modified-release formulation, e.g., to ensure steady release of the active ingredient from the matrix and avoid concentration bursts at the early release time points.

The pharmaceutical composition may be orally administered to a subject, such that a continuous therapeutically effective concentration of any of the compounds described herein (e.g., a compound of Formula (I) through (VII), or a pharmaceutically acceptable salt, solvate, stereoisomer, or prodrug thereof), is provided to the subject. In this regard, the disclosure provides novel and inventive formulations for oral administration comprising, e.g., optimal matrices discovered for the long-term steady release of any of the compounds of Formula (I) through (VII), or a pharmaceutically acceptable salt, solvate, stereoisomer, or prodrug thereof, with reduced sedative and psychotomimetic side effects.

In some embodiments, the pharmaceutical composition (e.g., a tablet composition formulated for oral administration such as a single-layer tablet composition), comprises any of the compounds described herein (e.g., a compound of Formula (I) through (VII), or a pharmaceutically acceptable salt, solvate, stereoisomer, or prodrug thereof), and a polymer. In some embodiments, the pharmaceutical composition includes: (i) a water-insoluble neutrally charged non-ionic matrix; and (ii) a polymer carrying one or more negatively charged groups.

In some embodiments, the tablet composition is a modified-release tablet adapted for sustained release and preferably maximum sustained release. In some embodiments, the release period of any of the compounds described herein (e.g., a compound of Formula (I) through (VII)), in the formulations of the disclosure is greater than 4 hours, greater than 6 hours, greater than 8 hours, greater than 10 hours, greater than 12 hours, greater than 16 hours, greater than 20 hours, greater than 24 hours, greater than 28 hours, greater than 32 hours, greater than 36 hours, greater than 48 hours.

In some embodiments, the tablet composition is adapted for tamper resistance. In some embodiments, the tablet composition comprises polyethylene oxide (PEO), e.g., MW about 2,000 to about 7,000 KDa, optionally in combination with HPMC. In some embodiments, the tablet composition may further comprise polyethylene glycol (PEG), e.g., PEG 8K. In some embodiments, the tablet composition may further comprise a polymer carrying one or more negatively charged groups, e.g., polyacrylic acid. In some embodiments, the tablet composition comprising PEO is further subjected to heating/annealing, e.g., extrusion conditions.

In some embodiments, the pharmaceutical composition comprises a combination of (i) a water-insoluble neutrally charged non-ionic matrix; (ii) a polymer carrying one or more negatively charged groups; and (iii) any of the compounds described herein (e.g., a compound of Formula (I) through (VII), or a pharmaceutically acceptable salt, solvate, stereoisomer, or prodrug thereof.

In some embodiments, the polymer carrying one or more negatively charged groups is selected from the group consisting of polyacrylic acid, polylactic acid, polyglycolic acid, polymethacrylate carboxylates, cation-exchange resins, clays, zeolites, hyaluronic acid, anionic gums, salts thereof, and mixtures thereof. In some embodiments, the anionic gum is selected from the group consisting of naturally occurring materials and semi-synthetic materials. In some embodiments, the naturally occurring material is selected from the group consisting of alginic acid, pectin, xanthan gum, carrageenan, locust bean gum, gum arabic, gum karaya, guar gum, and gum tragacanth. In some embodiments, the semi-synthetic material is selected from the group consisting of carboxymethyl-chitin and cellulose gum.

Moreover, without wishing to be bound by theory, in some embodiments, the role of the polymer carrying one or more negatively charged groups, e.g., moieties of acidic nature as in those of the acidic polymers described herein, surprisingly offers significant retention of any of the compounds described herein (e.g., a compound of Formula (I) through (VII), or a pharmaceutically acceptable salt, solvate, stereoisomer, or prodrug thereof), in the matrix. In some embodiments, this negative charge may be created in situ, for example, based on release of a proton due to pKa and under certain pH conditions or through electrostatic interaction/creation of negative charge. Further noting that acidic polymers may be the salts of the corresponding weak acids that will be the related protonated acids in the stomach; which, and without wishing to be bound by theory, will neutralize the charge and may reduce the interactions of any of the compounds described herein (e.g., a compound of Formula (I) through (VII), or a pharmaceutically acceptable salt, solvate, stereoisomer, or prodrug thereof), with the matrix. In addition, the release matrix may be further complemented by other inactive pharmaceutical ingredients to aid in preparation of the appropriate solid dose form such as fillers, disintegrants, flow improving agents, lubricants, colorants, taste maskers.

In some embodiments, the water-insoluble neutrally charged non-ionic matrix is selected from cellulose-based polymers such as HPMC, alone or enhanced by mixing with components selected from the group consisting of starches; waxes; neutral gums; polymethacrylates; PVA; PVA/PVP blends; and mixtures thereof.

In some embodiments, the cellulose-based polymer is hydroxypropyl methylcellulose (HPMC). In some embodiments, the tablet composition comprises about 10 to 70%, 20 to 60%, or 30 to 50% hydroxypropyl methylcellulose by weight, about 10 to 30%, or about 15 to 20% starch by weight, or any combination thereof.

In some embodiments, the disclosure provides a method of formulating any of the compounds described herein (e.g., a compound of Formula (I) through (VII), or a pharmaceutically acceptable salt, solvate, stereoisomer, or prodrug thereof), to ensure the steady release of a therapeutically effective concentration of any of the compounds from an oral tablet without neurologically toxic spikes, e.g., sedative or psychotomimetic toxic spikes, in plasma concentration of any of the compounds. In some embodiments, the method comprises the step of combining (i) a water-insoluble neutrally charged non-ionic matrix; (ii) a polymer carrying one or more negatively charged groups; and (iii) any of the compounds described herein (e.g., a compound of Formula (I) through (VII), or a pharmaceutically acceptable salt, solvate, stereoisomer, or prodrug thereof), to produce an orally administered tablet composition, e.g., single-layer. In some embodiments, the method comprises the step of combining (i) polyethylene oxide (PEO), e.g., MW about 2,000 to about 7,000 KDa, with HPMC, and (ii) any of the compounds described herein (e.g., a compound of Formula (I) through (VII), or a pharmaceutically acceptable salt, solvate, stereoisomer, or prodrug thereof), to produce an orally administered tablet composition, e.g., single-layer. In some embodiments, the method comprises the step of combining polyethylene oxide (PEO) with HPMC, and any of the compounds described herein (e.g., a compound of Formula (I) through (VII), or a pharmaceutically acceptable salt, solvate, stereoisomer, or prodrug thereof), the tablet composition may further comprise a polymer carrying one or more negatively charged groups, e.g., polyacrylic acid and/or may be further subjected to heating/annealing, e.g., extrusion conditions.

Disclosed herein are pharmaceutical compositions in modified release dosage forms, which comprise a compound as disclosed herein and one or more release controlling excipients or carriers as described herein. Suitable modified release dosage excipients include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients or carriers.

In some embodiments, provided is a modified release oral formulation. In some embodiments, the oral pharmaceutical composition is for low dose maintenance therapy that can be constructed using the compounds described herein, capitalizing on the ability of the phenethylamine-type compounds described herein to bind with anionic polymers.

In some embodiments, the pharmaceutical composition contains a compound of the present disclosure, which is an orally active, peripherally-restricted, 5-HT₂ agonist, for the treatment of autonomic nervous system disorders, including pulmonary disorders (e.g., asthma) and cardiovascular disorders (e.g., atherosclerosis).

Further disclosed herein are pharmaceutical compositions in enteric coated dosage forms, which comprise a compound as disclosed herein and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise a compound as disclosed herein and one or more release controlling excipients or carriers for use in an effervescent dosage form. The pharmaceutical compositions may also comprise non-release controlling excipients or carriers. Effervescent means that the dosage form, when mixed with liquid, including water, juice, saliva, etc., evolves a gas. In general, the effervescent dosage forms of the present disclosure comprise an organic acid and a source of carbon dioxide, referred to herein as an “effervescent couple.” Such effervescent dosage forms effervesce (evolve gas) through chemical reaction between the organic acid and the source of carbon dioxide, which takes place upon exposure to an aqueous environment, such as upon placement in water, juice, or other drinkable fluid, or from the aqueous environment in the oral cavity, such as saliva in the mouth. Specifically, the reaction between the organic acid and the source of carbon dioxide produces carbon dioxide gas upon contact with an aqueous medium such as water, juice, or saliva. While use of disintegrants are optional, effervescent dosage forms do not require a disintegrant as the evolution of the gas in situ facilitates the disintegration process.

For clarity, an “effervescent couple” refers to at least one organic acid and at least one source of carbon dioxide being contained in a dosage form, regardless of assembly—for example, the organic acid and the source of carbon dioxide can be admixed (as powders), layered on top of one another, agglomerated or otherwise “glued” together in granular form, or held separately from one another such as in separate layers within the dosage form. Further, the term “couple” in this context is not meant to be limited to only an organic acid and a source of carbon dioxide and is open to the inclusion of other materials unless specified otherwise; for example, effervescent agglomerates/granules made from bringing together (or “gluing”) an organic acid and a source of carbon dioxide may include other vehicles including binders (the “glue”) and the effervescent agglomerates/granules may nonetheless be referred to as an effervescent couple.

The organic acid may be a monoacid, a diacid, a triacid, a tetraacid, or may contain a higher number of acid groups. One organic acid or mixtures of organic acids may be used. In addition to an acid group(s) (e.g., one or more carboxylic acid moieties), the organic acid may also contain one or more hydroxyl functionalities as part of its structure (i.e., the organic acid may be a hydroxy acid). In some embodiments, the organic acid is an α-hydroxy acid. In some embodiments, the organic acid is a β-hydroxy acid. In some embodiments, the organic acid is a γ-hydroxy acid. Examples of hydroxy acids include, but are not limited to, glycolic acid, lactic acid, citric acid, tartaric acid, and malic acid. In some embodiments, the organic acid is citric acid and/or tartaric acid. In some embodiments, the organic acid is citric acid. In some embodiments, the organic acid is tartaric acid. In some embodiments, the organic acid is an enedioic acid, examples of which may include, but are not limited to, fumaric acid and maleic acid. In some embodiments, the organic acid is fumaric acid. In some embodiments, the organic acid is maleic acid. Mixtures and/or hydrates of the disclosed organic acid may also be used in the disclosed pharmaceutical compositions. In some embodiments, the organic acid is not a sulfonic acid (e.g., benzenesulfonic acid, camphorsulfonic acid, (+)-(1S)-camphor-10-sulfonic acid, ethane-1,2-disulfonic acid, ethanesulfonic acid, 2-hydroxy-ethanesulfonic acid, methanesulfonic acid, naphthalene-2-sulfonic acid, naphthalene-1,5-disulfonic acid, p-toluenesulfonic acid, ethanedisulfonic acid, etc.). In some embodiments, the organic acid is not a benzoic acid (e.g., benzoic acid, 4-acetamidobenzoic acid, 2-acetoxybenzoic acid, salicylic acid, 4-amino-salicylic acid, gentisic acid, etc.).

The source of carbon dioxide may include, but is not limited to, sodium bicarbonate, sodium carbonate, potassium carbonate, potassium bicarbonate, magnesium carbonate, calcium carbonate, and sesquicarbonate. The source of carbon dioxide can be used singly, or in combination. In some embodiments, the source of carbon dioxide is sodium bicarbonate. In some embodiments, the source of carbon dioxide is sodium carbonate. In some embodiments, the source of carbon dioxide is potassium carbonate. In some embodiments, the source of carbon dioxide is potassium bicarbonate. However, reactants which evolve oxygen or other gases besides carbon dioxide, and which are safe for human consumption, are also contemplated for use in the disclosed effervescent dosage forms, in addition to or in lieu of the source of carbon dioxide. While not wishing to be bound by theory, it is believed that the effervescence can help quickly break up the dosage form, and in some routes of administration such as intraoral routes, can help reduce the perception of grittiness by providing a distracting sensory experience of effervescence.

In some embodiments, the effervescent dosage form is to be reconstituted in a drinkable fluid such as water or juice, thereby forming an oral liquid dosage form (e.g., solution), prior to consumption. In some embodiments, the effervescent dosage form is to be placed in the oral cavity, where contact with the aqueous environment (saliva) causes disintegration/dissolution of the dosage form along with effervescence. Here, the contents of the effervescent dosage form may be converted into a liquid or semi-solid dosage form, such as a solution, syrup, or paste upon mixing with the saliva, and subsequently swallowed. Alternatively, the effervescent dosage form may be an intraoral dosage form, e.g., a buccal, lingual, or sublingual dosage form, whereby placement in the aqueous environment (saliva) of the oral cavity causes disintegration/dissolution of the dosage form along with effervescence, and pre-gastric absorption of the contents through the oral mucosa. Such pre-gastric absorption may provide for increased bioavailability and faster onset compared to oral administration through the gastrointestinal tract. In some embodiments, the effervescent dosage form is a sublingual dosage form to be disintegrated/dissolved under the tongue, whereby the contents (e.g., the compounds of the present disclosure) are absorbed through the mucous membrane beneath the tongue where they enter venous circulation. In some embodiments, the effervescent dosage form is a buccal dosage form to be disintegrated/dissolved in the buccal cavity, whereby the contents (e.g., the compounds of the present disclosure) are absorbed through the oral mucosa lining the mouth where they enter venous circulation. Effervescent dosage forms may be advantageous for the treatment of pediatric/adolescent patients or patients that have general difficulty swallowing traditional dosage forms such as general tablets or capsules, since effervescent dosage forms can be reconstituted into easy to swallow liquid or semi-solid dosage forms or taken intraorally.

When adapted for intraoral administration, it may be beneficial to formulate the effervescent dosage form with a bioadhesive agent, in addition to the effervescent couple. “Bioadhesive agents” are substances which promote adhesion or adherence to a biological surface, such as mucous membranes. For example, bioadhesive agents are themselves capable of adhering to a biological surface when placed in contact with that surface (e.g., mucous membrane) in order to enable compositions of the disclosure to adhere to that surface, which promotes more efficient transfer of the contents from the dosage form to the biological surface. A variety of polymers known in the art can be used as bioadhesive agents, for example polymeric substances, preferably with an average (weight average) molecular weight above 5,000 g/mol. It is preferred that such polymeric materials are capable of rapid swelling when placed in contact with an aqueous medium such a water or saliva, and/or are substantially insoluble in water at room temperature and atmospheric pressure. Examples of suitable bioadhesive agents include, but are not limited to, cyclodextrin, cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, carboxymethyl cellulose, modified cellulose gum and sodium carboxymethyl cellulose (NaCMC); starch derivatives such as moderately cross-linked starch, modified starch and sodium starch glycolate; acrylic polymers such as carbomer and its derivatives (polycarbophyl, Carbopol®, etc.); polyvinylpyrrolidone (PVP); polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); and crosscarmellose (e.g. crosscarmellose sodium). Such polymers may be crosslinked. Combinations of two or more bioadhesive agents can also be used.

An effervescent couple can be coated with a pharmaceutically acceptable excipient, e.g., with a binder, a protective coating such as a solvent protective coating, an enteric coating, an anti-caking agent, and/or a pH modifier to prevent premature reaction, e.g., with air, moisture, and/or other ingredients contained in the pharmaceutical composition. Each component of the effervescent couple, e.g., the organic acid and/or the source of carbon dioxide, can also individually be coated with a pharmaceutically acceptable excipient, e.g., with a binder, a protective coating such as a solvent protective coating, an enteric coating, an anti-caking agent, and/or a pH modifier to prevent premature reaction, e.g., with air, moisture, and/or other ingredients contained in the pharmaceutical composition. The effervescent couple can also be mixed with previously lyophilized particles, such as one or more pharmaceutically active ingredients coated with a solvent protective or enteric coating.

The effervescent dosage form may be prepared by methods known to those skilled in the art, including, but not limited to, slugging, direct compression, roller compaction, dry or wet granulation, fusion granulation, melt-granulation, vacuum granulation, and fluid bed spray granulation, any of which may be optionally followed by compression/tableting.

The pharmaceutical compositions disclosed herein may be formulated as non-effervescent or effervescent granules and powders. The non-effervescent or effervescent granules and powders may be reconstituted into a liquid dosage form, or alternatively, compressed to form tablet dosage forms which are either non-effervescent or effervescent, respectively. Pharmaceutically acceptable excipients used in the non-effervescent or effervescent granules or powders may include, but are not limited to, binders, granulators, fillers, diluents, sweetening agent, wetting agents, stabilizing agents, solubilizing agents, anti-caking agents, pH modifiers, or any other pharmaceutical vehicle described herein. In some embodiments, the pharmaceutically acceptable excipient comprises an organic acid, such as glycolic acid, lactic acid, citric acid, tartaric acid, malic acid, fumaric acid, and/or maleic acid.

Pharmaceutically acceptable excipients used in the effervescent granules or powders include an effervescent couple, i.e., an organic acid and a source of carbon dioxide. Effervescent powders may be produced by blending or admixing the organic acid and the source of carbon dioxide (the effervescent couple) and optionally any other desired pharmaceutically acceptable excipient. Effervescent granules may be produced by physically adhering or “gluing” the effervescent couple (the organic acid and the source of carbon dioxide) together using an edible or pharmaceutically acceptable binder such as polyvinylpyrrolidone, polyvinyl alcohol, L-leucine, polyethylene glycol, gum arabic, or the like, including combinations thereof. These types of granules are made by processes generically known as “wet granulation.” Granulating solvents such as ethanol and/or isopropyl alcohol are often used to aid this type of granulation process. Since the effervescent couple is physically bound together in the granule, the gas generating reaction is usually quite vigorous, leading to rapid dissolution times. Another type of “wet granulation” product that is specific to effervescent products is known as “fusion” type granules. These granules are formed by reacting the organic acid and source of carbon dioxide with a small amount of water (or sometimes a hydrous alcohol granulating solvent, such as various commercial grades of ethanol or isopropyl alcohol) in a highly controlled way. Since the effervescent reaction generates carbon dioxide, fusion granules tend to be quite porous, which decreases their density and also their dissolution time. Accordingly, effervescent granules prepared by wet granulation or fusion type processes may be desirable for making orodispersible dosage forms (ODxs) or other dosage forms where quick dissolving/disintegrating properties are sought. Effervescent tablet dosage forms prepared through tableting, e.g., compression, of effervescent granules or powders are also included in the present disclosure.

Additionally disclosed are pharmaceutical compositions in a dosage form that has an instant releasing component and at least one delayed releasing component, and is capable of giving a discontinuous release of the compound in the form of at least two consecutive pulses separated in time from about 0.1 up to about 24 hours (e.g., about 0.1, 0.5, 1, 2, 4, 6, 8, 10, 12, 14, 16, 18, 10, 22, or 24 hours). The pharmaceutical compositions comprise a compound as disclosed herein and one or more release controlling and non-release controlling excipients or carriers, such as those excipients or carriers suitable for a disruptable semipermeable membrane and as swellable substances.

Disclosed herein also are pharmaceutical compositions in a dosage form for oral administration to a subject, which comprise a compound disclosed herein (e.g., a compound of Formula (I) through (VII), or a pharmaceutically acceptable salt, solvate, stereoisomer, or prodrug thereof) and one or more pharmaceutically acceptable excipients enclosed in an intermediate reactive layer comprising a gastric juice-resistant polymeric layered material partially neutralized with alkali and having cation exchange capacity and a gastric juice-resistant outer layer.

The dosage form may be an immediate release (IR) dosage form, examples of which include, but are not limited to, an immediate release (IR) tablets or an immediate release (IR) capsule. In addition to the active ingredient (e.g., a compound of Formula (I) through (VII)), dosage forms adapted for immediate release may include one or more pharmaceutically acceptable excipients which readily disperse, dissolve, or otherwise breakdown in the gastric environment so as not to delay or prolong dissolution/absorption of the active ingredient(s). Examples of pharmaceutically acceptable excipients for immediate release dosage forms include, but are not limited to, one or more binders/granulators, matrix materials, fillers, diluents, disintegrants, dispersing agents, solubilizing agents, lubricants, and/or performance modifiers. In some embodiments, the immediate release (IR) dosage form is an immediate release (IR) tablet comprising one or more of microcrystalline cellulose, sodium carboxymethylcellulose, magnesium stearate, mannitol, crospovidone, and sodium stearyl fumarate. In some embodiments, the immediate release (IR) dosage form comprises microcrystalline cellulose, sodium carboxymethylcellulose, and magnesium stearate. In some embodiments, the immediate release (IR) dosage form comprises mannitol, crospovidone, and sodium stearyl fumarate.

The pharmaceutical compositions disclosed herein may be disclosed as soft or hard capsules, which can be made from gelatin, methylcellulose, starch, or calcium alginate. The hard gelatin capsule, also known as dry-filled capsule (DFC) or powder in capsule (PIC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

In some embodiments, the pharmaceutical compositions are in the form of immediate-release capsules for oral administration, and may further comprise cellulose, iron oxides, lactose, magnesium stearate, and sodium starch glycolate.

In some embodiments, the pharmaceutical compositions are in the form of delayed-release capsules for oral administration, and may further comprise cellulose, ethylcellulose, gelatin, hypromellose, iron oxide, and titanium dioxide.

In some embodiments, the pharmaceutical compositions are in the form of enteric coated delayed-release tablets for oral administration, and may further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In some embodiments, the pharmaceutical compositions are in the form of enteric coated delayed-release tablets for oral administration, and may further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The formulations of the disclosure comprise orally administered pharmaceutical compositions, such as tablet, capsule, caplets, gelcap and cap compositions, which may include uncoated tablets or coated tablets, caplets and caps (including film-coated, sugar-coated tablets, and gastro-resistant/enteric-coated tablets). The oral pharmaceutical compositions for oral use may include the active ingredients, e.g., any of the compounds described herein (e.g., a compound of Formula (I) through (VII)), mixed with pharmaceutically acceptable inactive excipients such as diluents, disintegrating agents, binding agents, lubricating agents, powder flow improving agent, wetting agents, sweetening agents, flavoring agents, coloring agents and preservatives. Moreover, oral pharmaceutical compositions of the disclosure are solid dosage forms intended for oral administration, e.g., obtained by dry granulation with single or multiple compressions of powders or granules. In some embodiments, the oral pharmaceutical compositions may be obtained by using wet granulation techniques. In some embodiments, the oral pharmaceutical compositions may be obtained by molding, heating/annealing, or extrusion techniques.

In some embodiments, the oral tablets are right circular solid cylinders, the end surfaces of which are flat or convex, and the edges of which may be beveled. In some embodiments, the surfaces are convex. In addition, they may have lines or break-marks (scoring), symbols or other markings.

In some embodiments, the break-mark(s) is/are intended to permit accurate subdivision of the tablet in order to provide doses of less than one tablet. In some embodiments, the tablet compositions comprise one or more excipients such as diluents, binders, disintegrating agents, glidants, lubricants, substances capable of modifying the behavior of the dosage forms and the active ingredient(s) in the gastrointestinal tract, coloring matter authorized by the appropriate national or regional authority and flavoring substances. When such excipients are used it is necessary to ensure that they do not adversely affect the stability, dissolution rate, bioavailability, safety or efficacy of the active ingredient(s); there must be no incompatibility between any of the components of the dosage form.

Coated tablets are tablets covered with one or more layers of mixtures of substances such as natural or synthetic resins, polymers, gums, fillers, sugars, plasticizers, polyols, waxes, coloring matters authorized by the appropriate national or regional authority, and flavoring substances. Such coating materials do not contain any active ingredient, e.g., any of the compounds described herein (e.g., a compound of Formula (I) through (VII), or a pharmaceutically acceptable salt, solvate, stereoisomer, or prodrug thereof). The tablets may be coated for a variety of reasons such as protection of the active ingredients from burst release from the matrix, air, moisture or light, masking of unpleasant tastes and odors or improvement of appearance. The substance used for coating may be applied as a solution or suspension.

In some embodiments, the manufacturing processes for the oral pharmaceutical compositions, e.g., tablets, meet the requirements of good manufacturing practices (GMP). In some embodiments, one or more measures are taken in the manufacture of oral pharmaceutical compositions selected from the following: ensure that mixing with excipients is carried out in a manner that ensures homogeneity; ensure that the oral pharmaceutical compositions possess a suitable mechanical strength to avoid crumbling or breaking on subsequent processing, e.g., coating, storage and distribution; minimize the degradation of the active ingredient; minimize the risk of microbial contamination; minimize the risk of cross-contamination. In addition, in the manufacture of scored tablets (tablets bearing a break-mark or marks) for which subdivision is intended in order to provide doses of less than one tablet measures are taken to: ensure the effectiveness of break-marks with respect to the uniformity of mass or content, as appropriate, of the subdivided parts so that the patient receives the intended dose.

When used for daily dosing, a suitable therapeutically effective dose will generally be in the range of about 0.00001 to about 10 mg per kilogram body weight of the subject per day, or about 0.01 to about 10 mg per kilogram body weight of the subject per day, or in the range of about 0.1 to about 5 mg per kilogram body weight per day, or in the range of about 0.5 to about 3 mg per kilogram body weight per day, or in the range of about 1 to about 2 mg per kilogram body weight per day. Additional details on techniques for formulation and administration are well described in the scientific and patent literature, see, e.g., the latest edition of Remington's Pharmaceutical Sciences, Maack Publishing Co, Easton Pa. (“Remington's”). After a pharmaceutical composition has been formulated in an acceptable excipient, it can be placed in an appropriate container and labeled for treatment of an indicated condition. For administration of the formulations comprising any of the compounds described herein (e.g., a compound of Formula (I) through (VII), or a pharmaceutically acceptable salt, solvate, stereoisomer, or prodrug thereof), such labeling would include, e.g., instructions concerning the amount, frequency, method of administration, treatment regimen and indications.

The pharmaceutical compositions disclosed herein may be disclosed in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein (e.g., a compound of Formula (I) through (VII)), and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates. In some embodiments, examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Cyclodextrins such as α-cyclodextrin, β-cyclodextrin, γ-cyclodextrin, methyl-β-cyclodextrin, hydroxyethyl β-cyclodextrin, hydroxypropyl-β-cyclodextrin, hydroxypropyl γ-cyclodextrin, sulfated β-cyclodextrin, sulfated α-cyclodextrin, sulfobutyl ether β-cyclodextrin, or other solubilized derivatives can also be advantageously used to enhance delivery of compositions described herein.

The pharmaceutical compositions disclosed herein for oral administration may be also disclosed in the forms of liposomes, micelles, microspheres, or nanosystems.

Liquid dosage forms such as aqueous solutions suitable for oral use can be prepared by dissolving the active ingredient (e.g., a compound of Formula (I) through (VII), or a pharmaceutically acceptable salt, solvate, stereoisomer, or prodrug thereof), in water and adding a suitable excipient(s) such as coloring agents, flavoring agents, stabilizing agents, and thickening agents, as desired. Aqueous suspensions suitable for oral use can be made by dispersing the finely divided active ingredient in water with viscous material, such as natural or synthetic gums, resins, methylcellulose, sodium carboxymethylcellulose, and other well-known suspending agents.

Also included are solid form preparations that are intended to be converted, shortly before use, to liquid dosage forms, including those intended for oral administration. Such liquid dosage forms include solutions, suspensions, and emulsions. These preparations may contain, in addition to the active ingredient, pharmaceutically acceptable excipients such as coloring agents, flavoring agents, stabilizing agents, buffering agents (buffers), artificial and natural sweeteners, dispersants, thickening agents, solubilizing agents, and the like. The pharmaceutical compositions disclosed herein may be disclosed as non-effervescent or effervescent, granules, tablets, and powders, to be reconstituted into a liquid dosage form prior to use. For example, oral liquid dosage forms may be prepared by reconstituting a solid dosage form disclosed herein into a pharmaceutically acceptable aqueous medium such as water, juice, or other drinkable fluid prior to use.

Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in any of the disclosed dosage forms.

The pharmaceutical compositions disclosed herein may be co-formulated with other active ingredients which do not impair the desired therapeutic action, or with substances that supplement the desired action, such as hydrocortisone.

In some embodiments, the oral dosage form (e.g., tablet composition) comprises a therapeutically effective amount of any of the compounds described herein for the treatment of pain. In some embodiments, the pain treated is cancer pain, e.g., refractory cancer pain. In some embodiments, the pain treated is post-surgical pain. In some embodiments, the pain treated is orthopedic pain. In some embodiments, the pain treated is back pain. In some embodiments, the pain treated is neuropathic pain.

In some embodiments, the pain treated is dental pain. In some embodiments, the pain treated is chronic pain. In some embodiments, the pain treated is chronic pain in opioid-tolerant patients. In some embodiments, the oral dosage form (e.g., tablet composition) comprises a therapeutically effective amount of any of the compounds described herein for use in treating acute pain (e.g., acute trauma pain).

In some embodiments, the oral dosage form (e.g., tablet composition) comprises a therapeutically effective amount of any of the compounds described herein for the treatment of depression.

In some embodiments, the oral dosage form (e.g., tablet composition) comprises a therapeutically effective amount of any of the compounds described herein for the treatment of brain injury.

In some embodiments, the oral dosage form (e.g., tablet composition) comprises a therapeutically effective amount of any of the compounds described herein for the treatment of stroke.

In some embodiments, the oral dosage form (e.g., tablet composition) comprises a therapeutically effective amount of any of the compounds described herein for use in treating migraine, e.g., with aura.

In some embodiments, the oral dosage form (e.g., tablet composition) comprises a therapeutically effective amount of any of the compounds described herein for use in treating refractory asthma.

In some embodiments, the oral dosage form (e.g., tablet composition) comprises a therapeutically effective amount of any of the compounds described herein for use in treating alcohol dependence.

In some embodiments, the oral dosage form (e.g., tablet composition) comprises a therapeutically effective amount of any of the compounds described herein for use in treating post traumatic stress disorder (PTSD).

In some embodiments, the oral dosage form (e.g., tablet composition) comprises a therapeutically effective amount of any of the compounds described herein for use in treating depression (e.g., treatment resistant depression (TRD) or bipolar depression).

In some embodiments, the oral dosage form (e.g., tablet composition) comprises a therapeutically effective amount of any of the compounds described herein for use in treating major depressive disorder (MDD).

In some embodiments, the oral dosage form (e.g., tablet composition) comprises a therapeutically effective amount of any of the compounds described herein for use in treating anxiety (e.g., generalized anxiety disorder). In some embodiments, the oral dosage form (e.g., tablet composition) comprises a therapeutically effective amount of any of the compounds described herein for use in treating comorbidities such as generalized anxiety disorder with depression.

In some embodiments, the oral dosage form (e.g., tablet composition) comprises a therapeutically effective amount of any of the compounds described herein for use in treating schizophrenia.

In some embodiments, the oral dosage form (e.g., tablet composition) comprises a therapeutically effective amount of any of the compounds described herein for use in treating bipolar disorder.

In some embodiments, the oral dosage form (e.g., tablet composition) comprises a therapeutically effective amount of any of the compounds described herein for use in treating suicidality or suicidal ideation.

In some embodiments, the oral dosage form (e.g., tablet composition) comprises a therapeutically effective amount of any of the compounds described herein for use in treating autism.

In some embodiments, the oral dosage form (e.g., tablet composition) comprises a therapeutically effective amount of any of the compounds described herein for use in treating diabetic neuropathy.

In some embodiments, the oral dosage form (e.g., tablet composition) comprises a therapeutically effective amount of any of the compounds described herein for use in treating neuropathic pain.

In some embodiments, the oral dosage form (e.g., tablet composition) comprises a therapeutically effective amount of any of the compounds described herein for use in treating levodopa-induced dyskinesia.

In some embodiments, the oral dosage form (e.g., tablet composition) comprises a therapeutically effective amount of any of the compounds described herein for use in treating or modulating a pseudobulbar effect or Bulbar function.

In some embodiments, the oral dosage form (e.g., tablet composition) comprises a therapeutically effective amount of any of the compounds described herein for use in treating Alzheimer's disease or conditions associated with Alzheimer's disease (e.g., Alzheimer's dementia or Alzheimer's agitation).

In some embodiments, the oral dosage form (e.g., tablet composition) comprises a therapeutically effective amount of any of the compounds described herein for use in treating tinnitus.

In some embodiments, the oral dosage form (e.g., tablet composition) comprises a therapeutically effective amount of any of the compounds described herein for use in treating a disease or disorder associated with a serotonin 5-HT₂ receptor.

In some embodiments, the disease or disorder is selected from the group consisting of central nervous system (CNS) disorders, including post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), bipolar and related disorders including bipolar I disorder, bipolar II disorder, cyclothymic disorder, obsessive-compulsive disorder (OCD), generalized anxiety disorder (GAD), social anxiety disorder, substance use disorders including alcohol use disorder, opioid use disorder, amphetamine use disorder, nicotine use disorder, and cocaine use disorder, anorexia nervosa, bulimia nervosa, binge eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, sexual dysfunction, chronic fatigue syndrome, Lyme disease, and obesity.

In some embodiments, the disease or disorder includes conditions of the autonomic nervous system (ANS).

In some embodiments, the disease or disorder includes pulmonary disorders including asthma and chronic obstructive pulmonary disorder (COPD).

In some embodiments, the disease or disorder includes cardiovascular disorders including atherosclerosis.

In some embodiments, the oral dosage form (e.g., tablet composition) is utilized as a 2-times a day (BID), 3-times a day (TID) or 4-times a day (QID) application. In some embodiments, the oral dosage form (e.g., tablet composition) is utilized as a once a day (QD) application. In some embodiments, the oral dosage form (e.g., tablet composition) is utilized as a nightly (QHS) application.

In some embodiments the oral dosage form (e.g., tablet composition) is utilized as an as needed (PRN) application.

In some embodiments, when administered in unit dosage form, the oral administration event may comprise one single unit dose (e.g., pill) or multiple unit doses (e.g., multiple pills) which together sum to the desired dosage.

### B. Parenteral Administration

The pharmaceutical compositions disclosed herein may be administered parenterally by injection, infusion, perfusion, or implantation, for local or systemic administration. Parenteral administration, as used herein, includes, but is not limited to, intravenous, intradermal, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

In some embodiments, the pharmaceutical composition is in the form of an injectable (liquid) dosage form (e.g., for intravenous, intramuscular, subcutaneous, etc. administration). In some embodiments, injectable (liquid) dosage forms (e.g., for intravenous, intramuscular, subcutaneous, etc. administration) are prepared by reconstituting a solid dosage form disclosed herein into a pharmaceutically acceptable liquid medium such as water, saline solutions, viscous aqueous solutions/suspensions, water-miscible vehicles (e.g., organic solvents such as N-methyl-2-pyrrolidone), etc. prior to use.

The pharmaceutical compositions intended for parenteral administration may include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate, borate, sulfate, and citrate buffers. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, methyl-β-cyclodextrin, hydroxypropyl-3-cyclodextrin/hydroxypropyl-β-cyclodextrin, sulfobutylether-β-cyclodextrin, and sulfobutylether 7-O-cyclodextrin (CAPTISOL®, CyDex, Lenexa, Kans.). Suitable thickening or viscosity building agents include, for example, polyvinyl alcohol, polyvinyl pyrrolidone, methyl cellulose, hydroxypropyl methylcellulose, hydroxyethyl cellulose, carboxymethyl cellulose (e.g., sodium carboxymethyl cellulose), hydroxypropyl cellulose, chondroitin sulfate and salts thereof, hyaluronic acid and salts thereof, including crosslinked variations of any of the forgoing, and combinations of the foregoing.

In some embodiments, the pharmaceutical composition is in an injectable (liquid) dosage form. In some embodiments, the injectable (liquid) dosage form comprises a compound of the present disclosure, an aqueous vehicle (e.g., isotonic saline), a buffering agent (e.g., a citric acid buffer), optionally a pH adjusting agent (e.g., sodium hydroxide), and optionally an isotonic agent. In some embodiments, the injectable (liquid) dosage form comprises a compound of the present disclosure, an aqueous vehicle (e.g., isotonic saline), and a pH adjusting agent (e.g., sodium hydroxide), wherein the injectable (liquid) dosage form is formulated without a buffering agent (e.g., a citric acid buffer). In some embodiments, the injectable (liquid) dosage form is prepared by reconstituting a solid dosage form comprising a compound of the present disclsoure, into an aqueous vehicle such as isotonic saline. Reconstitution of the solid dosage form, e.g., crystalline form of a compound of the present disclosure, can be performed immediately prior to use.

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art. The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

In some embodiments, the pharmaceutical compositions are disclosed as ready-to-use sterile solutions. In some embodiments, the pharmaceutical compositions are disclosed as sterile dry soluble products, including lyophilized powders and hypodermic tablets, to be reconstituted with an excipient (e.g., vehicle) prior to use. In some embodiments, the pharmaceutical compositions are disclosed as ready-to-use sterile suspensions. In some embodiments, the pharmaceutical compositions are disclosed as sterile dry insoluble products to be reconstituted with an excipient (e.g., vehicle) prior to use. In some embodiments, the pharmaceutical compositions are disclosed as ready-to-use sterile emulsions.

The pharmaceutical compositions may be formulated as a suspension, solid, semi-solid, or thixotropic liquid, for administration as an implanted depot or to generate a depot-like effect.

In some embodiments, the pharmaceutical compositions disclosed herein are dispersed in a solid inner matrix, which is surrounded by an outer polymeric membrane that is insoluble in body fluids but allows the active ingredient in the pharmaceutical compositions to diffuse through. Fatty acid salts of the compounds of Formula (I) through (VII) may be well-suited for such dosage forms. Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate. Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

In some embodiments, the pharmaceutical composition is in the form of a viscous aqueous solution/suspension for injection to provide a slow/sustained absorption or depot-like effect. Here, pharmaceutical excipients which build viscosity may be used, such as thickening or viscosity building agents including, but not limited to, polyvinyl alcohol, polyvinyl pyrrolidone, methyl cellulose, hydroxypropyl methylcellulose, hydroxyethyl cellulose, carboxymethyl cellulose (e.g., sodium carboxymethyl cellulose), hydroxypropyl cellulose, chondroitin sulfate and salts thereof, hyaluronic acid and salts thereof, and combinations of the foregoing. In some embodiments, the pharmaceutically acceptable excipient comprises sodium carboxymethyl cellulose, hyaluronic acid and salts thereof, or a combination thereof. Such viscous aqueous solution/suspension dosage forms may be particularly well suited for subcutaneous or intramuscular administration, where the active ingredient can be slowly released from the injection site and absorbed over sustained periods, generating a depot-like release effect. Further, crosslinked versions of any of the forgoing may be utilized. The rate of release of the active ingredient can be controlled through the extent of cross-linking of any of the thickening or viscosity building agents described herein, or by controlling the rate that any of the forgoing are crosslinked through use, amount, or type of crosslinking agent employed. For example, a slow/sustained absorption or depot-like effect can be achieved through use or formation of a crosslinked hyaluronic acid at the injection site. In some embodiments, administration of a viscous aqueous solution/suspension dosage form, e.g., via subcutaneous or intramuscular injection, provides a release period of about 1 hour, about 2 hours, about 3 hours, about 4 hours, about 5 hours, about 6 hours, about 7 hours, about 8 hours, or any range therebetween, or longer.

In some embodiments, the pharmaceutical composition is formulated with a pharmaceutically acceptable salt of a compound of Formula (I) through (VII) with poor aqueous solubility (e.g., a water solubility at 22° C. of less than 5 mg/mL, less than 4 mg/mL, less than 3 mg/mL, less than 2 mg/mL, less than 1 mg/mL, less than 0.5 mg/mL, less than 0.1 mg/mL), such as a fatty acid salt of a compound of Formula (I) through (VII). Examples of fatty acid salt forms include, but are not limited to, those formed by contacting a compound of Formula (I) through (VII) with adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, or caproic acid. Such pharmaceutical compositions may be particularly well suited for subcutaneous or intramuscular administration, where the active ingredient can slowly solubilize and be slowly released from the injection site and absorbed over sustained periods, generating a depot-like release effect. These “slow release” salts may be optionally formulated with thickening or viscosity building agents, e.g., in viscous aqueous solution/suspension formulations. In some embodiments, administration of a pharmaceutical composition formulated with a pharmaceutically acceptable salt of a compound of Formula (I) through (VII) with poor aqueous solubility, e.g., via subcutaneous or intramuscular injection, provides a release period of about 1 hour, about 2 hours, about 3 hours, about 4 hours, about 5 hours, about 6 hours, about 7 hours, about 8 hours, or any range therebetween, or longer.

### C. Topical Administration

The pharmaceutical compositions disclosed herein may be administered topically to the skin, orifices, or mucosa. The effects may be local or systemic. Topical administration, as described herein, includes, but is not limited to, conjuctival, intracorneal, intraocular, ophthalmic, auricular, transdermal, nasal (e.g., intranasal), vaginal, uretheral, respiratory, and rectal administration.

The pharmaceutical compositions disclosed herein may be formulated in any dosage form that is suitable for topical administration for local or systemic effect, including emulsions, solutions, suspensions, creams, gels, hydrogels, ointments, powders or dusting powders, dressings, elixirs, lotions, suspensions, tinctures, pastes, foams, films, aerosols, irrigations, sprays, suppositories, bandages, dermal patches, and inhalants. The topical formulation of the pharmaceutical compositions disclosed herein may contain the active ingredient(s) which may be mixed under sterile conditions with a pharmaceutically acceptable excipient, e.g., with any preservatives, buffers, absorption enhancers, propellants which may be required. Liposomes, micelles, microspheres, nanosystems, and mixtures thereof, may also be used.

Pharmaceutically acceptable excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The ointments, pastes, creams and gels may contain, in addition to an active ingredient(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient(s), excipients such as lactose, talc, silicic acid, aluminum hydroxide, calcium silicates and polyamide powder, or mixtures of these substances. Sprays, such as those used for (intra)nasal administration, can additionally contain customary propellants, such as fluorohydrocarbons, chlorofluorohydrocarbons and volatile unsubstituted hydrocarbons, such as butane and propane.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760, the entire contents of these patents are incorporated herein by reference in their entirety.

Transdermal delivery devices (e.g., patches) may be used. Such dosage forms have the added advantage of providing controlled delivery of active ingredient(s) to the body. That is, the compounds of the present disclosure (e.g., a compound of Formula (I) through (VII), or a pharmaceutically acceptable salt, solvate, stereoisomer, or prodrug thereof) can be administered via a transdermal patch at a steady state concentration, whereby the active ingredient(s) is gradually administered over time, thus avoiding drug spiking and adverse events/toxicity associated therewith.

Transdermal patch dosage forms herein may be formulated with various amounts of the active ingredient(s), depending on the disease/condition being treated, the active ingredient(s) employed, the permeation and size of the transdermal delivery device, the release time period, etc. For example, when formulated with a compound of Formula (I) through (VII), a unit dose preparation may be varied or adjusted e.g., from 5 mg, 10 mg, 15 mg, 20 mg, 25 mg, 30 mg, 35 mg, 40 mg, 45 mg, 50 mg, to 200 mg, 175 mg, 150 mg, 125 mg, 100 mg, 95 mg, 90 mg, 85 mg, 80 mg, 75 mg, 70 mg, 65 mg, 60 mg, 55 mg of the compound of Formula (I) through (VII) (active basis) or otherwise as deemed appropriate using sound medical judgment, according to the particular application and the potency of compound.

Transdermal patches formulated with the disclosed compounds may be suitable for microdosing or sub-psychedelic (also referred to herein as sub-psychoactive) dosing, to achieve durable therapeutic benefits, with decreased toxicity. In some embodiments, the compound of Formula (I) through (VII), or a pharmaceutically acceptable salt, solvate, stereoisomer, or prodrug thereof, is administered via a transdermal patch at sub-psychoactive (yet still potentially serotonergic concentrations) concentrations, for example, over an extended period such as over a 8, 24, 48, 72, 84, 96, or 168 hour time period.

In addition to the active ingredient(s), and any optional pharmaceutically acceptable excipient(s), the transdermal patch may also include one or more of a pressure sensitive adhesive layer, a backing, and a release liner, as is known to those of ordinary skill in the art.

Transdermal patch dosage forms can be made by dissolving or dispersing the compounds herein in the proper medium. In some embodiments, the compounds of the present disclosure may be dissolved/dispersed directly into a polymer matrix forming the pressure sensitive adhesive layer. Such transdermal patches are called drug-in-adhesive (DIA) patches. Preferred DIA patch forms are those in which the active ingredient(s) is distributed uniformly throughout the pressure sensitive adhesive polymer matrix. In some embodiments, the active ingredient(s) may be provided in a layer containing the active ingredient(s) plus a polymer matrix which is separate from the pressure sensitive adhesive layer. In any case, the compounds of the present disclosure may optionally be formulated with suitable excipient(s) such as carrier agents, permeation agents/absorption enhancers, humectants/crystallization inhibitors, etc. to increase the flux across the skin.

Examples of carrier agents may include, but are not limited to, C₈-C₂₂ fatty acids, such as oleic acid, undecanoic acid, valeric acid, heptanoic acid, pelargonic acid, capric acid, lauric acid, and eicosapentaenoic acid; C₈-C₂₂ fatty alcohols such as octanol, nonanol, oleyl alcohol, decyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; glycerol; ethyl acetate; acetoacetic ester; N-alkylpyrrolidone; cyclodextrins, such as α-cyclodextrin, β-cyclodextrin, γ-cyclodextrin, or derivatives such as 2-hydroxypropyl-β-cyclodextrin; and terpenes/terpenoids, such as limonene, linalool, myrcene, pinene such as α-pinene, caryophyllene, citral, eucolyptol, and the like; including mixtures thereof.

Examples of permeation agents/absorption enhancers include, but are not limited to, sulfoxides, such as dodecylmethylsulfoxide, octyl methyl sulfoxide, nonyl methyl sulfoxide, decyl methyl sulfoxide, undecyl methyl sulfoxide, 2-hydroxydecyl methyl sulfoxide, 2-hydroxy-undecyl methyl sulfoxide, 2-hydroxydodecyl methyl sulfoxide, and the like; surfactant-lecithin organogel (PLO), such as those formed from an aqueous phase with one or more of poloxamers, CARBOPOL and PEMULEN, a lipid phase formed from one or more of isopropyl palmitate and PPG-2 myristyl ether propionate, and lecithin; fatty acids, esters, and alcohols, such as oleyloleate and oleyl alcohol; keto acids such as levulinic acid; glycols and glycol ethers, such as diethylene glycol monoethyl ether; including mixtures thereof.

Examples of humectants/crystallization inhibitors include, but are not limited to, polyvinyl pyrrolidone-co-vinyl acetate, HPMC, polymethacrylate, and mixtures thereof.

The pressure sensitive adhesive layer may be formed from polymers including, but not limited to, acrylics (polyacrylates including alkyl acrylics), polyvinyl acetates, natural and synthetic rubbers (e.g., polyisobutylene), ethylenevinylacetate copolymers, polysiloxanes, polyurethanes, plasticized polyether block amide copolymers, plasticized styrene-butadiene rubber block copolymers, and mixtures thereof. The pressure-sensitive adhesive layer used in the transdermal patch of the present disclosure may be formed from an acrylic polymer pressure-sensitive adhesive, preferably an acrylic copolymer pressure sensitive adhesive. The acrylic copolymer pressure sensitive adhesive may be obtained by copolymerization of one or more alkyl (meth)acrylates (e.g., 2-ethylhexyl acrylate); aryl (meth)acrylates; arylalkyl (meth)acrylate; and (meth)acrylates with functional groups such as hydroxyalkyl (meth)acrylates (e.g., hydroxyethyl acrylate, 2-hydroxypropyl acrylate, 3-hydroxypropyl acrylate, 4-hydroxybutyl acrylate, 2-hydroxyethyl methacrylate, 2-hydroxypropyl methacrylate, 3-hydroxypropyl methacrylate, and 4-hydroxybutyl methacrylate), carboxylic acid containing (meth)acrylates (e.g., acrylic acid), and alkoxy (meth)acrylates (e.g., methoxyethyl acrylate); optionally with one or more copolymerizable monomers (e.g., vinylpyrrolidone, vinyl acetate, etc.). Specific examples of acrylic pressure-sensitive adhesives may include, but are not limited to, DURO-TAK products (Henkel) such as DURO-TAK 87-900A, DURO-TAK 87-9301, DURO-TAK 87-4098, DURO-TAK 87-2074, DURO-TAK 87-235A, DURO-TAK 87-2510, DURO-TAK 87-2287, DURO-TAK 87-4287, DURO-TAK 87-2516, DURO-TAK 387-2052, and DURO-TAK 87-2677.

The backing used in the transdermal patch of the present disclosure may include flexible backings such as films, nonwoven fabrics, Japanese papers, cotton fabrics, knitted fabrics, woven fabrics, and laminated composite bodies of a nonwoven fabric and a film. Such a backing is preferably composed of a soft material that can be in close contact with a skin and can follow skin movement and of a material that can suppress skin rash and other discomforts following prolonged use of the patch. Examples of the backing materials include, but are not limited to, polyethylene, polypropylene, polyethylene terephthalate, polybutylene terephthalate, polyethylene naphthalate, polystyrene, nylon, cotton, acetate rayon, rayon, a rayon/polyethylene terephthalate composite body, polyacrylonitrile, polyvinyl alcohol, acrylic polyurethane, ester polyurethane, ether polyurethane, a styrene-isoprene-styrene copolymer, a styrene-butadiene-styrene copolymer, a styrene-ethylene-propylene-styrene copolymer, styrene-butadiene rubber, an ethylene-vinyl acetate copolymer, or cellophane, for example. Preferred backings do not adsorb or release the active ingredient(s). In order to suppress the adsorption and release of the active ingredient(s), to improve transdermal absorbability of the active ingredient(s), and to suppress skin rash and other discomforts, the backing preferably includes one or more layers composed of the material above and has a water vapor permeability. Specific examples of backings may include, but are not limited to, 3M COTRAN products such as 3M COTRAN ethylene vinyl acetate membrane film 9702, 3M COTRAN ethylene vinyl acetate membrane film 9716, 3M COTRAN polyethylene membrane film 9720, 3M COTRAN ethylene vinyl acetate membrane film 9728, and the like.

The release liner used in the transdermal patch of the present disclosure may include, but is not limited to, a polyester film having one side or both sides treated with a release coating, a polyethylene laminated high-quality paper treated with a release coating, and a glassine paper treated with a release coating. The release coating may be a fluoropolymer, a silicone, a fluorosilicone, or any other release coating known to those of ordinary skill in the art. The release liner may have an uneven surface in order to easily take out the transdermal patch from a package. Examples of release liners may include, but are not limited to SCOTCHPAK products from 3M such as 3M SCOTCHPAK 9744, 3M SCOTCHPAK 9755, 3M SCOTCHPAK 9709, and 3M SCOTCHPAK 1022.

Other layers such as abuse deterrent layers formulated with one or more irritants (e.g., sodium lauryl sulfate, poloxamer, sorbitan monoesters, glyceryl monooleates, spices, etc.), may also be employed.

Methods disclosed herein using a transdermal patch dosage form provide for systemic delivery of small doses of active ingredient(s), preferably over extended periods of time such as up to 168 hour time periods, for example from 2 to 96 hours, or 4 to 72 hours, or 8 to 24 hours, or 10 to 18 hours, or 12 to 14 hours. In particular, the compound of Formula (I) through (VII) can be delivered in small, steady, and consistent doses such that deleterious or undesirable side-effects can be avoided. In some embodiments, the compound of Formula (I) through (VII) is administered transdermally at sub-psychoactive (yet still potentially serotonergic concentrations) concentrations.

An exemplary drug-in-adhesive (DIA) patch formulation may comprise 5 to 30 wt. % of a compound of Formula (I) through (VII), or a pharmaceutically acceptable salt, solvate, stereoisomer, or prodrug thereof, 30 to 70 wt. % pressure sensitive adhesive (e.g., DURO-TAK 387-2052, DURO-TAK 87-2677, and DURO-TAK 87-4098), 1 to 10 wt. % permeation agents/absorption enhancers (e.g., oleyloleate, oleyl alcohol, levulinic acid, diethylene glycol monoethyl ether, etc.), and 5 to 35 wt. % crystallization inhibitor (e.g., polyvinyl pyrrolidone-co-vinyl acetate, HPMC, polymethacrylate, etc.), each based on a total weight of the DIA patch formulation, though it should be understood that many variations are possible in light of the teachings herein.

Therefore, provided herein are methods of treating a disease or disorder, including those associated with a serotonin 5-HT₂ receptor, such as a central nervous system (CNS) disorder, a psychological disorder, or an autonomic nervous system (ANS), comprising administering a compound of the present disclosure (e.g., a compound of Formula (I) through (VII), or a pharmaceutically acceptable salt thereof) via a transdermal patch. Here, the compound of the present disclosure is one that is capable of diffusing from the matrix of the transdermal patch (e.g., from the pressure sensitive adhesive layer) across the skin of the subject and into the bloodstream of the subject. In some embodiments, the compound can be administered for treatment of CNS disease or other disorder. In some embodiments, the compound can be administered to treat depression including, but not limited to major depression, melancholic depression, atypical depression, or dysthymia. In some embodiments, the compound can be administered to treat psychological disorders including anxiety disorder, obsessive compulsive disorder, addiction (narcotic addiction, tobacco addiction, opioid addiction), alcoholism, depression and anxiety (chronic or related to diagnosis of a life-threatening or terminal illness), compulsive behavior, or a related symptom. In some embodiments, the disease or disorder is selected from the group consisting of central nervous system (CNS) disorders, including post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), bipolar and related disorders including bipolar I disorder, bipolar II disorder, cyclothymic disorder, obsessive-compulsive disorder (OCD), generalized anxiety disorder (GAD), social anxiety disorder, substance use disorders including alcohol use disorder, opioid use disorder, amphetamine use disorder, nicotine use disorder, and cocaine use disorder, anorexia nervosa, bulimia nervosa, binge eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, sexual dysfunction, chronic fatigue syndrome, Lyme disease, and obesity. In some embodiments, the disease or disorder may include conditions of the autonomic nervous system (ANS). In some embodiments, the disease or disorder may include pulmonary disorders (e.g., asthma and chronic obstructive pulmonary disorder (COPD). In some embodiments, the disease or disorder may include cardiovascular disorders (e.g., atherosclerosis).

Automatic injection devices offer a method for delivery of the compositions disclosed herein to patients. The compositions disclosed herein may be administered to a patient using automatic injection devices through a number of known devices, a non-limiting list of which includes transdermal, subcutaneous, and intramuscular delivery.

In some transdermal, subcutaneous, or intramuscular applications, a composition disclosed herein is absorbed through the skin. Passive transdermal patch devices often include an absorbent layer or membrane that is placed on the outer layer of the skin. The membrane typically contains a dose of a substance that is allowed to be absorbed through the skin to deliver the composition to the patient. Typically, only substances that are readily absorbed through the outer layer of the skin may be delivered with such transdermal patch devices.

Other automatic injection devices disclosed herein are configured to provide for increased skin permeability to improve delivery of the disclosed compositions. Non-limiting examples of structures used to increase permeability to improve transfer of a composition into the skin, across the skin, or intramuscularly include the use of one or more microneedles, which in some embodiments may be coated with a composition disclosed herein. Alternatively, hollow microneedles may be used to provide a fluid channel for delivery of the disclosed compositions below the outer layer of the skin. Other devices disclosed herein include transdermal delivery by iontophoresis, sonophoresis, reverse iontophoresis, or combinations thereof, and other technologies known in the art to increase skin permeability to facilitate drug delivery.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be disclosed in the forms of ointments, creams, and gels. Suitable ointment excipients include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. For example, a low melting wax, such as a mixture of fatty acid glycerides or cocoa butter, or other pharmaceutically acceptable excipient is first melted and the active ingredient is dispersed homogeneously therein, as by stirring. The molten homogeneous mixture is then poured into convenient sized molds, allowed to cool, and thereby to solidify. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein may be administered intranasally. The terms “nasal,” “intranasal,” and the like refers to a route of administration, or dosage forms adapted for a route of administration, wherein the pharmaceutical dosage form is taken to, or through, the nose (e.g., nasal cavity). Similarly, a “nasal delivery device” or an “intranasal delivery device” is intended to mean an apparatus that administers an active ingredient into the nasal cavity. In some embodiments, the intranasal dosage form may be in the form of an aqueous or non-aqueous solution, suspension, liposomal dispersion, emulsion, microemulsion or sol-gel. Non-limiting examples of intranasal administration include introduction of a solution or suspension in the form of a nasal spray or drops (direct instillation) or intranasal application of a gel, emulsion or ointment. Relative to an oral dosage form such as a tablet or capsule, intranasal delivery provides for rapid absorption, faster onset of therapeutic action and avoidance of first pass metabolism. The amount of active ingredient absorbed depends on many factors. These factors include, but are not limited to, the drug concentration, the drug delivery vehicle, mucosal contact time, the venous drainage of the mucosal tissues, the degree that the drug is ionized at the pH of the absorption site, the size of the drug molecule, and its relative lipid solubility.

The pharmaceutical compositions of the present disclosure for nasal administration include a compound of the present disclosure and optionally a pharmaceutically acceptable excipient including, but not limited to, permeation agents/absorption enhancers which promote nasal absorption of the active ingredient after nasal administration and agents to improve brain penetration of the drug following nasal administration, diluents, binders, lubricants, glidants, disintegrants, desensitizing agents, emulsifying agents, bioadhesive agents, solubilizing agents, suspending and dispersing agents, thickening or viscosity building agents, isotonic agents, pH adjusting agents, buffering agents, carriers, flavoring agents, sweetening agents, and mixtures thereof. In some embodiments, the active ingredient is present in the pharmaceutical composition in particulate form. In some embodiments, the particle size of the active ingredient is less than or equal to about 60 microns, which can help to ensure uniformity of any blends of the particles with other ingredients, or to provide an adequate dispersion in a liquid vehicle.

The transport of the active ingredient across normal mucosal surfaces (such as the nasal mucosa) can be enhanced by optionally combining it with a permeation agent/absorption enhancer. Examples of these permeation agents/absorption enhancers include, but are not limited to, cationic polymers, surface active agents, chelating agents, mucolytic agents, cyclodextrin, polymeric hydrogels, combinations thereof, and any other similar absorption promoting agents known to those of skill in the art. Representative examples of permeation agents/absorption enhancers include, but are not limited to, phospholipids, such as phosphatidylglycerol or phosphatidylcholine, lysophosphatidyl derivatives, such as lysophosphatidylethanolamine, lysophosphatidylcholine, lysophosphatidylglycerol, lysophosphatidylserine, or lysophosphatidic acid, polyols, such as glycerol or propylene glycol, fatty acid esters thereof such as glycerides, amino acids, and esters thereof, cyclodextrins, or others set forth herein. Gelling excipients or viscosity-increasing excipients can also be used.

The transport of the active ingredient across normal mucosal surfaces can also be enhanced by increasing the time in which the formulations adhere to the mucosal surfaces. Bioadhesive agents, for example, those which form hydrogels, exhibit muco-adhesion and controlled drug release properties and can be included in the intranasal compositions described herein. Representative bioadhesive agents capable of binding to the nasal mucosa include, but are not limited to, polycarbophil, polylysine, methylcellulose, sodium carboxymethylcellulose, hydroxypropylmethylcellulose, hydroxyethyl cellulose, pectin, Carbopol 934P, polyethylene oxide 600K, one or more poloxomers such as Pluronic F127 and/or Pluronic F-68, polyisobutylene (PIB), polyisoprene (PIP), polyvinyl pyrrolidone (PVP), polyvinyl alcohol (PVA), xanthan gum, guar gum, and locust bean gum. Other nasal delivery compositions are chitosan-based and are suitable to increase the residence time of the active ingredient on mucosal surfaces, which results in increasing its bioavailability. Thiolated polymeric vehicles that form covalent bonds with the cysteine-rich subdomains of the mucus membrane can also provide mucoadhesion, which prolongs the contact time between the active ingredient and the membrane.

The intranasal compositions can also include one or more preservatives. Representative preservatives include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenyl ethyl alcohol; organic acids or salts thereof such as benzoic acid, sodium benzoate, potassium sorbate, parabens; or complex forming agents such as EDTA.

Intranasal dosage forms may also include ion-exchange resins, e.g., microspheres, which carry suitable anionic groups such as carboxylic acid residues, carboxymethyl groups, sulfopropyl groups and methylsulfonate groups. Ion-exchange resins, such as cation exchangers, can also be used. For example, pharmaceutical compositions may be formulated with chitosan, which is partially deacetylated chitin, or poly-N-acetyl-D-glucosamine, or a pharmaceutically acceptable salt thereof such as hydrochloride, lactate, glutamate, maleate, acetate, formate, propionate, malate, malonate, adipate, or succinate. Examples of non-ion-exchange resins (e.g., microspheres) which may be used include, but are not limited to starch, gelatin, collagen and albumin.

The pharmaceutical composition can also include an appropriate pH adjusting agent, including, but not limited to, sodium hydroxide, hydrochloric acid, citric acid, lactic acid, glutamic acid, maleic acid, acetic acid, formic acid, propionic acid, malic acid, malonic acid, adipic acid, and succinic acid.

Other ingredients such as diluents are cellulose, microcrystalline cellulose, hydroxypropyl cellulose, starch, hydroxypropyl methyl cellulose, dicalcium phosphate, calcium sulfate, lactose, sorbitol, sucrose, inositol, kaolin, mannitol, sodium chloride, and powdered sugar and the like.

Isotonic agents to adjust the tonicity of the composition may be added, including, but not limited to, sodium chloride, glucose, dextrose, mannitol, sorbitol, lactose, and the like.

Acidic, neutral, or basic buffering agents can also be added to the intranasal composition to control the pH, including, but not limited to, phosphate buffers, acetate buffers, and citrate buffers.

In addition to using permeation agents/absorption enhancers, which increase the transport of the active ingredient through the mucosa, and bioadhesive agents, which prolong the contact time of the active agent along the mucosa, the administration of the active ingredient can be controlled by using controlled release formulations. There are numerous particulate drug delivery vehicles known to those of skill in the art which can include the active ingredients and deliver them in a controlled manner. Examples include particulate polymeric drug delivery vehicles, for example, biodegradable polymers, and particles formed of non-polymeric components. These particulate drug delivery vehicles can be in the form of powders, microparticles, nanoparticles, microcapsules, liposomes, and the like. Typically, if the active ingredient is in particulate form without added components, its release rate depends on the release of the active ingredient itself. Typically, the rate of absorption is enhanced by presenting the drug in a micronized form, wherein particles are below 20 microns in diameter. In contrast, if the active ingredient is in particulate form as a blend of the active agent and a polymer, the release of the active agent is controlled, at least in part, by the removal of the polymer, typically by dissolution, biodegradation, or diffusion from the polymer matrix. In some embodiments, the pharmaceutical composition is in the form of a viscous aqueous solution/suspension for intranasal administration to provide a slow/sustained release and absorption. Here, pharmaceutically acceptable excipients which build viscosity may be used, such as thickening or viscosity building agents including, but not limited to, polyvinyl alcohol, polyvinyl pyrrolidone, methyl cellulose, hydroxypropyl methylcellulose, hydroxyethyl cellulose, carboxymethyl cellulose (e.g., sodium carboxymethyl cellulose), hydroxypropyl cellulose, chondroitin sulfate and salts thereof, hyaluronic acid and salts thereof, including crosslinked variants of any of the forgoing, and combinations of the foregoing. In some embodiments, the pharmaceutically acceptable excipient comprises sodium carboxymethyl cellulose, hyaluronic acid and salts thereof, or a combination thereof. Such viscous aqueous solution/suspension dosage forms may be particularly well suited for intranasal dosage forms whereby the active ingredient is relatively short acting and/or where longer acting formulations are desirable, in that the active ingredient can be slowly released from the administration site and absorbed over sustained periods.

In some embodiments, the pharmaceutical composition is formulated with a pharmaceutically acceptable salt of a compound of Formula (I) through (VII) with poor aqueous solubility (e.g., a water solubility at 22° C. of less than 5 mg/mL, less than 4 mg/mL, less than 3 mg/mL, less than 2 mg/mL, less than 1 mg/mL, less than 0.5 mg/mL, less than 0.1 mg/mL), such as a fatty acid salt of a compound of Formula (I) through (VII). Examples of fatty acid salt forms include, but are not limited to, those formed by contacting a compound of Formula (I) with adipic (hexandioic) acid, lauric (dodecanoic) acid, linoleic acid, myristic (tetradecanoic) acid, capric (decanoic) acid, stearic (octadecanoic) acid, oleic acid, caprylic (octanoic) acid, palmitic (hexadecenoic) acid, sebacic acid, undecylenic acid, or caproic acid. Such pharmaceutical compositions may be particularly well suited for intranasal dosage forms whereby the active ingredient is relatively short acting and/or where longer acting formulations are desirable, in that the active ingredient can be slowly released from the administration site and absorbed over sustained periods.

Other intranasal dosage forms and methods contemplated herein are disclosed in van Woensel M, et al. Formulations for Intranasal Delivery of Pharmacological Agents to Combat Brain Disease: A New Opportunity to Tackle GBM?Cancers (Basel). 2013 Aug. 14; 5(3):1020-48, incorporated herein by reference in its entirety.

Intranasal delivery devices are known in the art. Thus, any device suitable for delivery of drug to nasal mucosa may be used. Non-limiting examples of devices useful for the administration of liquid dosage forms include vapor devices (e.g., vapor inhalers), drop devices (e.g., catheters, single-dose droppers, multi-dose droppers, and unit-dose pipettes), mechanical spray pump devices (e.g., squeeze bottles, multi-dose metered-dose spray pumps, and single/duo-dose spray pumps), bi-directional spray pumps (e.g., breath-actuated nasal delivery devices), gas-driven spray systems/atomizers (e.g., single- or multi-dose HFA or nitrogen propellant-driven metered-dose inhalers, including traditional and circumferential velocity inhalers), and electrically powered nebulizers/atomizers (e.g., pulsation membrane nebulizers, vibrating mechanical nebulizers, and hand-held mechanical nebulizers). Non-limiting examples of devices useful for the administration of powder compositions (e.g., lyophilized or otherwise dried pooled compositions) include, but are not limited to, mechanical powder sprayers (e.g., handactuated capsule-based powder spray devices and handactuated powder spray devices, hand actuated gel delivery devices), breath-actuated inhalers (e.g., single- or multi-dose nasal inhalers and capsule-based single- or multi-dose nasal inhalers), and insufllators (e.g., breath-actuated nasal delivery devices).

Use of metered sprays for intranasal delivery can also be accomplished by including the active ingredient in a solution or dispersion in a suitable medium which can be administered as a spray. Representative devices of this type are disclosed in the following patents, patent applications, and publications: WO2003026559, WO2002011800, WO200051672, WO2002068029, WO2002068030, WO2002068031, WO2002068032, WO2003000310, WO2003020350, WO2003082393, WO2003084591, WO2003090812, WO200041755, and the pharmaceutical literature (See e.g., Bell, A. Intranasal Delivery Devices, in Drug Delivery Devices Fundamentals and Applications, Tyle P. (ed), Dekker, New York, 1988), Remington's Pharmaceutical Sciences, Mack Publishing Co., 1975, all of which are incorporated herein by reference.

The pharmaceutical compositions may be disclosed in the form of an aerosol or solution for delivery using a pressurized container, pump, spray, atomizer, such as an atomizer using electrohydrodynamics to produce a fine mist, or nebulizer, alone or in combination with a suitable propellant, including, but not limited to, fluorohydrocarbons, chlorofluorohydrocarbons, and volatile unsubstituted hydrocarbons, such as butane, propane, 1,1,1,2-tetrafluoroethane, and/or 1,1,1,2,3,3,3-heptafluoropropane. The pharmaceutical compositions may also be disclosed as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or a surfactant, such as sorbitan trioleate, oleic acid, or an oligolactic acid.

The pharmaceutical compositions disclosed herein may be micronized to a size suitable for delivery, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions disclosed herein; a suitable powder base, such as lactose or starch; and a performance modifier, such as 1-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions disclosed herein for inhaled/intranasal administration may further comprise a suitable flavor, such as menthol and levomenthol, or sweeteners, such as saccharin or saccharin sodium.

The pharmaceutical compositions disclosed herein for topical administration may be formulated to be immediate release or modified release, including delayed-, sustained-, pulsed-, controlled-, targeted, and programmed release.

### D. Modified Release

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. Such modified release formulations may provide the steady release of a therapeutically effective concentration of any of the compounds of the present disclosure from the dosage form, without sedative or psychotomimetic toxic spikes in plasma concentration of any of the compounds. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

In some embodiments, the pharmaceutical composition comprises an amount of any of the compounds described herein and a matrix which provides a release rate of 0.05-2 mg/kg/h over a period of 12-24 hours, e.g., 24 hours.

In some embodiments, the pharmaceutical composition achieves a combined concentration of any of the compounds described herein (e.g., a compound of Formula (I) through (VII)), in plasma in the range of about 10-500 ng/ml, or about 10-300 ng/ml, or about 10-100 ng/ml, or about 10-20 ng/ml, or about 20-500 ng/ml, or about 30-400 ng/ml, or about 40-300 ng/ml, or about 50-100 ng/ml, and maintains this concentration for duration of the release period.

In some embodiments, the tablet composition is a modified-release tablet adapted for sustained release and preferably maximum sustained release. In some embodiments, the release period of any of the compounds described herein (e.g., a compound of Formula (I) through (VII)), in the formulations of the disclosure is greater than 4 hours, greater than 6 hours, greater than 8 hours, greater than 10 hours, greater than 12 hours, greater than 16 hours, greater than 20 hours, greater than 24 hours, greater than 28 hours, greater than 32 hours, greater than 36 hours, greater than 48 hours.

### 1. Matrix Controlled Release Devices

The pharmaceutical compositions disclosed herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In some embodiments, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In some embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT) (e.g., technology directed to a single-layer tablet, caplet or granules coated with an insoluble, asymmetric microporous membrane produced by controlled phase separation), extruding core system (ECS), elementary osmotic pump (EOP), and controlled-porosity osmotic pump (CPOP). The osmotic controlled release device may be in the form of a tablet, caplet or granules, for example. In general, such devices have at least two components: (a) the core which contains the active ingredient(s); and (b) a semipermeable membrane with at least one delivery port, which encapsulates the core. Single or multi-layer release systems may be utilized. The semipermeable membrane controls the influx of water to the core from an aqueous environment of use so as to cause drug release by extrusion through the delivery port(s). Without wishing to be bound by theory, because these systems use water osmotic pressure for the controlled delivery of the active ingredient, delivery rates are expected to be independent of gastrointestinal conditions.

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO) (or polyethylene glycol (PEG)), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the composition.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In some embodiments, the pharmaceutical compositions disclosed herein are formulated as AMT controlled-release dosage forms, which comprises an asymmetric osmotic membrane that coats a core comprising the active ingredient(s) and other pharmaceutically acceptable excipients. The AMT controlled-release dosage forms can be prepared according to conventional methods and techniques known to those skilled in the art, including direct compression, dry granulation, wet granulation, and a dip-coating method.

In some embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

In some embodiments, the disclosure provides a method of formulating any of the compounds described herein (e.g., a compound of Formula (I) through (VII), or a pharmaceutically acceptable salt, solvate, stereoisomer, or prodrug thereof), to ensure the steady release of a therapeutically effective concentration of any of the compounds from a modified release dosage form without sedative or psychotomimetic toxic spikes in plasma concentration of any of the compounds. In some embodiments, the method comprises formulation of any of the compounds described herein (e.g., a compound of Formula (I) through (VII), or a pharmaceutically acceptable salt, solvate, stereoisomer, or prodrug thereof), in an osmotic controlled release tablet. In these formulations, the single core layer containing any of the compounds described herein (e.g., a compound of Formula (I) through (VII), or a pharmaceutically acceptable salt, solvate, stereoisomer, or prodrug thereof), is surrounded by semi-permeable membrane with or without drug delivery orifice. In some embodiments, combination with the novel and inventive pharmaceutical compositions containing any of the compounds described herein (e.g., a compound of Formula (I) through (VII), or a pharmaceutically acceptable salt, solvate, stereoisomer, or prodrug thereof), of the disclosure and osmotic asymmetric-membrane technology or AMT (e.g., technology directed to a single-layer tablet coated with an insoluble, asymmetric microporous membrane produced by controlled phase separation) may be used to produce formulations useful in the methods and kits described herein.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 μm to about 3 mm, about 50 m to about 2.5 mm, or from about 100 m to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions disclosed herein may also be formulated to be targeted to a particular tissue, receptor, or other area of the body of the subject to be treated, including liposome-, resealed erythrocyte-, and antibody-based delivery systems.

### E. Inhalation Administration

The pharmaceutical compositions disclosed herein may be formulated for inhalation administration, e.g., for pulmonary absorption. Drugs, including psychedelic drugs, that can be used for inhalation administration include the compounds described herein (e.g., a compound of Formula (I) through (VII), or a pharmaceutically acceptable salt, solvate, stereoisomer, or prodrug thereof). Suitable preparations may include liquid form preparations such as those described above, e.g., solutions and emulsions, wherein the solvent or carrier is, for example, water, water/water-miscible vehicles such as water/propylene glycol solutions, or organic solvents, with optional buffering agents, which can be delivered as an aerosol, preferably a mist, with a carrier gas, such as air, oxygen, a mixture of helium and oxygen, or other gases and gas mixtures. The pharmaceutical compositions may also be formulated as a dry powder for insufflation, alone or in combination with an inert carrier such as lactose or phospholipids.

The pharmaceutical compositions may be in the form of an aerosol or solution for delivery using a pressurized container, pump, spray, atomizer, such as an atomizer using electrohydrodynamics to produce a fine mist, or nebulizer, alone or in combination with a suitable propellant, such as dichlorodifluoromethane, trichlorofluoromethane, dichlorotetrafluoroethane, a hydrofluoroalkane such as 1,1,1,2-tetrafluoroethane (HFA 134A) and 1,1,1,2,3,3,3-heptafluoropropane (HFA 227), carbon dioxide, perfluorinated hydrocarbons such as perflubron, and other suitable gases.

Aqueous solutions suitable for inhalation use can be prepared by dissolving the compound of Formula (I) through (VII), or a pharmaceutically acceptable salt, solvate, stereoisomer, or prodrug thereof, in water or other water-based medium. Suitable stabilizers and thickening agents can also be added. Emulsions suitable for inhalation use can be made by solubilizing the compound of Formula (I) through (VII), or a pharmaceutically acceptable salt, solvate, stereoisomer, or prodrug thereof, in an aqueous medium and dispersing the solubilized form in a hydrophobic medium, optionally with viscous material, such as natural or synthetic gums, resins, methylcellulose, sodium carboxymethylcellulose, and other suspending agents.

Solutions or suspensions, including those for use in a pressurized container, pump, spray, atomizer, or nebulizer, may be formulated to contain a surfactant or other appropriate co-solvent, or a suitable alternative agent for dispersing, solubilizing, or extending release of the active ingredient disclosed herein, and optionally a propellant. Such surfactants or co-solvents may include, but are not limited to, Polysorbate 20, 60, and 80; Pluronic F-68, F-84, and P-103; cyclodextrin; polyoxyl 35 castor oil; sorbitan trioleate, oleic acid, or an oligolactic acid. Surfactants and co-solvents may be optionally employed at a level between about 0.001%, about 0.01%, about 0.1%, about 1%, and about 5%, about 4%, about 3%, about 2% by weight, based on a total amount of the pharmaceutical composition, or any range therebetween. Viscosity greater than that of simple aqueous solutions may be desirable in some cases to decrease variability in dispensing the formulations, to decrease physical separation of components of an emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such viscosity building agents, when desirable, are typically employed at a level between about 0.001%, about 0.01%, about 0.1%, about 1%, and about 5%, about 4%, about 3%, about 2% by weight, based on a total amount of the pharmaceutical composition, or any range therebetween.

The compounds of the present disclosure can also be dissolved in organic solvents or aqueous mixtures of organic solvents. Organic solvents can be, for example, acetonitrile, chlorobenzene, chloroform, cyclohexane, 1,2-dichloroethane (DCE), dichloromethane (DCM), 1,2-dimethoxyethane, N,N-dimethylacetamide, N,N-dimethylformamide, 1,4-dioxane, 2-ethoxyethanol, ethylene glycol, formamide, hexane, methanol, ethanol, 2-methoxyethanol, methybutylketone, methylcyclohexane, N-methylpyrrolidone, nitromethane, pyridine, sulfolane, tetralin, toluene, 1,1,2-trichloroethylene, or xylene, and like, including combinations thereof. Organic solvents can belong to functional group categories such as ester solvents, ketone solvents, alcohol solvents, amide solvents, ether solvents, hydrocarbon solvents, etc. each of which can be used.

The compounds of the present disclosure (e.g., a compound of Formula (I) through (VII) or a pharmaceutically acceptable salt, solvate, stereoisomer, or prodrug thereof) can be delivered as an aerosol, preferably a mist, via inhalation, for systemic administration to the patient's central nervous system. Preferably, the aerosol is generated without externally added heat (this does not exclude minor temperature increases caused by the formation of the aerosol itself, such as with a vibrating mesh or other nebulizer. However, such minor temperature increases can often be offset by vaporization of the drug, which results in cooling of the composition). The compounds of the present disclosure can be delivered as an aerosol, preferably a mist, with a carrier, such as air, oxygen, or a mixture of helium and oxygen, or other gas mixtures including therapeutic gas mixtures. The carrier gas, e.g., air, oxygen, a mixture of helium and oxygen, or other gases and gas mixtures, can be heated to about 50° C. to about 60° C., or to about 55° C. to about 56° C. When a mixture of helium and oxygen is used as the carrier, the helium can be present in the mixture of oxygen and helium at about 50%, 60%, 70%, 80% or 90% by volume, and the oxygen can be present in the mixture at about 50%, 40%, 30%, or 10% by volume, or any range therebetween.

Inhalation delivery can further comprise administering a pretreatment inhalation therapy prior to administration of the aerosol comprising the compound of Formula (I) through (VII), or a pharmaceutically acceptable salt, solvate, stereoisomer, or prodrug thereof. The pretreatment can comprise administering via inhalation of a mixture of helium and oxygen heated to about 90° C., to about 92° C., to about 94° C., to about 96° C., to about 98° C., to about 100° C., to about 105° C., to about 110° C., to about 115° C., to about 120° C., or any range therebetween, to the patient. For example, an inhalation procedure may involve (i) administering via inhalation a mixture of helium and oxygen heated to about 90° C. to about 120° C. to the patient, followed by (ii) administering via inhalation a mixture of helium and oxygen heated to about 50° C. to about 60° C. and the aerosol comprising the compound of Formula (I) through (VII) or a pharmaceutically acceptable salt, solvate, stereoisomer, or prodrug thereof to the patient and then repeating steps (i) and (ii). Steps (i) and (ii) can be repeated 1, 2, 3, 4, 5, or more times.

The compounds of the present disclosure (e.g., compound of Formula (I) through (VII)) can, in some embodiments, be administered via aerosol inhalation at doses of about 1 μg to about 200 mg or more (or any range between about 1 μg to about 200 mg), e.g., about 1 μg, 2 μg, 5 μg, 6 μg, 10 μg, 13 μg, 15 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 400 μg, 500 μg, 1.0 mg, 2.0 mg, 3.0 mg, 4.0 mg, 5.0 mg, 6.0 mg, 7.0 mg, 8.0 mg, 9.0 mg, 10.0 mg, 20.0 mg, 30.0 mg, 40.0 mg, 50.0 mg, 60.0 mg, 70.0 mg, 80.0 mg, 90.0 mg, 100.0 mg, 150.0 mg, 200.0 mg, or more, per inhalation session. In some embodiments, a subject can have 1, 2, 3, 4, 5 or more inhalation sessions a day. In some embodiments, a subject can have 1, 2, 3, 4, 5 or more inhalation sessions every other day, once a week, twice a week, or three times a week.

In some embodiments, a subject can have 1, 2, 3, 4, 5 or more inhalation sessions every other month, twice a month, three times a month, or four times a month. In some embodiments, a subject can have 1, 2, 3, 4, 5, 6, 7, 8, or more inhalation sessions per treatment course, such as within a 28-day time period.

### Aerosols

An aerosol, preferably a mist, can be delivered using, air, oxygen, a mixture of helium and oxygen, or other gases and gas mixtures, as a carrier gas. The carrier gas can be delivered at room temperature or heated. In some embodiments, an aerosol, preferably a mist comprising a compound of Formula (I) through (VII) or a pharmaceutically acceptable salt, solvate, stereoisomer, or prodrug thereof is delivered via inhalation using heated helium-oxygen (HELIOX) mixtures. Due to very low viscosity of helium the helium-oxygen mixtures generate gaseous streams characterized by laminar flow that is a highly desirable feature for reaching out into the deep lung areas and reducing deposition of the drug in the respiratory tract, one of the major obstacles in dose delivery via inhalation. A patient can inhale a dissolved compound disclosed herein as a mist into an alveolar region of the patient's lungs. The compound of Formula (I) through (VII) can then be delivered to a fluid lining of the alveolar region of the lungs and can be systemically absorbed into patient blood circulation. Advantageously, these formulations can be effectively delivered to the blood stream upon inhalation to the alveolar regions of the lungs.

Devices suitable for delivery of heated or unheated carrier gas (e.g., air, oxygen, or helium-oxygen mixtures) include, for example, continuous mode nebulizers Flo-Mist (Phillips) and Hope (B&B Medical Technologies) and the accessories such as regulators, e.g., Medipure™ Heliox-LCQ System (PraxAir) and control box, e.g., Precision Control Flow (PraxAir). In some embodiments, a full delivery setup can be a device as described in, for example, Russian patent RU199823U1.

The term “heliox” as used herein refers to breathing gas mixtures of helium gas (He) and oxygen gas (O₂). In some embodiments, the heliox mixture can contain helium in the mixture of helium and oxygen at about 50%, 60%, 70%, 80% or 90% by volume, and contain oxygen in the mixture of helium and oxygen at about 50%, 40%, 30%, or 10% by volume, or any range therebetween. The heliox mixture can thus contain helium and oxygen in a volume ratio of 50:50, 60:40, 70:30, 80:20, 90:10, or any range therebetween. In some embodiments, heliox can generate less airway resistance through increased tendency to laminar flow and reduced resistance in turbulent flow.

The use of heat in heliox mixtures can further enhance drug delivery by increasing permeability of key physical barriers for drug absorption. Heating of mucosal surfaces can increase permeability by enhancing peripheral blood circulation and relaxing the interstitial junction, as well as other mechanisms. Helium has a thermal conductivity almost 10 times higher than oxygen and nitrogen and can facilitate heat transfer more efficiently. A dry heliox mixture can be used safely as a pretreatment step when warmed up to as high as 110° C., which can enable the dry heliox mixture to heat mucosal surfaces of the lung and respiratory tract more efficiently.

Various types of personal vaporizers are known in the art. In general, personal vaporizers are characterized by heating a solid drug or compound. Vaporizers can work by directly heating a solid drug or compound to a smoldering point. Vaporizing a solid or solid concentrate can be done by convection or conduction. Convection heating of solid concentrate involves a heating element coming into contact with water, or another liquid, which then vaporizes. The hot vapor in turn directly heats the solid or solid concentrate to a smoldering point, releasing a vapor to be inhaled by a user. Conduction heating involves direct contact between the solid or solid concentrate and the heating element, which brings the solid to a smoldering point, releasing vapor to be inhaled by a user. Though vaporizers present advantages over smoking in terms of lung damage, the drug/active ingredient that is vaporized can be substantially deteriorated by the vaporizing heat.

In some embodiments, the compound of Formula (I) through (VII), or a pharmaceutically acceptable salt, solvate, stereoisomer, or prodrug thereof, is delivered via a nebulizer, which generates an aqueous-droplet aerosol, preferably a mist, containing the compound, which is optionally combined with a heated helium-oxygen mixture. In some embodiments, the disclosed compounds are delivered via a nebulizer, which generates an aqueous-droplet aerosol, preferably a mist, containing the compound, which is combined with a driving gas comprising nitrous oxide. The driving gas comprising nitrous oxide may be nitrous oxide gas itself or a therapeutic gas mixture, such as a N₂O—O₂ mixture or a N₂O-air mixture. The therapeutic gas mixture may further include other gases such as one or more of N₂, Ar, CO₂, Ne, CH₄, He, Kr, H₂, Xe, H₂O (e.g., vapor), etc. In some embodiments, the driving gas is a therapeutic gas mixture comprising N₂O, which is present at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the therapeutic gas mixture, or any range in between. The presence of nitrous oxide (being an NMDA receptor antagonist) in (or as) the driving gas can augment the effect of the disclosed compounds and provide the ability to use lower doses thereof to obtain similar levels of effect.

For example, a preparation of compound of Formula (I) through (VII), or a pharmaceutically acceptable salt, solvate, stereoisomer, or prodrug thereof, can be placed into a liquid medium and put into an aerosol by a device, such as a nebulizer. In some embodiments, a nebulizer can be, for example, a pneumatic compressor nebulizer, an ultrasonic nebulizer, a vibrating mesh or horn nebulizer, or a microprocessor-controlled breath-actuated nebulizer. In some embodiments, a nebulizer device can be a device as described in, for example, Russian patent RU199823U1.

A nebulizer is a device that turns a drug, such as a compound of Formula (I) through (VII), in solution or suspension into a fine aerosol, such as a mist, for delivery to the lungs. A nebulizer can also be referred to as an atomizer. To atomize is to put a dissolved drug into an aerosol, such as a mist, form. To deliver a drug by nebulization, a drug can be dispersed in a liquid medium, for example, water, ethanol, or propylene glycol. Additionally, the disclosed compounds can be carried in an excipient such as, for example liposomes, polymers, emulsions, micelles, nanoparticles, or polyethylenimine (PEI). Liquid drug formations for nebulizers can be, for example, aqueous solutions or viscous solutions. After application of a dispersing forcer (e.g., jet of gas, ultrasonic waves, or vibration of mesh), the dissolved drug is contained within liquid droplets, which are then inhaled. A mist can contain liquid droplets containing the drug in air or another gaseous mixture (e.g., a mixture of helium and oxygen).

Jet nebulizers (also known as pneumatic nebulizers or compressor nebulizers) use compressed gas to make a mist. In some embodiments, a jet nebulizer is a microprocessor-controlled breath-actuated nebulizer, also called a breath-actuated nebulizer. A breath-actuated nebulizer creates a mist only when a patient is inhaling, rather than creating a mist continuously. A mist can be generated by, for example, passing air flow through a Venturi in a nebulizer bowl or cup. A Venturi is a system for speeding the flow of a fluid by constricting fluid in a cone shape tube. In the restriction, the fluid must increase its velocity, thereby reducing its pressure and producing a partial vacuum. As the fluid exits the constriction point, its pressure increases back to the ambient or pipe level pressure. This can form a low-pressure zone that pulls up droplets through a feed tube from a solution of drug in a nebulizer bowl, and in turn this creates a stream of atomized droplets, which flow to a mouthpiece. Higher air flows lead to a decrease in particle size and an increase in output. Due to droplets and solvent that saturates the outgoing gas, jet nebulizers can cool a drug solution in the nebulizer and increase solute concentration in the residual volume. A baffle in a nebulizer bowl or cup can be impacted by larger particles, retaining them and returning them to the solution in the nebulizer bowl or cup to be reatomized. Entrainment of air through a nebulizer bowl as the subject inhales can increase mist output during inspiration. Generation of a mist can occur with a smaller particle size distribution, but using smaller particle sizes can result in an increased nebulization time.

The unit of measurement generally used for droplet size is mass median diameter (MMD), which is defined as the average droplet diameter by mass. This unit can also be referred to as the mass mean aerodynamic diameter, or MMAD. The MMD droplet size for jet nebulizers can be about 1.0, 1.5, 2.0, 2.5, 3.0, 3.5, 4.0, 5.0, 6.0, 7.0, 8.0, 9.0, 10.0 μm or more (or any range between about 1.0 and 10.0 μm), which can be smaller than that of ultrasonic nebulizers.

Ultrasonic nebulizers generate mists by using the vibration of a piezoelectric crystal, which converts alternating current to high-frequency (about 1 to about 3 MHz) acoustic energy. The solution breaks up into droplets at the surface, and the resulting mist is drawn out of the device by the patient's inhalation or pushed out by gas flow through the device generated by a small compressor. Ultrasonic nebulizers can include large-volume ultrasonic nebulizers and small-volume ultrasonic nebulizers. Droplet sizes tend to be larger with ultrasonic nebulizers than with jet nebulizers. The MMD droplet size for ultrasonic nebulizers can be about 2.0, 2.5, 3.0, 3.5, 4.0, 4.5, 5.0, 5.5, 6.0, 6.5, 7.0, 7.5, 8.0, 9.0, 10.0 μm or more (or any range between about 2.0 and 10.0 μm). Ultrasonic nebulizers can create a dense mist, with droplets at about 100, 150, 200, 250, 300 μm/L or more.

Mesh nebulizer devices use the vibration of a piezoelectric crystal to indirectly generate a mist. Mesh nebulizers include, for example, active mesh nebulizers and passive mesh nebulizers. Active mesh nebulizers use a piezo element that contracts and expands on application of an electric current and vibrates a precisely drilled mesh in contact with the drug solution to generate a mist. The vibration of a piezoelectric crystal can be used to vibrate a thin metal plate perforated by several thousand holes. One side of the plate is in contact with the liquid to be atomized, and the vibration forces this liquid through the holes, generating a mist of tiny droplets. Passive mesh nebulizers use a transducer horn that induces passive vibrations in the perforated plate with tapered holes to produce a mist. Examples of active mesh nebulizers include the Aeroneb® (Aerogen, Galway, Ireland) and the eFlow® (PARI, Starnberg, Germany), while the Microair NE-U22® (Omron, Bannockburn, IL) is a passive mesh nebulizer. Mesh nebulizers are precise and customizable. By altering the pore size of the mesh, the device can be tailored for use with drug solutions of different viscosities, and the output rate changed. Use of this method of atomization can offer several advantages. The size of the droplets can be extremely precise because droplet size can be determined by the size of the holes in the mesh (which may be tailor-made to suit the application). Nebulizer meshes can be manufactured using methods such as electrodeposition, electroplating, and laser cutting to produce a liquid particle in gas in the respirable range. Mesh can be made of metal alloy. The metals used in mesh manufacture can include platinum, palladium, nickel, and stainless steel. The size of the droplet is about twice the size of the mesh hole. Mesh holes, therefore, can be about 0.1, 0.5, 1.0, 1.5, 2.0, 2.5, 3.0, 3.5, 4.0, 4.5, 5.0 μm or more (or any value in between about 0.1 and 5.0 μm). Mist generation in mesh nebulizers can vary based on the shape of the mesh, the material that the mesh is made of, and also the way that the mesh is created. In other words, different meshes can produce different sized liquid particles suspended in gas. Generally, MMD droplet size for mesh nebulizers can be about 1.0, 1.5, 2.0, 2.5, 3.0, 3.5, 4.0, 4.5, 5.0, 5.5., 6.0, 6.1, 6.2, 6.3, 6.4, 6.5, 6.6, 6.7, 6.8, 6.9, 7.0 μm or more (or any value in between about 1.0 and 7.0 μm).

Additionally, droplet size can be programmable. In particular, geometric changes can be made to a nebulizer to provide a specific desired droplet size. Additionally, droplet size can be controlled independently of droplet velocity. The volume of liquid atomized, and the droplet velocity can also be precisely controlled by adjusting the frequency and amplitude of the mesh vibration. Furthermore, the number of holes in the mesh and their layout on the mesh can be tailored. Mesh nebulizers can be powered either by electricity or by battery.

A mist output rate in standing cloud mL per minute (for any atomization methodology described herein) can range from, for example, 0.1, 0.2, 0.3, 0.4, 0.5, 0.6, 0.7, 0.8, 0.9 mL/minute or more (or any range between about 0.1 and 0.9 mL/minute) and the residual volume in any type of nebulizer reservoir can range from a about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mL or more (or any range between about 0.01 and 2.0 mL). Precise droplet size control can be advantageous since droplet size can correlate directly to kinetic drug release (KDR). Precise control of KDR can be achievable with precise control of droplet size. The compounds herein can be delivered via a mist using any methodology with an MMD droplet size of about 0.5, 1.0, 1.5, 2.0, 2.5, 3.0, 3.5, 4.0, 5.0, 6.0, 7.0, 8.0, 9.0, 10.0 μm or more (or any range between about 0.5 and 10.0 μm).

In some embodiments, the compounds of the present disclosure (e.g., a compound of Formula (I) through (VII), or a pharmaceutically acceptable salt, solvate, stereoisomer, or prodrug thereof) can be delivered via a continuous positive airway pressure (CPAP) or other pressure-assisted breathing device. A pressure-assisted breathing device forces a continuous column of compressed air or other gas at a fixed designated pressure against the face and nose of the patient, who is wearing a mask or nasal cap. When the patient's glottis opens to inhale, the pressure is transmitted throughout the airway, helping to open it. When the patient exhales, pressure from the deflating lungs and chest wall pushes air out against the continuous pressure, until the two pressures are equal. The air pressure in the airway at the end of exhalation is equal to the external air pressure of the machine, and this helps “splint” the airway open, allowing better oxygenation and airway recruitment. A pressure-assisted breathing device can be coupled with a means for introducing mist particles into the gas flow in the respiratory circuit and or a means for discontinuing the introduction of mist particles into the respiratory circuit when the patient exhales. See, e.g. U.S. Pat. No. 7,267,121.

In some embodiments, a mist can be delivered by a device such as a metered dose inhaler (MDI) (also referred to as a pressurized metered dose inhaler or pMDI), which generates an organic solvent-droplet mist containing a compound of Formula (I) through (VII), which is optionally combined with a heated helium-oxygen mixture. In some embodiments, the compound can be delivered via a metered dose inhaler, MDI. MDI devices can include a canister which contains the compound of Formula (I) through (VII) and a propellant, a metering valve which dispenses the medicament from the canister, an actuator body that receives the canister and which forms an opening for oral inhalation, and an actuator stem which receives the drug from the canister and directs it out the opening in the actuator body. A non-limiting example of a metering valve and actuator is Bespak's BK357 valve and actuator (orfice d=0.22 mm) by Recipharm. Moving the drug canister relative to the actuator body and actuator stem causes the metering valve to release the predetermined amount of the drug. In some embodiments, the compound of Formula (I) through (VII), or a pharmaceutically acceptable salt, solvate, stereoisomer, or prodrug thereof, can be dissolved in a liquid propellant mixture (sometimes including small amounts of a volatile organic solvent) stored in a pressurized container of the MDI. The “metered dose” is the dose that is prepackaged in a single-dose inhaler, or which in a multidose inhaler is automatically measured out of a reservoir in preparation for inhalation. MDI devices can be aided with spacers. An MDI spacer is a spacer that goes between the MDI and the mouth of a user of the MDI. An MDI spacer allows droplets in the atomized dose to settle out a bit and mix with air or other gas, thus allowing for more effective delivery of a metered dose into a user's lungs when inhaled. An MDI spacer assists in preventing a user from inhaling the metered dose directly from an MDI where the dose would be traveling so fast that the droplets of the atomized spray from the MDI hit and stick to the back of the user's throat rather than being inhaled into the user's lungs where the drug of the metered dose is designed to be delivered. MDI devices offer the advantage of regular dosing, which can be controlled in the manufacture of the drug.

Drugs can also be delivered by dry powder inhalers (DPI). In such DPI devices, the drug itself can form the powder or the powder can be formed from a pharmaceutically acceptable excipient or carrier and the drug is releasably bound to a surface of the carrier powder such that upon inhalation, the moisture in the lungs releases the drug from the surface to make the drug available for systemic absorption. In some embodiments, the compound of Formula (I) through (VII), or a pharmaceutically acceptable salt, solvate, stereoisomer, or prodrug thereof, is delivered by use of a dry powder inhaler (DPI). Depending on the compound or form used, the drug can be formed into the necessary powder itself, or can be releasably bound to a surface of a carrier powder. Such carrier powders are known in the art (see, e.g., H. Hamishehkar, et al., “The Role of Carrier in Dry Powder Inhaler”, Recent Advances in Novel Drug Carrier Systems, 2012, pp. 39-66).

DPI is generally formulated as a powder mixture of coarse carrier particles and micronized drug particles with aerodynamic particle diameters of 1-5 μm (see e.g., Iida, Kotaro, et al. “Preparation of dry powder inhalation by surface treatment of lactose carrier particles” Chemical and pharmaceutical bulletin 51.1 (2003): 1-5). Carrier particles are often used to improve drug particle flowability, thus improving dosing accuracy and minimizing the dose variability observed with drug formulations alone while making them easier to handle during manufacturing operations. Carrier particles should have several characteristics such as physico-chemical stability, biocompatibility and biodegradability, compatible with the drug substance and must be inert, available and economical. The choice of carrier particle (both content and size) is well within the purview of one of ordinary skill in the art. The most common carrier particles are made of lactose or other sugars, with α-lactose monohydrate being the most common lactose grade used in the inhalation field for such particulate carriers.

Delivery with Helium Oxygen Mixtures

Systemic delivery of the compounds of the present disclosure (e.g., a compound of Formula (I) through (VII), or a pharmaceutically acceptable salt, solvate, stereoisomer, or prodrug thereof) can be carried out via inhalation of an aerosol comprising the compound and a carrier gas such as air, oxygen, helium, a mixture of helium and oxygen (i.e., a heliox mixture), other gases or other gas mixtures. In particular, the compounds of the present disclosure can be delivered to a patient's CNS. Accordingly, methods of treating various central nervous system (CNS) diseases and other conditions are described herein, in which doses can be optimized for individual patients' metabolism and treatment needs. In some embodiments, the carrier gas can be heated. The method can further comprise using a device containing a balloon with an oxygen-helium mixture equipped with a reducer and a mask connected to each other by a gas or air connecting tube, which contains an additional heating element capable of heating the gas mixture up to 120° C., a nebulizer with a vibrating porous plate or mesh, ensuring the passage of droplets with a size of less than 5 microns through it, and a disinfection unit.

In some embodiments, the compound of Formula (I) through (VII) or a pharmaceutically acceptable salt, solvate, stereoisomer, or prodrug thereof is delivered to the lower respiratory tract, for instance, to a pulmonary compartment such as alveoli, alveolar ducts and/or bronchioles. From there, the drug can enter the blood stream and travel to the central nervous system. In some embodiments, inhalation of a mist can deliver the compound of Formula (I) through (VII) or a pharmaceutically acceptable salt, solvate, stereoisomer, or prodrug thereof to the patient's CNS without passing through the liver. Administration via inhalation can allow gaseous drugs or those dispersed in a liquid or a mist, to rapidly reach the blood stream, bypassing first-pass metabolism. First-pass metabolism, also known as “first-pass effect” or “presystemic metabolism” describes drugs that enter the liver and undergo extensive biotransformation.

In some embodiments, the present disclosure provides a treatment step, in which the compound of Formula (I) through (VII) or a pharmaceutically acceptable salt, solvate, stereoisomer, or prodrug thereof can be administered to a patient in need thereof by administering via inhalation a mixture of helium and oxygen heated to about 50° C., 51° C., 52° C., 53° C., 54° C., 55° C., 56° C., 57° C., 58° C., 59° C., 60° C., or more (or any range between 50° C. to 60° C.) and the atomized compound. In some embodiments, a mist or vapor of the compound can have a particle size from about 0.1 microns to about 10 microns (e.g., about 10, 5, 4, 3, 2, 1, 0.1 or less microns). In some embodiments, atomization is performed via a nebulizer creating an inhalant that is a mist. In some embodiments, the atomized compound is driven down the patient delivery line by the patient's inhalation. In some embodiments, the atomized compound is driven down the patient delivery line by the patient's inhalation using a carrier gas. The carrier gas can be air, oxygen, a mix of oxygen and helium, heated air, heated oxygen, a heated helium and oxygen mixture, among others.

In some embodiments, the treatment step can be preceded by a pretreatment step. In some embodiments, the pretreatment step can comprise first administering a pretreatment inhalation therapy prior to administration of the mist of the compound of Formula (I) through (VII) or a pharmaceutically acceptable salt, solvate, stereoisomer, or prodrug thereof. In some embodiments, the pretreatment inhalation step can comprise (i) administering via inhalation air, oxygen, or mixture of helium and oxygen heated to about 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., or more (or any range between about 90° C. and 120° C.) and no drug, and then (ii) administering a treatment step of inhalation air, oxygen, a mix of oxygen and helium, heated air, heated oxygen, or heated helium and oxygen mixture, with the atomized compound of Formula (I) through (VII) or a pharmaceutically acceptable salt, solvate, stereoisomer, or prodrug thereof. Heated air, heated oxygen, or heated helium and oxygen mixture, in combination with the atomized compound of the present disclosure, can be heated to about 50° C., 51° C., 52° C., 53° C., 54° C., 55° C., 56° C., 57° C., 58° C., 59° C., 60° C., or more (or any range between about 50° C. and 60° C.).

In some embodiments of the present disclosure, a pretreatment step (i) and a treatment step (ii) can be repeated 0, 1, 2, 3, 4, 5, or more times. In some embodiments of the present disclosure, steps (i) and (ii) can be repeated 0, 1, 2, 3, 4, 5, or more times followed by the treatment step, which can be repeated 0, 1, 2, 3, 4, 5, or more times. In some embodiments of the present disclosure, the treatment step can be repeated 0, 1, 2, 3, 4, 5, or more times with no pretreatment step.

Treatment, with optional pretreatment, can be administered once a week, twice a week, once a day, twice a day, three times a day or more, a defined number of treatments per treatment course (e.g., 1, 2, 3, or 4 treatments per treatment course) or other treatment schedules as set forth herein.

A drug delivery procedure can comprise an inhaled priming no-drug hot heliox mixture to effectively preheat the mucosal bed followed by inhaling an atomized compound of the present disclosure, again driven by the heated heliox, but at lower temperatures, that are now dictated by lower heat tolerance to the wet vs. dry inhaled gas stream. Consequently, this procedure can be conducted in multiple repeated cycles, wherein a target PK and drug exposure is controlled by the concentration of the drug, temperature, flow rate of the helium oxygen mixture, composition of the mixture, number and durations of cycles, time and combinations of the above.

In some embodiments, the present disclosure provides methods of treating a central nervous system (CNS) disorder or psychological disorder comprising administering via inhalation a heated mixture of helium and oxygen heated and an atomized compound of Formula (I) through (VII), or a pharmaceutically acceptable salt, solvate, stereoisomer, or prodrug thereof. The treatment can alleviate one or more symptoms of the disorder. In some embodiments, the compound of the present disclosure can be administered for treatment of CNS disease or other disorder. In some embodiments, the compound of the present disclosure can be administered to treat depression including, but not limited to major depression, melancholic depression, atypical depression, or dysthymia. In some embodiments the compound of the present disclosure can be administered to treat psychological disorders including anxiety disorder, obsessive compulsive disorder, addiction (narcotic addiction, tobacco addiction, opioid addiction), alcoholism, depression and anxiety (chronic or related to diagnosis of a life-threatening or terminal illness), compulsive behavior, or a related symptom.

In some embodiments, the disease or disorder is selected from the group consisting of central nervous system (CNS) disorders, including post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), bipolar and related disorders including bipolar I disorder, bipolar II disorder, cyclothymic disorder, obsessive-compulsive disorder (OCD), generalized anxiety disorder (GAD), social anxiety disorder, substance use disorders including alcohol use disorder, opioid use disorder, amphetamine use disorder, nicotine use disorder, and cocaine use disorder, anorexia nervosa, bulimia nervosa, binge eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, sexual dysfunction, chronic fatigue syndrome, Lyme disease, and obesity. In some embodiments, the disease or disorder may include conditions of the autonomic nervous system (ANS). In some embodiments, the disease or disorder may include pulmonary disorders (e.g., asthma and chronic obstructive pulmonary disorder (COPD). In some embodiments, the disease or disorder may include cardiovascular disorders (e.g., atherosclerosis).

The methods of delivering a compound of the present disclosure to the CNS (systemic drug delivery) via nebulizer (including, for example, using a heated helium-oxygen mixture), can lead to advantageous improvements in multiple PK parameters as compared to oral delivery. In particular, the compound can cross the blood brain barrier and be delivered to the brain. As compared to oral delivery, the method of delivering a compound of the present disclosure to the CNS via nebulizer, optionally with a heated heliox mixture, can increase bioavailability by at least about 10%, 25%, 30%, 35%, 40%, 50%, 55%, 60%, 65%, 70%, 80%, 85%, 90%, 95%, 99%, 99.9%, or more as compared to oral delivery. The method of delivering a compound of the present disclosure to the CNS via nebulizer as described herein, can reduce Tₘₐₓ by at least 30%, 40%, 50%, 55%, 60%, 65%, 70%, 80%, 85%, 90%, 95%, 99%, 99.9%, or more as compared to oral delivery. In some embodiments, the method of delivering the compound of the present disclosure to the CNS via nebulizer as described herein, can increase Cₘₐₓ by at least 10%, 25%, 30%, 35%, 40%, 50%, 55%, 60%, 65%, 70%, 80%, 85%, 90%, 95%, 99%, 99.9%, or more as compared to oral delivery. Furthermore, a method of delivering a compound of the present disclsoure to the CNS via nebulizer as described herein, can allow clinical protocols enabling dose titration and more controlled exposure. Controlled exposure enables adjusting the patient experience and providing overall improved therapeutic outcomes.

In some embodiments, a system is provided for administering a compound of the present disclosure (e.g., a compound of Formula (I) through (VII), or a pharmaceutically acceptable salt, solvate, stereoisomer, or prodrug thereof) that includes a container comprising a solution of the compound and a nebulizer physically coupled or co-packaged with the container and adapted to produce a mist of the solution having a particle size from about 0.1 microns to about 10 microns (e.g., about 10, 5, 4, 3, 2, 1, 0.1 or less microns).

Combination Therapy with an NMDA Receptor Antagonist

Also disclosed are combination drug therapies based on administration of both a compound of Formula (I) through (VII), or a pharmaceutically acceptable salt, solvate, stereoisomer, or prodrug thereof, (as a 5-HT_{2A} receptor agonist) and a N-methyl-D-aspartate (NMDA) receptor antagonist. The combination drug therapy may enhance activity and improve patient experience when treating diseases or disorders associated with 5-HT_{2A} and/or NMDA receptors (e.g., a neuropsychiatric disease or disorder, a central nervous system (CNS) disorder, a psychological disorder, etc.), for example, by providing therapeutic efficacy while reducing or eliminating psychiatric adverse effects such as acute psychedelic crisis (bad trip) and dissociative effects from hallucinogens (out of body experience).

Non-limiting examples of NMDA receptor antagonists include, but are not limited to, ketamine, nitrous oxide, memantine, amantadine, dextromethorphan (DXM), phencyclidine (PCP), methoxetamine (MXE), dizocilpine (MK-801), esmethadone, or a combination thereof. In particular, nitrous oxide (N₂O), commonly known as laughing gas, is a rapid and effective analgesic gas that has a fast onset and rarely produces side effects when administered under proper medical supervision. Nitrous oxide is also a dissociative inhalant known to cause euphoria during inhalation. Prominent effects of nitrous oxide are increased feelings of euphoria, a heightened pain threshold, and involuntary laughing. Furthermore, unlike ketamine, nitrous oxide is not addictive. For these reasons, the use of nitrous oxide as the NMDA receptor antagonist is preferred.

In some embodiments, the combination drug therapy involves providing the compound of Formula (I) through (VII), or a pharmaceutically acceptable salt, solvate, stereoisomer, or prodrug thereof, and the NMDA receptor antagonist as a single dosage form for administration to a patient (e.g., each is combined to provide a single aerosol that is inhaled by the patient; or each is combined into a single transdermal patch and delivered transdermally or subcutaneously to the patient). For example, when the NMDA receptor antagonist is nitrous oxide, the compound of Formula (I) through (VII), or a pharmaceutically acceptable salt, solvate, stereoisomer, or prodrug thereof, may be present in the liquid phase of the aerosol, while the nitrous oxide may be present in the gas phase of the aerosol. The nitrous oxide (or therapeutic gas mixture comprising nitrous oxide) may be used in the generation of the aerosol or as a carrier gas used to deliver a generated aerosol to the patient. When a generated aerosol is combined with a carrier gas, the carrier gas becomes a part of the gas phase of the aerosol, i.e., the liquid phase of the aerosol becomes entrained in/diluted by the carrier gas. In some embodiments, the combination drug therapy involves providing the compound of Formula (I) through (VII), or a pharmaceutically acceptable salt, solvate, stereoisomer, or prodrug thereof, and the NMDA receptor antagonist as separate dosage forms. For example, the compound of Formula (I) through (VII), or a pharmaceutically acceptable salt, solvate, stereoisomer, or prodrug thereof, may be provided as an aerosol, preferably a mist, while the NMDA receptor antagonist is provided separately as a therapeutic gas mixture. Alternatively, the compound of Formula (I) through (VII), or a pharmaceutically acceptable salt, solvate, stereoisomer, or prodrug thereof, may be provided as an injectable (e.g., intravenous, intradermal, etc.), bolus, infusion, perfusion, etc., while the NMDA receptor antagonist is provided as a therapeutic gas mixture for inhalation delivery.

The co-action of the compound of Formula (I) through (VII) (as a 5-HT_{2A} receptor agonist) and a NMDA receptor antagonist (e.g., nitrous oxide, ketamine, etc.) may provide multiple benefits. For example, the NMDA receptor antagonist may control and/or reduce the activating effects of the 5-HT₂Rs, thereby reducing the risk of overstimulation and occurrences of psychiatric adverse effects such as acute psychedelic crisis. Additionally, administration of the NMDA receptor antagonist may enable the use of a reduced therapeutic dose of the compound of Formula (I) through (VII), thereby decreasing the likelihood of a negative patient experience or dose-dependent side effects. Similarly, administration of the compound of Formula (I) through (VII) may reduce the amount of NMDA receptor antagonist necessary for a therapeutic effect, which in the case of NMDA receptor antagonists such as nitrous oxide may alleviate certain side effects such as induced involuntary laughter and the general feelings of anxiety associated therewith. Thus, it is believed that co-administration would reduce the likelihood of a negative experience from the psychedelic administration, either because less psychedelic would be administered or the NMDA receptor antagonist (e.g., nitrous oxide, ketamine, etc.) would enable more efficient functioning of the psychedelic. Similarly, such co-administration would reduce the time or amount of NMDA receptor antagonist (e.g., nitrous oxide, ketamine, etc.) necessary for a therapeutic effect.

NMDA receptor antagonists (e.g., nitrous oxide) and 5-HT_{2A} receptor agonists function via different pharmacological pathways. However, both pathways appear to ultimately converge in a cascade at mTOR (mammalian target of rapamycin, or mechanistic target of rapamycin). Thus, a shared mechanism of action appears to exist between NMDA receptor antagonists and 5-HT_{2A} receptor agonists. Specifically, mTOR's signaling pathway may be modulated by 5-HT_{2A} receptor activation and NMDA antagonism. Without being bound by theory, such modulation of the mTOR pathway may underpin the immediate and long-lasting therapeutic and synergistic benefits of combined administration of both agents. As such, in some embodiments, administration of both agents at psychedelic or sub-psychedelic doses enables therapeutic efficacy without or minimizing psychiatric adverse effects.

In addition, it has been found that atrophy of neurons in the prefrontal cortex (PFC) plays a key role in the pathophysiology of depression and related disorders. The ability to promote both structural and functional plasticity in the PFC has been hypothesized to underlie the fast-acting antidepressant properties of the dissociative anesthetic ketamine but also the long-lasting effect after a single administration. Without being bound by theory, it is believed that the combination drug therapy disclosed herein may function by synergistically increasing neuritogenesis and spinogenesis, including increased density of dendritic spines, thereby providing or contributing to long-lasting therapeutic benefits.

A ratio of the compound of Formula (I) through (VII) and the NMDA receptor antagonist administered in the combination drug therapy may vary depending on the patient (i.e., subject), the identity of the active ingredient(s) selections of the combination, the dosage form(s), and the specific disease or condition being treated. It should be understood that a specific ratio of the combination for any particular patient will depend upon a variety of factors, such as the activity of the specific compounds employed, the age, sex, general health of the patient, time of administration, rate of excretion, and the severity of the particular disease or condition being treated. In some embodiments, a weight ratio of the compound of Formula (I) through (VII) and the NMDA receptor antagonist administered to the patient may range from about 1:100 to about 100:1, or any range therebetween, e.g., from about 1:75, from about 1:50, from about 1:40, from about 1:30, from about 1:20, from about 1:10, from about 1:8, from about 1:6, from about 1:5, from about 1:4, from about 1:3, from about 1:2, from about 2:3, from about 1:1, and up to about 100:1, up to about 75:1, up to about 50:1, up to about 40:1, up to about 30:1, up to about 20:1, up to about 10:1, up to about 8:1, up to about 6:1, up to about 5:1, up to about 4:1, up to about 3:1, up to about 2:1. Ratios outside of this range may also be employed, in certain circumstances.

The combination drug therapy is intended to embrace administration of the compound of Formula (I) through (VII) or a pharmaceutically acceptable salt, solvate, stereoisomer, or prodrug thereof and the NMDA receptor antagonist (e.g., nitrous oxide) in a sequential manner, that is, wherein each active ingredient is administered at a different time, as well as administration of these active ingredients, or at least two of the active ingredients, in a concurrent manner. Concurrent administration can be accomplished, for example, by administering to the subject a single dosage form having a fixed ratio of each active ingredient or in multiple, single dosage forms for each of the active ingredients. Administration of the compound of Formula (I) through (VII) or a pharmaceutically acceptable salt, solvate, stereoisomer, or prodrug thereof and a NMDA receptor antagonist (e.g., nitrous oxide), whether in a single dosage form or separate dosage forms, can be carried out by any administration route set forth herein. In some embodiments, both the compound of Formula (I) through (VII) or a pharmaceutically acceptable salt, solvate, stereoisomer, or prodrug thereof and the NMDA receptor antagonist are administered via inhalation, preferably in aerosol (e.g., mist) form. In some embodiments, the compound of Formula (I) through (VII) or a pharmaceutically acceptable salt, solvate, stereoisomer, or prodrug thereof is administered intravenously (IV), and the NMDA receptor antagonist is administered via inhalation. In some embodiments, the compound of Formula (I) through (VII) or a pharmaceutically acceptable salt, solvate, stereoisomer, or prodrug thereof is administered orally, and the NMDA receptor antagonist is administered via inhalation. In some embodiments, both the compound of Formula (I) through (VII) or a pharmaceutically acceptable salt, solvate, stereoisomer, or prodrug thereof and the NMDA receptor antagonist are administered transdermally or subcutaneously. The compositions for inhalation such as pharmaceutically acceptable excipients, etc. for the single or separate dosage forms are set forth herein.

In some embodiments, the NMDA receptor antagonist used in the combination drug therapy is nitrous oxide. Nitrous oxide may be administered alone, or as a therapeutic gas mixture, e.g., N₂O and O₂; N₂O and air; N₂O and medical air (medical air being 78% nitrogen, 21% oxygen, 1% other gases); N₂O and a N₂/O₂ mix; N₂O and O₂ enriched medical air; N₂O and a He/O₂ mix etc. Thus, in addition to nitrous oxide and oxygen, the therapeutic gas mixture may further include other gases such as one or more of N₂, Ar, CO₂, Ne, CH₄, He, Kr, H₂, Xe, H₂O (e.g., vapor), etc. For example, nitrous oxide may be administered using a blending system that combines N₂O, O₂ and optionally other gases from separate compressed gas cylinders into a therapeutic gas mixture which is delivered to a patient via inhalation. Alternatively, the therapeutic gas mixture containing nitrous oxide may be packaged, for example, in a pressurized tank or in small, pressurized canisters which are easy to use and/or portable. The blending system and/or pressurized tanks/canisters may be adapted to fluidly connect to an inhalation device such as a device capable of generating an aerosol of the compound of Formula (I) through (VII) or a pharmaceutically acceptable salt, solvate, stereoisomer, or prodrug thereof. Nitrous oxide itself, or the therapeutic gas mixture comprising nitrous oxide may be used for the generation of the aerosol (i.e., as the gas phase component of the aerosol) or as a carrier gas to facilitate the transfer of a generated aerosol to a patient's lungs. In some embodiments, N₂O is present in the therapeutic gas mixture at a concentration ranging from 5 vol %, from 10 vol %, from 15 vol %, from 20 vol %, from 25 vol %, from 30 vol %, from 35 vol %, from 40 vol %, from 45 vol %, and up to 75 vol %, up to 70 vol %, up to 65 vol %, up to 60 vol %, up to 55 vol %, up to 50 vol %, relative to a total volume of the therapeutic gas mixture. The therapeutic gas mixture containing nitrous oxide can be administered over any desired duration, e.g., 5 minutes, 10 minutes 15 minutes, 20 minutes, 30 minutes, 40 minutes, 45 minutes, 50 minutes, 60 minutes, 90 minutes, 120 minutes, 150 minutes, 180 minutes, or any range therebetween.

In some embodiments, the compound of Formula (I) through (VII) or a pharmaceutically acceptable salt, solvate, stereoisomer, or prodrug thereof and the NMDA receptor antagonist (e.g., nitrous oxide) are each delivered by aerosol inhalation, as a single dosage form or as separate dosage forms. The aerosol, preferably a mist, may be generated by any capable device (e.g., a pressurized container, pump, spray, atomizer, or nebulizer), such as those devices disclosed herein, with or without the use of a propellant. When nitrous oxide is administered concurrently with the compound of Formula (I) through (VII) or a pharmaceutically acceptable salt, solvate, stereoisomer, or prodrug thereof, the nitrous oxide may dually act as a carrier gas or propellant for the aerosol generation and as a therapeutic agent (an NMDA receptor antagonist).

In some embodiments, the delivery device is an inhalation delivery device for delivery of the combination of the compound of Formula (I) through (VII) or a pharmaceutically acceptable salt, solvate, stereoisomer, or prodrug thereof and nitrous oxide by inhalation to a patient in need thereof, comprising an inhalation outlet portal for administration of the combination to the patient; a container configured to deliver nitrous oxide, e.g., in a therapeutic gas mixture, to the inhalation outlet portal; and a device configured to generate and deliver an aerosol comprising the compound of Formula (I) through (VII) or a pharmaceutically acceptable salt, solvate, stereoisomer, or prodrug thereof to the inhalation outlet portal. In some embodiments, the inhalation outlet portal is selected from a mouthpiece or a mask covering the patient's nose and mouth. In some embodiments, the device configured to generate and deliver the aerosol to the inhalation outlet portal is a nebulizer. In some embodiments, the nebulizer is a jet nebulizer and the nitrous oxide gas, alone, or in combination with other gases (therapeutic gas mixture containing nitrous oxide), acts as a driving gas for the jet nebulizer. Accordingly, nitrous oxide delivered using a nebulizer (e.g., jet nebulizer) may dually act as a therapeutic agent and as a driving gas to entrain the nebulized form of the compound of Formula (I) through (VII) or a pharmaceutically acceptable salt, solvate, stereoisomer, or prodrug thereof.

In some embodiments, the device is a dual delivery device configured to administer the compound of Formula (I) through (VII) or a pharmaceutically acceptable salt, solvate, stereoisomer, or prodrug thereof, preferably in the form of an aerosol, and to simultaneously administer a controlled amount of nitrous oxide, either alone or as a therapeutic gas mixture. Any of the above aerosol delivery devices can be used for such a device, with the addition of a source of nitrous oxide (or a source of a therapeutic gas mixture containing nitrous oxide) configured to provide a metered, controlled dose/flow rate of nitrous oxide through the same administration outlet as the aerosol delivery device. In some embodiments, the driving gas for the nebulization of the compound of Formula (I) through (VII) or a pharmaceutically acceptable salt, solvate, stereoisomer, or prodrug thereof is the nitrous oxide or therapeutic gas mixture containing nitrous oxide.

Any of the delivery devices above, e.g., controlled release device, implant, patch, pump, depot, inhaler, inhalation delivery device, etc., can be optionally manufactured with smart technology, e.g., electronics, configured to provide remote activation and operational control of the drug delivery. The remote activation can be performed via computer or mobile app. To ensure security, the remote activation device can be password encoded. This technology enables a healthcare provider to perform telehealth sessions with a patient, during which the healthcare provider can remotely activate and administer the compound of Formula (I) through (VII), or a pharmaceutically acceptable salt, solvate, stereoisomer, or prodrug thereof, via the desired delivery device while supervising the patient on the televisit.

### Kits

In some embodiments, a kit is provided for the treatment of a subject comprising 1) a pharmaceutical composition disclosed herein comprising any of the compounds described herein (e.g., a compound of Formula (I) through (VII), or a pharmaceutically acceptable salt, solvate, stereoisomer, or prodrug thereof), such as an oral dosage form (e.g., a pill or single-layered orally administered tablet), a parenteral dosage form (e.g., an injectable), a topical/transdermal dosage form, a modified release dosage form, or a dosage form to be delivered via inhalation, and (2) instructions for use in the treatment, prevention or management of a disease, disorder or condition described herein.

In some embodiments, the disease, disorder or condition is pain, examples of which include, but are not limited to, cancer pain, e.g., refractory cancer pain, post-surgical pain, orthopedic pain, back pain, neuropathic pain, dental pain, chronic pain, and chronic pain in opioid-tolerant patients. In some embodiments, the disease, disorder or condition is associated with a serotonin 5-HT₂ receptor. In some embodiments, the disease, disorder or condition is selected from the group consisting of central nervous system (CNS) disorders, including post-traumatic stress disorder (PTSD), major depressive disorder (MDD), treatment-resistant depression (TRD), suicidal ideation, suicidal behavior, major depressive disorder with suicidal ideation or suicidal behavior, non-suicidal self-injury disorder (NSSID), bipolar and related disorders including bipolar I disorder, bipolar II disorder, cyclothymic disorder, obsessive-compulsive disorder (OCD), generalized anxiety disorder (GAD), social anxiety disorder, substance use disorders including alcohol use disorder, opioid use disorder, amphetamine use disorder, nicotine use disorder, and cocaine use disorder, anorexia nervosa, bulimia nervosa, binge eating disorder, Alzheimer's disease, cluster headache and migraine, attention deficit hyperactivity disorder (ADHD), pain and neuropathic pain, aphantasia, childhood-onset fluency disorder, major neurocognitive disorder, mild neurocognitive disorder, sexual dysfunction, chronic fatigue syndrome, Lyme disease, and obesity. In some embodiments, the disease, disorder or condition includes conditions of the autonomic nervous system (ANS). In some embodiments, the disease, disorder or condition includes pulmonary disorders including asthma and chronic obstructive pulmonary disorder (COPD). In some embodiments, the disease, disorder or condition includes cardiovascular disorders including atherosclerosis. In some embodiments, the disease, disorder or condition is brain injury. In some embodiments, the disease, disorder or condition is depression. In some embodiments, the disease, disorder or condition is migraine, e.g., with aura. In some embodiments, the disease, disorder or condition is refractory asthma. In some embodiments, the disease, disorder or condition is stroke. In some embodiments, the disease, disorder or condition is alcohol dependence.

In some embodiments, the instructions for use form an integrated component of the packaging for the pharmaceutical composition, e.g., single-layered orally administered tablet composition.

In some embodiments, the disclosure features an oral, modified-release pharmaceutical composition for oral administration to a subject for treating the subject diagnosed with, suffering from or susceptible to a disease, disorder or condition, such as those for which phenethylamine treatment may be indicated, considered or recommended, wherein the subject is in need of treatment with said oral, modified-release pharmaceutical composition, said oral, modified-release pharmaceutical composition comprising:
(a) a compound described herein (e.g., a compound of Formula (I) through (VII), or a pharmaceutically acceptable salt, solvate, stereoisomer, or prodrug thereof) in an effective amount for treating, preventing and/or managing the disease, disorder, or condition in the subject; and
(b) a pharmaceutically acceptable excipient;
whereby, upon oral administration of the modified-release pharmaceutical composition to the subject, a steady release of said compound from the modified-release pharmaceutical composition is maintained so that no neurologically toxic spike in the subject's plasma occurs during the release period of said drug from said pharmaceutical composition.
   Compliance with Monographs

In some embodiments, the pharmaceutical compositions of the disclosure conform to certain industry accepted monographs to afford compliance with the Federal Food Drug and Cosmetic Act. In particular, the pharmaceutical compositions of the disclosure conform and are considered acceptable under visual inspection, uniformity of mass analysis, uniformity of content analysis, and/or dissolution/disintegration analysis all of which are established by a relevant monograph.

In some embodiments, throughout manufacturing certain procedures are validated and monitored by carrying out appropriate in-process controls. These are designed to guarantee the effectiveness of each stage of production. In-process controls during tablet production may include the moisture content of the final lubricated blend, the size of granules, the flow of the final mixture and, where relevant, the uniformity of mass of tablet cores before coating. In-process controls during tablet production may also include the dimensions (thickness, diameter), uniformity of mass, hardness and/or crushing force, friability, disintegration or dissolution rate (for example, for modified-release tablets) of the finished dosage form. Suitable test methods that may be used to demonstrate certain of these attributes arc known in the art.

In some embodiments, packaging maybe or is required to be adequate to protect the pharmaceutical compositions, including tablets, from light, moisture and damage during transportation.

In some embodiments, the commercially available formulation (e.g., kit) complies with the labeling requirements established under Good Manufacturing Practices (GMP). Such label includes:
(1) the name of the pharmaceutical product;
(2) the name(s) of the active ingredient(s); International Nonproprietary Names (INN) should be used wherever possible;
(3) the amount of the active ingredient(s) in each tablet and the number of tablets in the container;
(4) the batch (lot) number assigned by the manufacturer;
(5) the expiry date and, when required, the date of manufacture;
(6) any special storage conditions or handling precautions that may be necessary;
(7) directions for use, warnings, and precautions that may be necessary;
(8) the name and address of the manufacturer or the person responsible for placing the product on the market;
(9) for scored tablets where the directions for use include subdivision to provide doses of less than one tablet, the label should also include: the storage conditions for and the period of use of those subdivided part(s) not immediately taken or administered.

In some embodiments, the pharmaceutical compositions, e.g., tablets, can withstand handling, including packaging and transportation, without losing their integrity.

### EXAMPLES

I. Synthetic routes

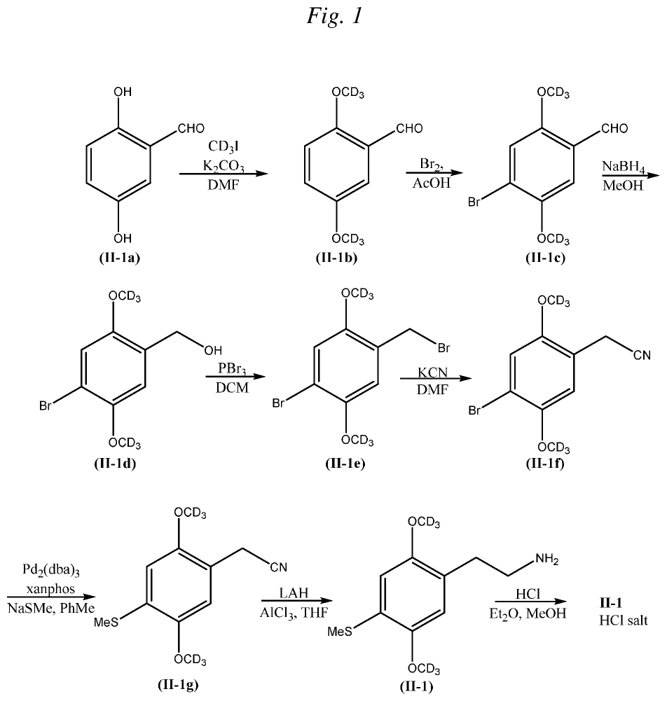

Compounds of the present disclosure and Reference Compounds may generally be prepared according to, or analogous to, the following synthetic procedures, which are depicted in FIGS. 1-30.

### Example 1

### 2-(2,5-bis(methoxy-d3)-4-(methylthio)phenyl)ethan-1-amine (II-1)

Synthesis of 2-(2,5-bis(methoxy-d3)-4-(methylthio)phenyl)ethan-1-amine (II-1) was carried out according to FIG. 1.

To a solution of commercially available 2,5-dihydroxybenzaldehyde II-la (2.00 g, 14.5 mmol, 1 eq) in anhydrous DMF (15 mL) was added potassium carbonate (8.01 g, 57.9 mmol, 4 eq) and iodomethane-d3 (2.7 mL, 43.4 mmol, 3 eq) under N₂ at room temperature. After stirring overnight, the reaction mixture was diluted with DCM (200 mL) and washed with water (3×100 mL) then brine (50 mL). The combined organic phase was dried over Na₂SO₄ and concentrated. The crude material was purified on silica (Hex to 20% EtOAc/Hex) to afford II-1b (2.13 g, 85% yield) as a white solid; the structure was confirmed by ¹H NMR in CDCl₃.

To a solution of II-1b (2.13 g, 12.4 mmol, 1 eq) in glacial AcOH (25 mL) was slowly added bromine (0.70 mL, 13.6 mmol, 1.1 eq) at room temperature. After 3 days, the reaction mixture was cooled to 0° C. and ice water (100 mL) slowly added. The precipitate was collected by filtration and washed with water. The crude material was purified on silica (Hex to 20% EtOAc/Hex) to afford II-1c (1.15 g, 37% yield) as a beige solid; the structure was confirmed by ¹H NMR in CDCl₃.

To a solution of II-1c (1.15 g, 4.58 mmol, 1 eq) in anhydrous MeOH (15 mL) was added sodium borohydride (173 mg, 4.58 mol, 1 eq) portion wise over 5 min at 0° C. under N₂ before allowing to warm to room temperature. After 1 h, the reaction mixture was slowly poured in to 1M HCl (25 mL) and extracted with DCM (3×25 mL). The combined organic phase was washed with brine (10 mL), dried over Na₂SO₄, and concentrated to afford II-1d (1.06 g, 92% yield) as a white solid. The crude material was used in the next step without further purification; the structure was confirmed by ¹H NMR in CDCl₃.

To a solution of PBr₃ (0.40 mL, 4.15 mmol, 1 eq) in anhydrous DCM (28 mL) at 0° C. under N₂ was slowly added II-1d (1.05 g, 4.15 mmol, 1 eq) in anhydrous DCM (17 mL). The reaction was allowed to warm to room temperature and stir for 1 h before cooling to 0° C. and slowly adding water (25 mL). The mixture was extracted with DCM (3×25 mL) and the combined organic phase was washed with brine (25 mL), dried over Na₂SO₄, and concentrated to afford II-1e (1.24 g, 94% yield) as a white solid. The crude material was used in the next step without further purification; the structure was confirmed by ¹H NMR in CDCl₃.

To a solution of II-1e (1.24 g, 3.92 mmol, 1 eq) in anhydrous DMF (8 mL) was added potassium cyanide (0.51 g, 7.83 mmol, 2 eq) at room temperature under N₂. After heating the reaction at 80° C. for 3 days, the reaction mixture was diluted with DCM (25 mL) and washed with water (4×25 mL) then brine (25 mL). The organic phase was dried over Na₂SO₄ and concentrated. The crude material was purified on silica (Hex to 50% EtOAc/Hex) to afford II-1f (0.33 g, 32% yield) as a white solid; the structure was confirmed by ¹H NMR in CDCl₃.

To a dried pressure vessel was added II-1f (330 mg, 1.26 mmol, 1 eq), Xantphos (80 mg, 0.14 mmol, 0.11 eq) and Pd₂(dba)₃ (115 mg, 0.13 mmol, 0.10 eq) and vessel purged with N₂ before adding anhydrous PhMe (sparged with N₂, 25 mL). After stirring 20 min at room temperature, NaSMe (177 mg, 2.52 mmol, 2 eq) was added, vessel purged with N₂ and sealed. After heating at 120° C. overnight, the reaction mixture was filtered through Celite with DCM and concentrated. The crude material was purified on silica (Hex to 10% EtOAc/Hex) to afford II-1g (0.13 g, 44% yield) as a beige solid; the structure was confirmed by ¹H NMR in CDCl₃.

To a solution of AlCl₃ (145 mg, 1.09 mmol, 2 eq) in anhydrous Et₂O(5.5 mL) at 0° C. under N₂ was added LiAlH₄ (62 mg, 1.64 mmol, 3 eq). After 10 min, II-1g (125 mg, 0.55 mmol, 1 eq) in anhydrous THF (5.5 mL) was slowly added and reaction allowed to warm to room temperature. After 3 h, the reaction was carefully quenched with water (0.6 mL), 2M NaOH (1.2 mL), and water (1.8 mL). The mixture was poured into water (25 mL), extracted with DCM (3×25 mL), and the combined organic phase was washed with brine (25 mL), dried over Na₂SO₄, and concentrated. The crude material was purified on silica (DCM to 90:10:1 DCM/MeOH/NH₄OH) to afford II-1 (96 mg, 75% yield) as a white solid; the structure was confirmed by ¹H NMR in CDCl₃.

To a solution of II-1 (96 mg, 0.41 mmol, 1 eq) in a mixture of anhydrous MeOH (4.1 mL) and anhydrous Et₂O (4.1 mL) was added anhydrous HCl (2M in Et₂O, 0.23 mL, 0.45 mmol, 1.1 eq) dropwise at room temperature under N₂. After 10 min, the reaction mixture was concentrated, solid washed with Et₂O, and dried to afford II-1 as an HCl salt (105 mg, 95% yield) as a white solid; the structure was confirmed by ¹H NMR in DMSO-d6.

### Example 2

### 2-(2,5-bis(methoxy-d3)-4-methylphenyl)ethan-1-amine (II-2)

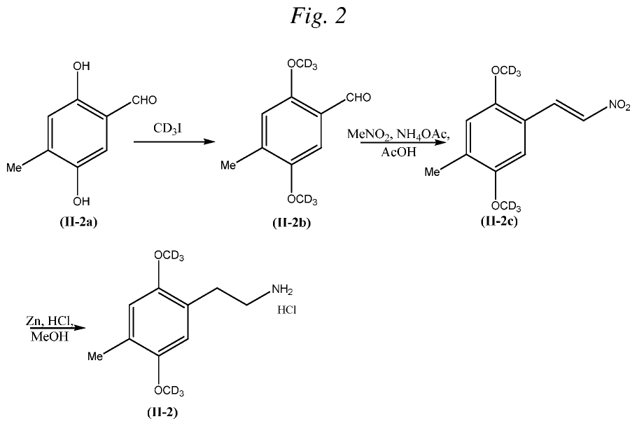

Synthesis of 2-(2,5-bis(methoxy-d3)-4-methylphenyl)ethan-1-amine (II-2) is carried out according to FIG. 2, using a modified general procedure as reported by Shulgin (Shulgin, A., and Shulgin, Ann. (1991) Pihkal: a chemical love story, Transform Press, Berkeley, CA) and later modified by Maresh (Maresh, J. J., Ralko, A. A., Speltz, T. E., Burke, J. L., Murphy, C. M., Gaskell, Z., Girel, J. K., Terranova, E., Richtscheidt, C., and Krzeszowiec, M. (2014) Chemoselective Zinc/HCl Reduction of Halogenated beta-Nitrostyrenes: Synthesis of Halogenated Dopamine Analogues, Synlett 25, 2891-2894). Bis-alkylation of 2,5-dihydroxy-4-methylbenzaldehyde II-2a with CD₃I provides intermediate II-2b, which is then subjected to a nitroaldol condensation with nitromethane in buffered acidic conditions to form the β-nitrostyrene II-2c. Subsequent bis-reduction of the nitro group and alkene with zinc dust in methanol containing hydrochloric acid yields the final product (II-2), as an HCl salt. The structure of the product will be confirmed by ¹H NMR.

### Example 3

### 2-(4-(tert-butyl)-2,5-bis(methoxy-d3)phenyl)ethan-1-amine (II-3)

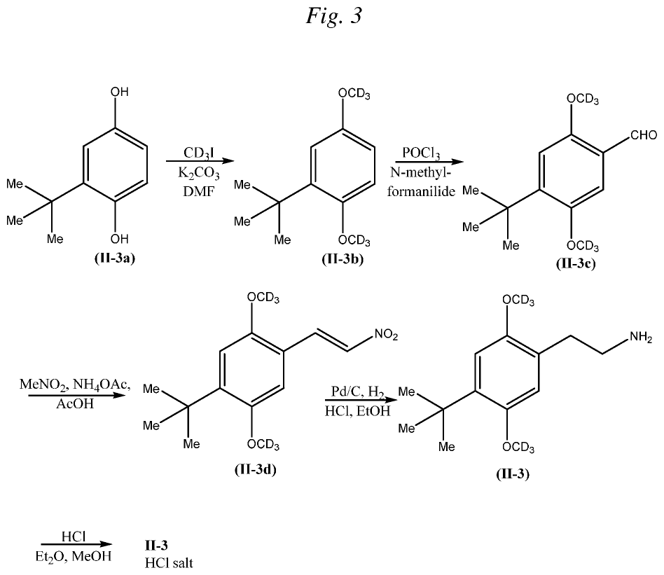

Synthesis of 2-(4-(tert-butyl)-2,5-bis(methoxy-d3)phenyl)ethan-1-amine (II-3) was carried out according to FIG. 3.

To a solution of commercially available tert-butylhydroquinone II-3a (3.0 g, 18.0 mmol, 1 eq) in anhydrous DMF (18 mL) was added potassium carbonate (10.0 g, 72.2 mmol, 4 eq) and iodomethane-d₃ (2.8 mL, 45.1 mmol, 2.5 eq) under N₂ at room temperature. After stirring 3 days, the reaction mixture was diluted with DCM (200 mL) and washed with water (3×100 mL) then brine (50 mL). The combined organic phase was dried over Na₂SO₄ and concentrated. The crude material was purified on silica (Hex to 10% EtOAc/Hex) to afford II-3b (2.8 g, 78% yield) as a tan oil; the structure was confirmed by ¹H NMR in CDCl₃.

To POCl₃ (5.6 mL, 60.2 mmol, 4.3 eq) was slowly added N-methylformanilide (6.4 mL, 51.8 mmol, 3.7 eq) at room temperature under N₂. After 1 h, II-3b (2.80 g, 14.0 mmol, 1 eq) was slowly added and reaction heated to 70° C. After 2 h, the reaction mixture was carefully poured into ice water (100 mL) and extracted with EtOAc (3×50 mL). The combined organic phase was washed with brine (50 mL), dried over Na₂SO₄, and concentrated. The crude material was purified on silica (Hex to 10% EtOAc/Hex) to afford II-3c (2.02 g, 63% yield) as a white solid; the structure was confirmed by ¹H NMR in CDCl₃.

To II-3c (2.02 g, 8.85 mmol, 1 eq) was added anhydrous ammonium acetate (680 mg, 8.85 mmol, 1 eq), nitromethane (2.4 mL, 44.24 mmol, 5 eq), acetic acid (glacial, 7.1 mL, 123.9 mmol, 14 eq) and activated 3 Å molecular sieves (4.3 g wet weight) under N₂ at room temperature. The reaction mixture was heated at 95° C. for 1 h. The molecular sieves were removed, reaction mixture poured into brine (50 mL) and extracted with EtOAc (3×50 mL). The combined organic phase was washed with water (50 mL), then brine (50 mL), dried over Na₂SO₄, and concentrated. The crude material was purified on silica (Hex to 10% EtOAc/Hex) to afford II-3d (1.73 g, 72% yield) as a yellow solid; the structure was confirmed by ¹H NMR in CDCl₃.

To a mixture of II-3d (1.73 g, 6.38 mmol, 1 eq) and palladium (5% by wt.) on carbon (1.36 g, 0.64 mmol, 0.10 eq) in anhydrous EtOH (90 mL) was added concentrated HCl (12.1 M, 1.05 mL, 12.76 mmol, 2 eq) and system degassed with N₂ before adding a hydrogen balloon. After 4 h at room temperature, hydrogen replaced with nitrogen atmosphere and reaction mixture filtered through Celite with MeOH. The reaction mixture was basified with MeOH/NH₄OH solution and concentrated. The crude material was purified on silica (DCM to 90:10:1 DCM/MeOH/NH₄OH) to afford II-3 (498 mg, 32% yield) as a colorless wax; the structure was confirmed by ¹H NMR in CDCl₃.

To a solution of II-3 (498 mg, 2.05 mmol, 1 eq) in a mixture of anhydrous MeOH (20 mL) and anhydrous Et₂O (20 mL) was added anhydrous HCl (2M in Et₂O, 1.13 mL, 2.25 mmol, 1.1 eq) dropwise at room temperature under N₂. After 10 min, the reaction mixture was concentrated, solid washed with Et₂O, and dried to afford II-3 as an HCl salt (512 mg, 89% yield) as a white solid; the structure was confirmed by ¹H NMR in DMSO-d₆.

### Example 4

### 2-(4-cyclopentyl-2,5-bis(methoxy-d3)phenyl)ethan-1-amine (II-4)

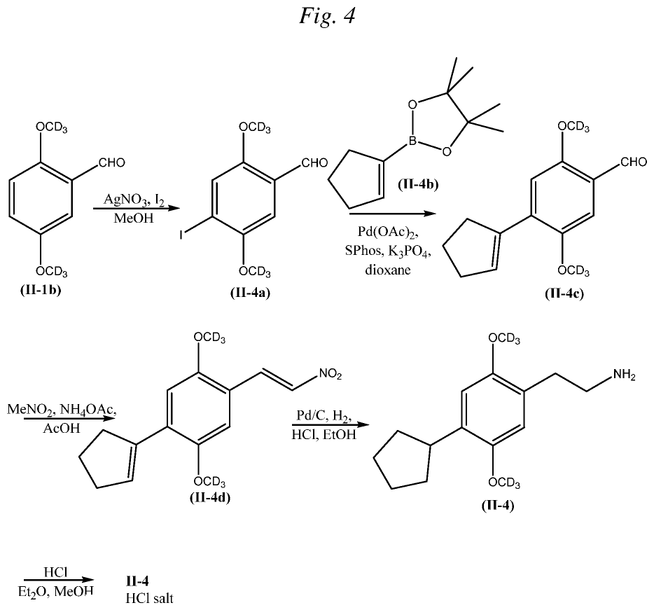

Synthesis of 2-(4-cyclopentyl-2,5-bis(methoxy-d3)phenyl)ethan-1-amine (II-4) was carried out according to FIG. 4.

To a solution of II-1b (5.24 g, 30.4 mmol, 1 eq) in anhydrous MeOH (203 mL) was added AgNO₃ (5.17 g, 30.4 mmol, 1 eq) and iodine (8.50 g, 33.5 mmol, 1.1 eq) at room temperature under N₂. After 1 day, precipitate removed by filtration, filtrate poured into aqueous Na₂S₂O₅ (1M, 250 mL) and extracted with DCM (3×100 mL). The combined organic phase was washed with brine (50 mL), dried over Na₂SO₄, and concentrated to afford II-4a (7.27 g, 80% yield) as a beige solid. The crude material was used in the next step without further purification; the structure was confirmed by ¹H NMR in CDCl₃.

To a pressure vessel was added II-4a (2.00 g, 6.71 mmol, 1 eq), SPhos (303 mg, 0.74 mmol, 0.11 eq), and Pd(OAc)₂ (151 mg, 0.67 mmol, 0.10 eq). The vessel was purged with N₂ before adding anhydrous 1,4-dioxane (67 mL), 2M aqueous K₃PO₄ (13.4 mL, 26.8 mmol, 4 eq), and cyclopentene-1-boronic acid, pinacol ester (II-4b, 2.60 g, 13.42 mmol, 2 eq) and mixture sparged with N₂ at room temperature. After sealing vessel, the reaction was heated at 100° C. overnight. The reaction mixture was filtered through Celite with DCM and concentrated. The crude material was purified on silica (Hex to 5% EtOAc/Hex) to afford II-4c (1.24 g, 78% yield) as a yellow solid; the structure was confirmed by ¹H NMR in CDCl₃.

To II-4c (1.24 g, 5.20 mmol, 1 eq) was added anhydrous ammonium acetate (401 mg, 5.20 mmol, 1 eq), nitromethane (1.41 mL, 26.02 mmol, 5 eq), acetic acid (glacial, 4.2 mL, 72.8 mmol, 14 eq) and activated 3 Å molecular sieves (2.5 g wet weight) under N₂ at room temperature. The reaction mixture was heated at 95° C. for 1 h. The molecular sieves were removed, reaction mixture poured into brine (30 mL) and extracted with EtOAc (3×30 mL). The combined organic phase was washed with water (30 mL), then brine (30 mL), dried over Na₂SO₄, and concentrated. The crude material was purified on silica (Hex to 5% EtOAc/Hex) to afford II-4d (231 mg, 16% yield) as an orange solid; the structure was confirmed by ¹H NMR in CDCl₃.

To a mixture of II-4d (231 mg, 0.82 mmol, 1 eq) and palladium (5% by wt.) on carbon (262 mg, 0.12 mmol, 0.15 eq) in anhydrous EtOH (12 mL) was added concentrated HCl (12.1 M, 0.14 mL, 1.64 mmol, 2 eq) and system degassed with N₂ before adding a hydrogen balloon. After 4 h at room temperature, hydrogen replaced with nitrogen atmosphere and reaction mixture filtered through Celite with MeOH. The reaction mixture was basified with MeOH/NH₄OH solution and concentrated. The crude material was purified on silica (DCM to 90:10:1 DCM/MeOH/NH₄OH) to afford II-4 (109 mg, 52% yield) as a white solid; the structure was confirmed by ¹H NMR in CDCl₃.

To a solution of II-4 (109 mg, 0.43 mmol, 1 eq) in a mixture of anhydrous MeOH (4 mL) and anhydrous Et₂O (4 mL) was added anhydrous HCl (2M in Et₂O, 0.23 mL, 0.47 mmol, 1.1 eq) dropwise at room temperature under N₂. After 10 min, the reaction mixture was concentrated, solid washed with Et₂O, and dried to afford II-4 as an HCl salt (123 mg, 99% yield) as a white solid; the structure was confirmed by ¹H NMR in DMSO-d₆.

### Example 5

### 2-(4-bromo-2,5-bis(methoxy-d3)phenyl)ethan-1,1,2,2-d4-1-amine (II-14)

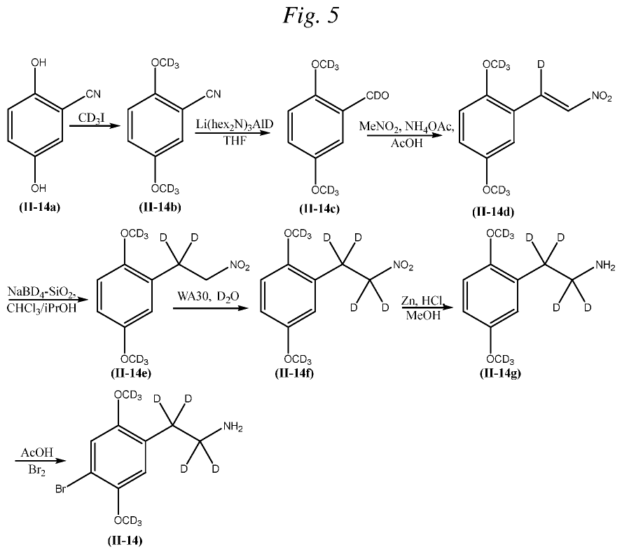

Synthesis of 2-(4-bromo-2,5-bis(methoxy-d3)phenyl)ethan-1,1,2,2-d4-1-amine (II-14) is carried out according to FIG. 5, using a modified general procedure as reported by Shulgin (Shulgin, A., and Shulgin, Ann. (1991) Pihkal: a chemical love story, Transform Press, Berkeley, CA) and later modified by Maresh (Maresh, J. J., Ralko, A. A., Speltz, T. E., Burke, J. L., Murphy, C. M., Gaskell, Z., Girel, J. K., Terranova, E., Richtscheidt, C., and Krzeszowiec, M. (2014) Chemoselective Zinc/HCl Reduction of Halogenated beta-Nitrostyrenes: Synthesis of Halogenated Dopamine Analogues, Synlett 25, 2891-2894). Bis-alkylation of 2,5-dihydroxybenzonitrile (II-14a) with CD₃I forms intermediate II-14b, which is then deuterated by reduction using lithium tris(dihexylamino)aluminum deuteride (Li(hex₂N)₃AlD) to the deuterated benzaldehyde II-14c using a method developed by Cha (Cha, J. S., Lee, S. E., and Lee, H. S., 1992, Selective Conversion of Aromatic Nitriles to Aldehydes by Lithium Tris(Dihexylamino)Aluminum Hydride, Org Prep Proced Int 24, 331-334). β-nitrostyrene II-14d is formed using a nitroaldol condensation with nitromethane in buffered acidic conditions. Subsequent treatment with sodium borodeuteride and silicone dioxide selectively reduces the alkene to intermediate II-14e (Sinhababu, A. K., and Borchardt, R. T. (1983) Silica Gel-Assisted Reduction of Nitrostyrenes to 2-Aryl-1-Nitroalkanes with Sodium-Borohydride, Tetrahedron Letters 24, 227-230), followed by deuterium exchange at the α-position using the basic resin WA30 and deuterated water developed by Yamada (Yamada, T., Kuwata, M., Takakura, R., Monguchi, Y., Sajiki, H., and Sawama, Y. (2018) Organocatalytic Nitroaldol Reaction Associated with Deuterium-Labeling, Adv Synth Catal 360, 637-641), which forms intermediate II-14f. Reduction of the nitro group with zinc dust in methanol containing hydrochloric acid yields intermediate II-14g. Selective bromination then affords final product (II-14). The structure of the product will be confirmed by ¹H NMR.

### Example 6

### 2-(2,5-dimethoxy-4-(methyl-d3)phenyl)ethan-1-amine (III-1)

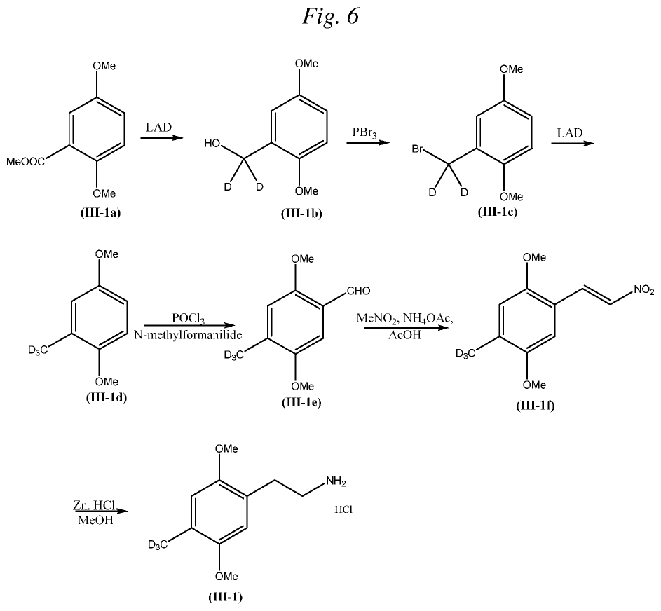

Synthesis of 2-(2,5-dimethoxy-4-(methyl-d3)phenyl)ethan-1-amine (III-1) is carried out according to FIG. 6, using a modified general procedure as reported by Shulgin (Shulgin, A., and Shulgin, Ann. (1991) Pihkal: a chemical love story, Transform Press, Berkeley, CA) and later modified by Maresh (Maresh, J. J., Ralko, A. A., Speltz, T. E., Burke, J. L., Murphy, C. M., Gaskell, Z., Girel, J. K., Terranova, E., Richtscheidt, C., and Krzeszowiec, M. (2014) Chemoselective Zinc/HCl Reduction of Halogenated beta-Nitrostyrenes: Synthesis of Halogenated Dopamine Analogues, Synlett 25, 2891-2894). Reduction of methyl 2,5-dimethoxybenzoate (III-la) with lithium aluminum deuteride (LAD) provides benzyl alcohol intermediate III-1b, which is subsequently converted to benzyl bromide intermediate III-1c upon reaction with PBr₃. Further reaction with LAD provides intermediate III-id, which is selectively formylated with N-methylformanilide and POCl₃. The resulting benzaldehyde III-1e undergoes a nitroaldol condensation with nitromethane in buffered acidic conditions to form the β-nitrostyrene III-1f. Subsequent bis-reduction of the nitro group and alkene with zinc dust in methanol containing hydrochloric acid yields the final product (III-1), as an HCl salt. The structure of the product will be confirmed by ¹H NMR.

### Example 7

### 2-(2,5-dimethoxy-4-(2-(methyl-d3)propan-2-yl-1,1,1,3,3,3-d6)phenyl)ethan-1-amine (III-2)

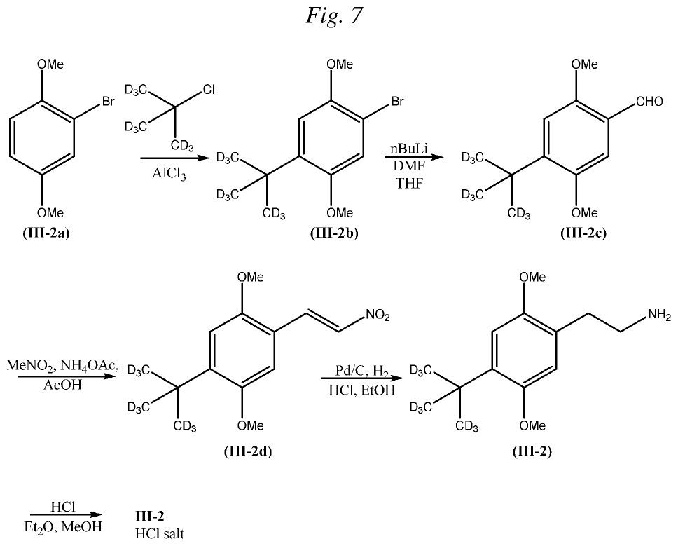

Synthesis of 2-(2,5-dimethoxy-4-(2-(methyl-d3)propan-2-yl-1,1,1,3,3,3-d6)phenyl)ethan-1-amine (III-2) was carried out according to FIG. 7.

To a solution of commercially available 1-bromo-2,5-dimethoxybenzene III-2a (1.50 mL, 9.84 mmol, 1 eq) and tert-butylchloride-d₉ (5.0 g, 49.2 mmol, 5 eq) at 0° C. under N₂ was added AlCl₃ (0.53 g, 3.94 mmol, 0.4 eq) portion wise over 5 min. After 4 h, the reaction mixture was carefully poured into ice water (100 mL) and extracted with EtOAc (3×50 mL). The combined organic phase was washed with brine (25 mL), dried over Na₂SO₄, and concentrated. The crude material was purified on silica (Hex to 5% EtOAc/Hex) to afford III-2b (1.06 g, 38% yield) as a beige solid; the structure was confirmed by ¹H NMR in CDCl₃.

To a solution of III-2b (1.05 g, 3.72 mmol, 1 eq) in anhydrous THF (12 mL) at −78° C. under N₂ was added n-BuLi (1.6 M in hexane, 3.5 mL, 5.58 mmol, 1.5 eq) dropwise. After 30 min, anhydrous DMF (2.9 mL, 37.2 mmol, 10 eq) was added dropwise and reaction allowed to warm to room temperature. After 3 h, the reaction was quenched with saturated aqueous NH₄Cl (20 mL) and diluted with DCM (150 mL). The organic phase was washed with water (25 mL) then brine (25 mL), dried over Na₂SO₄, and concentrated. The crude material was purified on silica (Hex to 10% EtOAc/Hex) to afford III-2c (0.44 g, 51% yield) as a beige solid; the structure was confirmed by ¹H NMR in CDCl₃.

To III-2c (0.44 g, 1.90 mmol, 1 eq) was added anhydrous ammonium acetate (150 mg, 1.90 mmol, 1 eq), nitromethane (0.51 mL, 9.5 mmol, 5 eq), acetic acid (glacial, 1.53 mL, 26.6 mmol, 14 eq) and activated 3 Å molecular sieves (0.93 g wet weight) under N₂ at room temperature. The reaction mixture was heated at 95° C. for 1 h. The molecular sieves were removed, reaction mixture poured into brine (20 mL) and extracted with EtOAc (3×20 mL). The combined organic phase was washed with water (20 mL), then brine (20 mL), dried over Na₂SO₄, and concentrated. The crude material was purified on silica (Hex to 10% EtOAc/Hex) to afford III-2d (0.40 g, 76% yield) as a yellow solid; the structure was confirmed by ¹H NMR in CDCl₃.

To a mixture of III-2d (397 mg, 1.45 mmol, 1 eq) and palladium (5% by wt.) on carbon (468 mg, 0.22 mmol, 0.15 eq) in anhydrous EtOH (21 mL) was added concentrated HCl (12.1 M, 0.24 mL, 2.90 mmol, 2 eq) and system degassed with N₂ before adding a hydrogen balloon. After 4 h at room temperature, hydrogen replaced with nitrogen atmosphere and reaction mixture filtered through Celite with MeOH. The reaction mixture was basified with MeOH/NH₄₀H solution and concentrated. The crude material was purified on silica (DCM to 90:10:1 DCM/MeOH/NH₄OH) to afford III-2 (199 mg, 56% yield) as a white solid; the structure was confirmed by ¹H NMR in CDCl₃.

To a solution of III-2 (199 mg, 0.808 mmol, 1 eq) in a mixture of anhydrous MeOH (8 mL) and anhydrous Et₂O (8 mL) was added anhydrous HCl (2M in Et₂O, 0.44 mL, 0.889 mmol, 1.1 eq) dropwise at room temperature under N₂. After 10 min, the reaction mixture was concentrated, solid washed with Et₂O, and dried to afford III-2 as an HCl salt (212 mg, 93% yield) as a white solid; the structure was confirmed by ¹H NMR in DMSO-d₆.

### Example 8

### 2-(2,5-dimethoxy-4-(methylthio)phenyl)ethan-1,1-d2-1-amine (IV-1)

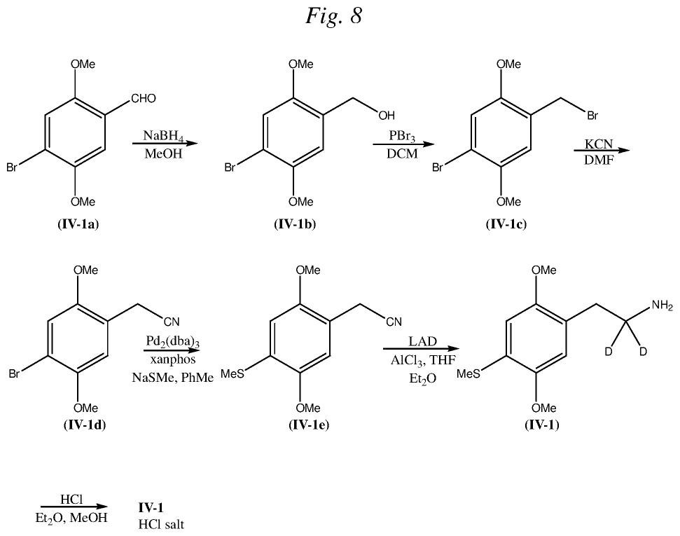

Synthesis of 2-(2,5-dimethoxy-4-(methylthio)phenyl)ethan-1,1-d2-1-amine (IV-1) was carried out according to FIG. 8.

To a solution of commercially available 4-bromo-2,5-dihydroxybenzaldehyde IV-la (5.00 g, 20.4 mmol, 1 eq) in anhydrous MeOH (68 mL) was added sodium borohydride (0.77 g, 20.4 mol, 1 eq) portionwise over 5 min at 0° C. under N₂ before allowing to warm to room temperature. After 1 h, the reaction mixture was slowly poured in to 1M HCl (100 mL) and extracted with DCM (3×100 mL). The combined organic phase was washed with brine (50 mL), dried over Na₂SO₄, and concentrated to afford IV-1b (4.78 g, 95% yield) as a white solid. The crude material was used in the next step without further purification; the structure was confirmed by ¹H NMR in CDCl₃.

To a solution of PBr₃ (1.84 mL, 19.4 mmol, 1 eq) in anhydrous DCM (130 mL) at 0° C. under N₂ was slowly added IV-1b (4.78 g, 19.4 mmol, 1 eq) in anhydrous DCM (75 mL). The reaction was allowed to warm to room temperature and stir for 1 h before cooling to 0° C. and slowly adding water (100 mL). The mixture was extracted with DCM (3×100 mL) and the combined organic phase was washed with brine (100 mL), dried over Na₂SO₄, and concentrated to afford IV-1c (5.76 g, 96% yield) as a white solid. The crude material was used in the next step without further purification; the structure was confirmed by ¹H NMR in CDCl₃.

To a solution of IV-1c (5.76 g, 18.6 mmol, 1 eq) in anhydrous DMF (19 mL) was added potassium cyanide (2.42 g, 37.2 mmol, 2 eq) at room temperature under N₂. After heating the reaction at 70° C. for 2 days, the reaction mixture was diluted with DCM (200 mL) and washed with water (4×100 mL) then brine (100 mL). The organic phase was dried over Na₂SO₄ and concentrated. The crude material was purified on silica (Hex to 50% EtOAc/Hex) to afford IV-1d (0.67 g, 14% yield) as a white solid; the structure was confirmed by ¹H NMR in CDCl₃.

To a dried pressure vessel was added IV-1d (460 mg, 1.80 mmol, 1 eq), Xantphos (115 mg, 0.20 mmol, 0.11 eq) and Pd₂(dba)₃ (165 mg, 0.18 mmol, 0.10 eq) and vessel purged with N₂ before adding anhydrous PhMe (sparged with N₂, 18 mL). After stirring 20 min at room temperature, NaSMe (189 mg, 2.70 mmol, 1.5 eq) was added, vessel purged with N₂ and sealed. After heating at 130° C. overnight, the reaction mixture was filtered through Celite with DCM and concentrated. The crude material was purified on silica (Hex to 20% EtOAc/Hex) to afford IV-1e (0.18 g, 44% yield) as a beige solid; the structure was confirmed by ¹H NMR in CDCl₃.

To a solution of AlCl₃ (211 mg, 1.59 mmol, 2 eq) in anhydrous Et₂O (7.9 mL) at 0° C. under N₂ was added LiAlD₄ (100 mg, 2.38 mmol, 3 eq). After 10 min, IV-1e (177 mg, 0.79 mmol, 1 eq) in anhydrous THF (7.9 mL) was slowly added and reaction allowed to warm to room temperature. After 3 h, the reaction was carefully quenched with water (1 mL), 2M NaOH (2 mL), and water (3 mL). The mixture was poured into water (25 mL), extracted with DCM (3×25 mL), and the combined organic phase was washed with brine (25 mL), dried over Na₂SO₄, and concentrated. The crude material was purified on silica (DCM to 90:10:1 DCM/MeOH/NH₄OH) to afford IV-1 (49 mg, 27% yield) as a white solid; the structure was confirmed by ¹H NMR in CDCl₃.

To a solution of IV-1 (68 mg, 0.30 mmol, 1 eq) in a mixture of anhydrous MeOH (3 mL) and anhydrous Et₂O (3 mL) was added anhydrous HCl (2M in Et₂O, 0.16 mL, 0.33 mmol, 1.1 eq) dropwise at room temperature under N₂. After 10 min, the reaction mixture was concentrated, solid washed with Et₂O, and dried to afford IV-1 as an HCl salt (75 mg, 95% yield) as a white solid; the structure was confirmed by ¹H NMR in DMSO-d6.

### Example 9

### 2-(2,5-dimethoxy-4-(propylthio)phenyl)ethan-1,1-d2-1-amine (IV-2)

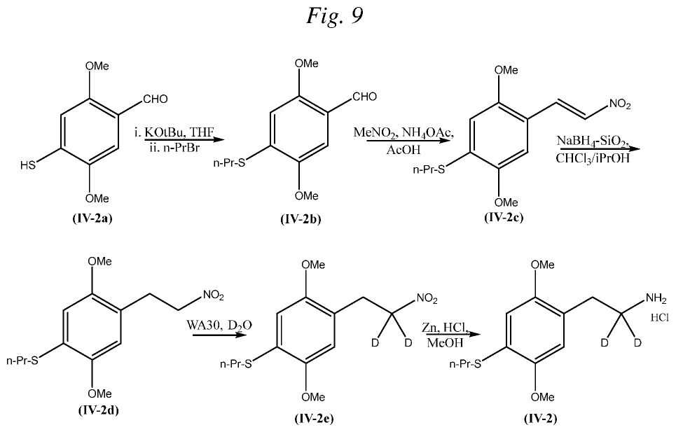

Synthesis of 2-(2,5-dimethoxy-4-(propylthio)phenyl)ethan-1,1-d2-1-amine (IV-2) is carried out according to FIG. 9, using a modified general procedure as reported by Shulgin (Shulgin, A., and Shulgin, Ann. (1991) Pihkal: a chemical love story, Transform Press, Berkeley, CA) and later modified by Maresh (Maresh, J. J., Ralko, A. A., Speltz, T. E., Burke, J. L., Murphy, C. M., Gaskell, Z., Girel, J. K., Terranova, E., Richtscheidt, C., and Krzeszowiec, M. (2014) Chemoselective Zinc/HCl Reduction of Halogenated beta-Nitrostyrenes: Synthesis of Halogenated Dopamine Analogues, Synlett 25, 2891-2894). 4-mercapto-2,5-dimethoxybenzaldehyde (IV-2a) is alkylated by first deprotonating the thiol using potassium tert-butoxide in THF, followed by the addition of n-propyl bromide. The resulting benzaldehyde IV-2b undergoes a nitroaldol condensation with nitromethane in buffered acidic conditions to form the β-nitrostyrene IV-2c. Subsequent treatment with sodium borohydride and silicone dioxide selectively reduces the alkene, forming intermediate IV-2d, followed by deuterium exchange at the α-position using the basic resin WA30 and deuterated water developed by Yamada (Yamada, T., Kuwata, M., Takakura, R., Monguchi, Y., Sajiki, H., and Sawama, Y. (2018) Organocatalytic Nitroaldol Reaction Associated with Deuterium-Labeling, Adv Synth Catal 360, 637-641) to form intermediate IV-2e. Reduction of the nitro group with zinc dust in methanol containing hydrochloric acid yields the final product (IV-2), as an HCl salt. The structure of the product will be confirmed by ¹H NMR.

### Example 10

### 2-(2,5-dimethoxy-4-(trifluoromethyl)phenyl)ethan-1,1-d2-1-amine (IV-3)

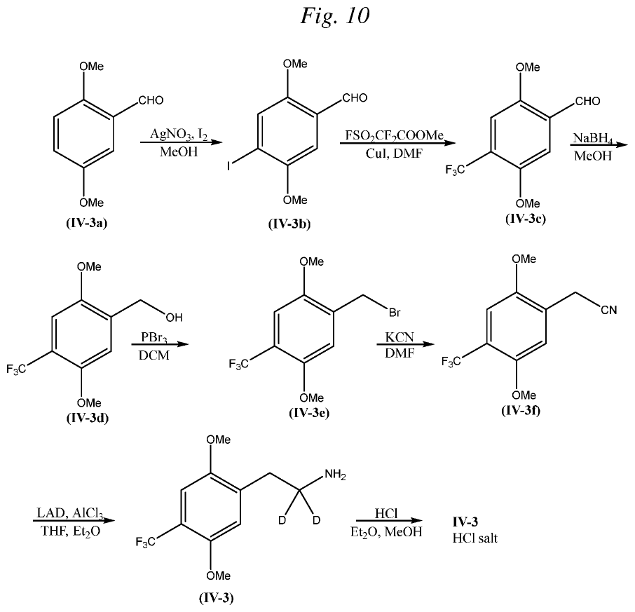

Synthesis of 2-(2,5-dimethoxy-4-(trifluoromethyl)phenyl)ethan-1,1-d2-1-amine (IV-3) was carried out according to FIG. 10.

To a solution of commercially available 2,5-dimethoxybenzaldehyde IV-3a (5.00 g, 30.1 mmol, 1 eq) in anhydrous MeOH (200 mL) was added AgNO₃ (5.11 g, 30.1 mmol, 1 eq) and iodine (8.40 g, 33.1 mmol, 1.1 eq) at room temperature under N₂. After 2 days, precipitate removed by filtration and filtrate poured into aqueous Na₂S₂O₅ (1M, 250 mL). The resulting precipitate was collected by filtration, washed with water then hexane, and dried to afford IV-3b (7.80 g, 89% yield) as a beige solid. The crude material was used in the next step without further purification; the structure was confirmed by ¹H NMR in CDCl₃.

To a mixture of IV-3b (2.00 g, 6.85 mmol, 1 eq) and CuI (196 mg, 1.03 mmol, 0.15 eq) in anhydrous DMF (14 mL) was added methyl 2,2-difluoro-2-(fluorosulfonyl)acetate (1.75 mL, 13.70 mmol, 2 eq) at room temperature under N₂. After heating at 70° C. overnight, the reaction mixture was filtered through Celite with DCM, organic phase washed with water (3×50 mL) then brine (50 mL), dried over Na₂SO₄, and concentrated. The crude material was purified on silica (Hex to 20% EtOAc/Hex) to afford IV-3c (1.41 g, 88% yield) as a white solid; the structure was confirmed by ¹H NMR in CDCl₃.

To a solution of IV-3c (1.41 g, 6.02 mmol, 1 eq) in anhydrous MeOH (20 mL) was added sodium borohydride (0.23 g, 6.02 mol, 1 eq) portion wise over 5 min at 0° C. under N₂ before allowing to warm to room temperature. After 1 h, the reaction mixture was slowly poured in to 1M HCl (50 mL) and extracted with DCM (3×50 mL). The combined organic phase was washed with brine (20 mL), dried over Na₂SO₄, and concentrated to afford IV-3d (1.38 g, 97% yield) as a white solid. The crude material was used in the next step without further purification; the structure was confirmed by ¹H NMR in CDCl₃.

To a solution of PBr₃ (0.55 mL, 5.84 mmol, 1 eq) in anhydrous DCM (39 mL) at 0° C. under N₂ was slowly added IV-3d (1.38 g, 5.84 mmol, 1 eq) in anhydrous DCM (24 mL). The reaction was allowed to warm to room temperature and stir for 1 h before cooling to 0° C. and slowly adding water (25 mL). The mixture was extracted with DCM (3×25 mL) and the combined organic phase was washed with brine (25 mL), dried over Na₂SO₄, and concentrated to afford IV-3e (1.09 g, 62% yield) as a white solid. The crude material was used in the next step without further purification; the structure was confirmed by ¹H NMR in CDCl₃.

To a solution of IV-3e (1.09 g, 3.64 mmol, 1 eq) in anhydrous DMF (7.3 mL) was added potassium cyanide (475 mg, 7.29 mmol, 2 eq) at room temperature under N₂. After heating the reaction at 100° C. overnight, the reaction mixture was diluted with DCM (20 mL) and washed with water (4×20 mL) then brine (20 mL). The organic phase was dried over Na₂SO₄ and concentrated. The crude material was purified on silica (Hex to 20% EtOAc/Hex) to afford IV-3f (113 mg, 13% yield) as a white solid; the structure was confirmed by ¹H NMR in CDCl₃.

To a solution of AlCl₃ (123 mg, 0.992 mmol, 2 eq) in anhydrous Et₂O (4.6 mL) at 0° C. under N₂ was added LiAlD₄ (58 mg, 1.383 mmol, 3 eq). After 10 min, IV-3f (113 mg, 0.461 mmol, 1 eq) in anhydrous THF (4.6 mL) was slowly added and reaction allowed to warm to room temperature. After 3 h, the reaction was carefully quenched with water (1 mL), 2M NaOH (2 mL), and water (3 mL). The mixture was poured into water (25 mL), extracted with DCM (3×25 mL), and the combined organic phase was washed with brine (25 mL), dried over Na₂SO₄, and concentrated. The crude material was purified on silica (DCM to 90:10:1 DCM/MeOH/NH₄OH) to afford IV-3 (66 mg, 60% yield) as a beige solid; the structure was confirmed by ¹H NMR in CDCl₃.

To a solution of IV-3 (66 mg, 0.263 mmol, 1 eq) in a mixture of anhydrous MeOH (3 mL) and anhydrous Et₂O (3 mL) was added anhydrous HCl (2M in Et₂O, 0.15 mL, 0.289 mmol, 1.1 eq) dropwise at room temperature under N₂. After 10 min, the reaction mixture was concentrated, solid washed with Et₂O, and dried to afford IV-3 as an HCl salt (86 mg, 100% yield) as a white solid; the structure was confirmed by ¹H NMR in DMSO-d6.

### Example 11

### 2-(4-bromo-2,5-dimethoxyphenyl)ethan-1,1-d₂-1-amine (IV-5)

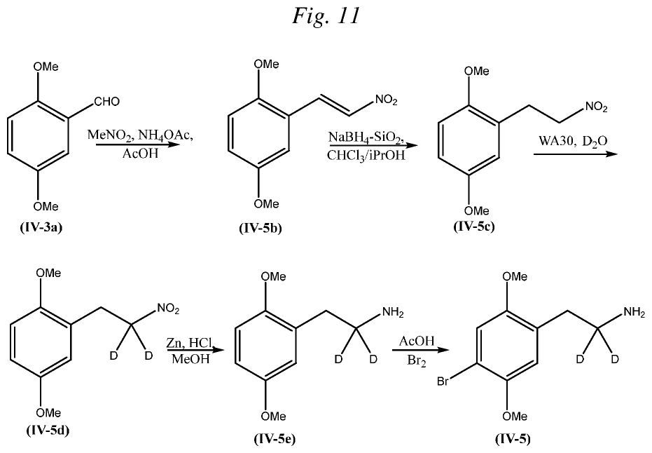

Synthesis of 2-(4-bromo-2,5-dimethoxyphenyl)ethan-1,1-d₂-1-amine (IV-5) is carried out according to FIG. 11, using a modified general procedure as reported by Shulgin (Shulgin, A., and Shulgin, Ann. (1991) Pihkal: a chemical love story, Transform Press, Berkeley, CA) and later modified by Maresh (Maresh, J. J., Ralko, A. A., Speltz, T. E., Burke, J. L., Murphy, C. M., Gaskell, Z., Girel, J. K., Terranova, E., Richtscheidt, C., and Krzeszowiec, M. (2014) Chemoselective Zinc/HCl Reduction of Halogenated beta-Nitrostyrenes: Synthesis of Halogenated Dopamine Analogues, Synlett 25, 2891-2894). 2,5-Dimethoxybenzaldehyde (IV-5a) undergoes a nitroaldol condensation using nitromethane in buffered acidic conditions to form the β-nitrostyrene IV-5b. Subsequent treatment with sodium borohydride and silicone dioxide selectively reduces the alkene to intermediate IV-5c followed by deuterium exchange at the α-position using the basic resin WA30 and deuterated water developed by Yamada (Yamada, T., Kuwata, M., Takakura, R., Monguchi, Y., Sajiki, H., and Sawama, Y. (2018) Organocatalytic Nitroaldol Reaction Associated with Deuterium-Labeling, Adv Synth Catal 360, 637-641) to form intermediate IV-5d. Reduction of the nitro group with zinc dust in methanol containing hydrochloric acid yields intermediate IV-5e. Selective bromination then affords final product (IV-5). The structure of the product will be confirmed by ¹H NMR.

### Example 12

### 2-(4-bromo-2,5-dimethoxyphenyl)ethan-1,1,2,2-d4-1-amine (IV-12)

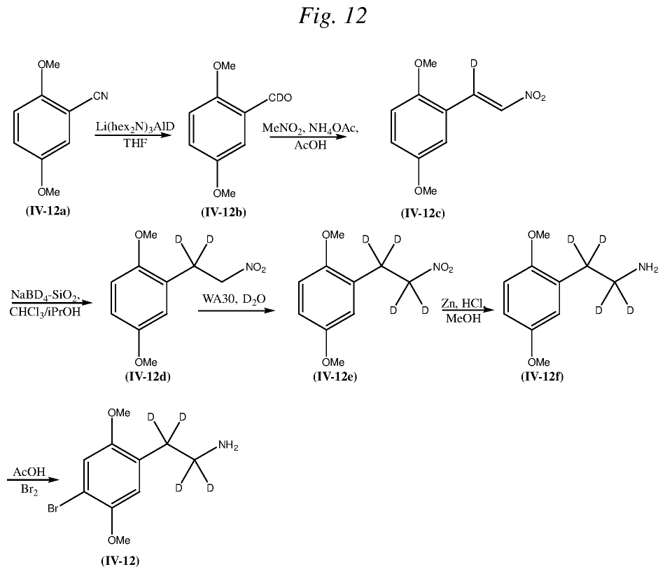

Synthesis of 2-(4-bromo-2,5-dimethoxyphenyl)ethan-1,1,2,2-d4-1-amine (IV-12) is carried out according to FIG. 12, using a modified general procedure as reported by Shulgin (Shulgin, A., and Shulgin, Ann. (1991) Pihkal: a chemical love story, Transform Press, Berkeley, CA) and later modified by Maresh (Maresh, J. J., Ralko, A. A., Speltz, T. E., Burke, J. L., Murphy, C. M., Gaskell, Z., Girel, J. K., Terranova, E., Richtscheidt, C., and Krzeszowiec, M. (2014) Chemoselective Zinc/HCl Reduction of Halogenated beta-Nitrostyrenes: Synthesis of Halogenated Dopamine Analogues, Synlett 25, 2891-2894). The starting material IV-12a is deuterated by reduction using lithium tris(dihexylamino)aluminum deuteride (Li(hex₂N)₃AlD) to the deuterated benzaldehyde IV-12b using a method developed by Cha (Cha, J. S., Lee, S. E., and Lee, H. S., 1992, Selective Conversion of Aromatic Nitriles to Aldehydes by Lithium Tris(Dihexylamino)Aluminum Hydride, Org Prep Proced Int 24, 331-334). β-nitrostyrene IV-12c is formed using a nitroaldol condensation with nitromethane in buffered acidic conditions. Subsequent treatment with sodium borodeuteride and silicone dioxide selectively reduces the alkene to intermediate IV-12d (Sinhababu, A. K., and Borchardt, R. T. (1983) Silica Gel-Assisted Reduction of Nitrostyrenes to 2-Aryl-1-Nitroalkanes with Sodium-Borohydride, Tetrahedron Letters 24, 227-230), followed by deuterium exchange at the α-position using the basic resin WA30 and deuterated water developed by Yamada (Yamada, T., Kuwata, M., Takakura, R., Monguchi, Y., Sajiki, H., and Sawama, Y. (2018) Organocatalytic Nitroaldol Reaction Associated with Deuterium-Labeling, Adv Synth Catal 360, 637-641) to form intermediate IV-12e. Reduction of the nitro group with zinc dust in methanol containing hydrochloric acid yields intermediate IV-12f. Selective bromination then affords final product (IV-12). The structure of the product will be confirmed by 1H NMR.

### Example 13

### 2-(2,5-dimethoxy-4-((trifluoromethyl)thio)phenyl)ethan-1-amine (I-1)

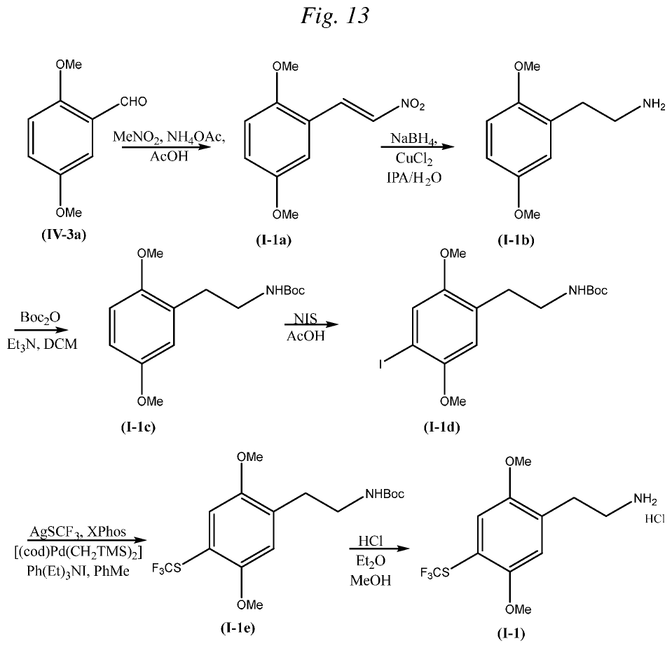

Synthesis of 2-(2,5-dimethoxy-4-((trifluoromethyl)thio)phenyl)ethan-1-amine (I-1) was carried out according to FIG. 13.

To commercially available 2,5-dimethoxybenzaldehyde IV-3a (10.0 g, 60.2 mmol, 1 eq) was added anhydrous ammonium acetate (4.64 g, 60.2 mmol, 1 eq), nitromethane (16.3 mL, 301 mmol, 5 eq), acetic acid (glacial, 48 mL, 843 mmol, 14 eq) and activated 3 Å molecular sieves (27 g wet weight) under N₂ at room temperature. The reaction mixture was heated at 95° C. for 1 h. The molecular sieves were removed, reaction mixture poured into brine (200 mL) and extracted with DCM (3×200 mL). The combined organic phase was washed with water (100 mL), then brine (100 mL), dried over Na₂SO₄, and concentrated to afford I-1a (12.0 g, 95% yield) as an orange solid. The crude material was used in the next step without further purification; the structure was confirmed by ¹H NMR in CDCl₃.

To a mixture of IPA (250 mL) and water (125 mL) at 0° C. was added I-la (12.0 g, 57.2 mmol, 1 eq), and the mixture was heated to 50° C. Then, NaBH₄ (16.2 g, 429 mmol, 7.5 eq) and an aqueous solution of CuCl₂ (2M, 2.86 mL, 5.72 mmol, 0.10 eq) were added portion wise, then gradually heated to 80° C. and held for 1 h. The reaction mixture was then poured into aqueous NaOH (2M, 300 mL) and extracted with DCM (3×500 mL). The combined organic phase was washed with brine (100 mL), dried over Na₂SO₄, and concentrated. The crude material was purified on silica (DCM to 90:10:1 DCM/MeOH/NH₄OH) to afford I-1b (4.65 g, 45% yield) as a brown oil; the structure was confirmed by ¹H NMR in CDCl₃.

To a solution of I-1b (4.65 g, 25.7 mmol, 1 eq) and Et₃N (10.7 mL, 77.0 mmol, 3 eq) in DCM (51 mL) at room temperature under N₂ was slowly added Boc₂O (11.2 g, 51.3 mmol, 2 eq) in DCM (26 mL). After stirring O/N, reaction mixture was poured into brine (100 mL), extracted with DCM (3×100 mL), dried over Na₂SO₄, and concentrated. The crude material was purified on silica (Hex to 30% EtOAc/Hex) to afford I-1c (5.46 g, 76% yield) as a white solid; the structure was confirmed by ¹H NMR in CDCl₃.

To a solution of I-1c (5.06 g, 18.0 mmol, 1 eq) in glacial AcOH (140 mL) at room temperature under N₂ was added N-iodosuccinimide (5.06 g, 22.5 mmol, 1.25 eq). After 5 h, reaction mixture poured into aqueous Na₂S₂O₅ (1M, 200 mL) and extracted with DCM (3×200 mL). The combined organic phase was washed with brine (100 mL), dried over Na₂SO₄ and concentrated. The crude material was purified on silica (Hex to 20% EtOAc/Hex) to afford I-1d (6.10 g, 88% yield) as a white solid; the structure was confirmed by ¹H NMR in CDCl₃.

To a dried pressure vessel was added I-1d (2.00 g, 4.91 mmol, 1 eq), XPhos (396 mg, 0.83 mmol, 0.17 eq) and [(cod)Pd(CH₂TMS)₂](288 mg, 0.74 mmol, 0.15 eq) and vessel purged with N₂ before adding anhydrous PhMe (sparged with N₂, 25 mL). The mixture was then heated at 110° C. for 1 min before adding AgSCF₃ (1.33 g, 6.38 mmol, 1.3 eq) and Ph(Et)₃NI (1.95 g, 6.38 mmol, 1.3 eq), purging with N₂, and sealing pressure vessel. After heating 110° C. for 2 days, reaction mixture filtered through silica with DCM/EtOAc and concentrated. The crude material was purified on silica (Hex to 20% EtOAc/Hex) to afford I-1e (0.84 g, 74% yield) as a beige solid; the structure was confirmed by ¹H NMR in CDCl₃.

To a solution of I-1e (500 mg, 1.31 mmol, 1 eq) in a mixture of anhydrous MeOH (6.6 mL) and anhydrous Et₂O (6.6 mL) was added anhydrous HCl (2M in Et₂O, 3.3 mL, 6.55 mmol, 5 eq) dropwise at room temperature under N₂. After stirring overnight, the reaction mixture was concentrated, solid washed with Et₂O, and dried to afford I-1 as an HCl salt (410 mg, 98% yield) as a white solid; the structure was confirmed by ¹H NMR in DMSO-d₆.

### Example 14

### 2-(2,5-dimethoxy-4-((3,3,3-trifluoropropyl)thio)phenyl)ethan-1-amine (I-2)

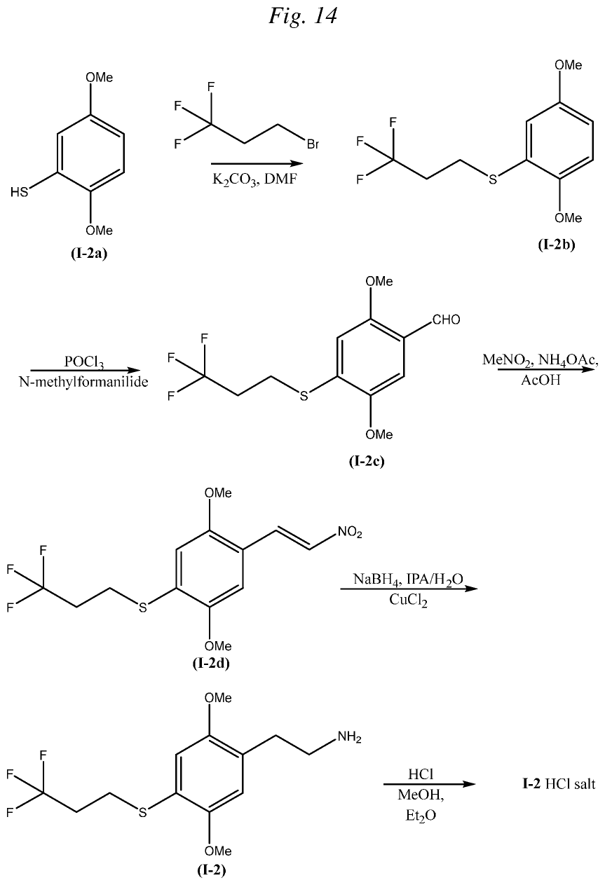

Synthesis of 2-(2,5-dimethoxy-4-((3,3,3-trifluoropropyl)thio)phenyl)ethan-1-amine (I-2) was carried out according to FIG. 14.

To a solution of commercially available 2,5-dimethoxythiophenol I-2a (0.38 mL, 2.51 mmol, 1 eq) in anhydrous DMF (2.5 mL) at room temperature under N₂ was added potassium carbonate (520 mg, 3.76 mmol, 1.5 eq) and 3-bromo-1,1,1-trifluoropropane (0.40 mL, 3.76 mmol, 1.5 eq). After stirring overnight, reaction mixture poured into water (30 mL) and extracted with DCM (3×30 mL). The combined organic phase was washed with water (2×30 mL) then brine (10 mL), dried over Na₂SO₄, and concentrated. The crude material was purified on silica (Hex to 10% EtOAc/Hex) to afford I-2b (0.658 g, 98% yield) as a colorless oil; the structure was confirmed by ¹H NMR in CDCl₃.

To POCl₃ (0.98 mL, 10.50 mmol, 4.3 eq) was slowly added N-methylformanilide (1.11 mL, 9.03 mmol, 3.7 eq) at room temperature under N₂. After 1 h, I-2b (0.65 g, 2.44 mmol, 1 eq) was slowly added and reaction heated to 70° C. After 2 h, the reaction mixture was carefully poured into ice water (20 mL) and extracted with DCM (3×20 mL). The combined organic phase was washed with brine (10 mL), dried over Na₂SO₄, and concentrated. The crude material was purified on silica (Hex to 10% EtOAc/Hex) to afford I-2c (0.64 g, 89% yield) as a white solid; the structure was confirmed by ¹H NMR in CDCl₃.

To I-2c (0.64 g, 2.16 mmol, 1 eq) was added anhydrous ammonium acetate (166 mg, 2.16 mmol, 1 eq), nitromethane (0.58 mL, 10.8 mmol, 5 eq), acetic acid (glacial, 1.75 mL, 30.2 mmol, 14 eq) and activated 3 Å molecular sieves (1.2 g wet weight) under N₂ at room temperature. The reaction mixture was heated at 95° C. for 1 h. The molecular sieves were removed, reaction mixture poured into brine (20 mL) and extracted with DCM (3×20 mL). The combined organic phase was washed with water (10 mL), then brine (10 mL), dried over Na₂SO₄, and concentrated. The crude material was purified on silica (Hex to 20% EtOAc/Hex) to afford I-2d (0.63 g, 86% yield) as an orange solid; the structure was confirmed by ¹H NMR in CDCl₃.

To a mixture of IPA (6 mL) and water (3 mL) at room temperature was added I-2d (0.63 g, 1.87 mmol, 1 eq), and the mixture was heated to 50° C. Then, NaBH₄ (0.53 g, 14.0 mmol, 7.5 eq) and an aqueous solution of CuCl₂ (2M, 0.10 mL, 0.187 mmol, 0.10 eq) were slowly added portion wise, then gradually heated to 80° C. and held for 1 h. The reaction mixture was then poured into aqueous NaOH (2M, 30 mL) and extracted with DCM (3×30 mL). The combined organic phase was washed with brine (30 mL), dried over Na₂SO₄, and concentrated. The crude material was purified on silica (DCM to 90:10:1 DCM/MeOH/NH₄OH) to afford I-2 (50 mg, 9% yield) as a yellow oil; the structure was confirmed by ¹H in CDCl₃.

To a solution of I-2 (73 mg, 0.236 mmol, 1 eq) in a mixture of anhydrous MeOH (2.4 mL) and anhydrous Et₂O (2.4 mL) was added anhydrous HCl (2M in Et₂O, 0.13 mL, 0.260 mmol, 1.1 eq) dropwise at room temperature under N₂. After 10 min, the reaction mixture was concentrated, solid washed with Et₂O, and dried to afford I-2 as an HCl salt (79 mg, 97% yield) as a beige solid; the structure was confirmed by ¹H NMR in DMSO-d₆.

### Example 15

### 2-(4-((3,3-difluoropropyl)thio)-2,5-dimethoxyphenyl)ethan-1-amine (I-3)

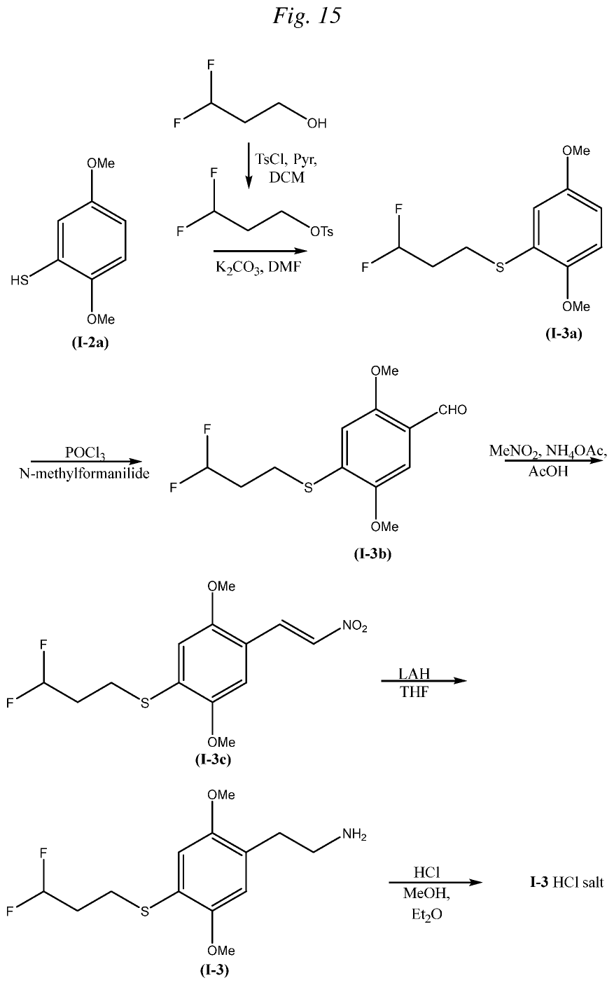

Synthesis of 2-(4-((3,3-difluoropropyl)thio)-2,5-dimethoxyphenyl)ethan-1-amine (I-3) was carried out according to FIG. 15.

To a solution of 3,3-difluoropropanol (0.64 g, 6.66 mmol, 1 eq) in anhydrous DCM (6.7 mL) at 0° C. under N₂ was added 4-toluenesulfonyl chloride (1.90 g, 9.99 mmol, 1.5 eq) followed slowly by pyridine (1.2 mL, 14.65 mmol, 2.2 eq). After 5 h, the reaction mixture was diluted with Et₂O(100 mL) and washed consecutively with water (40 mL), 1M aqueous HCl (40 mL), saturated aqueous NaHCO₃ (40 mL), and brine (40 mL). The organic phase was dried over Na₂SO₄ and concentrated. The crude material was purified on silica (Hex to DCM) to afford 3,3-difluoropropyl 4-methylbenzenesulfonate (1.33 g, 80% yield) as a colorless oil; the structure was confirmed by ¹H NMR in CDCl₃. To a solution of commercially available 2,5-dimethoxythiophenol I-2a (0.73 mL, 4.83 mmol, 1 eq) in anhydrous DMF (4.8 mL) at room temperature under N₂ was added potassium carbonate (0.73 g, 5.31 mmol, 1.1 eq) and 3,3-difluoropropyl 4-methylbenzenesulfonate (1.33 g, 5.31 mmol, 1.1 eq). After 2 days, reaction mixture poured into water (50 mL) and extracted with DCM (3×50 mL). The combined organic phase was washed with water (2×30 mL) then brine (20 mL), dried over Na₂SO₄, and concentrated. The crude material was purified on silica (Hex to 10% EtOAc/Hex) to afford I-3a (1.14 g, 95% yield) as a colorless oil; the structure was confirmed by ¹H NMR in CDCl₃.

To POCl₃ (2.39 mL, 25.63 mmol, 4.3 eq) was slowly added N-methylformanilide (2.72 mL, 22.05 mmol, 3.7 eq) at room temperature under N₂. After 1 h, I-3a (1.48 g, 5.96 mmol, 1 eq) was slowly added and reaction heated to 70° C. After 2 h, the reaction mixture was carefully poured into ice water (50 mL) and extracted with DCM (3×50 mL). The combined organic phase was washed with brine (20 mL), dried over Na₂SO₄, and concentrated. The crude material was purified on silica (Hex to 20% EtOAc/Hex). The impurity in the combined fractions was removed by trituration with EtOAc/Hex to afford I-3b (1.25 g, 76% yield) as a white solid; the structure was confirmed by ¹H NMR in CDCl₃.

To I-3b (1.25 g, 4.52 mmol, 1 eq) was added anhydrous ammonium acetate (0.35 g, 4.52 mmol, 1 eq), nitromethane (1.2 mL, 22.6 mmol, 5 eq), acetic acid (glacial, 3.6 mL, 63.3 mmol, 14 eq) and activated 3 Å molecular sieves (2.5 g wet weight) under N₂ at room temperature. The reaction mixture was heated at 95° C. for 1 h. The molecular sieves were removed, reaction mixture poured into brine (30 mL) and extracted with DCM (3×30 mL). The combined organic phase was washed with water (10 mL), then brine (10 mL), dried over Na₂SO₄, and concentrated. The crude material was purified on silica (Hex to 50% EtOAc/Hex) to afford I-3c (1.02 g, 71% yield) as an orange solid; the structure was confirmed by ¹H NMR in CDCl₃.

To a suspension of LiAlH₄ (0.708 g, 18.65 mmol, 6.5 eq) in anhydrous THF (75 mL) at room temperature under N₂ was slowly added I-3c (0.916 g, 2.87 mmol, 1 eq) in anhydrous THF (15 mL) and reaction heated at reflux overnight. The mixture was cooled to 0° C. and carefully quenched with water (0.7 mL), 2M aqueous NaOH (1.4 mL), then water (2.1 mL) and poured into water (100 mL). The mixture was extracted with DCM (3×100 mL) and the combined organic phase was washed with brine (50 mL), dried over Na₂SO₄, and concentrated. The crude material was purified on silica (DCM to 90:10:1 DCM/MeOH/NH₄OH) to afford I-3 (0.376 g, 45% yield) as a yellow oil; the structure was confirmed by ¹H NMR in CDCl₃.

To a solution of I-3 (415 mg, 1.42 mmol, 1 eq) in a mixture of anhydrous MeOH (14 mL) and anhydrous Et₂O (14 mL) was added anhydrous HCl (2M in Et₂O, 0.79 mL, 1.57 mmol, 1.1 eq) dropwise at room temperature under N₂. After 10 min, the reaction mixture was concentrated, solid washed with Et₂O, and dried to afford I-3 as an HCl salt (446 mg, 96% yield) as a beige solid; the structure was confirmed by ¹H NMR and ¹⁹F NMR in DMSO-d₆.

### Example 16

### 2-(4-((3-fluoropropyl)thio)-2,5-dimethoxyphenyl)ethan-1-amine (I-4)

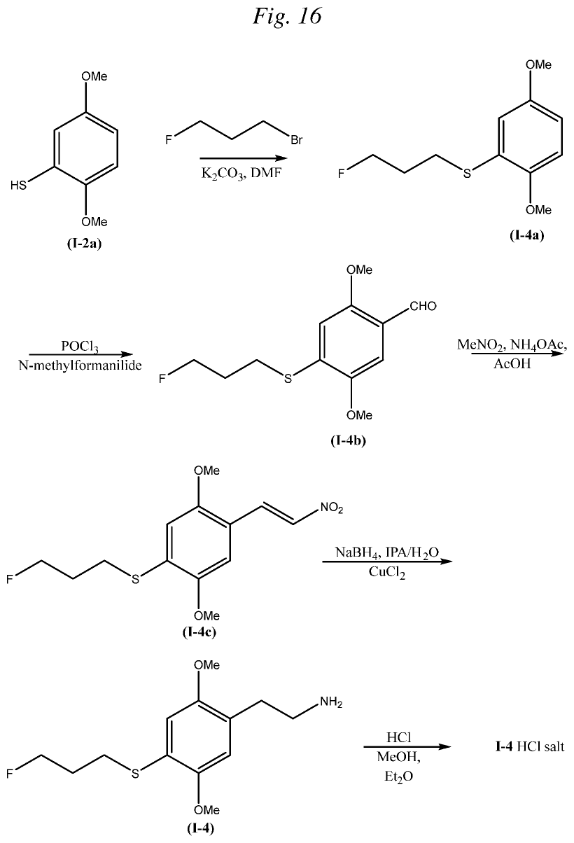

Synthesis of 2-(4-((3-fluoropropyl)thio)-2,5-dimethoxyphenyl)ethan-1-amine (I-4) was carried out according to FIG. 16.

To a solution of commercially available 2,5-dimethoxythiophenol I-2a (0.87 mL, 5.82 mmol, 1 eq) in anhydrous DMF (5.8 mL) at room temperature under N₂ was added potassium carbonate (1.21 g, 8.73 mmol, 1.5 eq) and 1-bromo-3-fluoropropane (0.80 mL, 8.73 mmol, 1.5 eq). After stirring overnight, reaction mixture poured into water (50 mL) and extracted with DCM (3×50 mL). The combined organic phase was washed with water (2×30 mL) then brine (20 mL), dried over Na₂SO₄, and concentrated. The crude material was purified on silica (Hex to 10% EtOAc/Hex) to afford I-4a (1.28 g, 95% yield) as a colorless oil; the structure was confirmed by ¹H NMR in CDCl₃.

To POCl₃ (2.23 mL, 23.9 mmol, 4.3 eq) was slowly added N-methylformanilide (2.54 mL, 20.6 mmol, 3.7 eq) at room temperature under N₂. After 1 h, I-4a (1.28 g, 5.56 mmol, 1 eq) was slowly added and reaction heated to 70° C. After 2 h, the reaction mixture was carefully poured into ice water (30 mL) and extracted with DCM (3×30 mL). The combined organic phase was washed with brine (10 mL), dried over Na₂SO₄, and concentrated. The crude material was purified on silica (Hex to 10% EtOAc/Hex). The impurity in the combined fractions was removed by trituration with EtOAc/Hex to afford I-4b (1.11 g, 77% yield) as a white solid; the structure was confirmed by ¹H NMR in CDCl₃.

To I-4b (1.11 g, 4.30 mmol, 1 eq) was added anhydrous ammonium acetate (331 mg, 4.30 mmol, 1 eq), nitromethane (1.2 mL, 21.5 mmol, 5 eq), acetic acid (glacial, 3.5 mL, 60.2 mmol, 14 eq) and activated 3 Å molecular sieves (2.3 g wet weight) under N₂ at room temperature. The reaction mixture was heated at 95° C. for 1 h. The molecular sieves were removed, reaction mixture poured into brine (40 mL) and extracted with DCM (3×40 mL). The combined organic phase was washed with water (20 mL), then brine (20 mL), dried over Na₂SO₄, and concentrated. The crude material was purified on silica (Hex to 20% EtOAc/Hex) to afford I-4c (1.00 g, 77% yield) as a yellow solid; the structure was confirmed by ¹H NMR in CDCl₃.

To a mixture of IPA (9 mL) and water (4.5 mL) at room temperature under N₂ was added I-4c (0.80 g, 2.65 mmol, 1 eq), and the mixture was heated to 50° C. Then, NaBH₄ (0.75 g, 19.9 mmol, 7.5 eq) and an aqueous solution of CuCl₂ (2M, 0.13 mL, 0.265 mmol, 0.10 eq) were slowly added portion wise, then gradually heated to 80° C. and held for 1 h. The reaction mixture was then poured into aqueous NaOH (2M, 30 mL) and extracted with DCM (3×30 mL). The combined organic phase was washed with brine (30 mL), dried over Na₂SO₄, and concentrated. The crude material was purified on silica (DCM to 90:10:1 DCM/MeOH/NH₄OH) to afford I-4 (0.35 g, 48% yield) as a colorless oil; the structure was confirmed by ¹H NMR in CDCl₃.

To a solution of I-4 (435 mg, 1.59 mmol, 1 eq) in a mixture of anhydrous MeOH (15 mL) and anhydrous Et₂O (15 mL) was added anhydrous HCl (2M in Et₂O, 0.88 mL, 1.75 mmol, 1.1 eq) dropwise at room temperature under N₂. After 10 min, the reaction mixture was concentrated, solid washed with Et₂O, and dried to afford I-4 as an HCl salt (416 mg, 84% yield) as a beige solid; the structure was confirmed by ¹H NMR in DMSO-d₆.

### Example 17

### 2-(2,5-dimethoxy-4-(3,3,3-trifluoropropoxy)phenyl)ethan-1-amine (I-5)

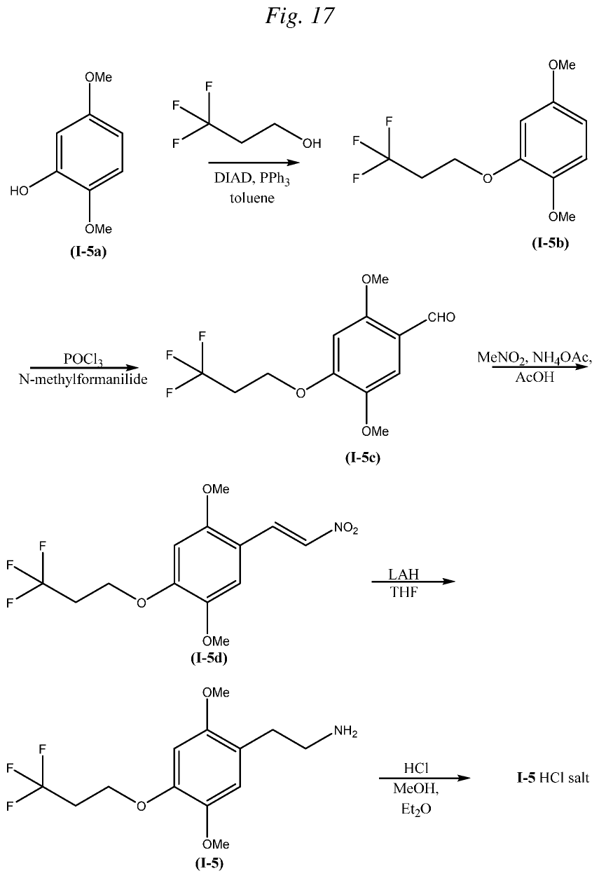

Synthesis of 2-(2,5-dimethoxy-4-(3,3,3-trifluoropropoxy)phenyl)ethan-1-amine (I-5) was carried out according to FIG. 17.

To a solution of commercially available 2,5-dimethoxyphenol I-5a (0.60 g, 3.89 mmol, 1 eq) and 3,3,3-trifluoro-1-propanol (0.41 mL, 4.67 mmol, 1.2 eq) in anhydrous DCM (12 mL) at 0° C. under N₂ was added PPh₃ (1.22 g, 4.67 mmol, 1.2 eq) in anhydrous DCM (5 mL) dropwise followed by DIAD (0.84 mL, 4.28 mmol, 1.1 eq) in anhydrous DCM (4 mL) dropwise. After 30 min, reaction allowed to warm to room temperature and stir overnight. Reaction mixture was concentrated and purified on silica (Hex to 30% EtOAc/Hex) to afford I-5b (0.34 g, 35% yield) as a white solid; the structure was confirmed by ¹H NMR in CDCl₃.

To POCl₃ (0.85 mL, 9.11 mmol, 4.3 eq) was slowly added N-methylformanilide (0.97 mL, 7.84 mmol, 3.7 eq) at room temperature under N₂. After 1 h, I-5b (0.53 g, 2.12 mmol, 1 eq) was slowly added and reaction heated to 70° C. After 2 h, the reaction mixture was carefully poured into ice water (50 mL) and extracted with DCM (3×50 mL). The combined organic phase was washed with brine (20 mL), dried over Na₂SO₄, and concentrated. The crude material was purified on silica (Hex to 30% EtOAc/Hex). The impurity in the combined fractions was removed by trituration with EtOAc/Hex to afford I-5c (0.49 g, 83% yield) as a white solid; the structure was confirmed by ¹H NMR in CDCl₃.

To I-5c (0.68 g, 2.44 mmol, 1 eq) was added anhydrous ammonium acetate (0.19 g, 2.44 mmol, 1 eq), nitromethane (0.66 mL, 12.2 mmol, 5 eq), acetic acid (glacial, 2.0 mL, 34.2 mmol, 14 eq) and activated 3 Å molecular sieves (1.4 g wet weight) under N₂ at room temperature. The reaction mixture was heated at 95° C. for 1 h. The molecular sieves were removed, reaction mixture poured into brine (30 mL) and extracted with DCM (3×30 mL). The combined organic phase was washed with water (10 mL), then brine (10 mL), dried over Na₂SO₄, and concentrated. The crude material was purified on silica (Hex to 50% EtOAc/Hex) to afford I-5d (0.49 g, 62% yield) as an orange solid; the structure was confirmed by ¹H NMR in CDCl₃.

To a suspension of LiAlH₄ (299 mg, 7.89 mmol, 6.5 eq) in anhydrous THF (40 mL) at room temperature under N₂ was slowly added I-5d (390 mg, 1.21 mmol, 1 eq) in anhydrous THF (6 mL) and reaction heated at reflux overnight. The mixture was cooled to 0° C. and carefully quenched with water (0.3 mL), 2M aqueous NaOH (0.6 mL), then water (0.9 mL) and poured into water (50 mL). The mixture was extracted with DCM (3×50 mL) and the combined organic phase was washed with brine (20 mL), dried over Na₂SO₄, and concentrated. The crude material was purified on silica (DCM to 90:10:1 DCM/MeOH/NH₄OH) to afford I-5 (85 mg, 24% yield) as a beige solid; the structure was confirmed by ¹H NMR in CDCl₃.

To a solution of I-5 (107 mg, 0.365 mmol, 1 eq) in a mixture of anhydrous MeOH (3.7 mL) and anhydrous Et₂O(3.7 mL) was added anhydrous HCl (2M in Et₂O, 0.20 mL, 0.40 mmol, 1.1 eq) dropwise at room temperature under N₂. After 10 min, the reaction mixture was concentrated, solid washed with Et₂O, and dried to afford I-5 as an HCl salt (127 mg, 100% yield) as a beige solid; the structure was confirmed by ¹H NMR and ¹⁹F NMR in DMSO-d₆.

### Example 18

### 2-(4-(3,3-difluoropropoxy)-2,5-dimethoxyphenyl)ethan-1-amine (I-6)

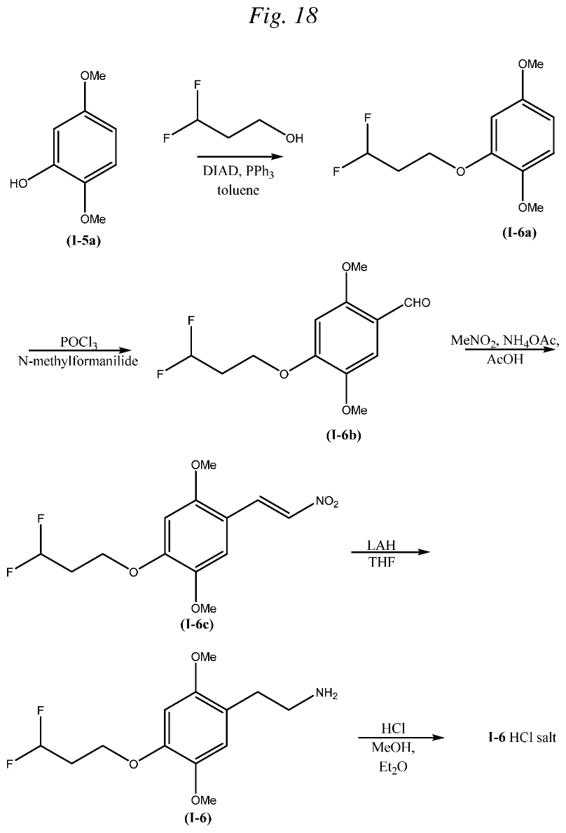

Synthesis of 2-(4-(3,3-difluoropropoxy)-2,5-dimethoxyphenyl)ethan-1-amine (I-6) was carried out according to FIG. 18.

To a solution of PPh₃ (2.00 g, 7.63 mmol, 1.4 eq) in anhydrous DCM (7.6 mL) at 0° C. under N₂ was added DIAD (1.4 mL, 7.09 mmol, 1.3 eq) in anhydrous DCM (5.9 mL) dropwise followed by 3,3-difluoro-1-propanol (0.73 g, 7.63 mmol, 1.4 eq) in anhydrous DCM (7.6 mL) and finally commercially available 2,5-dimethoxyphenol I-5a (0.84 g, 5.45 mmol, 1 eq) in anhydrous DCM (11 mL). After 30 min, reaction allowed to warm to room temperature and stir for 3 h. Reaction mixture was concentrated and purified on silica (Hex to 20% EtOAc/Hex) to afford I-6a (0.91 g, 72% yield) as a colorless oil; the structure was confirmed by ¹H NMR in CDCl₃.

To POCl₃ (1.95 mL, 20.9 mmol, 4.3 eq) was slowly added N-methylformanilide (2.22 mL, 18.0 mmol, 3.7 eq) at room temperature under N₂. After 1 h, I-6a (1.13 g, 4.87 mmol, 1 eq) was slowly added and reaction heated to 70° C. After 2 h, the reaction mixture was carefully poured into ice water (50 mL) and extracted with DCM (3×50 mL). The combined organic phase was washed with brine (20 mL), dried over Na₂SO₄, and concentrated. The crude material was purified on silica (Hex to 10% EtOAc/Hex). The impurity in the combined fractions was removed by trituration with EtOAc/Hex to afford I-6b (1.01 g, 80% yield) as a white solid; the structure was confirmed by ¹H NMR in CDCl₃.

To I-6b (1.01 g, 3.88 mmol, 1 eq) was added anhydrous ammonium acetate (0.30 g, 3.88 mmol, 1 eq), nitromethane (1.10 mL, 19.4 mmol, 5 eq), acetic acid (glacial, 3.1 mL, 54.3 mmol, 14 eq) and activated 3 Å molecular sieves (2.2 g wet weight) under N₂ at room temperature. The reaction mixture was heated at 95° C. for 1 h. The molecular sieves were removed, reaction mixture poured into brine (30 mL) and extracted with DCM (3×30 mL). The combined organic phase was washed with water (10 mL), then brine (10 mL), dried over Na₂SO₄, and concentrated. The crude material was purified on silica (Hex to 50% EtOAc/Hex) to afford I-6c (0.66 g, 57% yield) as an orange solid; the structure was confirmed by ¹H NMR in CDCl₃.

To a suspension of LiAlH₄ (456 mg, 12.0 mmol, 6.5 eq) in anhydrous THF (60 mL) at room temperature under N₂ was slowly added I-6c (560 mg, 1.85 mmol, 1 eq) in anhydrous THF (9 mL) and reaction heated at reflux overnight. The mixture was cooled to 0° C. and carefully quenched with water (0.5 mL), 2M aqueous NaOH (1 mL), then water (1.5 mL) and poured into water (50 mL). The mixture was extracted with DCM (3×50 mL) and the combined organic phase was washed with brine (20 mL), dried over Na₂SO₄, and concentrated. The crude material was purified on silica (DCM to 90:10:1 DCM/MeOH/NH₄OH) to afford I-6 (295 mg, 58% yield) as a colorless oil; the structure was confirmed by ¹H NMR in CDCl₃.

To a solution of I-6 (340 mg, 1.24 mmol, 1 eq) in a mixture of anhydrous MeOH (12 mL) and anhydrous Et₂O (12 mL) was added anhydrous HCl (2M in Et₂O, 0.68 mL, 1.36 mmol, 1.1 eq) dropwise at room temperature under N₂. After 10 min, the reaction mixture was concentrated, solid washed with Et₂O, and dried to afford I-6 as an HCl salt (375 mg, 97% yield) as a beige solid; the structure was confirmed by ¹H NMR and ¹⁹F NMR in DMSO-d₆.

### Example 19

### 2-(4-(3-fluoropropoxy)-2,5-dimethoxyphenyl)ethan-1-amine (I-7)

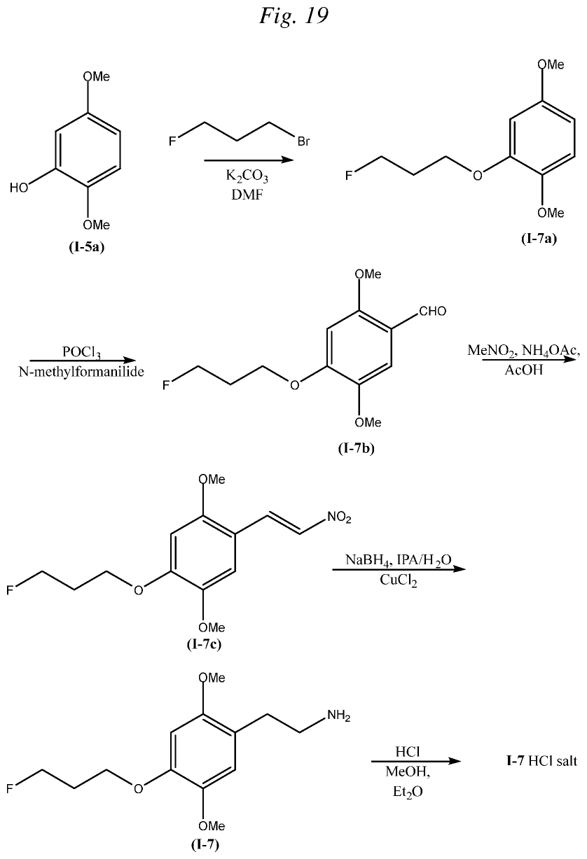

Synthesis of 2-(4-(3-fluoropropoxy)-2,5-dimethoxyphenyl)ethan-1-amine (I-7) was carried out according to FIG. 19.

To a solution of commercially available 2,5-dimethoxyphenol I-5a (0.80 g, 5.19 mmol, 1 eq) in anhydrous DMF (5.2 mL) at room temperature under N₂ was added potassium carbonate (1.08 g, 7.79 mmol, 1.5 eq) and 1-bromo-3-fluoropropane (0.71 mL, 7.79 mmol, 1.5 eq). After 2 days, reaction mixture poured into water (50 mL) and extracted with DCM (3×50 mL). The combined organic phase was washed with water (2×30 mL) then brine (20 mL), dried over Na₂SO₄, and concentrated. The crude material was purified on silica (Hex to 20% EtOAc/Hex) to afford I-7a (0.93 g, 84% yield) as a colorless oil; the structure was confirmed by ¹H NMR in CDCl₃.

To POCl₃ (2.21 mL, 23.7 mmol, 4.3 eq) was slowly added N-methylformanilide (2.52 mL, 20.4 mmol, 3.7 eq) at room temperature under N₂. After 1 h, I-7a (1.18 g, 5.51 mmol, 1 eq) was slowly added and reaction heated to 70° C. After 2 h, the reaction mixture was carefully poured into ice water (30 mL) and extracted with DCM (3×30 mL). The combined organic phase was washed with brine (10 mL), dried over Na₂SO₄, and concentrated. The crude material was purified on silica (Hex to 50% EtOAc/Hex) to afford I-7b (1.18 g, 88% yield) as a beige solid; the structure was confirmed by ¹H NMR in CDCl₃.

To I-7b (1.18 g, 4.87 mmol, 1 eq) was added anhydrous ammonium acetate (375 mg, 4.87 mmol, 1 eq), nitromethane (1.32 mL, 24.4 mmol, 5 eq), acetic acid (glacial, 3.91 mL, 68.2 mmol, 14 eq) and activated 3 Å molecular sieves (2.7 g wet weight) under N₂ at room temperature. The reaction mixture was heated at 95° C. for 1 h. The molecular sieves were removed, reaction mixture poured into brine (40 mL) and extracted with DCM (3×40 mL). The combined organic phase was washed with water (20 mL), then brine (20 mL), dried over Na₂SO₄, and concentrated. The crude material was purified on silica (Hex to 50% EtOAc/Hex) to afford I-7c (1.07 g, 77% yield) as an orange solid; the structure was confirmed by ¹H NMR in CDCl₃.

To a mixture of IPA (13 mL) and water (6.5 mL) at room temperature under N₂ was added I-7c (1.07 g, 3.75 mmol, 1 eq), and the mixture was heated to 50° C. Then, NaBH₄ (1.06 g, 28.1 mmol, 7.5 eq) and an aqueous solution of CuCl₂ (2M, 0.19 mL, 0.375 mmol, 0.10 eq) were slowly added portion wise, then gradually heated to 80° C. and held for 1 h. The reaction mixture was then poured into aqueous NaOH (2M, 50 mL) and extracted with DCM (3×50 mL). The combined organic phase was washed with brine (30 mL), dried over Na₂SO₄, and concentrated. The crude material was purified on silica (DCM to 90:10:1 DCM/MeOH/NH₄OH) to afford I-7 (535 mg, 55% yield) as a colorless oil; the structure was confirmed by ¹H NMR in CDCl₃.

To a solution of I-7 (535 mg, 2.08 mmol, 1 eq) in a mixture of anhydrous MeOH (20 mL) and anhydrous Et₂O (20 mL) was added anhydrous HCl (2M in Et₂O, 1.14 mL, 2.29 mmol, 1.1 eq) dropwise at room temperature under N₂. After 10 min, the reaction mixture was concentrated, solid washed with Et₂O, and dried to afford I-7 as an HCl salt (579 mg, 95% yield) as a beige solid; the structure was confirmed by ¹H NMR in DMSO-d₆.

### Examples 20-25

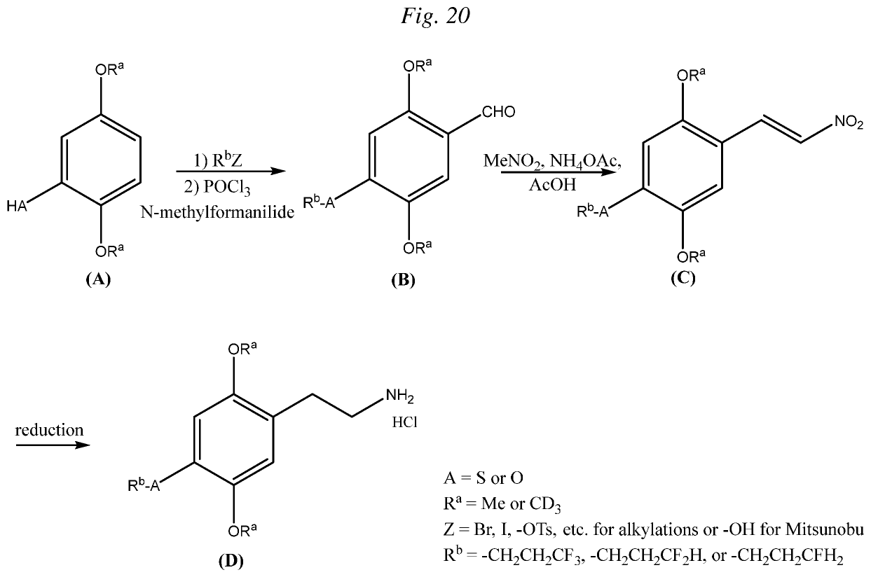

A general procedure which can be used to access Examples 20-25 is shown in FIG. 20, using a modified general procedure as reported by Shulgin (Shulgin, A., and Shulgin, Ann. (1991) Pihkal: a chemical love story, Transform Press, Berkeley, CA) and later modified by Maresh (Maresh, J. J., Ralko, A. A., Speltz, T. E., Burke, J. L., Murphy, C. M., Gaskell, Z., Girel, J. K., Terranova, E., Richtscheidt, C., and Krzeszowiec, M. (2014) Chemoselective Zinc/HCl Reduction of Halogenated beta-Nitrostyrenes: Synthesis of Halogenated Dopamine Analogues, Synlett 25, 2891-2894). Suitable starting material (A) is alkylated (e.g., using potassium tert-butoxide in THF, potassium carbonate in DMF, etc.) with a suitable fluoroalkyl halide or tosylate (R^{b}Z, where Z is Br, I, Cl, OTs, etc.), or alternatively subjected to Mitsunobu conditions (e.g., DIAD, PPh₃, toluene) using a suitable fluoroalcohol (R^{b}Z, where Z is —OH). Selective formylation with POCl₃ and N-methylformanilide then affords intermediate B. Intermediate B undergoes a nitroaldol condensation with nitromethane in buffered acidic conditions to form the β-nitrostyrene C. Subsequent bis-reduction of the nitro group and alkene under suitable reducing conditions (e.g., zinc dust in methanol containing hydrochloric acid, sodium borohydride/CuCl₂, lithium aluminum hydride, etc.) yields the final product (D), optionally as the free base or a suitable salt (e.g., HCl salt).

### Example 20

2-(2,5-bis(methoxy-d3)-4-((3,3,3-trifluoropropyl)thio)phenyl)ethan-1-amine (II-16). The structure of the product will be confirmed by ¹H NMR.

### Example 21

2-(4-((3,3-difluoropropyl)thio)-2,5-bis(methoxy-d3)phenyl)ethan-1-amine (II-17). The structure of the product will be confirmed by ¹H NMR.

### Example 22

2-(4-((3-fluoropropyl)thio)-2,5-bis(methoxy-d3)phenyl)ethan-1-amine (II-18). The structure of the product will be confirmed by ¹H NMR.

### Example 23

2-(2,5-bis(methoxy-d3)-4-(3,3,3-trifluoropropoxy)phenyl)ethan-1-amine (II-19). The structure of the product will be confirmed by ¹H NMR.

### Example 24

2-(4-(3,3-difluoropropoxy)-2,5-bis(methoxy-d3)phenyl)ethan-1-amine (II-20). The structure of the product will be confirmed by ¹H NMR.

### Example 25

2-(4-(3-fluoropropoxy)-2,5-bis(methoxy-d3)phenyl)ethan-1-amine (II-21). The structure of the product will be confirmed by ¹H NMR.

### Example 26

### 2-(4-((difluoromethyl)thio)-2,5-dimethoxyphenyl)ethan-1-amine (I-8)

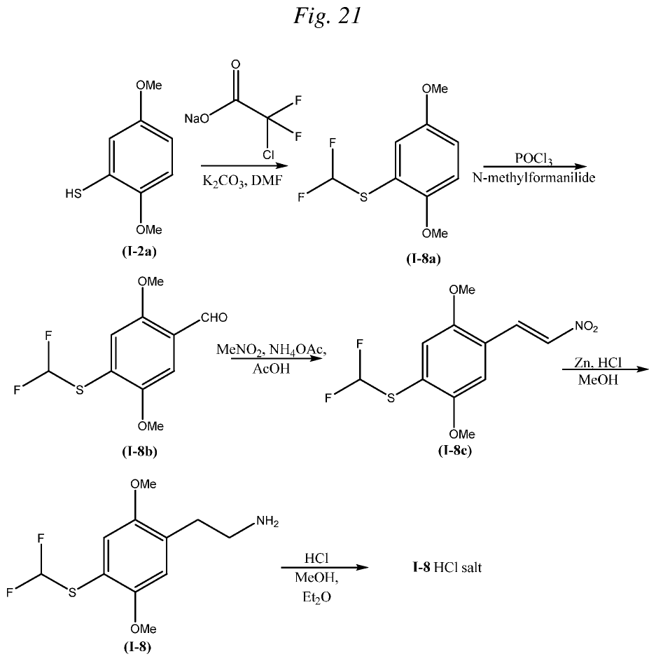

Synthesis of 2-(4-((difluoromethyl)thio)-2,5-dimethoxyphenyl)ethan-1-amine (I-8) was carried out according to FIG. 21.

To a solution of potassium carbonate (6.91 g, 50.0 mmol, 1.5 eq) and sodium chlorodifluoroacetate (10.15 g, 66.6 mmol, 2 eq) in anhydrous DMF (130 mL) at room temperature under N₂ was added commercially available 2,5-dimethoxythiophenol I-2a (5.0 mL, 33.3 mmol, 1 eq). The reaction was heated at 95° C. for 30 min, cooled to room temperature, and stirred with hexane (200 mL) and water (200 mL) for 15 min. The mixture was diluted with hexane (100 mL), washed with water (4×100 mL) then brine (100 mL), dried over Na₂SO₄, and concentrated. The crude material was purified on silica (Hex to 10% EtOAc/Hex) to afford I-8a (5.29 g, 72% yield) as a colorless oil; the structure was confirmed by ¹H NMR in CDCl₃.

To POCl₃ (10.7 mL, 115.2 mmol, 6 eq) was slowly added N-methylformanilide (14.2 mL, 115.2 mmol, 6 eq) at room temperature under N₂. After 15 min, I-8a (4.23 g, 19.2 mmol, 1 eq) was slowly added and the reaction heated to 70° C. After stirring overnight, the reaction mixture was carefully poured into ice water (100 mL) and extracted with DCM (3×100 mL). The combined organic phase was washed with brine (30 mL), dried over Na₂SO₄, and concentrated. The crude material was purified on silica (Hex to 10% EtOAc/Hex) to afford I-8b (2.38 g, 50% yield) as a beige solid; the structure was confirmed by ¹H NMR in CDCl₃.

To I-8b (2.73 g, 11.0 mmol, 1 eq) was added anhydrous ammonium acetate (0.85 g, 11.0 mmol, 1 eq), nitromethane (3.0 mL, 55.0 mmol, 5 eq), acetic acid (glacial, 8.8 mL, 154.0 mmol, 14 eq) and activated 3 Å molecular sieves (4.9 g wet weight) under N₂ at room temperature. The reaction mixture was heated at 95° C. for 1 h. The molecular sieves were removed, reaction mixture poured into brine (50 mL) and extracted with DCM (3×50 mL). The combined organic phase was washed with water (20 mL), then brine (20 mL), dried over Na₂SO₄, and concentrated. The crude material was purified on silica (Hex to 50% EtOAc/Hex) to afford I-8c (2.82 g, 88% yield) as an orange solid; the structure was confirmed by ¹H NMR in CDCl₃.

To anhydrous MeOH (100 mL) at 0° C. under N₂ was added I-8c (2.52 g, 8.65 mmol, 1 eq), zinc (6.80 g, 103.8 mmol, 12 eq), and concentrated HCl (12.1 M, 17.2 mL, 207.6 mmol, 24 eq) portionwise over 1 h. Reaction mixture allowed to warm to room temperature and stir for 4 h before cooling to 0° C. and adding additional zinc (6.80 g, 103.8 mmol, 12 eq) and concentrated HCl (12.1 M, 17.2 mL, 207.6 mmol, 24 eq) portionwise over 30 min. After warming to room temperature and stirring overnight, the excess zinc was removed, reaction mixture cooled to 0° C., basified with 20% NH₄OH/MeOH, and partially concentrated. The mixture was poured into water (200 mL) and extracted with DCM (3×200 mL). The combined organic phase was washed with brine (100 mL), dried over Na₂SO₄, and concentrated. The crude material was purified on silica (DCM to 90:10:1 DCM/MeOH/NH₄OH) to afford I-8 (0.71 g, 31% yield) as a brown oil; the structure was confirmed by ¹H NMR in CDCl₃.

To a solution of I-8 (70 mg, 0.266 mmol, 1 eq) in a mixture of anhydrous MeOH (2.7 mL) and anhydrous Et₂O (2.7 mL) was added anhydrous HCl (2M in Et₂O, 0.15 mL, 0.292 mmol, 1.1 eq) dropwise at room temperature under N₂. After 10 min, the reaction mixture was concentrated, solid washed with Et₂O, and dried to afford I-8 as an HCl salt (86 mg, 100% yield) as a beige solid; the structure was confirmed by ¹H NMR and ¹⁹F NMR in DMSO-d₆.

### Example 27

### 2-(2,5-dimethoxy-4-((2,2,3,3-tetrafluoropropyl)thio)phenyl)ethan-1-amine (I-10)

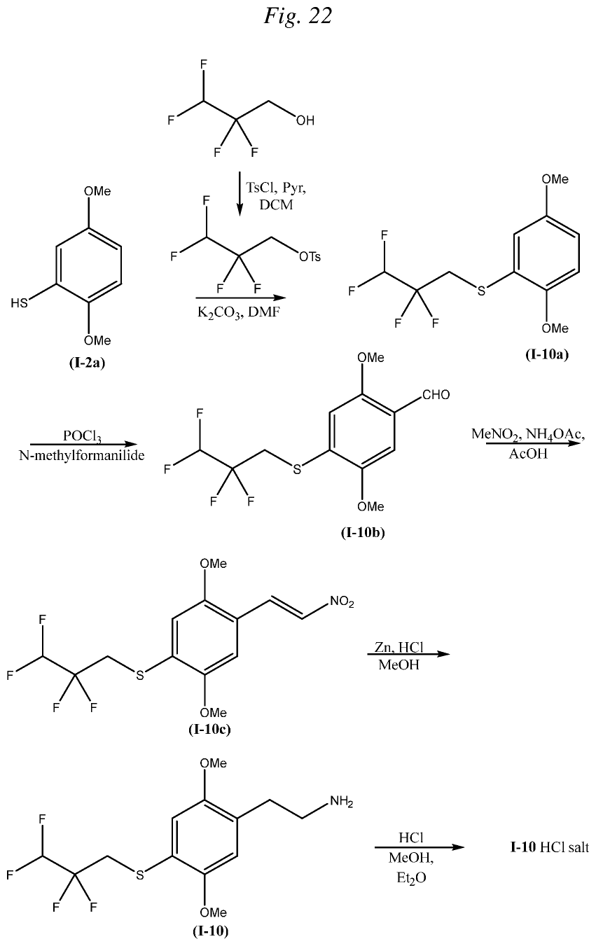

Synthesis of 2-(2,5-dimethoxy-4-((2,2,3,3-tetrafluoropropyl)thio)phenyl)ethan-1-amine (I-10) was carried out according to FIG. 22.

To a solution of 2,2,3,3-tetrafluoro-1-propanol (0.73 mL, 8.22 mmol, 1 eq) in anhydrous DCM (9 mL) at 0° C. under N₂ was added 4-toluenesulfonyl chloride (2.35 g, 12.33 mmol, 1.5 eq) followed slowly by pyridine (1.46 mL, 18.08 mmol, 2.2 eq). After warming to room temperature overnight, the reaction mixture was diluted with Et₂O (100 mL) and washed consecutively with water (40 mL), 1M aqueous HCl (40 mL), saturated aqueous NaHCO₃ (40 mL), and brine (40 mL). The organic phase was dried over Na₂SO₄ and concentrated. The crude material was purified on silica (Hex to DCM) to afford 2,2,3,3-tetrafluoropropyl 4-methylbenzenesulfonate (1.86 g, 79% yield) as a colorless oil; structure was confirmed by ¹H NMR in CDCl₃). To a solution of commercially available 2,5-dimethoxythiophenol I-2a (0.88 mL, 5.87 mmol, 1 eq) in anhydrous DMF (6 mL) at room temperature under N₂ was added potassium carbonate (0.89 g, 6.46 mmol, 1.1 eq) and 2,2,3,3-tetrafluoropropyl 4-methylbenzenesulfonate (1.85 g, 6.46 mmol, 1.1 eq). After 1 day, reaction mixture poured into water (50 mL) and extracted with DCM (3×50 mL). The combined organic phase was washed with water (2×30 mL) then brine (20 mL), dried over Na₂SO₄, and concentrated. The crude material was purified on silica (Hex to 20% EtOAc/Hex) to afford I-10a (1.43 g, 86% yield) as a colorless oil; the structure was confirmed by ¹H NMR in CDCl₃.

To POCl₃ (2.0 mL, 21.6 mmol, 4.3 eq) was slowly added N-methylformanilide (2.3 mL, 18.6 mmol, 3.7 eq) at room temperature under N₂. After 1 h, I-10a (1.43 g, 5.03 mmol, 1 eq) was slowly added and the reaction heated to 70° C. After 2 h, the reaction mixture was carefully poured into ice water (50 mL) and extracted with DCM (3×50 mL). The combined organic phase was washed with brine (20 mL), dried over Na₂SO₄, and concentrated. The crude material was purified on silica (Hex to 20% EtOAc/Hex) to afford I-10b (1.66 g, 100% yield) as a colorless oil; the structure was confirmed by ¹H NMR in CDCl₃.

To I-10b (1.66 g, 5.32 mmol, 1 eq) was added anhydrous ammonium acetate (0.41 g, 5.32 mmol, 1 eq), nitromethane (1.5 mL, 26.6 mmol, 5 eq), acetic acid (glacial, 4.3 mL, 74.5 mmol, 14 eq) and activated 3 Å molecular sieves (2.9 g wet weight) under N₂ at room temperature. The reaction mixture was heated at 95° C. for 1 h. The molecular sieves were removed, reaction mixture poured into brine (30 mL) and extracted with DCM (3×30 mL). The combined organic phase was washed with water (10 mL), then brine (10 mL), dried over Na₂SO₄, and concentrated. The crude material was purified on silica (Hex to 20% EtOAc/Hex) to afford I-10c (1.14 g, 60% yield) as a yellow solid; the structure was confirmed by ¹H NMR in CDCl₃.

To anhydrous MeOH (27 mL) at 0° C. under N₂ was added I-10c (0.57 g, 1.60 mmol, 1 eq), zinc (1.23 g, 19.3 mmol, 12 eq), and concentrated HCl (12.1 M, 3.20 mL, 38.5 mmol, 24 eq) portionwise over 20 min. Reaction mixture allowed to warm to room temperature and stir for 2 h before adding additional zinc (1.23 g, 19.3 mmol, 12 eq) and concentrated HCl (12.1 M, 3.20 mL, 38.5 mmol, 24 eq) portionwise over 20 min. After 2 h, the excess zinc was removed, reaction mixture cooled to 0° C., basified with 20% NH₄OH/MeOH, and partially concentrated. The mixture was poured into water (50 mL) and extracted with DCM (3×50 mL). The combined organic phase was washed with brine (20 mL), dried over Na₂SO₄, and concentrated. The crude material was purified on silica (DCM to 90:10:1 DCM/MeOH/NH₄OH) to afford I-10 (0.36 g, 69% yield) as a beige solid; the structure was confirmed by ¹H NMR in CDCl₃.

To a solution of I-10 (165 mg, 0.50 mmol, 1 eq) in a mixture of anhydrous MeOH (2.5 mL) and anhydrous Et₂O (2.5 mL) was added anhydrous HCl (2M in Et₂O, 0.28 mL, 0.55 mmol, 1.1 eq) dropwise at room temperature under N₂. After 10 min, the reaction mixture was concentrated, solid washed with Et₂O, and dried to afford I-10 as an HCl salt (168 mg, 92% yield) as a beige solid; the structure was confirmed by ¹H NMR and ¹⁹F NMR in DMSO-d6.

### Example 28

### 2-(2,5-dimethoxy-4-(prop-2-yn-1-ylthio)phenyl)ethan-1-amine (I-11)

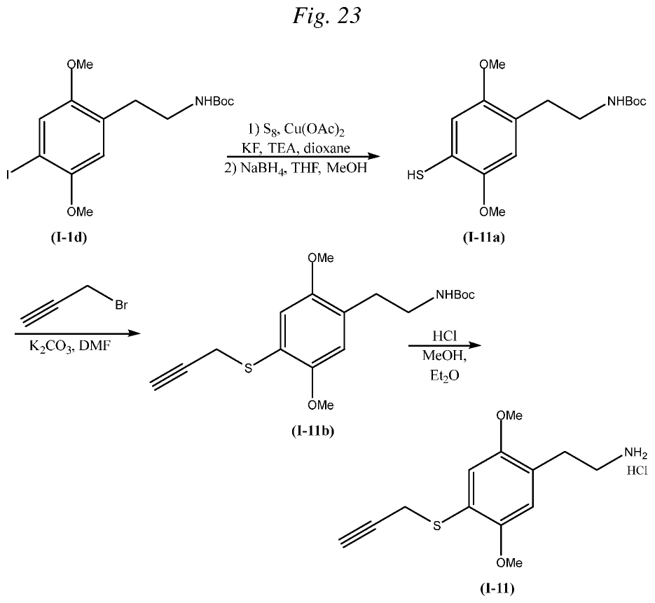

Synthesis of 2-(2,5-dimethoxy-4-(prop-2-yn-1-ylthio)phenyl)ethan-1-amine (I-11) was carried out according to FIG. 23.

To a dried pressure vessel was added I-1d (200 mg, 0.491 mmol, 1 eq), sulfur (63 mg, 0.246 mmol, 0.5 eq), Cu(OAc)₂ (9 mg, 0.049 mmol, 0.10 eq), and potassium fluoride (29 mg, 0.491 mmol, 1 eq) and vessel purged with N₂ before adding anhydrous 1,4-dioxane (sparged with N₂, 1 mL) and triethylamine (0.14 mL, 0.982 mmol, 2 eq) at room temperature. The pressure vessel was sealed and reaction heated at 110° C. After 2 days, reaction mixture was poured into water (20 mL) and extracted with DCM (3×20 mL). The combined organic phase was washed with brine (20 mL), dried over Na₂SO₄, and concentrated. The crude material was passed through silica (Hex to 50% EtOAc/Hex) to afford an impure intermediate (110 mg) as a brown oil, which was combined with NaBH₄ (27 mg, 0.70 mmol, 4 eq) in anhydrous THF (1 mL) under N₂ and heated to reflux before slowly adding anhydrous MeOH (0.10 mL) over 30 min. Reaction mixture held at reflux for 3 h before being poured into saturated aqueous NH₄Cl (20 mL) and water (20 mL). The mixture was extracted with DCM (3×25 mL), combined organic phase washed with brine (10 mL), dried over Na₂SO₄, and concentrated to afford I-11a (112 mg, 71% yield over 2 steps) as a tan solid. The crude material was promptly used in the next step without further purification; the structure was confirmed by ¹H NMR in CDCl₃.

To a solution of I-11a (100 mg, 0.32 mmol, 1 eq) in anhydrous DMF (0.32 mL) at room temperature under N₂ was added potassium carbonate (88 mg, 0.64 mmol, 2 eq) and propargyl bromide (80% by wt.) (54 μL, 0.48 mmol, 1.5 eq). After stirring overnight, reaction mixture poured into water (10 mL) and extracted with DCM (3×10 mL). The combined organic phase was washed with brine (10 mL), dried over Na₂SO₄, and concentrated. The crude material was purified on silica (Hex to 30% EtOAc/Hex) to afford I-11b (57 mg, 51% yield) as a white solid; the structure was confirmed by ¹H NMR in CDCl₃.

To a solution of I-11b (57 mg, 0.162 mmol, 1 eq) in a mixture of anhydrous MeOH (0.8 mL) and anhydrous Et₂O(0.8 mL) was added anhydrous HCl (1M in Et₂O, 0.65 mL, 0.649 mmol, 4 eq) dropwise at room temperature under N₂. After stirring overnight, the reaction mixture was concentrated, solid washed with Et₂O, and dried to afford I-11 as an HCl salt (41 mg, 88% yield) as a white solid; the structure was confirmed by ¹H NMR in DMSO-d₆.

### Example 29

### 2-(4-cyclopentyl-2,5-dimethoxyphenyl)ethan-1-amine (I-13)

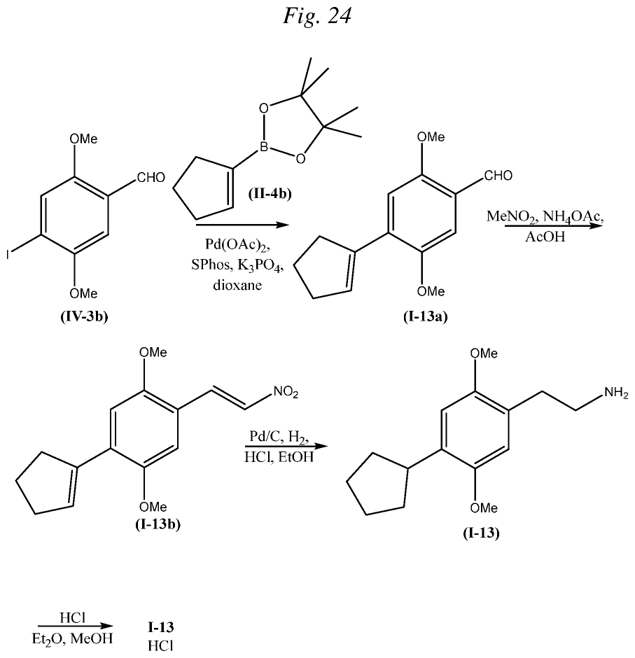

Synthesis of 2-(4-cyclopentyl-2,5-dimethoxyphenyl)ethan-1-amine (I-13) was carried out according to FIG. 24.

To a pressure vessel was added IV-3b (2.00 g, 6.71 mmol, 1 eq), SPhos (303 mg, 0.74 mmol, 0.11 eq), and Pd(OAc)₂ (151 mg, 0.67 mmol, 0.10 eq). The vessel was purged with N₂ before adding anhydrous 1,4-dioxane (67 mL), 2M aqueous K₃PO₄ (13.4 mL, 26.8 mmol, 4 eq), and cyclopentene-1-boronic acid, pinacol ester (II-4b, 2.60 g, 13.42 mmol, 2 eq) and mixture sparged with N₂ at room temperature. After sealing vessel, the reaction was heated at 100° C. overnight. The reaction mixture was filtered through Celite with DCM and concentrated. The crude material was purified on silica (Hex to 5% EtOAc/Hex) to afford I-13a (1.24 g, 78% yield) as a yellow solid; the structure was confirmed by ¹H NMR in CDCl₃.

To I-13a (1.24 g, 5.20 mmol, 1 eq) was added anhydrous ammonium acetate (401 mg, 5.20 mmol, 1 eq), nitromethane (1.41 mL, 26.02 mmol, 5 eq), acetic acid (glacial, 4.2 mL, 72.8 mmol, 14 eq) and activated 3 Å molecular sieves (2.5 g wet weight) under N₂ at room temperature. The reaction mixture was heated at 95° C. for 1 h. The molecular sieves were removed, reaction mixture poured into brine (30 mL) and extracted with EtOAc (3×30 mL). The combined organic phase was washed with water (30 mL), then brine (30 mL), dried over Na₂SO₄, and concentrated. The crude material was purified on silica (Hex to 5% EtOAc/Hex) to afford I-13b (231 mg, 16% yield) as an orange solid; the structure was confirmed by ¹H NMR in CDCl₃.

To a mixture of I-13b (231 mg, 0.82 mmol, 1 eq) and palladium (5% by wt.) on carbon (262 mg, 0.12 mmol, 0.15 eq) in anhydrous EtOH (12 mL) was added concentrated HCl (12.1 M, 0.14 mL, 1.64 mmol, 2 eq) and system degassed with N₂ before adding a hydrogen balloon. After 4 h at room temperature, hydrogen replaced with nitrogen atmosphere and reaction mixture filtered through Celite with MeOH. The reaction mixture was basified with MeOH/NH₄OH solution and concentrated. The crude material was purified on silica (DCM to 90:10:1 DCM/MeOH/NH₄OH) to afford I-13 (109 mg, 52% yield) as a white solid; the structure was confirmed by ¹H NMR in CDCl₃.

To a solution of I-13 (109 mg, 0.43 mmol, 1 eq) in a mixture of anhydrous MeOH (4 mL) and anhydrous Et₂O (4 mL) was added anhydrous HCl (2M in Et₂O, 0.23 mL, 0.47 mmol, 1.1 eq) dropwise at room temperature under N₂. After 10 min, the reaction mixture was concentrated, solid washed with Et₂O, and dried to afford I-13 as an HCl salt (123 mg, 99% yield) as a white solid; the structure was confirmed by ¹H NMR in DMSO-d₆.

### Example 30

### 2-(2,5-bis(methoxy-d3)-4-((trifluoromethyl)thio)phenyl)ethan-1-amine (II-10)

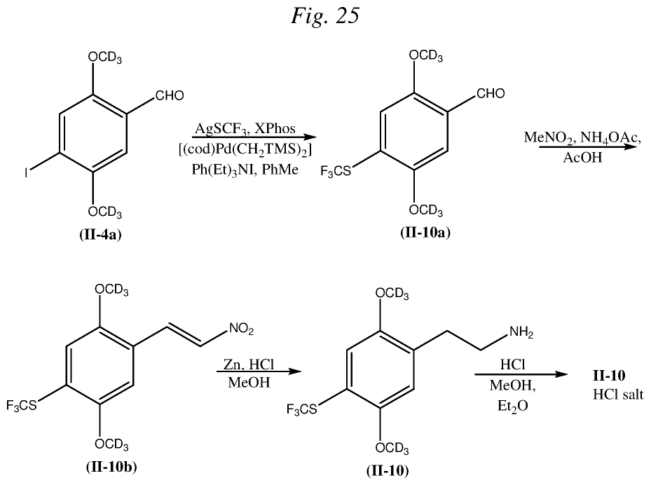

Synthesis of 2-(2,5-bis(methoxy-d3)-4-((trifluoromethyl)thio)phenyl)ethan-1-amine was carried out according to FIG. 25.

To a dried pressure vessel was added II-4a (0.94 g, 3.14 mmol, 1 eq), XPhos (164 mg, 0.35 mmol, 0.11 eq) and [(cod)Pd(CH₂TMS)₂](122 mg, 0.31 mmol, 0.10 eq) and vessel purged with N₂ before adding anhydrous PhMe (sparged with N₂, 6.3 mL). The mixture was then heated at 110° C. for 1 min before adding AgSCF₃ (0.85 g, 4.08 mmol, 1.3 eq) and Ph(Et)₃NI (1.25 g, 4.08 mmol, 1.3 eq), purging with N₂, and sealing pressure vessel. After heating 80° C. overnight, reaction mixture filtered through silica with DCM/EtOAc and concentrated. The crude material was purified on silica (Hex to 5% EtOAc/Hex) to afford II-10a (0.21 g, 25% yield) as a beige solid; the structure was confirmed by ¹H NMR in CDCl₃.

To II-10a (0.55 g, 2.02 mmol, 1 eq) was added anhydrous ammonium acetate (156 mg, 2.02 mmol, 1 eq), nitromethane (0.55 mL, 10.10 mmol, 5 eq), acetic acid (glacial, 1.62 mL, 28.28 mmol, 14 eq) and activated 3 Å molecular sieves (0.9 g wet weight) under N₂ at room temperature. The reaction mixture was heated at 95° C. for 1 h. The molecular sieves were removed, reaction mixture poured into brine (20 mL) and extracted with DCM (3×20 mL). The combined organic phase was washed with water (10 mL), then brine (10 mL), dried over Na₂SO₄, and concentrated. The crude material was purified on silica (Hex to 5% EtOAc/Hex) to afford II-10b (0.29 g, 45% yield) as a yellow solid; the structure was confirmed by ¹H NMR in CDCl₃.

To anhydrous MeOH (3.7 mL) at −10° C. under N₂ was added II-10b (0.29 g, 0.91 mmol, 1 eq), zinc (0.72 g, 10.96 mmol, 12 eq), and concentrated HCl (12.1 M, 1.81 mL, 21.92 mmol, 24 eq) portion wise over 20 min. Reaction mixture allowed to stir at 0° C. for 4 h. The excess zinc was removed and reaction mixture cooled to 0° C. before basifying with saturated methanolic NaOH. The mixture was diluted with DCM, dried over MgSO₄, filtered and concentrated. The crude material was purified on silica (DCM to 90:10:1 DCM/MeOH/NH₄OH) to afford II-10 (93 mg, 35% yield) as a white solid; the structure was confirmed by ¹H NMR in CDCl₃.

To a solution of II-10 (93 mg, 0.324 mmol, 1 eq) in a mixture of anhydrous MeOH (3.2 mL) and anhydrous Et₂O(3.2 mL) was added anhydrous HCl (2M in Et₂O, 0.18 mL, 0.356 mmol, 1.1 eq) dropwise at room temperature under N₂. After 10 min, the reaction mixture was concentrated, solid washed with Et₂O, and dried to afford II-10 as an HCl salt (104 mg, 99% yield) as a white solid; the structure was confirmed by ¹H NMR in DMSO-d₆.

### Example 31

### 2-(2,5-bis(methoxy-d3)-4-(trifluoromethyl)phenyl)ethan-1-amine (II-23)

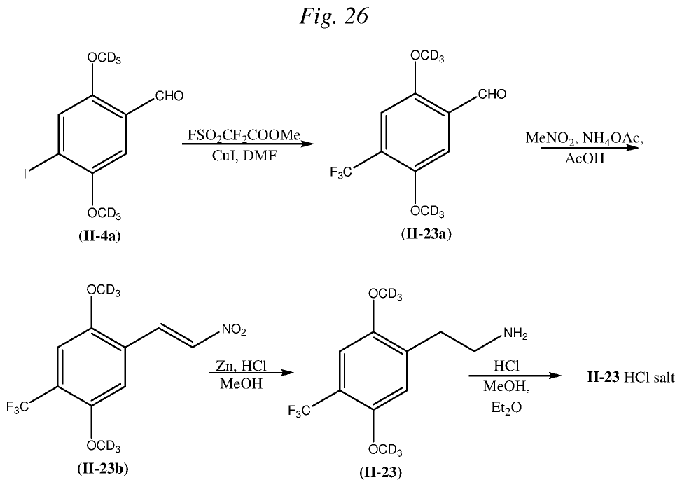

Synthesis of 2-(2,5-bis(methoxy-d3)-4-(trifluoromethyl)phenyl)ethan-1-amine was carried out according to FIG. 26.

To a mixture of II-4a (1.50 g, 5.03 mmol, 1 eq) and CuI (145 mg, 0.76 mmol, 0.15 eq) in anhydrous DMF (10 mL) was added methyl 2,2-difluoro-2-(fluorosulfonyl)acetate (1.3 mL, 10.06 mmol, 2 eq) at room temperature under N₂. After heating at 70° C. overnight, the reaction mixture was filtered through Celite with DCM, organic phase washed with water (3×50 mL) then brine (50 mL), dried over Na₂SO₄, and concentrated. The crude material was purified on silica (Hex to 20% EtOAc/Hex) to afford II-23a (1.14 g, 94% yield) as a white solid; the structure was confirmed by ¹H NMR in CDCl₃.

To II-23a (1.14 g, 4.75 mmol, 1 eq) was added anhydrous ammonium acetate (366 mg, 4.75 mmol, 1 eq), nitromethane (1.29 mL, 23.73 mmol, 5 eq), acetic acid (glacial, 3.81 mL, 66.5 mmol, 14 eq) and activated 3 Å molecular sieves (1.8 g wet weight) under N₂ at room temperature. The reaction mixture was heated at 95° C. for 1 h. The molecular sieves were removed, reaction mixture poured into brine (30 mL) and extracted with EtOAc (3×30 mL). The combined organic phase was washed with water (30 mL), then brine (30 mL), dried over Na₂SO₄, and concentrated to afford II-23b (1.18 g, 88% yield) as an orange solid. The crude material was used in the next step without further purification; the structure was confirmed by ¹H NMR in CDCl₃.

To anhydrous MeOH (4.2 mL) at −10° C. under N₂ was added II-23b (600 mg, 2.12 mmol, 1 eq), zinc (2.08 g, 31.8 mmol, 15 eq), and concentrated HCl (12.1 M, 5.25 mL, 63.6 mmol, 30 eq) portionwise over 30 min. Reaction mixture allowed to stir at 0° C. for 3 h then at room temperature overnight. The excess zinc was removed and reaction mixture cooled to 0° C. before basifying with aqueous 2M NaOH. The mixture was poured into water (100 mL) and extracted with EtOAc (3×100 mL). The combined organic phase was washed with brine (20 mL), dried over Na₂SO₄, and concentrated. The crude material was purified on silica (DCM to 90:10:1 DCM/MeOH/NH₄OH) to afford II-23 (76 mg, 14% yield) as a beige solid; the structure was confirmed by ¹H NMR in CDCl₃.

To a solution of II-23 (70 mg, 0.27 mmol, 1 eq) in a mixture of anhydrous MeOH (3 mL) and anhydrous Et₂O (3 mL) was added anhydrous HCl (2M in Et₂O, 0.15 mL, 0.30 mmol, 1.1 eq) dropwise at room temperature under N₂. After 10 min, the reaction mixture was concentrated, solid washed with Et₂O, and dried to afford II-23 as an HCl salt (83 mg, 100% yield) as a beige solid; the structure was confirmed by ¹H NMR in DMSO-d6.

### Example 32

### N-(cyclopropylmethyl)-2-(2,5-dimethoxy-4-((trifluoromethyl)thio)phenyl)ethan-1-amine (V-1)

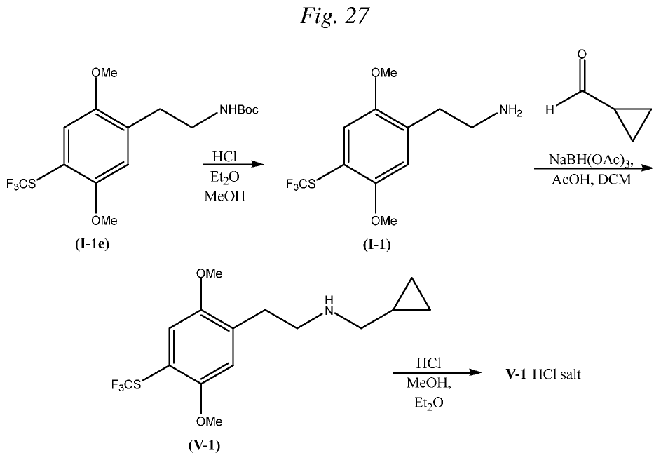

Synthesis of N-(cyclopropylmethyl)-2-(2,5-dimethoxy-4-((trifluoromethyl)thio)phenyl)ethan-1-amine (V-1) was carried out according to FIG. 27.

To a solution of I-1e (284 mg, 0.745 mmol, 1 eq) in a mixture of anhydrous MeOH (3.7 mL) and anhydrous Et₂O (3.7 mL) was added anhydrous HCl (2M in Et₂O, 1.86 mL, 3.723 mmol, 5 eq) dropwise at room temperature under N₂. After stirring overnight, the reaction mixture was concentrated, solid washed with Et₂O, and dried. The isolated HCl salt was then basified with saturated aqueous NaHCO₃ (25 mL) and extracted with DCM (3×25 mL). The combined organic phase was washed with brine (10 mL), dried over Na₂SO₄, and concentrated to afford I-1 (189 mg, 90% yield) as a white solid. The crude material was used in the next step without further purification; the structure was confirmed by ¹H NMR in CDCl₃.

To a solution of I-1 (55 mg, 0.196 mmol, 1 eq) in anhydrous DCM (0.78 mL) was added cyclopropanecarboxaldehyde (15 μL, 0.196 mmol, 1 eq), glacial AcOH (23 μL, 0.392 mmol, 2 eq), and sodium triacetoxyborohydride (80% by wt.) (78 mg, 0.294 mmol, 1.5 eq) at room temperature under N₂. After 3 h, reaction mixture poured into saturated aqueous NaHCO₃ (25 mL) and extracted with DCM (3×25 mL). The combined organic phase was washed with brine (10 mL), dried over Na₂SO₄, and concentrated. The crude material was purified on silica (DCM to 90:10:1 DCM/MeOH/NH₄OH) to afford V-1 (8 mg, 12% yield) as a colorless oil; the structure was confirmed by ¹H NMR in CDCl₃.

To a solution of V-1 (8 mg, 0.024 mmol, 1 eq) in a mixture of anhydrous MeOH (0.25 mL) and anhydrous Et₂O (0.25 mL) was added anhydrous HCl (2M in Et₂O, 13 μL, 0.026 mmol, 1.1 eq) dropwise at room temperature under N₂. After 10 min, the reaction mixture was concentrated, solid washed with Et₂O, and dried to afford V-1 as an HCl salt (9.5 mg, 100% yield) as a white solid; the structure was confirmed by ¹H NMR in DMSO-d6.

### Example 34

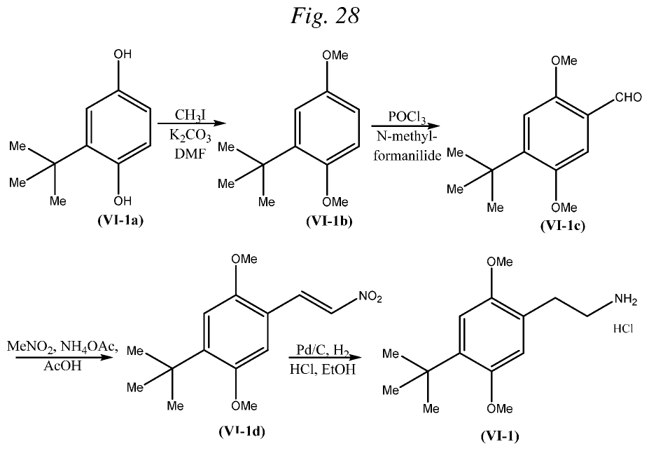

Synthesis of 2-(4-(tert-butyl)-2,5-dimethoxyphenyl)ethan-1-amine (VI-1) was carried out according to FIG. 28.

Bis-alkylation of 2-(tert-butyl)benzene-1,4-diol (VI-la) with CH₃I afforded intermediate VI-1b (35% yield). Selective formylation with POCl₃ and N-methylformanilide afforded benzaldehyde intermediate VI-1c (79% yield). The resulting benzaldehyde VI-1c underwent a nitroaldol condensation with nitromethane in buffered acidic conditions to form the β-nitrostyrene VI-1d (69% yield). Subsequent bis-reduction of the nitro group and alkene with Pd/C, H₂, ethanol containing hydrochloric acid yielded the final product (VI-1, 37% yield), as an HCl salt. The structure of the product was confirmed by ¹H NMR.

### Example 33

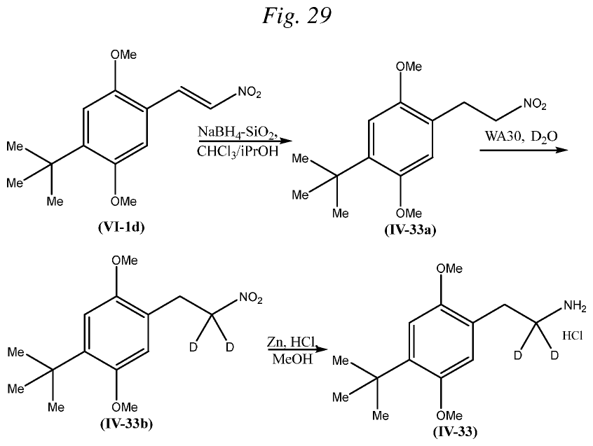

Synthesis of 2-(4-(tert-butyl)-2,5-dimethoxyphenyl)ethan-1,1-d2-1-amine (IV-33) is carried out according to FIG. 29. Starting from (VI-1d), treatment with sodium borohydride and silicone dioxide selectively reduces the alkene to intermediate IV-33a (Sinhababu, A. K., and Borchardt, R. T. (1983) Silica Gel-Assisted Reduction of Nitrostyrenes to 2-Aryl-1-Nitroalkanes with Sodium-Borohydride, Tetrahedron Letters 24, 227-230). Deuterium exchange at the α-position is next performed using the basic resin WA30 and deuterated water developed by Yamada (Yamada, T., Kuwata, M., Takakura, R., Monguchi, Y., Sajiki, H., and Sawama, Y. (2018) Organocatalytic Nitroaldol Reaction Associated with Deuterium-Labeling, Adv Synth Catal 360, 637-641) to form intermediate IV-33b. Reduction of the nitro group with zinc dust in methanol containing hydrochloric acid yields final product (IV-33). The structure of the product will be confirmed by ¹H NMR.

### Reference Compound 1

Synthesis of 2-(2,5-dimethoxy-4-methylphenyl)ethan-1-amine (Reference Compound 1) (“2C-D”).

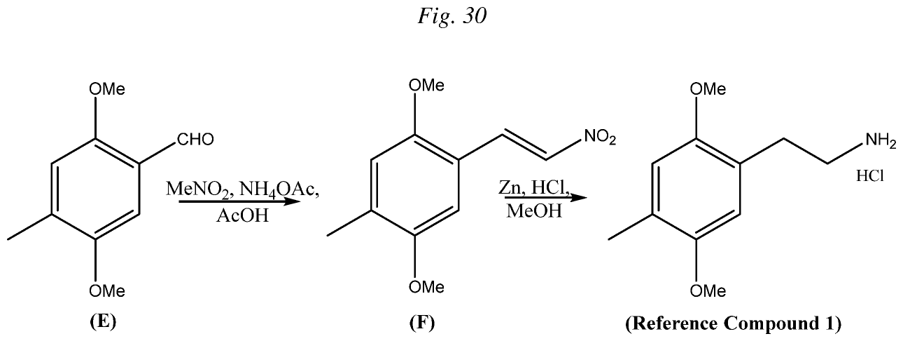

Synthesis of 2-(2,5-dimethoxy-4-methylphenyl)ethan-1-amine (Reference Compound 1) is carried out according to FIG. 30. 2,5-dimethoxy-4-methylbenzaldehyde (E) is subjected to a nitroaldol condensation with nitromethane in buffered acidic conditions to form the β-nitrostyrene (F). Subsequent bis-reduction of the nitro group and alkene with zinc dust in methanol containing hydrochloric acid yielded the final product (Reference Compound 1), as an HCl salt. The structure of the product will be confirmed by ¹H NMR.

### Reference Compound 2

2-(4-bromo-2,5-dimethoxyphenyl)ethan-1-amine (Reference Compound 2) (“2C—B”) is commercially available and was purchased from Cayman Chemical Co.

### Reference Compound 3

2-(2,5-dimethoxy-4-(methylthio)phenyl)ethan-1-amine (Reference Compound 3) (“2C-T”), HCl salt, is commercially available and was purchased from Cayman Chemical Co.

### Reference Compound 4

2-(2,5-dimethoxy-4-(trifluoromethyl)phenyl)ethan-1-amine (Reference Compound 4) (“2C-TFM”), HCl salt, is commercially available and was purchased from Cayman Chemical Co.

### II. Formulations

### Preparation of Ion-Exchange Resin Complex

Freebase of the compounds are complexed with a strong cation-exchange resin (sodium form, Amberlite IRP69, Rohm & Haas, Sodium Polystyrene Sulfonate, pharmaceutical grade USP, particle size 75-150 microns). The maximum load of the above resin is known to be about 5 meqv/g. In a typical procedure, the compound free base (10 mmol) is dissolved in 20 ml of ethanol. To this solution, 2 g of the IRP69 resin (washed with 3×50 ml of ethanol) is added at room temperature using a magnetic stirrer and kept stirring for 2 h. The resin is then filtered and washed with ethanol (2×20 ml). Compound release from the resin complex is studied using a Type I (basket) dissolution apparatus at pH 1 (0.1 M HCl) and 7.4 (0.1 M phosphate buffer). The compound concentration is determined by HPLC using an Agilux 1100 setup and UV detection.

In an acidic environment, the compound release is believed to be a fast process, with >90% of the drug leaching out within 30 min. At neutral pH, the release is believed to be substantially slower, for example, about 50% of the drug to be released in 1 h and about 80% at 2 h.

### Preparation of Beads of Ion Exchange Resin Complex Coated by Enteric Coating

### Seal Coating

Compound-ion-exchange resin Complex beads are seal-coated at a 2% weight gain using Opadry 03K19229 coating (contains hydroxypropylmethyl cellulose, triacetin and talc; Colorcon, NJ, USA, reconstituted at 6% solids) in a hydro-alcoholic solvent system (88:12, isopropanol: water) on a Niro-Aeromatic STREA 1 fluidized bed machine equipped with a Wurster coating module (bottom feed).

### Enteric Coating

The resulting beads are then coated using Opadry Enteric 940 white coating (poly [methacrylic acid, methyl methacrylate (1:1) type A; Colorcon, NJ, USA). Coating dispersions are reconstituted at 10% solids in a hydro-alcoholic solvent system (88:12, isopropanol: water) and applied to either a 5 or 12% weight gain. Enteric coating of placebo tablets is carried out without the preceding seal coating step. Samples are drawn at 5, 6, 7, 8, 10 and 12% weight gains.

### Drug Release Testing

Drug release at low pH is determined using a Type I apparatus 1 (basket), at 100 rpm. In the first stage, the dissolution medium is 1000 ml of 0.1 N HCl at 37° C. (±0.5° C.) and the bead load 2 g. After 1 h of operation in this medium, an aliquot is withdrawn and the drug content is determined by HPLC to be less than 1% total, confirming the integrity of the applied enteric coating. Drug release at neutral pH is determined using a Type I apparatus 1 (basket), at 100 rpm. In the second stage, the dissolution medium is 1000 mL of 0.1 M phosphate buffer at pH 7.4 at 37° C. (±0.5° C.) and a 2 g bead load. The medium aliquots are withdrawn at 15, 30, 60, 90, and 120 min, and the drug content is determined by HPLC using an Agilux 1100 setup and UV detection. The release of the drug is believed to be ca. 50% at 1 h and about 80% at 2 h, with a release profile over time similar to the uncoated resin beads.

### Manufacturing of Orally Disintegrating Tablets

Orally disintegrated tablets are designed by incorporating micro-beads of the drug-ion exchange resin complex with extended release characteristics into a matrix of the fast orally disintegrating components that help dispersing active material in the oral cavity and facilitate subsequent swallowing without use of water for more convenient administration of the drug.

Compounds are formulated into an orally disintegrating release tablet form, composition PI-ODT-1 by dry granulation using sugar based, fast-disintegrating matrix Pharmaburst 500 (SPI, PA, USA). 100 g of Pharmaburst 500 is mixed up with 20 g of the enterically coated compound-ion-exchange resin complex beads and sieved via 40 mesh sieves, to break agglomerates, and then the mixture is blended in a 400 ml tube blender for 15 minutes at 200 rev/min. After blending, magnesium stearate (200 mg) is added and blended for additional 3 minutes. The 250 mg convex-shaped tablets containing about 20 mg of active compound are compressed using a TDP tablet press and 9 mm dye. By applying a compression force of 8 kN, tablets of the hardness in the range 10-15 kP are generated. The tablet disintegration time is determined to be 60-75 seconds. The tablet dissolution is carried out in a Type II dissolution apparatus (paddle) (Distek Premiere 5100 Dissolution System, Distek Inc., North Brunswick, USA) at 100 rpm, 37° C., using 1×PBS buffer, pH=6.8 as an immersion media. At predetermined time intervals, 1 ml samples are withdrawn (not replaced), filtered and assayed. The amount of compound released is measured by HPLC using an Agilent 1100 setup (Nagy, J., and Veress, T., 2016, HPLC Analysis of Hallucinogenic Mushroom Alkaloids (Psilocin and Psilocybin) Applying Hydrophilic Interaction Chromatography (HILIC), J Forensic Res 7, 356). Solutions of known concentrations of compound are used to calculate the amount of drug released.

### Fatty Acid Salts of Compounds

Compound free bases (10 mmol) are dissolved in 30 ml of acetone and 10 mmol of decanoic acid is added and mixed for 5 min. A white crystalline precipitate of the 1:1 salt is formed upon cooling the mixture in a refrigerator overnight. The salt composition will be confirmed by elemental analysis.

### Preparation of Transdermal Skin Patches and In Vitro Permeation Studies

Compound I-1 is formulated in a drug-in-adhesive (DIA) patch to enable a steady delivery of the sub-psychedelic dose of the drug over extended periods of time. The formulation is optimized for a once-a-day application (24 h drug release). The DIA patch contains the drug, pressure sensitive acrylic-based adhesive, backing film, and release liner. In the preparation, 1.5 g of Compound I-1 and 6.0 g of DURO-TAK 87-900A (acrylate copolymer pressure sensitive adhesive; Henkel) are dissolved in 30 ml of methanol, the solution is agitated at room temperature for 30 min, and spread on a silicone-coated release liner (SCOTCHPAK; 3M, St. Paul, USA) with a micrometer adjustable film applicator to give a wet film thickness of 300 micron. They are kept at room temperature for 5 min and then in an oven at 75° C. for 30 min to remove any residual solvents. The patches are then laminated with backing membrane (COTRAN; 3M, St. Paul, USA), cut into appropriate sizes, packed in aluminum-foil, and stored at room temperature. The final dry thickness of the DIA matrix is 90 mm.

The in vitro permeation of the drug patch across human cadaver skins (Biopredic, USA) is evaluated using Franz diffusion cells. A circular transdermal patch is pressed on the skin with the adhesive side facing the stratum corneum. The receptor cells are filled with PBS containing 6% (w/v) Brij 98. The diffusion cell area for all in vitro studies is 1.77 cm². The diffusion cells are maintained at 32° C., and the solution in the receptor cells is stirred continuously at 600 rpm. At designated time points (2, 4, 6, 8, 10, 12, and 24 h), 0.5 ml of the solution in the receptor cell is withdrawn and replaced with an equal volume of fresh receptor medium. The concentrations in the samples are determined by LC-MS/MS analysis. A flux in the range of 20-25 microg/h/cm² should translate to the therapeutically relevant exposures in human subjects.

### III. Compound Testing

### Radioligand Competition Binding

Serotonin (5-HTR) affinities were determined by radioligand competition binding as previously described (for 5-HT_{1(A,B,D,E,F)} and 5-HT_{2(A,B,C)} receptor affinities, see Canal, C. E., Cordova-Sintjago, T., Liu, Y., Kim, M. S., Morgan, D., and Booth, R. G., 2013, Molecular pharmacology and ligand docking studies reveal a single amino acid difference between mouse and human serotonin 5-HT_{2A} receptors that impacts behavioral translation of novel 4-phenyl-2-dimethylaminotetralin ligands, J Pharmacol Exp Ther 347, 705-716; Armstrong, J. L., Casey, A. B., Saraf, T. S., Mukherjee, M., Booth, R. G., and Canal, C. E., 2020, (S)-5-(2′-Fluorophenyl)-N,N-dimethyl-1,2,3,4-tetrahydronaphthalen-2-amine, a Serotonin Receptor Modulator, Possesses Anticonvulsant, Prosocial, and Anxiolytic-like Properties in an Fmr1 Knockout Mouse Model of Fragile X Syndrome and Autism Spectrum Disorder, ACS Pharmacol Transl Sci 3, 509-523, Saraf, T. S., Felsing, D. E., Armstrong, J. L., Booth, R. G., and Canal, C. E., 2021, Evaluation of lorcaserin as an anticonvulsant in juvenile Fmr1 knockout mice, Epilepsy Res 175, 106677), with minor modifications.

cDNA plasmids encoding human 5-HTRs were procured from the cDNA Resource Center. Human embryonic kidney cells (HEK293, ATCC CRL-1573) were grown in a cell incubator in 100 mm dishes with antibiotic-free Dulbecco's modified Eagle's medium containing 10% fetal bovine serum. Cells were transfected at ˜85% confluency with 5-15 μg cDNA using one of various transfection reagents (e.g., TransIT-2020 reagent, Mirus Bio, Madison, Wisconsin). After approximately 48 hours, cell membrane was collected via homogenization in 50 mM tris buffer and centrifugation steps. For all experiments, serotonin (5-HT) hydrochloride was used as a positive control, and an appropriate high affinity, cold ligand (e.g., mianserin hydrochloride, 10 μM, for 5-HT₂Rs) was used to define non-specific binding. Each cell membrane homogenate expressing a 5-HTR was incubated at room temperature in 96-well plates with [³H]lysergic acid diethylamide ([³H]LSD) or [³H]ketanserin between 0.5 and 1 nM or [³H]mesulergine at appr. 3 nM for labeling 5-HT₂Rs, or [³H]5-carboxamidotryptine (5-CT) between 0.2 and 1 nM for labeling 5-HT_{1A,1B,1D}Rs in the absence or presence of test articles (e.g., at 10 concentrations from 0.3 nM to 10 μM in half-log units) in a buffer for 90 min. Following equilibration, each sample was filtered rapidly under vacuum through fiberglass filters presoaked in a buffer and washed by vacuum several times with an ice-cold buffer. Filters were soaked with scintillation fluid and counts per minute were detected using photodetectors (Microbeta Scintillation Counter).

The K_{d} for [³H]LSD was set to 0.7-1.0 nM (depending on the 5-HT₂R subtype labeled), for [³H]ketanserin was set to 1.6 nM for 5-HT_{2A}, for [³H]mesulergine was set to 2.92 nM for 5-HT_{2C}, for [³H]5-CT was set to 0.2-2.0 nM (depending on the 5-HT₁R subtype labeled), and the IC₅₀ values were computed using nonlinear, least squares regression analyses and then converted to binding affinity (Kᵢ) values using the Cheng-Prusoff equation (GraphPad Prism 9.0, San Diego, California). Data shown are the results from all experiments combined. As is known, G protein-coupled receptors (GPCR), including the 5-HT_{2A}R, can exist in multiple conformations, and ligands, typically agonist ligands, have unique affinities for these conformations of the receptor (Kenakin, T., 2017, Theoretical Aspects of GPCR-Ligand Complex Pharmacology, Chem Rev 117, 1, 4-20). The competition binding data from some of the tested compounds fit best to a two-site fit Kᵢ model. The data point for −10 samples is total specific binding (no compound present), and −4 was interpolated to complete the binding curves to 0 specific binding.

In another series of 5-HT_{1A,2A,2B} radioligand competition binding assays, the conditions were as follows: For 5-HT_{1A} assays, cell membrane homogenates (˜30 μg protein) derived from HEK-293 cells expressing human 5-HT_{1A}Rs were incubated with 0.5 nM [³H]8-hydroxy-2-(di-n-propylamino)tetralin ([³H]8-OH-DPAT) for 60 min at room temperature in the presence or absence of the test article. Non-specific binding was determined in the presence of 10 μM 8-OH-DPAT. Samples were then analyzed as described above, with only immaterial differences. For 5-HT_{2A} radioligand binding assays, cell membrane homogenates (30 μg protein) derived from HEK-293 cells expressing human 5-HT_{2A}Rs were incubated with 0.1 nM [¹²⁵I](±)2,5-dimethoxy-4-iodoamphetamine ([¹²⁵I](±)DOI) for 60 min at room temperature in the presence or absence of the test article (see Mulheron, J. G., Casanas, S. J., Arthur, J. M., Garnovskaya, M. N., Gettys, T. W. and Raymond, J. R., 1994, Human 5-HT_{1A} receptor expressed in insect cells activates endogenous GO-like G protein, J Biol Chem 269, 12954). Non-specific binding was determined in the presence of 1 μM (±)DOI. Samples were then analyzed as described above. For 5-HT_{2B} radioligand binding assays, cell membrane homogenates (10 μg protein) derived from Chinese hamster ovary (CHO) cells expressing human 5-HT_{2B}Rs were incubated with 0.2 nM [¹²⁵I](±)DOI for 60 min at room temperature in the presence or absence of the test article (see Bryant, H. U., Nelson, D. L., Button, D., Cole, H. W., Baez, M. B., Lucaites, V. L., Wainscott, D. B., Whitesitt, C., Reel, J., Simon, R. and Koppel, G. A., 1996, A novel class of 5-HT₂ A receptor antagonist: aryl aminoguanidines, Life Sci 15, 1259). Non-specific binding was determined in the presence of 1 μM (±)DOI. Samples were then analyzed as described above (see Choi, D. S., Birraux, G., Launay, J. M. and Maroteaux, L., 1994, The human serotonin 5-HT_{2B} receptor: pharmacological link between 5-HT2 and 5-HT1D receptors, FEBS Lett 352, 393).

### Receptor Function Assays

5-HT_{2A,2B,2C} receptor-mediated Gq activation—canonical signaling pathways—[phosphoinositide hydrolysis leading to the production of inositol phosphate 1 (IP1, IPOne, Cisbio assay) or direct G-protein activation using TRUPATH technology]were measured as previously described (Porter, R. H. P., Benwell, K. R., Lamb, H., Malcolm, C. S., Allen, N. F., Revell, D. F., Adams, D. R. and Sheardown, M. J., 1999, Functional characterization of agonists at recombinant human 5-HT2a, 5HT2b and 5-HT2c receptors in CHO-K1 cells, Brit J Pharmacol 128, 13; Olsen, R. H. J., DiBerto, J. F., English, J. G. et al. TRUPATH, an open-source biosensor platform for interrogating the GPCR transducerome. Nat Chem Biol 16, 841-849 (2020). https://doi.org/10.1038/s41589-020-0535-8) by measuring IP1 production using Fluorescence Resonance Energy Transfer (FRET) technology or by measuring Galpha and Gbeta/gamma dissociation using bioluminescent resonance energy transfer (BRET) technology. Briefly, for the IPOne assay, for 5-HT_{2A}Rs, CHO-K1 or HEK-293 cells were incubated with test articles and 5-HT (positive control) in stimulation buffer containing LiCl. After incubation, cells were lysed and fluorescence acceptor and donor were added. FRET was measured after 60 min at room temperature using a microplate reader (Envision, Perkin Elmer). TRUPATH assays followed a similar workflow, except that different reagents were used, and LiCl was not included in the samples; the protocol was based on the report by Olsen, R. H. J. et al., cited above. The results are expressed as a percent of control response to 10 μM 5-HT and data were fit to non-linear curves to calculate potencies (e.g., EC₅₀) and efficacies (e.g., Eₘₐₓ), relative to positive controls (e.g., 5-HT).

### In Vitro Liver Metabolism

In vitro stability was determined in Human (HLM), Rat (RLM), Mouse (MsLM), and dog (DLM) Liver Microsomes. All liver microsomes were purchased from a commercial supplier (e.g. XenoTech). The reaction mixture, minus NADPH, was prepared as described below. The test compound was added into the reaction mixture at a final concentration of 1 μM or co-dosed with their deuterated analog(s) at 1 μM each. The control compound, testosterone, was run simultaneously with the test compound in a separate reaction. The reaction mixture (without cofactor) was equilibrated in a shaking water bath at 37° C. for 5 minutes. The reaction was initiated by the addition of the cofactor, and the mixture was incubated in a shaking water bath at 37° C. Aliquots (150 μL) were withdrawn at 0, 5, 10, 20, 30, 60, and 120 minutes. Test compound and testosterone samples were immediately combined with 150 μL of ice-cold acetonitrile (ACN) containing 0.1% formic acid and internal standard to terminate the reaction. The samples were then mixed and centrifuged to precipitate proteins. All samples were assayed by LC-MS/MS using electrospray ionization. The peak area response ratio (PARR) of analyte to internal standard at each time point was compared to the PARR at time 0 to determine the percent remaining at each time point. Half-lives were calculated using GraphPad software, fitting to a single-phase exponential decay equation.

### Reaction Composition:

| | | |
|---|---|---|
| Liver Microsomes | 0.5 | mg/mL |
| NADPH (cofactor) | 1 | mM |
| Potassium Phosphate, pH 7.4 | 100 | mM |
| Magnesium Chloride | 5 | mM |
| Test compound (each, if co-dosed) | 1 | μM |

### Recombinant Human MAO-A Activity

hrMAO-A and MAO control were purchased from XenoTech. The reaction mixture was prepared as described below. The test compound was added into the reaction mixture at a final concentration of 1 μM or co-dosed with their deuterated analog at 1 μM each. The positive control kynuramine (25 μM) was run simultaneously with the test compound in a separate reaction. The reaction mixture (without test compounds or kynuramine) was equilibrated in a shaking water bath at 37° C. for 5 minutes. The reaction was initiated by the addition of the test compound or kynuramine, and the mixture was incubated in a shaking water bath at 37° C. Aliquots (150 μL) of the test compound reaction mixture were withdrawn at 0, 5, 10, 20, 30, 60, and 120 minutes. Aliquots (150 μL) of the positive control reaction mixture were withdrawn at 0 and 30 minutes. Test compound(s) and kynuramine samples were immediately combined with 150 μL of ice-cold 100% acetonitrile containing 0.1% formic acid and internal standard (0.2 μM metoprolol) to terminate the reaction. The samples were then mixed and centrifuged to precipitate proteins. All samples were assayed by LC-MS/MS. The peak area response ratio (PARR) of analyte to internal standard at each time point was compared to the PARR at time 0 to determine the percent remaining at each time point. Half-lives were calculated using GraphPad software, fitting to a single-phase exponential decay equation.

### Reaction Composition:

| | | |
|---|---|---|
| hrMAO | 0.02 | mg/mL |
| Potassium Phosphate, pH 7.4 | 100 | mM |
| Magnesium Chloride | 5 | mM |
| Test compound (each, if co-dosed) | 1 | μM |

### In Vivo 5-HT2 Activity and Pharmacokinetic Screening

Protocols were followed according to Canal C E, Morgan D. Head-twitch response in rodents induced by the hallucinogen 2,5-dimethoxy-4-iodoamphetamine: a comprehensive history, a re-evaluation of mechanisms, and its utility as a model. Drug Test Anal. 2012 July-August; 4(7-8):556-76; Saraf, T. S., Felsing, D. E., Armstrong, J. L., Booth, R. G., and Canal, C. E., 2021, Evaluation of lorcaserin as an anticonvulsant in juvenile Fmr1 knockout mice, Epilepsy Res 175, 106677. Mice were housed in standard laboratory cages with ad libitum access to food and water and were acclimated to the vivarium for at least one week prior to testing in a procedure room. In a typical experiment, mice were injected 3 mg/kg of test article subcutaneously. Mice were placed immediately thereafter in a clear, polycarbonate box (46×20×20 cm), and HTRs were counted at various time points after administration using hand-held tally counters. In a separate experiment, plasma and brain samples were collected at 15 and 160 minutes after administration of test compounds to measure plasma and brain concentrations. Quantification was performed using LC-MS/MS method (LLOQ, 9 nM).

### Results In Vitro and In Vivo Pharmacodynamics and Pharmacokinetics Screening

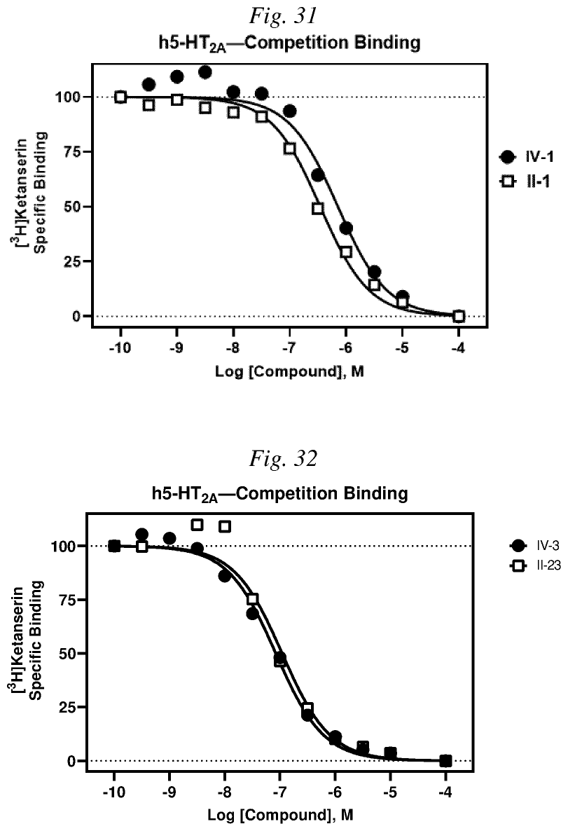

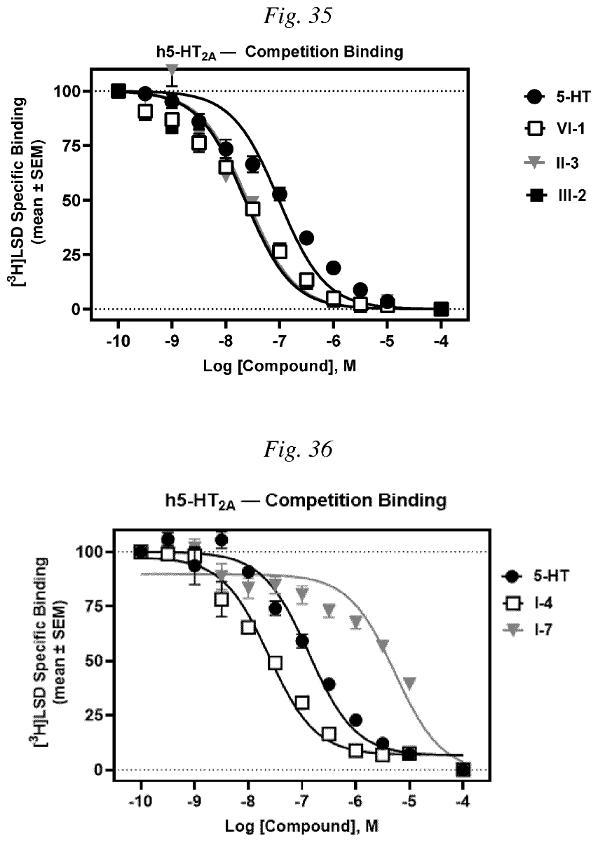

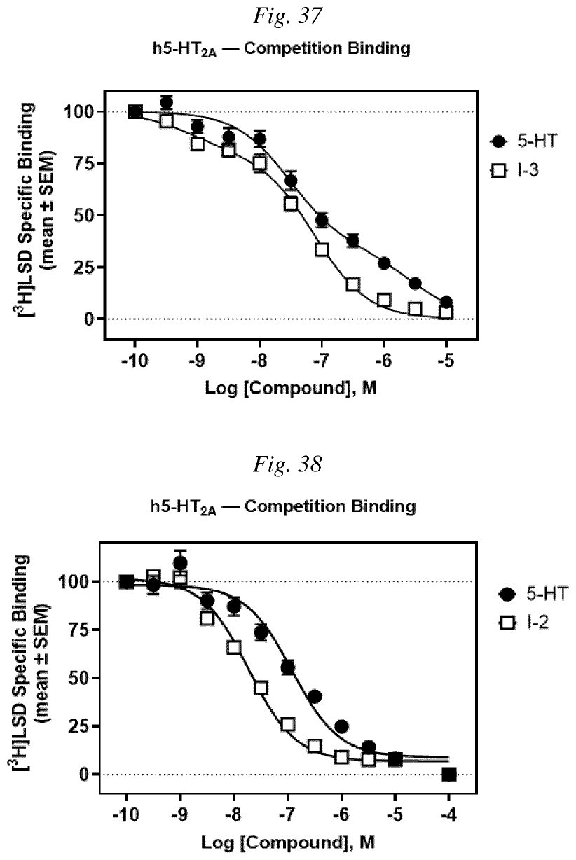

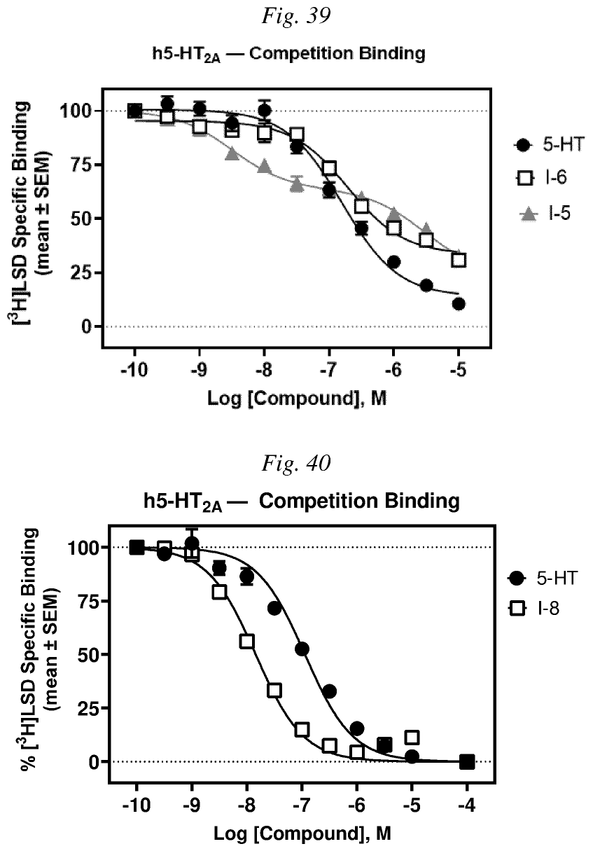

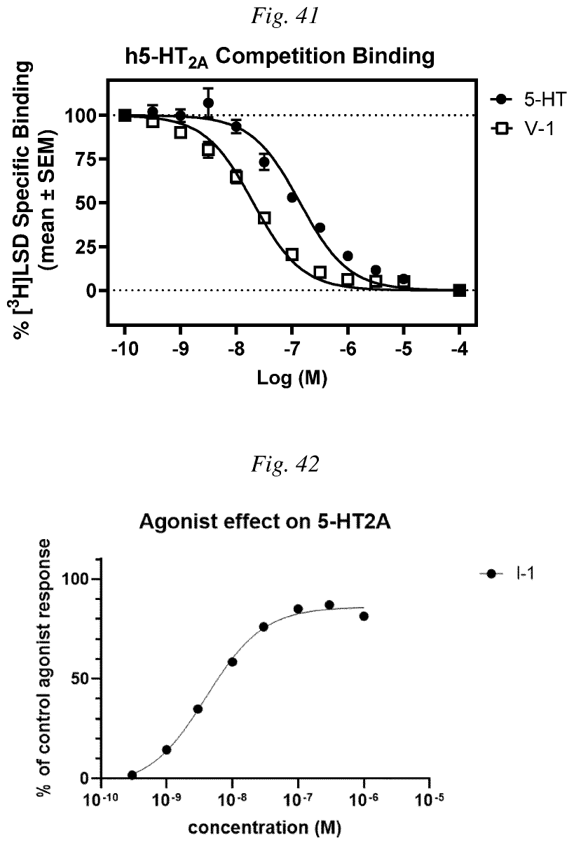

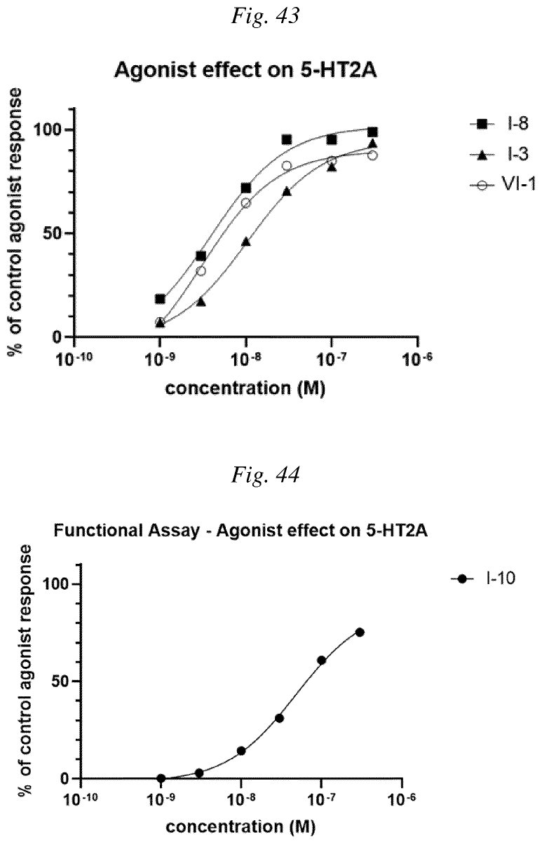

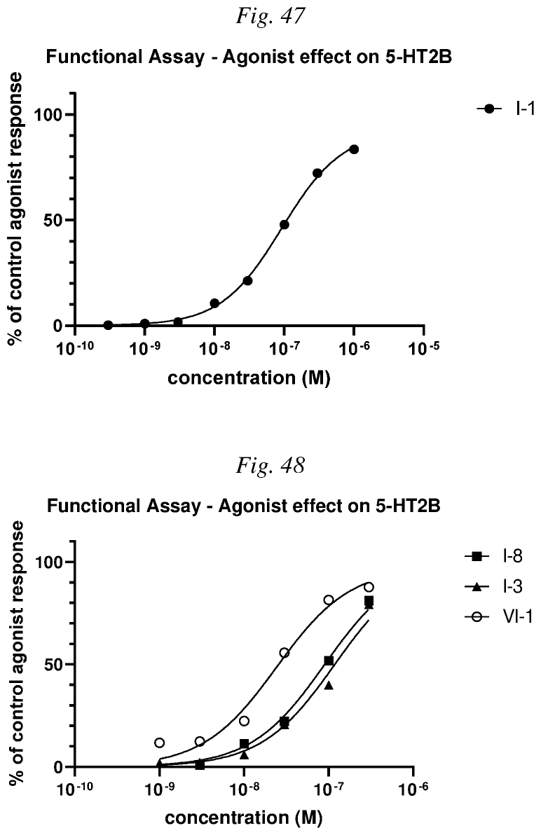

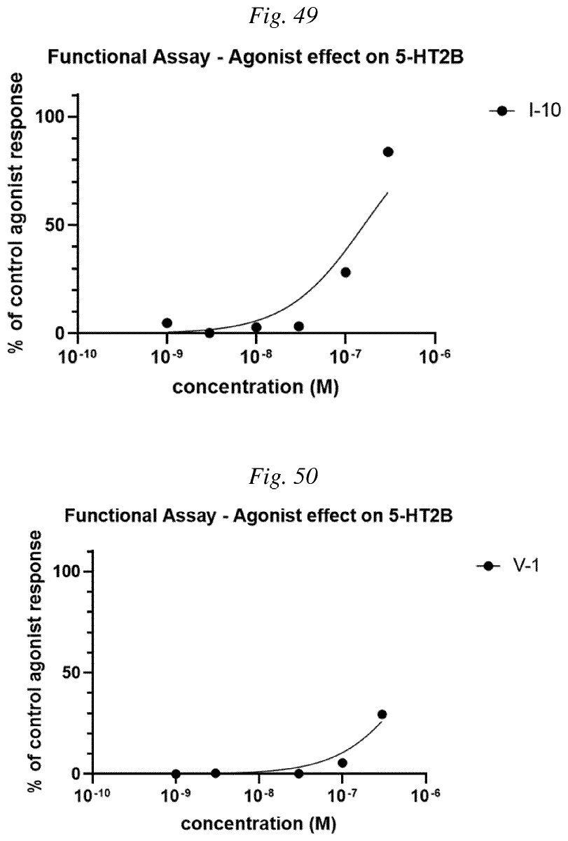

A summary of screening results is shown in Tables 2-5, and graphically in FIGS. 31-51.

TABLE 2

| | | 5-HT_{2A}, | | 5-HT_{2B}, | 5-HT_{2C}, |
|---|---|---|---|---|---|
| Compound | LogP | Kᵢ (nM) | | Kᵢ (nM)*** | Kᵢ (nM) |
| Reference | 1.55 | 296** | | — | — |
| Compound 3 | | | | | |
| Reference | 2.03 | 25** | | — | 35**** |
| Compound 4 | | | | | |
| IV-1 | 1.55 | 357** | | — | — |
| II-1 | 1.55 | 174** | | — | — |
| IV-3 | 2.03 | 42** | | — | 23**** |
| II-23 | 2.03 | 55** | | — | — |
| I-1 | 3.2 | 2.5^{a} | (54^{b})** | 6.7 | 0.55*** |
| | | 0.35*** | | — | — |
| II-10 | 3.2 | 0.54^{a} | (35^{b})** | — | — |
| I-13 | 2.68 | 150* | | — | — |
| II-4 | 2.68 | 202* | | — | — |
| VI-1 | 2.81 | 9.9* | | — | — |
| II-3 | 2.81 | 11* | | — | — |
| III-2 | 2.81 | 10* | | — | — |
| I-4 | 1.84 | 12* | | — | — |
| I-3 | 2.15 | 0.4^{a} | (44^{b})* | — | — |

| | | | | |
|---|---|---|---|---|
| I-2 | 3.53 | 8.5* | — | — |
| I-7 | 1.28 | 2601* | — | — |
| I-6 | 1.58 | n.c. | — | — |
| I-5 | 3.24 | n.c. | — | — |
| I-8 | 2.25 | 6.3* | — | — |
| V-1 | 4.44 | 10* | 79* | 0.98^{a}(74.9^{b})* |
| I-11 | 1.76 | 1.3* | — | — |

^{a}Kᵢ (nM) at the high-affinity active state
^{b}Kᵢ (nM) at the low-affinity inactive state
n.c. = not calculated
*Radioligand: [³H]LSD;
*Radioligand: [³H]ketanserin;
***Radioligand: [¹²⁵](±)DOI;
****Radioligand: [³H]mesulergine

TABLE 3

| | | | | | | | | | Inhibition | Inhibition |
|---|---|---|---|---|---|---|---|---|---|---|
| | | 5-HT_{2A} | | 5-HT_{2B} | | 2A/2B | 5-HT_{2C} | | of binding | of binding |
| | | EC₅₀ | 5-HT_{2A} | EC₅₀ | 5-HT_{2B} | EC₅₀ | EC₅₀ | 5-HT_{2C} | at 5HT_{1A}*, | at 5HT_{1A}*, |
| Compound | LogP | (nM)^{a} | Eₘₐₓ^{a} | (nM)^{a} | Eₘₐₓ^{a} | Selectivity | (nM)^{a} | Eₘₐₓ^{a} | 100 nM | 1 uM |
| I-1 | 3.2 | 4.1 | 86% | 88 | 84% | 21.5 | 7.3 | 99% | 55% | 90% |
| VI-1 | 2.81 | 4.2 | 87% | 25 | 87% | 6 | — | — | — | — |
| I-3 | 2.15 | 10 | 93% | 160 | 100%^{b} | 16 | — | — | 10% | 63% |
| I-8 | 2.25 | 5.3 | 99% | 67.9 | 87% | 12.8 | — | — | 38% | 84% |
| I-10 | 2.47 | 89 | 76% | 240 | 84% | 2.7 | — | — | — | — |
| V-1 | 4.44 | 35.7 | 31% | >1000 | 100%^{b} | n.c. | — | — | — | — |

^{a}IP1 assay
^{b}constrained to 100%
n.c. = not calculated
*Radioligand: [³H]8-OH-DPAT

TABLE 4

| | | HTR | HTR | Plasma, | Plasma, | Brain | Brain, | brain/plasma |
|---|---|---|---|---|---|---|---|---|
| | | 15-25 | 160-170 | 15 min | 160 min | 15 min | 160 min | ratio, |
| Compound | LogP | min | min | (μM) | (μM) | (μM) | (μM) | 160 min |
| Reference | 1.55 | 19 ## | — | — | — | — | — | — |
| Compound | | | | | | | | |
| 3 | | | | | | | | |
| II-1 | 1.55 | 21 ## | — | — | — | — | — | — |
| I-1 | 3.2 | 27.4 # | 30.4 ### | 0.5 (10 | 0.3 | 1.3 (10 | 9.6 | 32.4 |
| | | | | min) | | min) | | |
| II-10 | 3.2 | 25.6 ## | — | 0.5 (10 | 0.36 | 1.3 (10 | 8.8 | 24.4 |
| | | | | min) | | min) | | |
| I-13 | 2.68 | 2.7 | — | — | — | — | — | — |
| VI-1 | 2.81 | 24.5 | 17.7 | 0.25 | 0.08 | 2.9 | 4.6 | 57.1 |
| I-3 | 2.15 | 5 | 2.5 | 0.7 | 0.055 | 0.67 | 0.51 | 9.3 |
| I-8 | 2.25 | 33.4 | 17.4 | 0.3 | 0.03 | 1.9 | 2.2 | 74 |
| I-10 | 2.47 | 3.4 | 2.7 | 0.9 | 0.10 | 0.9 | 1.1 | 12.3 |
| V-1 | 4.44 | 3 #### | 4.42 | — | — | — | — | — |
| I-11 | 1.76 | 24.5 #### | 16.7 | — | — | — | — | — |

# Calculated using 0-5 min;
## Measured 0-15 min;
### Calculated using 3 min time bin;
#### Calculated using 0-30 min

TABLE 5

| | | RLM, t_{1/2} | HLM, t_{1/2} | MsLM, t_{1/2} | DLM, t_{1/2} | MAO-A, t_{1/2} |
|---|---|---|---|---|---|---|
| Compound | LogP | (min) | (min) | (min) | (min) | (min) |
| Reference | 1.55 | 5.2 | — | — | — | >120 |
| Compound 3 | | | | | | |
| Reference | 2.03 | 7.7 | — | — | — | 87.7 |
| Compound 4 | | | | | | |
| IV-1 | 1.55 | 5.3 | — | — | — | — |
| II-1 | 1.55 | 5.7 | — | — | — | — |
| IV-3 | 2.03 | 11.1 | — | — | — | 102 |
| II-23 | 2.03 | 21.4 | — | — | — | >120 |
| I-1 | 3.2 | 55.2 | >120 | >120 | >120 | >120 |
| II-10 | 3.2 | 59.3 | >120 | >120 | >120 | >120 |
| I-13 | 2.68 | 7.8 | — | — | — | >120 |
| II-4 | 2.68 | 7.5 | — | — | — | >120 |
| VI-1 | 2.81 | 2.4 | — | — | — | — |
| II-3 | 2.81 | 2.4 | — | — | — | — |
| III-2 | 2.81 | 2.5 | — | — | — | — |
| I-4 | 1.84 | 11 | — | — | — | 45 |
| I-3 | 2.15 | 111 | — | — | — | — |
| I-2 | 3.53 | 24.8 | — | — | — | 30.9 |
| I-7 | 1.28 | >120 | — | — | — | >120 |
| I-6 | 1.58 | >120 | — | — | — | — |
| I-5 | 3.24 | >120 | — | — | — | — |

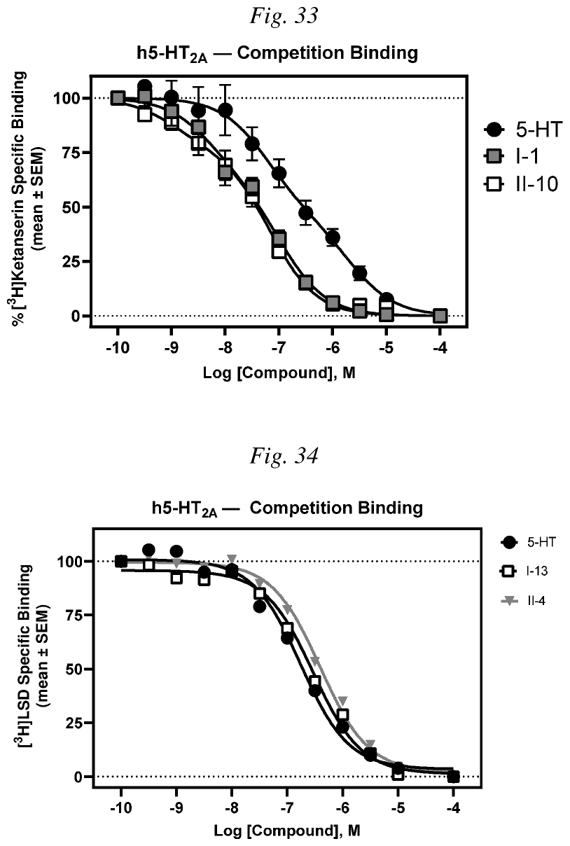

As shown in Tables 2-4 and FIG. 33, it was found that compounds I-1 and TI-10 have superior affinity at 5-HT_{2A} receptors compared to the reference compounds and a significantly higher LogP value. The latter translated to a brain:plasma ratio of 24 to 32-fold 160 minutes after system injection of 3 mg/kg, showing that both compounds have high brain penetration for targeting brain receptors, with the potential to decrease peripheral liabilities.

Fluoro-substituted thioalkoxy analogs 1-2, 1-3, 1-4, 1-8, and I-10 demonstrated high affinity for serotonin 5-HT_{2A} receptors (see also FIG. 40 for a competition binding graph related to compound 1-8). It was further found that both compounds I-1 and II-10 are pronounced agonists of serotonin 5-HT_{2A}Rs (see e.g., FIG. 42)—there were no significant differences in affinity between deuterated and non-deuterated compounds, suggesting deuteration does not alter pharmacodynamics.

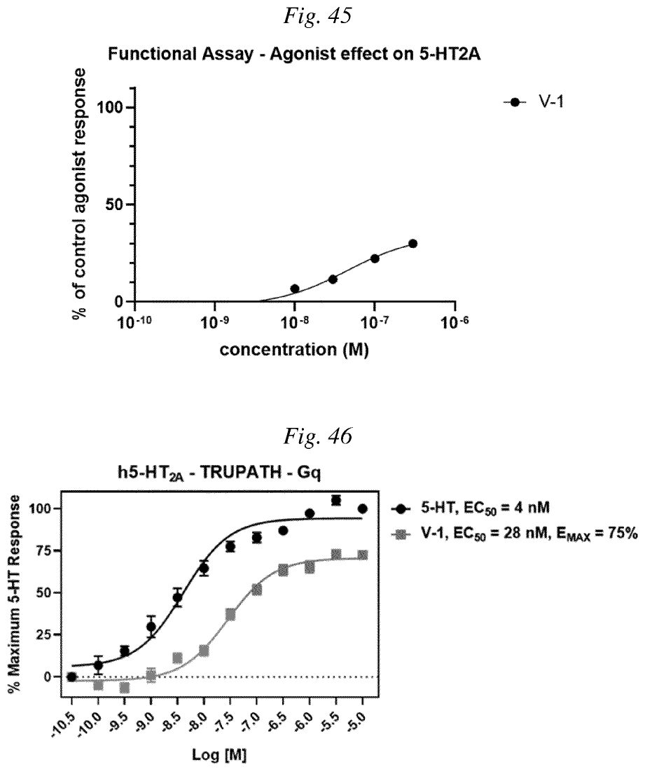

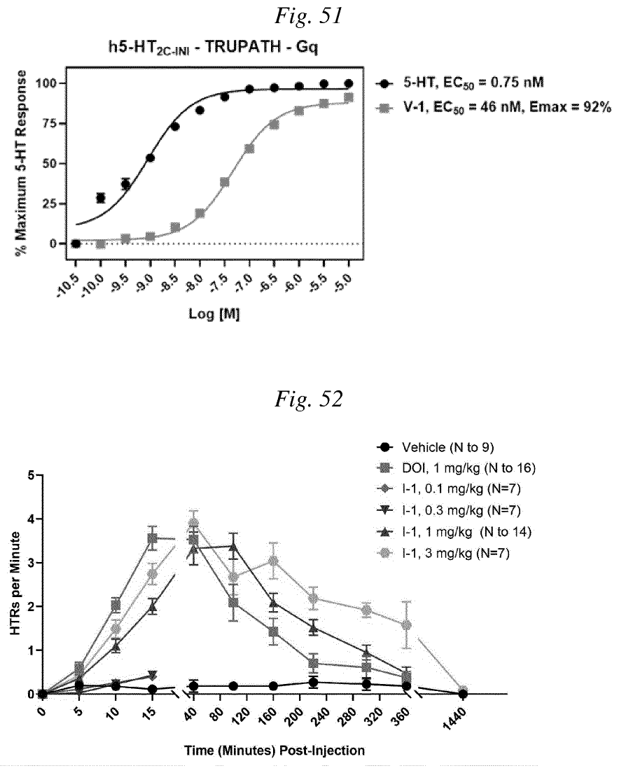

Compounds I-5, I-6, I-7, I-11, I-13, II-1, II-3, II-4, II-23, III-2, IV-1, IV-3, V-1, VI-1 were also synthesized and tested for pharmacodynamic and pharmacokinetic activity. The 5-HT_{2A}R activity of most of these compounds were like I-1, with Kᵢ values lower than 150 nM, while I-5, I-6, I-7, II-1, II-4, and IV-1 showed affinity higher than 150 nM. Notably, I-13 had appreciable affinity at 5-HT_{2A}R but it did not elicit an HTR like the other compounds. An unexpected discovery was that V-1 was a potent 5-HT_{2A}R agonist (Kᵢ=10 nM, EC₅₀=28 (TRUPATH) to 35 (IP1) nM, and Eₘₐₓ=31 (IP11) to 75% (TRUPATH, relative to 5-HT) (see FIGS. 45 and 46), and a potent 5-HT_{2C}R agonist (EC₅₀=46 nM, Eₘₐₓ=92%, relative to 5-HT)(TRUPATH) (see FIG. 51). Despite potent 5-HT_{2A/2C}R agonist activity, at 3 mg/kg, V-1 elicited an HTR to a much lower extent than DOI (1 mg/kg) and other compounds in this series, e.g., I-1. These data provide evidence that V-1 is 5-HT_{2A}R agonist that may have a low propensity to cause psychedelic effects.

Alkyne analog I-22 and nitrile analog I-25 were also synthesized and tested for 5-HT_{2C}R agonist activity, and both compounds were shown to have 5-HT_{2C}R potency with EC₅₀ values of 13.1 nM and 35.6 nM, respectively, and near-full agonism (Eₘₐₓ of 84% for both compounds, relative to 5-HT, TRUPATH).

### In Vitro Pharmacodynamics of Compound I-1

Off-target activity at 79 proteins including ion channels, G-protein coupled receptors (GPCRs), and transporters was tested for compound I-1. ‘Percent displacement of radioligand’ (or ‘inhibition’ or ‘competition’ for orthosteric binding sites) results from a single 10 μM test concentration of I-1 in comparison to DOI are shown in Table 6. I-1 (10 μM) produced >80% inhibition (defined here as “appreciable affinity”) at 5-HT_{1A}, 5-HT_{1B}, 5-HT_{2A}, 5-HT_{2B}, 5-HT_{2C}, 5-HT₆, and 5-HT₇R subtypes, rat α2-adrenergic receptors, human acetylcholine muscarinic (mACh) M1, M4, M5Rs and human L-type Ca2+ channels (verapamil site). At 10 μM, I-1 inhibited 97% of radioligand bound to rat alpha2 receptors. Thus, its affinity was tested at each of the human alpha2 receptors; its Kᵢ at alpha2A, alpha2B, and alpha2C receptors ([³H]rauwolscine-labeled) was 66, 1024, and 108 nM, respectively. Further testing showed that I-1 was a partial agonist at human alpha2A receptors with an EC₅₀ of 165 nM.

The receptor binding (10 μM affinity screens or binding potencies “Kᵢ” of compounds at targets) and for 5-HT₂ receptor subtypes, the receptor function (EC₅₀ and Eₘₐₓ), show that racemic DOI ((±)-DOI, also referred to herein as “DOI”), a prototypical phenethylamine serotonergic psychedelic, and I-1 have similar pharmacodynamic properties, with a few surprising, notable exceptions (Table 6).

5-HT₂ Receptor Activity. DOI and I-1 bound to and activated each of the human serotonin 2 (5-HT₂) receptor subtypes with high potency, i.e., at nM concentrations. The DOI data corroborate published reports [Canal C. E.; Morgan, D., Head-twitch response in rodents induced by the hallucinogen 2,5-dimethoxy-4-iodoamphetamine: a comprehensive history, a re-evaluation of mechanisms, and its utility as a model. Drug Test Anal 2012, 4 (7-8), 556-76. Ray, T. S., Psychedelics and the human receptorome. PLoS One 2010, 5 (2), e9019]. I-1 displayed measurable pharmacological selectivity for 5-HT_{2A} and 5-HT_{2C} over 5-HT_{2B}. Its binding potencies (Kᵢs determined via agonist ([¹²⁵I]DOI) radioligand binding assays) at 5-HT_{2A}, 5-HT_{2B}, and 5-HT_{2C}Rs were 0.35, 6.7, and 0.55 nM, respectively, showing it has ˜19-fold binding selectivity for 5-HT_{2A} over 5-HT_{2B}Rs and ˜12-fold binding selectivity for 5-HT_{2C} over 5-HT_{2B}Rs. Similarly, its activation potencies (EC₅₀'s determined in functional assays measuring inositol phosphate 1 (IP1) production/Gαq signaling) at 5-HT_{2A}, 5-HT_{2B}, and 5-HT_{2C}Rs were 4.1, 88, and 7.3 nM, showing it has ˜21-fold activation selectivity for 5-HT_{2A} over 5-HT_{2B}Rs and ˜12-fold activation selectivity for 5-HT_{2C} over 5-HT_{2B}Rs. In comparison, DOI had 5.4-fold and 7.3-fold binding and activation selectivity, respectively, for 5-HT_{2A} over 5-HT_{2B}, and 3.1-fold and 2-fold binding and activation selectivity, respectively, for 5-HT_{2C} over 5-HT_{2B}. DOI and I-1 were near full agonists at 5-HT_{2A}Rs (Eₘₐₓ values ˜86% relative to 5-HT), near full agonists at 5-HT_{2B}R, (Eₘₐₓ values ˜98% and ˜84%, respectively, relative to 5-HT), and full agonists at 5-HT_{2C}Rs (Eₘₐₓ values ˜108% and ˜99%, respectively, relative to 5-HT).

Off-target Affinity Screens at 10 μM. “Percent displacement of radioligand” (or “inhibition” or “competition” for orthosteric binding sites) tests from a single 10 μM test concentration of DOI and I-1 were performed at all other proteins tested. These 10 μM tests are affinity screens. From the 10 μM tests, one can deduce that the test compounds do or do not bind well to the relevant receptors, but do not provide specific data on the actual affinity (i.e., Kᵢ, K_{d}, or K_{b}) or function (i.e., EC₅₀, IC₅₀, Eₘₐₓ, or IMAX) at canonical signaling pathways of the compounds at the receptors. For example, for rat α2-adrenergic receptors (using rat cerebral cortex tissue, subtype not identified), at 10 μM, I-1 inhibited 97.3%, and DOI inhibited 81.9% of the antagonist radioligand bound to the receptors. Thus, one can conclude I-1 and DOI have affinity for rat α2-adrenergic receptors, and that I-1 has a higher affinity than DOI at them. At human cannabinoid 1 (CB1) receptors, however, DOI and I-1 inhibited less than 50% of radioligand bound to them, and therefore it can be concluded they do not bind (“no binding,” NB) meaningfully at CB1 receptors. Full dose-response tests, i.e., tests of a full set of concentrations (e.g., 1, 3, 10, 30, 100, 300, 1,000, 3,000, and 10,000 nM concentrations, tested in triplicates) are typically assessed to determine and report Kᵢ (affinity) and EC₅₀ plus Eₘₐₓ (function) properties.

Shared Off-Targets of DOI and I-1. The off-targets shared by DOI and I-1, wherein each compound inhibited ≥80% radioligand binding in 10 μM screens, were rat α2-adrenergic receptors (subtype α2A, α2B, and/or α2C not identified) and human serotonin 5-HT_{1A} receptors. See Table 6 for exact inhibition numbers.

Divergent Off-Targets of DOI and I-1 show Unexpected Effects of I-1. There was a substantial divergence between DOI and I-1 in their binding to a relatively small number of other receptors. DOI inhibited ≥80% radioligand binding to human β2-adrenergic receptors at 10 μM. I-1, distinctly, inhibited ≥80% radioligand binding to human serotonin 5-HT_{1B}, 5-HT₆, 5-HT₇, muscarinic M1, M4, M5 receptors and L-type Ca2+ channels (verapamil site).

As is well-described in the literature, DOI was a highly selective, potent 5-HT_{2A}, 5-HT_{2B}, and 5-HT_{2C}R agonist. Even at 10 μM, it only inhibited ˜82% of radioligand binding to “three” other targets (human 5-HT_{1A} and β2 and rat α2 (subtype not identified)), suggesting it has μM affinity at them. I-1 was also a potent 5-HT₂ agonist, but at 10 μM, it inhibited >80% radioligand binding to nine other targets (see Table 6). I-1 inhibited 97.3% of radioligand binding to rat α2-adrenergic receptors (subtype not identified) and 98.1% of radioligand binding to human serotonin 5-HT_{1A} receptors. I-1 indeed demonstrated functional potency at 5-HT_{1A} receptors with an EC₅₀ of 88 nM signifying at least partial agonist activity. I-1 inhibited <90% of radioligand binding to the other seven off-targets.

5-HT_{1A} agonists have anxiolytic and other beneficial properties, and α2-adrenergic agonists effectively treat opioid withdrawal, pain, ADHD, and hypertension. In conclusion, I-1 has unique pharmacodynamic properties, with distinct selectivity and activity at a subset of GPCRs.

TABLE 6

| Receptor (species, h = human; r = | | |
|---|---|---|
| rat; m = mouse; gp = guinea pig) | DOI (HCl) | I-1 (HCl) |
| *5-HT_{2A} (h) | Kᵢ = 0.28 nM ([¹²⁵I]DOI) | Kᵢ = 0.35 nM ([¹²⁵I]DOI) |
| | EC₅₀ = 2.2 nM | EC₅₀ = 4.1 nM |
| | Eₘₐₓ ~86% relat. 5-HT | Eₘₐₓ ~86% relat. 5-HT |
| | (IP1 assay) | (IP1 assay) |
| *5-HT_{2B} (h) | Kᵢ = 1.5 nM ([¹²⁵I]DOI) | Kᵢ = 6.7 nM ([¹²⁵I]DOI) |
| | EC₅₀ = 16 nM | EC₅₀ = 88 nM |
| | Eₘₐₓ ~98% relat. 5-HT | Eₘₐₓ ~84% relat. 5-HT |
| | (IP1 assay) | (IP1 assay) |
| *5-HT_{2C} (h) | Kᵢ = 0.48 nM ([¹²⁵I]DOI) | Kᵢ = 0.55 nM ([¹²⁵I]DOI) |
| | EC₅₀ = 8 nM | EC₅₀ = 7.3 nM |
| | Eₘₐₓ ~108% relat. 5-HT | Eₘₐₓ~99% relat. 5-HT |
| | (IP1 assay) | (IP1 assay) |
| *5-HT_{1A} (h) | 81.3% inhibition at 10 μM | 98.1% inhibition at 10 μM |
| | (agonist radioligand) | (agonist radioligand) |
| *Adrenergic α2 (subtype not | 81.9% inhibition at 10 μM | 97.3% inhibition at 10 μM |
| identified) (rat cortex) | (antagonist radioligand) | (antagonist radioligand) |
| *5-HT₆ (h) | 64.4% inhibition at 10 μM | 89.8% inhibition at 10 μM |
| | (agonist radioligand) | (agonist radioligand) |
| *Muscarinic M1 (h) | 71.8% inhibition at 10 μM | 88.1% inhibition at 10 μM |
| | (antagonist radioligand) | (antagonist radioligand) |
| *Muscarinic M4 (h) | 75.7% inhibition at 10 μM | 86.3% inhibition at 10 μM |
| | (antagonist radioligand) | (antagonist radioligand) |
| *5-HT₇ (h) (subtype “a” or “b” | 58.4% inhibition at 10 μM | 86.2% inhibition at 10 μM |
| not specified) | (agonist radioligand) | (agonist radioligand) |
| *Calcium Channel L-Type, | 55.5% inhibition at 10 μM | 84.6% inhibition at 10 μM |
| Phenylalkylamine, verapamil site | (antagonist radioligand) | (antagonist radioligand) |
| (rat cortex) | | |
| *Muscarinic M5 (h) | 62.6% inhibition at 10 μM | 83.6% inhibition at 10 μM |
| | (antagonist radioligand) | (antagonist radioligand) |
| *5-HT_{1B} (h) | 52.7% inhibition at 10 μM | 82.9% inhibition at 10 μM |
| | (antagonist radioligand) | (antagonist radioligand) |
| *Muscarinic M3 (h) | 60.4% inhibition at 10 μM | 79% inhibition at 10 μM |
| | (antagonist radioligand) | (antagonist radioligand) |
| *Dopamine D3 (h) | NB | 70.4% inhibition at 10 μM |
| | | (antagonist radioligand) |
| *Na+ channel (site 2) | 53.5% inhibition at 10 μM | 68.4% inhibition at 10 μM |
| (rat cortex) | (antagonist radioligand) | (antagonist radioligand) |
| *sigma (non-selective) (h) | 61.7% inhibition at 10 μM | 64.1% inhibition at 10 μM |
| | (agonist radioligand) | (agonist radioligand) |
| *Dopamine D1 (h) | NB | 57% inhibition at 10 μM |
| | | (antagonist radioligand) |
| *Adrenergic β2 (h) | 82.2% inhibition at 10 μM | 52.2% inhibition at 10 μM |
| | (antagonist radioligand) | (antagonist radioligand) |
| 5-HT transporter (h) | 76.2% inhibition at 10 μM | NB |
| | (antagonist radioligand) | |
| μ-opioid (h) | 65% inhibition at 10 μM | NB |
| | (agonist radioligand) | |
| Histamine H1 (h) | 64.6% inhibition at 10 μM | NB |
| | (antagonist radioligand) | |
| 5-HT_{1D} | NR | NR |
| 5-HT_{1E} | NR | NR |
| 5-HT_{1F} | NR | NR |
| 5-HT₃ (h) | NB (antagonist) | NB (antagonist) |
| 5-HT₄ | NR | NR |
| 5-HT₅ₐ (h) | NB (agonist) | NB (agonist) |
| Adenosine A1 (h) | NB | NB |
| Adenosine A2A (h) | NB | NB |
| Adenosine A3 (h) | NB | NB |
| α1 (non-selective) (r) | NB | NB |
| Adrenergic β1 (h) | NB | NB |
| Angiotensin AT1 (h) | NB | NB |
| Angiotensin AT2 (h) | NB | NB |
| BZD (central) (rat cortex) | NB | NB |
| BZD (peripheral) (rat heart) | NB | NB |
| BB (nonselective) (rat cortex) | NB | NB |
| B2 (h) | NB | NB |
| Calcitonin Gene-Related Peptide | NB | NB |
| CGRP1 (h) | | |
| Cannabinoid CB1 (h) | NB | NB |
| Cholecystokinin CCK1 (CCKA) | NB | NB |
| (h) | | |
| Cholecystokinin CCK2 (CCKB) | NB | NB |
| (h) | | |
| D2S (h) | NB | NB |
| D4.4 (h) | NB | NB |
| D5 (h) | NB | NB |
| Endothelin ETA (h) | NB | NB |
| Endothelin ETB (h) | NB | NB |
| GABA (non-selective) | NB | NB |
| (rat cortex) | | |
| Galanin GAL1 (h) | NB | NB |
| Galanin GAL2 (h) | NB | NB |
| Platelet-Derived Growth Factor | NB | NB |
| (PDGF) (m) | | |
| CXCR2 (Interleukin-8B) (h) | NB | NB |
| Chemokine CCR1 (h) | NB | NB |
| TNF-α (h) | NB | NB |
| Histamine H2 (h) | NB | NB |
| Melanocortin MC4 (h) | NB | NB |
| Melatonin MT1 (h) | NB | NB |
| Muscarinic M2 (h) | NB | NB |
| Tachykinin NK1 (h) | NB | NB |
| Tachykinin NK2 (h) | NB | NB |
| Tachykinin NK3 (h) | NB | NB |
| Neuropeptide Y Y1 (h) | NB | NB |
| Neuropeptide Y Y2 (h) | NB | NB |
| Neurotensin NT1 (h) | NB | NB |
| δ-opioid (h) | NB | NB |
| κ-opioid (h) | NB | NB |
| Orphanin ORL1 (h) | NB | NB |
| PAC1 (Pituitary adenylate | NB | NB |
| cyclase-activating polypeptide, | | |
| PACAP) (h) | | |
| PPARγ (h) | NB | NB |
| PCP (rat cortex) | NB | NB |
| Prostaglandin EP2 (h) | NB | NB |
| Prostaglandin EP4 (h) | NB | NB |
| Prostacyclin IP (PGI2) (h) | NB | NB |
| P2X (ATP) (rat urinary bladder) | NB | NB |
| P2Y (ATP) (rat cortex) | NB | NB |
| Somatostatin SST (non- | NB | NB |
| selective) (m) | | |
| Glucocorticoid GR (h) | NB | NB |
| VPAC1 (vasoactive intestinal | | |
| peptide, VIP1) (h) | | |
| Vasopressin V1a (h) | NB | NB |
| Potassium KV channel | NB | NB |
| (rat cortex) | | |
| Calcium SKCa channel | NB | NB |
| (rat cortex) | | |
| Chloride Cl— channel (GABA- | Decreased binding by ~38% | Decreased binding by ~48% |
| gated) (rat cortex) | | |
| norepinephrine transporter (h) | NB | NB |
| dopamine transporter (h) | NB | NB |

Affinity profiles of DOI and I-1 and receptor function profiles of the same compounds at human 5-HT_{2A}, 5-HT_{2B}, and 5-HT_{2C} receptors. DOI and I-1 were also tested for binding to targets at 10 μM, except for 5-HT₂Rs, where full dose-response studies and full dose-response functional assays (IP1) were performed to calculate Kᵢ, EC₅₀, and Eₘₐₓ values.
*represents significant test compound binding as defined by >50% inhibition of radioligand binding

### In Vivo Pharmacodynamics of Compound I-1

5-HT₂ agonist activity and relative duration of action in vivo of test compounds were assessed using the HTR assay in male, adult C57BL/6J mice (Jackson Laboratories). Mice were housed in standard laboratory cages with ad libitum access to food and water, and were acclimated to the vivarium for at least one week prior to testing in a procedure room. On the day of testing, mice were acclimated to the procedure room in their home cages for ≥60 min before administration of test compounds. Mice were injected subcutaneously, placed immediately thereafter in a clear, polycarbonate box (46×20×20 cm), and HTRs were counted at 5, 10, 15, 40, 120, 160, 200, 240, 280, 320, 360, and 1440 min, using hand-held tally counters, by two trained observers who were blind to treatment.

In another experiment, C57BL/6J mice were moved to the behavior room 30 min prior to administration of the test compound. Test compound was administered either s.c. or p.o. at 1 to 10 mg/kg immediately prior to testing and HTR activity was assessed from 1-30 min and again from 120-150 by blind experienced testers in real-time.

As shown in FIG. 52, compound I-1 elicited a robust dose-dependent HTR, suggesting in vivo engagement and activation of cortical 5-HT₂Rs; its HTR-eliciting effect persisted longer than the 5-HT₂ agonist psychedelic phenethylamine (±)-2,5-dimethoxy-4-iodoamphetamine (DOI) administered subcutaneously at 1 mg/kg. I-1 produced a dose-dependent increase in the HTR in mice.

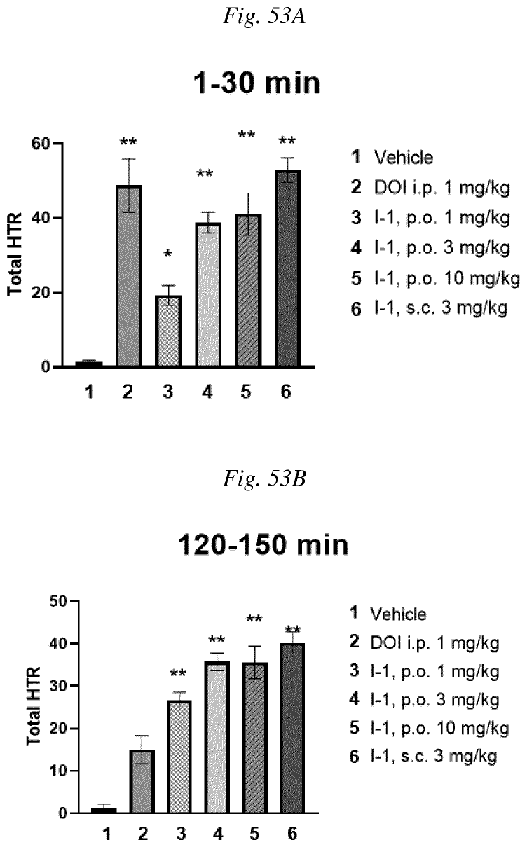

It was also shown that I-1 is orally active. Oral administration of I-1 at doses of 1, 3 and 10 mg/kg elicited the HTR in C57BL/6J mice, with effects that mirrored those observed after s.c administration (FIGS. 53A and 53B). Furthermore, orally administered I-1 elicited the HTRs for a greater duration than DOI. These data provide supportive evidence that I-1 is orally active and engages central 5-HT_{2A}Rs with durable effects in a dose-dependent manner.

### Pharmacokinetic (PK) Profiles of I-1 in Rats and Mice

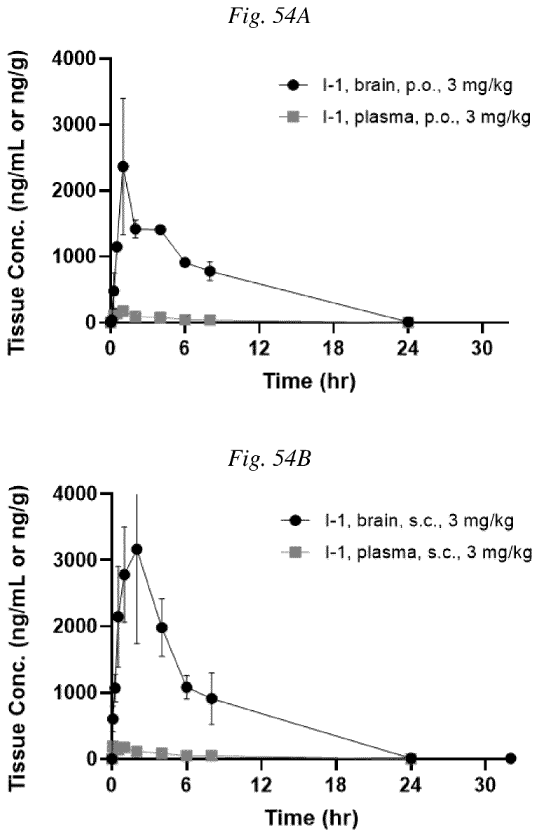

PK Profile in Mice. To evaluate the full PK profile for I-1 in mice, plasma and brain samples were determined in male C57BL/6J mice following subcutaneous (s.c.) and oral (p.o.) administration at 3 mg/kg (see Table 7 and FIGS. 54A and 54B). In a typical experiment, blood was collected via cardiac puncture (N=3 animals per time point, 9 time points) up to 24 hours post dose. Plasma and brain concentrations were determined using LC-MS/MS and PK parameters were determined using a non-compartmental analysis (NCA) using Phoenix WinNonlin Pro (v8.3.1, Pharsight, Inc, USA) software.

It was shown that in mice, independent of route of administration, I-1 was rapidly absorbed into systemic circulation with levels of I-1 observed at the first timepoint taken (0.083 hr). I-1 distribution into the central nervous system was rapid with levels also observed in brain tissue at the first timepoint taken. Maximum mean plasma concentrations (Cₘₐₓ) of I-1 after p.o. and s.c. administration at 3 mg/kg were 175 ng/mL at 1 hr and 188 ng/mL at 0.08 hr, respectively. Mean terminal half-life was 3.30 hr and 3.92 hr after p.o. and s.c. administration, respectively, with corresponding area under the curve (AUC_{INF}) values of 784 and 1002 hr*ng/mL, respectively.

In comparison, the maximum mean brain concentrations occurred at 1 and 2 hr, with peak brain I-1 values of 2366 ng/g and 3165 ng/g after p.o. and s.c. administration, respectively. Mean terminal half-life was 2.73 hr and 3.15 hr post p.o. and s.c. administration, respectively. The mean AUC_{INF} values were 16171 hr*ng/g and 22487 hr*ng/g p.o. and s.c. administration, respectively.

In summary, at a dose of 3 mg/kg, I-1 was absorbed quickly into the mouse systemic circulation after a s.c. administration with peak plasma concentrations occurring at 0.08 hr. In contrast, peak plasma concentration occurred at 1 hr post p.o. administration. The relative plasma oral bioavailability compared to the subcutaneous was 78.3%. Independent of route of administration, brain peak levels occurred between 1 and 2 hr. I-1 concentration were 25.8- to 30.7-fold higher (AUC_{INF}) in brain compared to plasma. Clearance of compound I-1 from plasma and brain was similar. Across route of administration and tissue, the elimination T_{1/2} ranged from 2.73 to 3.92 hours.

TABLE 7
Mean pharmacokinetic parameters following oral and subcutaneous
administration of compound I-1 (3 mg/kg) in C57BL/6J mice

| | Compound I-1 | | | |
|---|---|---|---|---|
| Matrix | Plasma | | Brain | |
| Parameters/Route | p.o. | s.c. | p.o. | s.c. |
| Dose (mg/kg) | 3 | 3 | 3 | 3 |
| Cₘₐₓ (ng/mL) | 175 | 188 | 2366 | 3165 |
| Tₘₐₓ (hr) | 1 | 0.08 | 1 | 2 |
| T_{1/2} (hr) | 3.30 | 3.92 | 2.73 | 3.15 |
| AUCₗₐₛₜ (hr*ng/mL) | 625 | 731 | 16133 | 22454 |
| AUC_{INF}_{_{—}}_{obs} (hr*ng/mL) | 784 | 1002 | 16171 | 22487 |
| MRTₗₐₛₜ (hr) | 3.01 | 2.96 | 5.35 | 4.89 |
| % F_{relative po} (AUC_{INF}) | 78.3 | NA | NA | NA |
| Brain/Plasma ratio (AUC_{INF}) | NA | NA | 25.8 | 30.7 |

PK profile in Rats. To evaluate the full PK profile for I-1 in rats, plasma and brain pharmacokinetic and urinary excretion of I-1 in male Wistar rats after IV dose at 1 mg/kg and an oral (p.o.) dose at 5 mg/kg administration were determined. Plasma, brain, and urine samples were collected at specified time points after single dose administration of I-1 and quantification of I-1 in the samples was accomplished using LC-MS/MS. The pharmacokinetic parameters were determined as described above.

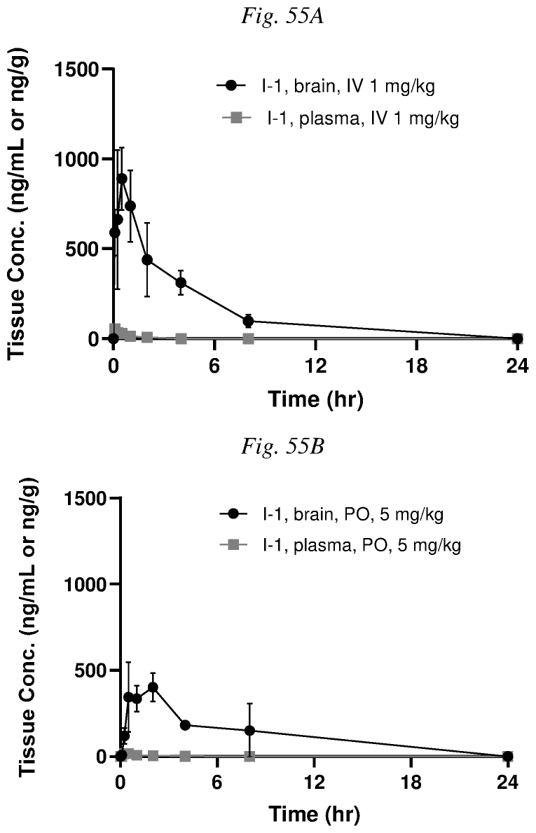

The results are presented in Table 8 and FIGS. 55A and 55B. Male rats were observed to have mild to moderate-grade head twitching immediately after IV administration and 1.5 hr after oral dosing. I-1 was rapidly absorbed after p.o. administration with plasma and brain concentrations observed at the first time points taken (0.083 hr) followed by a single elimination phase. I-1 mean maximum plasma concentrations (Cₘₐₓ) after p.o. administration was 18.6 ng/mL at 0.5 hr. The extrapolated concentration at time 0 (C₀) after IV administration was 67.2 ng/mL. The corresponding mean area under curve (AUC_{INF}) values were 50.7 and 49.7 hr*ng/mL for IV and p.o., respectively. Mean terminal half-lives (T_{1/2}) were 0.78 and 3.53 hr after IV and p.o., respectively. The 4 hr timepoint was below the limit of quantitation (5.02 ng/mL) after IV dosing, and thus a well-defined terminal phase was not possible which may affect the interpretation of the intravenous half-life. Observed systemic clearance (Cl) and volume of distribution (V_{z}) after IV administration were 19.7 L/hr/kg and 22.2 L/kg, respectively and absolute oral bioavailability was 19.1%.

After IV and p.o. administration, Cₘₐₓ for I-1 brain concentrations were 890 ng/g at 0.5 hr and 402 at 2.0 hr; AUC_{INF} was 3259 and 2880 hr*ng/g; mean terminal half-lives were 2.44 and 4.71 hr. The brain to plasma AUC_{INF} ratio ranged from 57.9 to 64.3 across route of administration and dose.

The mean total amount of I-1 recovered in urine after IV administration was 718 ng (n=2) which equates to a low percentage recovery of I-1 at 0.07%. Renal clearance was calculated to be 0.0682 L/hr/kg.

In summary, administration of I-1 to male rats presented with Cₘₐₓ values after oral administration at 1.5 hr post dose. Total systemic clearance was approximately 6-fold higher than total hepatic blood flow (3.3 L/hr/kg; Davies B, Morris T. Physiological parameters in laboratory animals and humans. Pharm Res. 1993, 10(7), 1093-5) and clearance from plasma and brain was similar. Renal excretion of I-1 was less than 1% of the total clearance. The volume of distribution was approximately 32-fold higher than total body water (0.7 L/kg). The absolute oral bioavailability was moderate at 19.1%. I-1 concentration was 57.9- to 64.3-fold higher (AUC_{INF}) in brain compared to plasma. Lastly, the elimination T₁/2 after oral administration was 3.53 and 4.71 hr for plasma and brain, respectively.

TABLE 8
Mean pharmacokinetic parameters following intravenous
and oral administration of I-1 (1 and 5 mg/kg,
respectively) in Wistar rats

| | I-1 | | | |
|---|---|---|---|---|
| Matrix | Plasma | | Brain | |
| Route | IV | p.o. | IV | p.o. |
| Dose (mg/kg) | 1 | 5 | 1 | 5 |
| C₀ (ng/mL) | 67.2 | NA | NA | NA |
| Cₘₐₓ (ng/mL) | 54.1 | 18.6 | 890 | 402 |
| Tₘₐₓ (hr) | 0.083 | 0.5 | 0.5 | 2 |
| T_{1/2} (hr) | 0.78 | 3.53 | 2.44 | 4.71 |
| AUCₗₐₛₜ (hr*ng/mL) | 42.2 | 40.3 | 2914 | 1856 |
| AUC_{INF} (hr*ng/mL) | 50.7 | 49.7 | 3259 | 2880 |
| V_{z} (L/kg) | 22.2 | NA | NA | NA |
| Cl (L/hr/kg) | 19.7 | NA | NA | NA |
| MRTₗₐₛₜ (hr) | 0.62 | 2.25 | 2.54 | 3.3 |
| % F (AUC_{INF}) | NA | 19.1 | NA | NA |
| Brain/Plasma (AUC_{INF}) | NA | NA | 64.3 | 57.9 |

In another rat PK study, plasma, brain, and cerebrospinal fluid (CSF) distribution of I-1 was evaluated in Sprague-Dawley male rats after intravenous (IV) administration at 1 mg/kg. Plasma, brain, and CSF samples were collected at specified time points up to 8 hr postdose after single dose administration of I-1. Blood samples were collected via cardiac puncture and the animals were euthanized via CO₂ inhalation after blood samples were collected. The CSF samples were collected via cisterna magna puncture with the brain tissue extracted after decapitation (N=4 animals per time point). The concentrations of compound I-1 in plasma, with K₂EDTA as the anticoagulant, brain tissue, and CSF were determined by LC-MS/MS (LLOQ, 0.3 ng/mL). PK parameters for plasma, CSF, and brain tissue were calculated as described above.

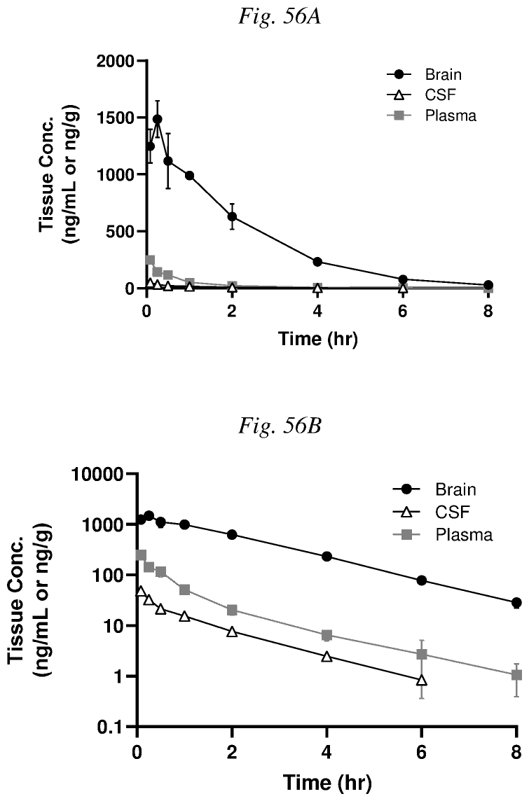

The results are presented in Table 9 and FIGS. 56A and 56B. It was shown that administration of I-1 to male Sprague-Dawley rats presented with Cₘₐₓ of 250, 1490 and 48.7 ng/mL for plasma, brain, and CSF, respectively. Distribution into the CNS was rapid with Tₘₐₓ values of 0.25 and 0.083 hr, brain and CSF, respectively.

Total systemic clearance was approximately 1.4-fold higher than total hepatic blood flow (3.31 L/hr/kg; Davies B, Morris T. Physiological parameters in laboratory animals and humans. Pharm Res. 1993, 10(7), 1093-5). Clearance from brain and CSF were similar to and paralleled that of plasma suggesting passive diffusion of I-1 from the CNS into the systemic circulation. The volume of distribution was approximately 8.1-fold higher than total body water (0.7 L/kg, Davies B, Morris T. Physiological parameters in laboratory animals and humans. Pharm Res. 1993, 10(7), 1093-5). I-1 concentrations were 15.6-fold higher (AUC_{INF}) in brain compared to plasma. The CSF concentrations were only 20% of that found in plasma. Concomitantly CSF concentrations were 1.6% of brain concentrations. The CSF/brain ratio corroborates brain protein binding experiments in which the free fraction of compound I-1 in dilute brain homogenate was 1.4%.

TABLE 9
Mean pharmacokinetic parameters following IV administration
of I-1 (1 mg/kg) in male Sprague-Dawley rats

| | Compound I-1 | | |
|---|---|---|---|
| Matrix | Plasma | Brain | CSF |
| Parameters/Route | IV | IV | IV |
| Dose (mg/kg) | 1 | 1 | 1 |
| T_{1/2} (hr) | 1.53 | 1.34 | 1.26 |
| Tₘₐₓ (hr) | 0.083 | 0.25 | 0.083 |
| Cₘₐₓ (ng/mL) | 250 | 1490 | 48.7 |
| C₀ (ng/mL) | 330 | NA | NA |
| Tₗₐₛₜ (hr) | 8 | 8 | 6 |
| AUCₗₐₛₜ (hr*ng/mL) | 207 | 3220 | 49.9 |
| AUC_{INF} (hr*ng/mL) | 210 | 3280 | 51.4 |
| CL (L/hr/kg) | 4.77 | NA | NA |
| MRT_{INF} (hr) | 1.19 | 1.98 | 1.51 |
| Vₛₛ (L/kg) | 5.67 | NA | NA |
| Brain/Plasma (AUC_{INF}) | NA | 15.6 | NA |
| CSF/plasma (AUC_{INF}) | NA | NA | 0.2 |
| CSF/Brain (AUC_{INF}) | NA | NA | 0.016 |

### Effects of I-1 on Changes in Brain Markers of Neuroplasticity in Mice

To evaluate the effects of I-1 on brain markers of neuroplasticity, male C57BL/6J mice were dosed as described in Table 10.

TABLE 10
Experimental groups for the evaluation of
brain markers of neuroplasticiy in mice

| Group | Treatment Days 1-3 | Treatment Day 4 | n |
|---|---|---|---|
| A | Vehicle (Saline) s.c. | Vehicle (Saline) s.c. | 10 |
| B | Vehicle (Saline) s.c. | I-1 (3 mg/kg) s.c. | 10 |
| C | I-1 (0.3 mg/kg) s.c. | I-1 (0.3 mg/kg) s.c. | 10 |
| D | Vehicle (Saline) s.c. | Ketamine (10 mg/kg) i.p. | 10 |

On Day 4, the animals were euthanized 2 hours post-dose. The left frontal cortex was dissected and stored in RNAlater. Gene expression analysis was performed on these samples for three housekeeping genes (Hprt, B2m and Actb) plus 10 genes of interest, shown in the literature to be upregulated by acute or chronic treatment with serotonergic psychedelics and/or ketamine: brain derived neurotrophic factor (bdnf), Dlg4 (psd-95), Synapsin 1 (Syn1), homer1, Creb1, Insulin growth factor 1 (Igf1), mTOR, cFos, Early growth response 2 (Egr2) and Neuroplastin (Nptn).

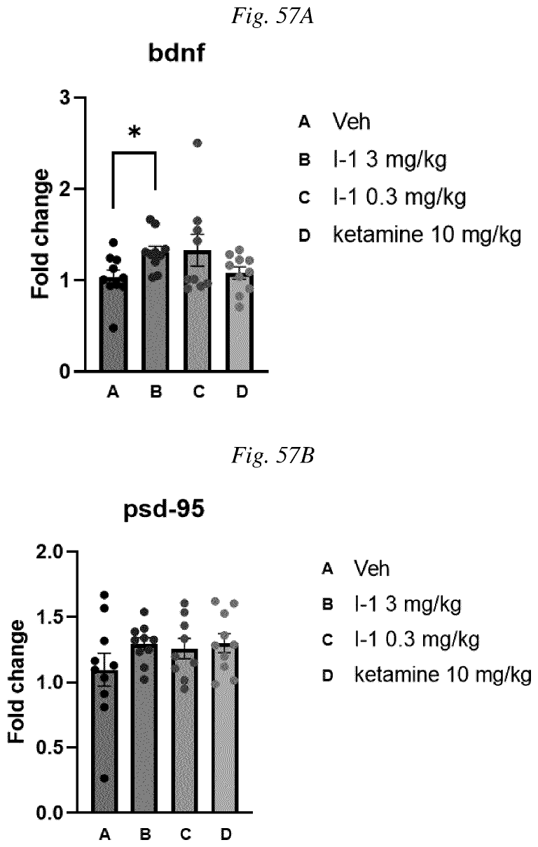

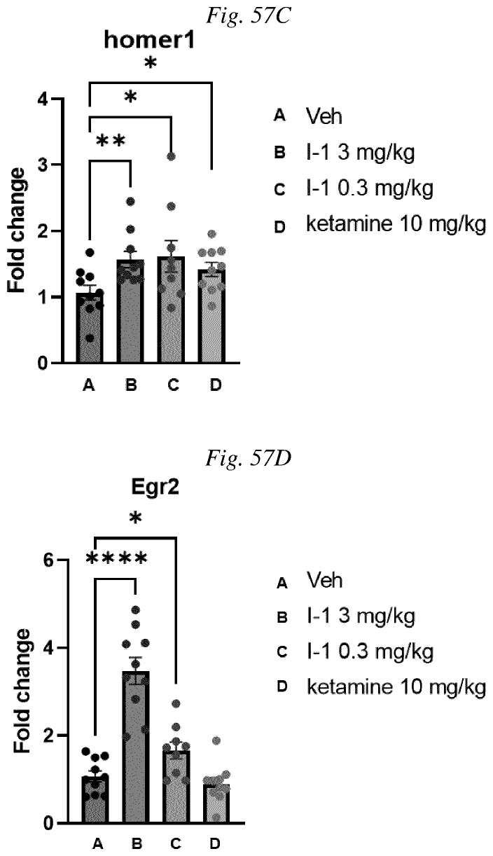

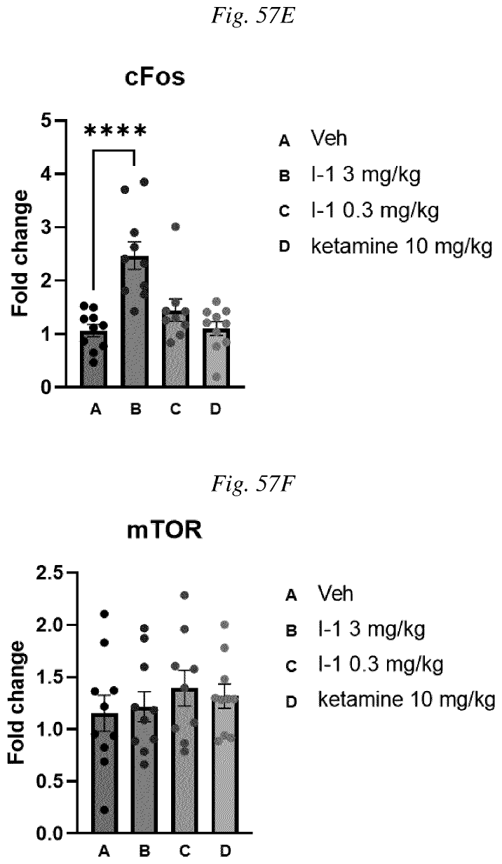

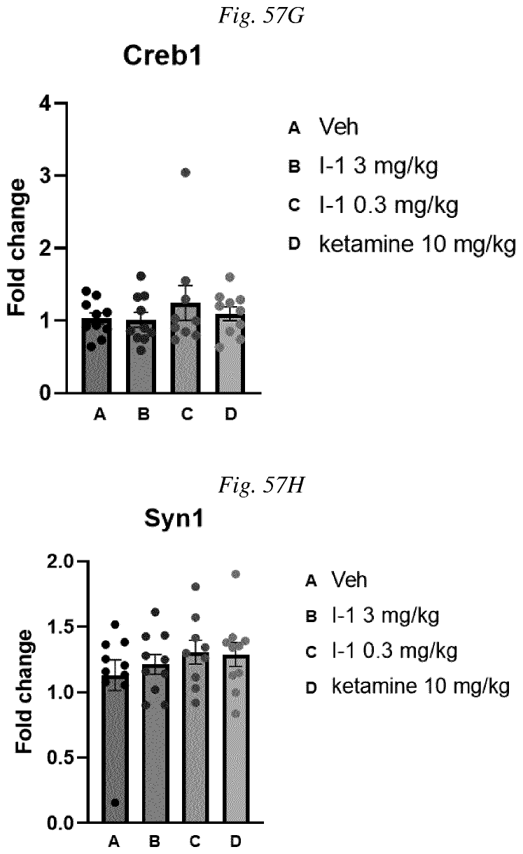

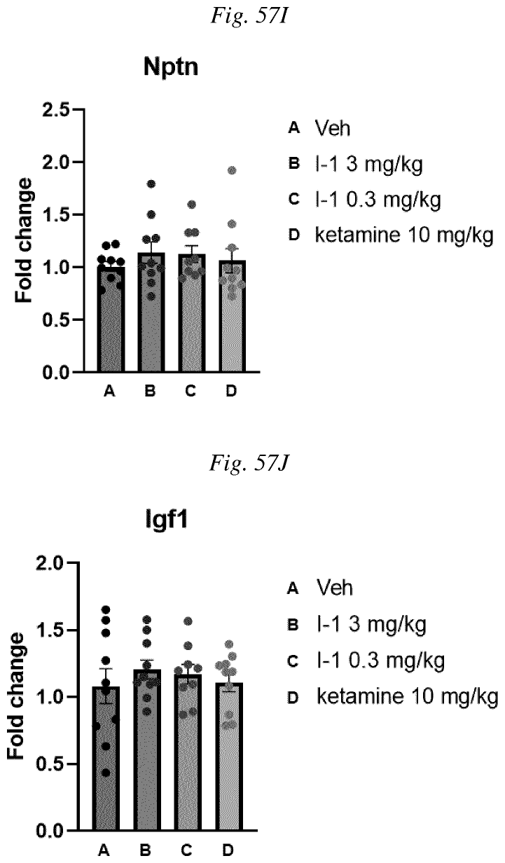

Data were log transformed and analyzed by one-way analysis of variance. Each treatment was compared to vehicle by multiple comparisons. The results are presented in FIGS. 57A-57J. Increased expression of bdnf, homer1, Egr2 (krox20) and cFos mRNA were observed in the group treated once with 3 mg/kg I-1 (group B), indicating that acute treatment with I-1 induced rapid transcriptional changes in the frontal cortex in the acute phase (2 h) after treatment. In particular, increased expression of cFos, an immediate early gene which is associated with neuronal activity, was observed indicating that acute treatment with 3 mg/kg I-1 increased neuronal firing in the frontal cortex (FIG. 57E). Moreover, increased homer1 expression, which is associated with the formation of postsynaptic scaffolds that cluster and colocalize NMDAR and mGluR was observed (FIG. 57C); and increased Egr2 expression, a growth factor that has been proposed to be a marker for psychedelic activation of 5-HT_{2A} receptors (Gonzalez-Maeso et al., Transcriptome fingerprints distinguish hallucinogenic and nonhallucinogenic 5-hydroxytryptamine 2A receptor agonist effects in mouse somatosensory cortex. J Neurosci. 2003; 23(26), 8836-43) was observed (FIG. 57D). Upregulation of bdnf, a neurotrophin that regulates the survival and growth of neurons and influences synaptic efficiency and plasticity, was also observed (FIG. 57A).

Increased expression of homer1 and Egr2 mRNA were observed in the group treated four times with a sub-psychedelic 0.3 mg/kg I-1 (group C)(FIGS. 57C and 57D), indicating that subchronic treatment with I-1 induced transcriptional changes to genes involved in postsynaptic glutamatergic receptor formation (homer1), that were measurable in the acute phase (2 h) after treatment.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

Accordingly, the preceding merely illustrates the principles of the methods and compositions. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the following.

1. A compound having a structure of Formula (I): 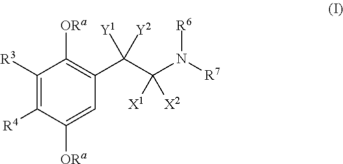
or a pharmaceutically acceptable salt, solvate, stereoisomer, or prodrug thereof,
wherein:
X¹ and X² are independently hydrogen or deuterium;
Y¹ and Y² are independently hydrogen or deuterium;
R³ is hydrogen or deuterium;
R⁴ is —SCF₂H, —SCFH₂, —SCH₂CF₂CF₂H, —OCF₃, —OCF₂H, —OCFH₂, —OCH₂CF₂CF₂H, -cyclopentyl (—C₅H₉), or -A(CF₂)ₘ(CH₂)ₙQ, wherein A is S, O, CH₂, or CF₂, m is 0 to 3, n is 0 to 6, and Q is —C≡CH or —C≡N;
R⁶ and R⁷ are independently hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl; or alternatively R⁶ and R⁷ together with the nitrogen atom attached thereto are optionally joined to form a substituted or unsubstituted heterocycloalkyl; and
each R^{a} is independently a substituted or unsubstituted C₁-C₆ alkyl.

2. The compound of claim 1, wherein Y¹ and Y² are hydrogen.

3. The compound of claim 1, wherein R³ is hydrogen.

4. The compound of claim 1, wherein X¹ and X² are hydrogen.

5. The compound of claim 1, wherein X¹ and X² are deuterium.

6. The compound of claim 1, wherein each R^{a} is —CH₃ or —CD₃.

7. The compound of claim 1, wherein R⁴ is —SCF₂H, —SCH₂CF₂CF₂H, or —SCH₂CH₂CH₂C≡CH.

8. The compound of claim 1, having a structure of Formula (II): 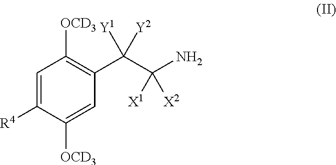
or a pharmaceutically acceptable salt, solvate, stereoisomer, or prodrug thereof,
wherein:
X¹ and X² are independently hydrogen or deuterium;
Y¹ and Y² are independently hydrogen or deuterium; and
R⁴ is —SCF₂H, —SCFH₂, —SCH₂CF₂CF₂H, —OCF₃, —OCF₂H, —OCFH₂, —OCH₂CF₂CF₂H, -cyclopentyl (—C₅H₉), or -A(CF₂)ₘ(CH₂)ₙQ, wherein A is S, O, CH₂, or CF₂, m is 0 to 3, n is 0 to 6, and Q is —C≡CH or —C≡N.

9. The compound of claim 8, wherein X¹ and X² are hydrogen.

10. The compound of claim 8, wherein R⁴ is —SCF₂H, —SCH₂CF₂CF₂H, or —SCH₂CH₂CH₂C≡CH.

11. The compound of claim 8, wherein R⁴ is —SCF₂H.

12-14. (canceled)

15. The compound of claim 1, having a structure of Formula (IV): 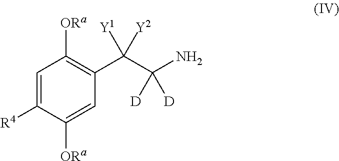
or a pharmaceutically acceptable salt, solvate, stereoisomer, or prodrug thereof,
wherein:
Y¹ and Y² are independently hydrogen or deuterium;
R⁴ is —SCF₂H, —SCFH₂, —SCH₂CF₂CF₂H, —OCF₃, —OCF₂H, —OCFH₂, —OCH₂CF₂CF₂H, -cyclopentyl (—C₅H₉), or -A(CF₂)ₘ(CH₂)ₙQ, wherein A is S, O, CH₂, or CF₂, m is 0 to 3, n is 0 to 6, and Q is —C≡CH or —C≡N; and
each R^{a} is independently a substituted or unsubstituted C₁-C₆ alkyl.

16. A compound having a structure of Formula (V): 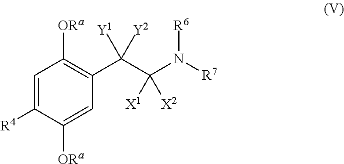
or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁴ is halogen, a substituted or unsubstituted C₁-C₆ alkyl, a substituted or unsubstituted C₃-C₁₀ cycloalkyl, a substituted or unsubstituted alkenyl, a substituted or unsubstituted alkynyl, —OR^{b}, or —SR^{b};
R⁶ is hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl;
with the proviso that at least one of X¹, X², Y¹, Y², R³, R⁴, R⁶, R⁷, and R^{a} comprises deuterium and/or R⁴ is selected from the group consisting of —SCF₃, —SCF₂H, —SCFH₂, —SCH₂CH₂CF₃, —SCH₂CH₂CF₂H, —SCH₂CH₂CFH₂, —SCH₂CF₂CF₂H, —OCF₃, —OCF₂H, —OCFH₂, —OCH₂CH₂CF₃, —OCH₂CH₂CF₂H, —OCH₂CH₂CFH₂, —OCH₂CF₂CF₂H, -cyclopentyl (—C₅H₉), and -A(CF₂)ₘ(CH₂)ₙQ, wherein A is S, O, CH₂, or CF₂, m is 0 to 3, n is 0 to 6, and Q is —C≡CH or —C≡N.

17. The compound of claim 16, wherein R⁶ is hydrogen, and R⁷ is an unsubstituted C₁-C₆ alkyl, a C₁-C₆ alkyl substituted with one or more deuterium atoms, a C₁-C₆ alkyl substituted with one or more fluorine atoms, or a C₁-C₆ alkyl substituted with a substituted or unsubstituted cycloalkyl.

18. The compound of claim 16, which is selected from the group consisting of 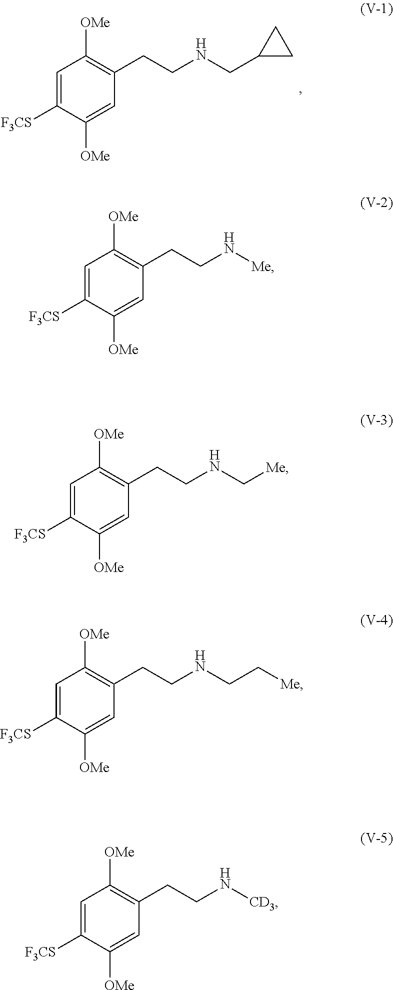 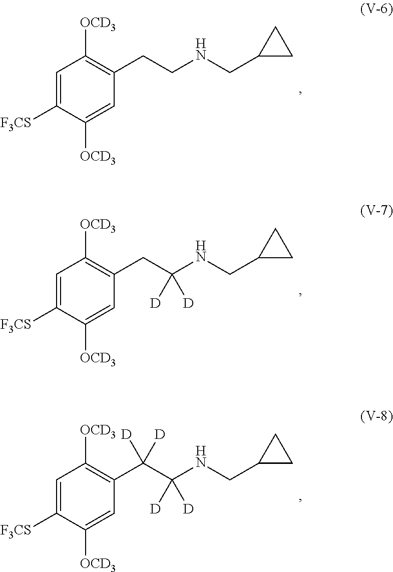 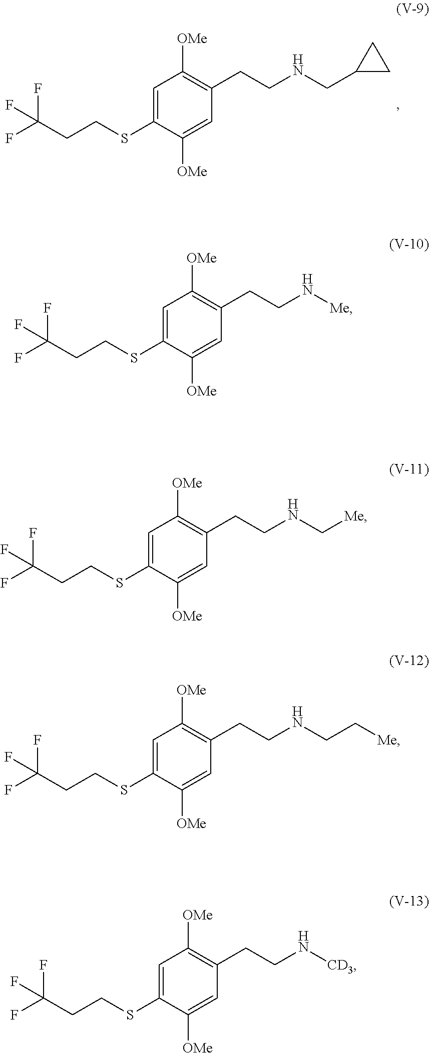 [SMILES: COc1cc(SCCC(F)(F)F)c(OC)cc1CCNCC1CC1 ; CNCCc1cc(OC)c(SCCC(F)(F)F)cc1OC ; CCNCCc1cc(OC)c(SCCC(F)(F)F)cc1OC ; CCCNCCc1cc(OC)c(SCCC(F)(F)F)cc1OC ; [2H]C([2H])([2H])NCCc1cc(OC)c(SCCC(F)(F)F)cc1OC] 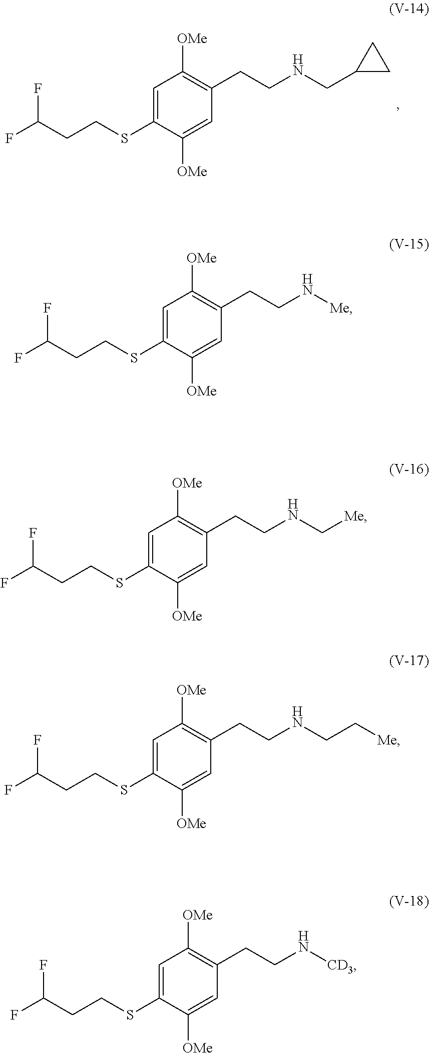 [SMILES: COc1cc(SCCC(F)F)c(OC)cc1CCNCC1CC1 ; CNCCc1cc(OC)c(SCCC(F)F)cc1OC ; CCNCCc1cc(OC)c(SCCC(F)F)cc1OC ; CCCNCCc1cc(OC)c(SCCC(F)F)cc1OC ; [2H]C([2H])([2H])NCCc1cc(OC)c(SCCC(F)F)cc1OC] 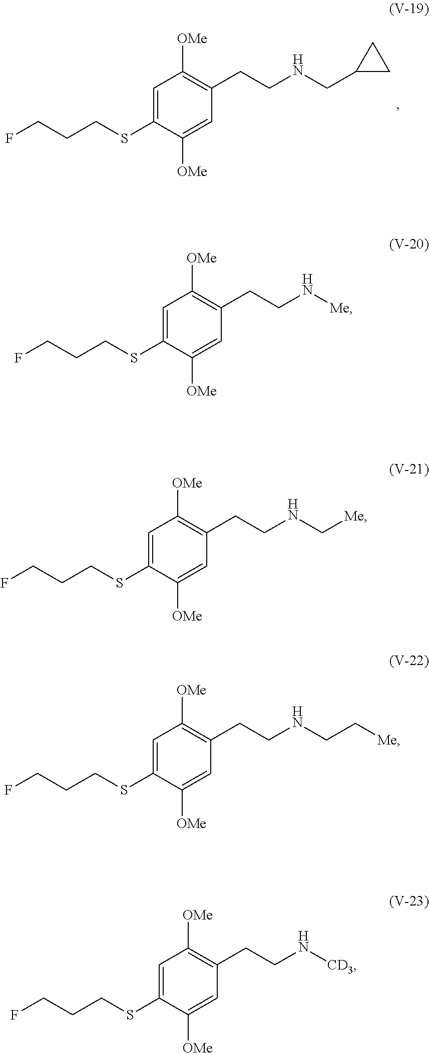 [SMILES: COc1cc(SCCCF)c(OC)cc1CCNCC1CC1 ; CNCCc1cc(OC)c(SCCCF)cc1OC ; CCNCCc1cc(OC)c(SCCCF)cc1OC ; CCCNCCc1cc(OC)c(SCCCF)cc1OC ; [2H]C([2H])([2H])NCCc1cc(OC)c(SCCCF)cc1OC] 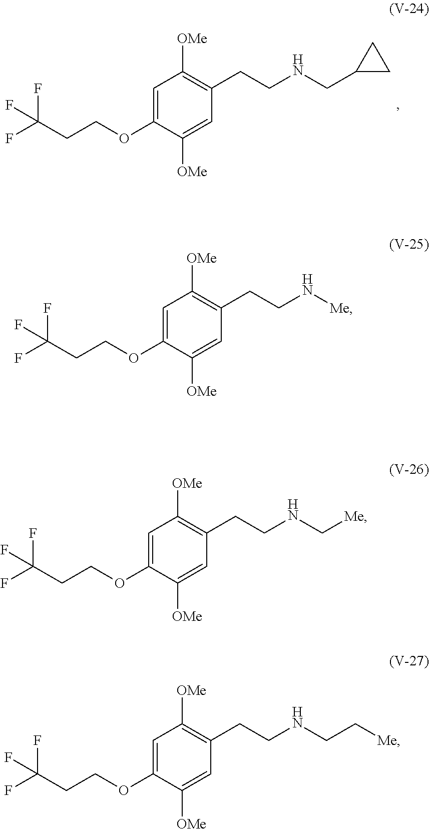 [SMILES: COc1cc(OCCC(F)(F)F)c(OC)cc1CCNCC1CC1 ; CNCCc1cc(OC)c(OCCC(F)(F)F)cc1OC ; CCNCCc1cc(OC)c(OCCC(F)(F)F)cc1OC ; CCCNCCc1cc(OC)c(OCCC(F)(F)F)cc1OC] 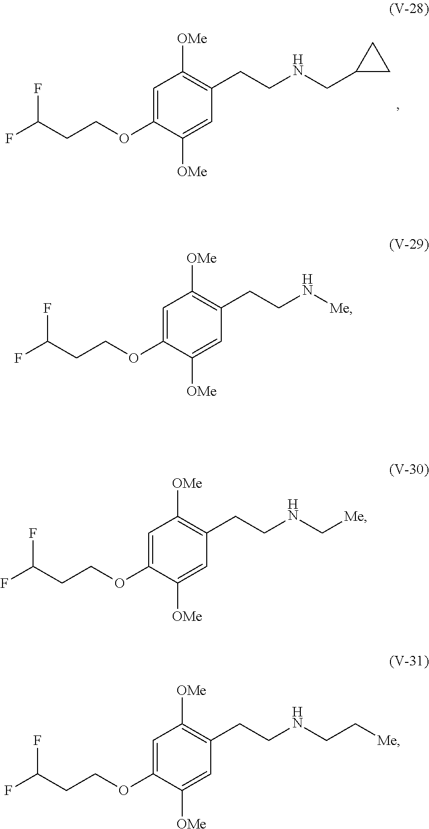 [SMILES: COc1cc(OCCC(F)F)c(OC)cc1CCNCC1CC1 ; CNCCc1cc(OC)c(OCCC(F)F)cc1OC ; CCNCCc1cc(OC)c(OCCC(F)F)cc1OC ; CCCNCCc1cc(OC)c(OCCC(F)F)cc1OC] 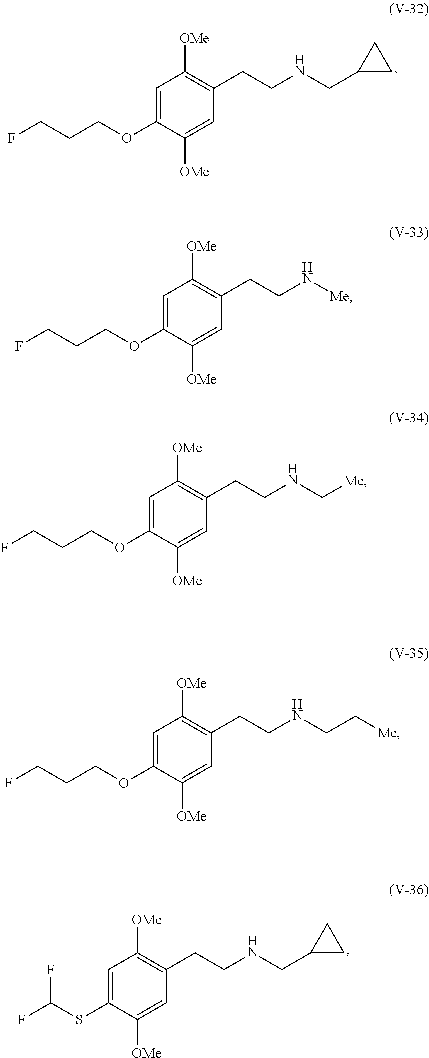 [SMILES: COc1cc(OCCCF)c(OC)cc1CCNCC1CC1 ; CNCCc1cc(OC)c(OCCCF)cc1OC ; CCNCCc1cc(OC)c(OCCCF)cc1OC ; CCCNCCc1cc(OC)c(OCCCF)cc1OC ; COc1cc(SC(F)F)c(OC)cc1CCNCC1CC1] 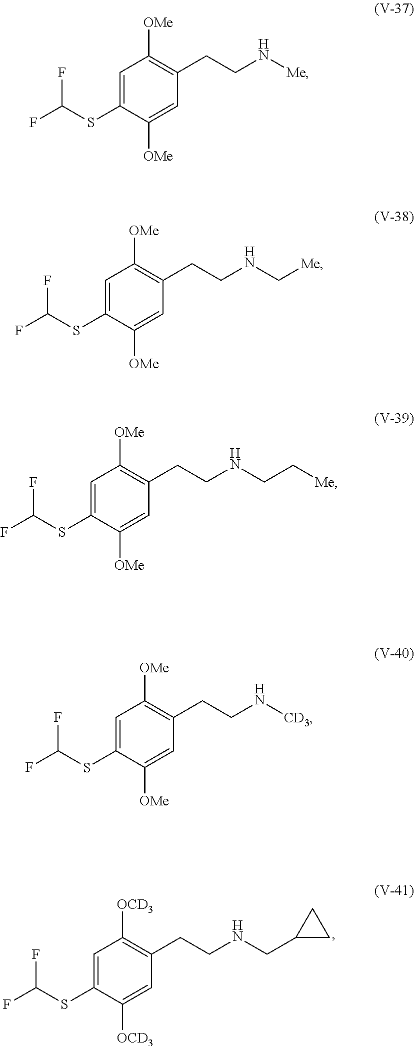 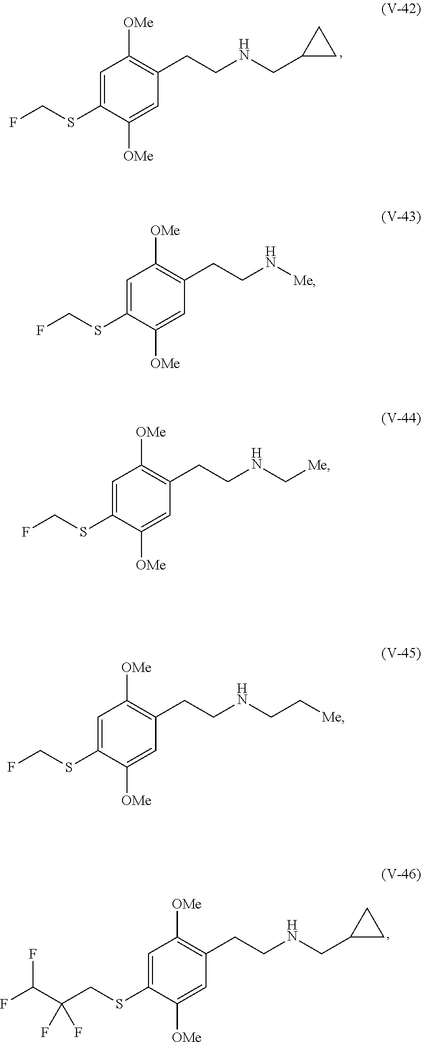 [SMILES: COc1cc(SCF)c(OC)cc1CCNCC1CC1 ; CNCCc1cc(OC)c(SCF)cc1OC ; CCNCCc1cc(OC)c(SCF)cc1OC ; CCCNCCc1cc(OC)c(SCF)cc1OC ; COc1cc(SCC(F)(F)C(F)F)c(OC)cc1CCNCC1CC1] 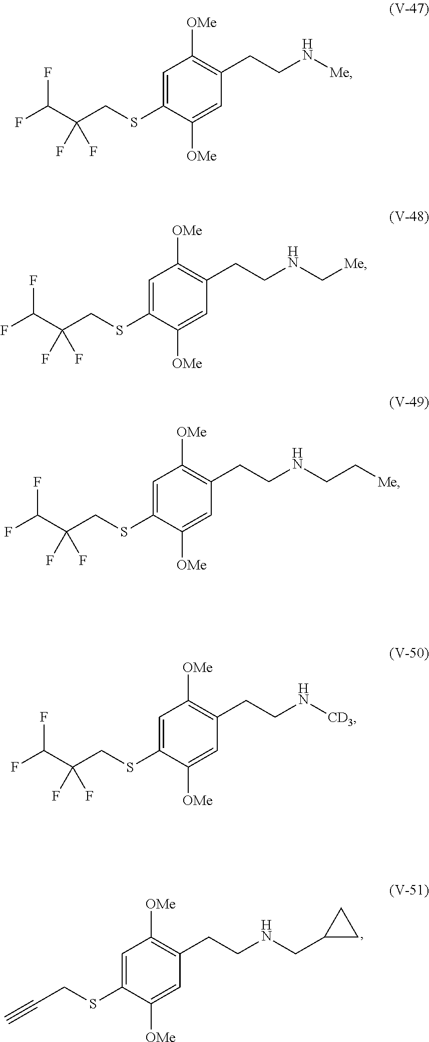 [SMILES: CNCCc1cc(OC)c(SCC(F)(F)C(F)F)cc1OC ; CCNCCc1cc(OC)c(SCC(F)(F)C(F)F)cc1OC ; CCCNCCc1cc(OC)c(SCC(F)(F)C(F)F)cc1OC ; [2H]C([2H])([2H])NCCc1cc(OC)c(SCC(F)(F)C(F)F)cc1OC ; C#CCSc1cc(OC)c(CCNCC2CC2)cc1OC] 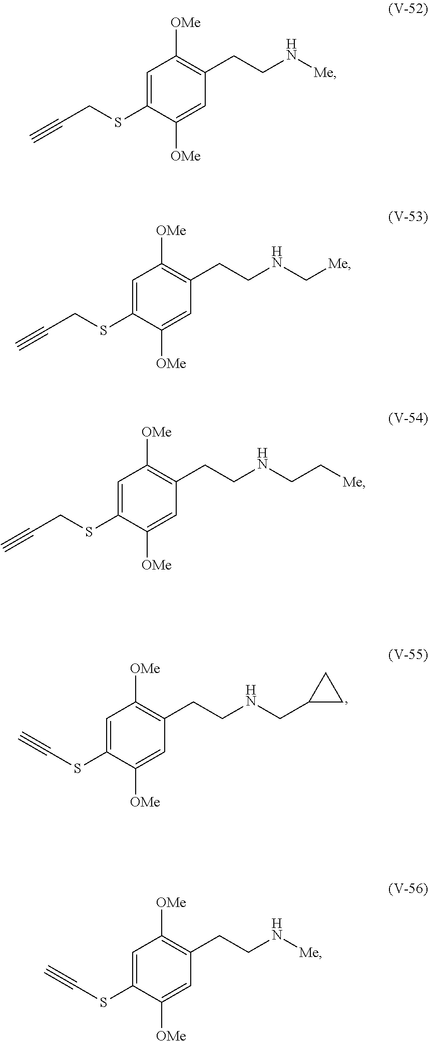 [SMILES: C#CCSc1cc(OC)c(CCNC)cc1OC ; C#CCSc1cc(OC)c(CCNCC)cc1OC ; C#CCSc1cc(OC)c(CCNCCC)cc1OC ; C#CSc1cc(OC)c(CCNCC2CC2)cc1OC ; C#CSc1cc(OC)c(CCNC)cc1OC] 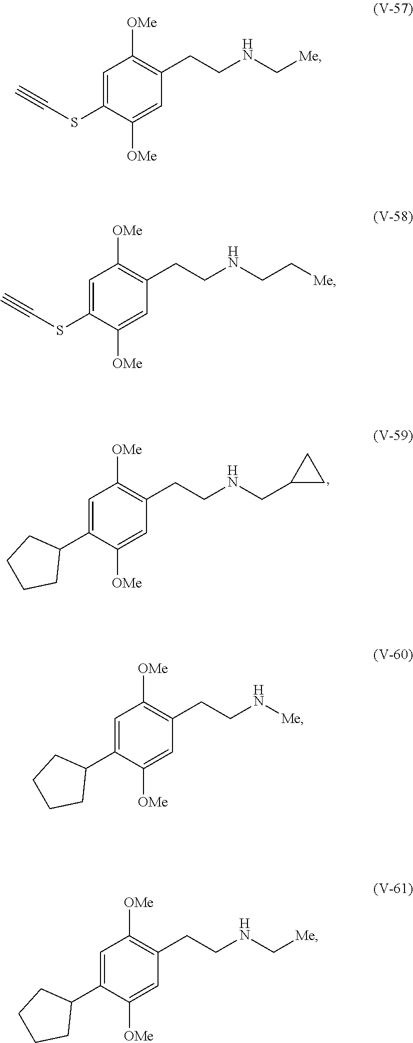 [SMILES: C#CSc1cc(OC)c(CCNCC)cc1OC ; C#CSc1cc(OC)c(CCNCCC)cc1OC ; COc1cc(C2CCCC2)c(OC)cc1CCNCC1CC1 ; CNCCc1cc(OC)c(C2CCCC2)cc1OC ; CCNCCc1cc(OC)c(C2CCCC2)cc1OC] 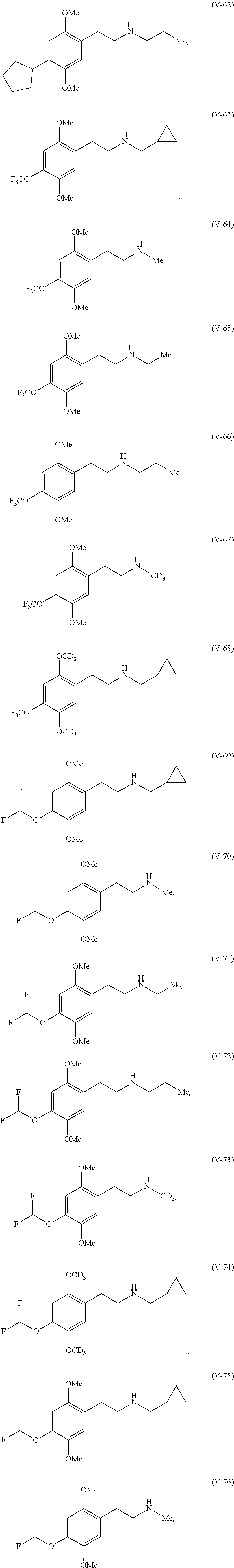 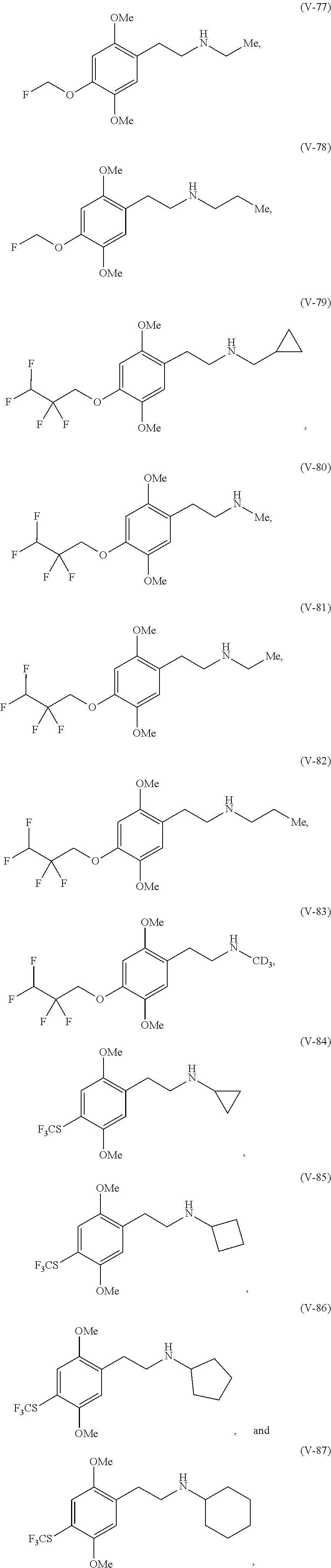
or a pharmaceutically acceptable salt, solvate, or prodrug thereof.

19-96. (canceled)

97. A compound having a structure of Formula (VI): 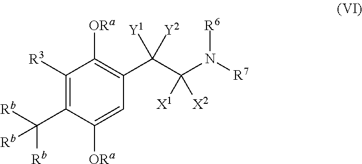
or a pharmaceutically acceptable salt, solvate, stereoisomer, or prodrug thereof,
wherein:
X¹ and X² are independently hydrogen or deuterium;
Y¹ and Y² are independently hydrogen or deuterium;
R³ is hydrogen or deuterium;
R⁶ and R⁷ are independently hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl; or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
each R^{b} is independently a substituted or unsubstituted C₁-C₂ alkyl; or alternatively two R^{b}s together with the carbon atom attached thereto are optionally joined to form a substituted or unsubstituted cycloalkyl.

98. The compound of claim 97, which is selected from the group consisting of 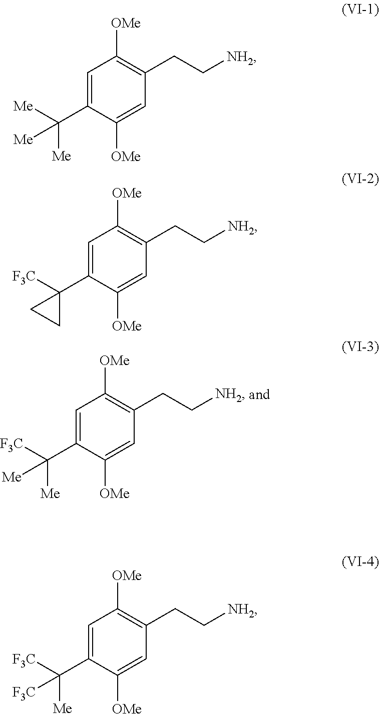
or a pharmaceutically acceptable salt, solvate, or prodrug thereof.

99-100. (canceled)

101. A compound having a structure of Formula (VII): 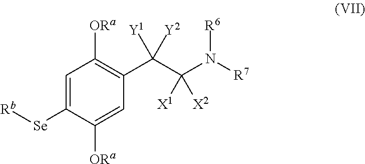
or a pharmaceutically acceptable salt, solvate, stereoisomer, or prodrug thereof,
wherein:
X¹ and X² are independently hydrogen or deuterium;
Y¹ and Y² are independently hydrogen or deuterium;
R⁶ is hydrogen, a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
R⁷ is a substituted or unsubstituted C₁-C₆ alkyl, a substituted or unsubstituted alkenyl, a substituted or unsubstituted alkynyl, a substituted or unsubstituted cycloalkyl, a substituted or unsubstituted heterocycloalkyl, a substituted or unsubstituted aryl, or a substituted or unsubstituted heteroaryl;
or alternatively R⁶ and R⁷ together with the nitrogen atom attached thereto are optionally joined to form a substituted or unsubstituted heterocycloalkyl;
each R^{a} is independently a substituted or unsubstituted C₁-C₆ alkyl; and
R^{b} is hydrogen, deuterium, a substituted or unsubstituted C₁-C₆ alkyl, a substituted or unsubstituted alkenyl, a substituted or unsubstituted alkynyl, or a substituted or unsubstituted C₃-C₁₀ cycloalkyl.

102. The compound of claim 101, which is selected from the group consisting of 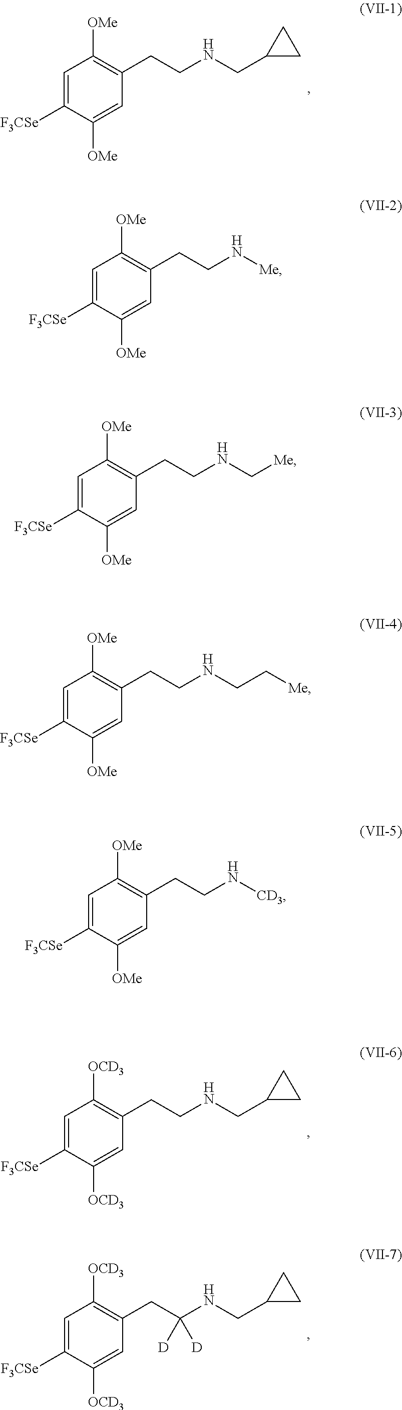 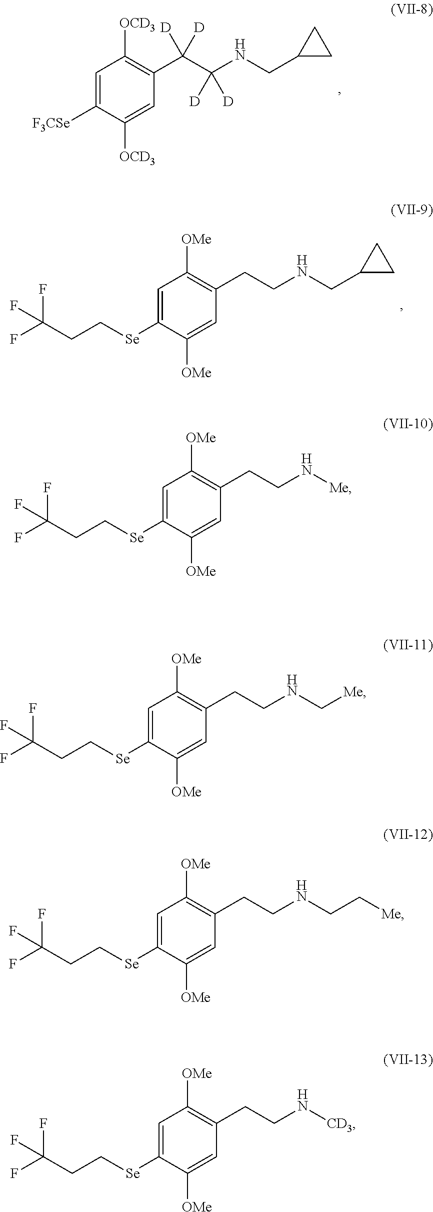 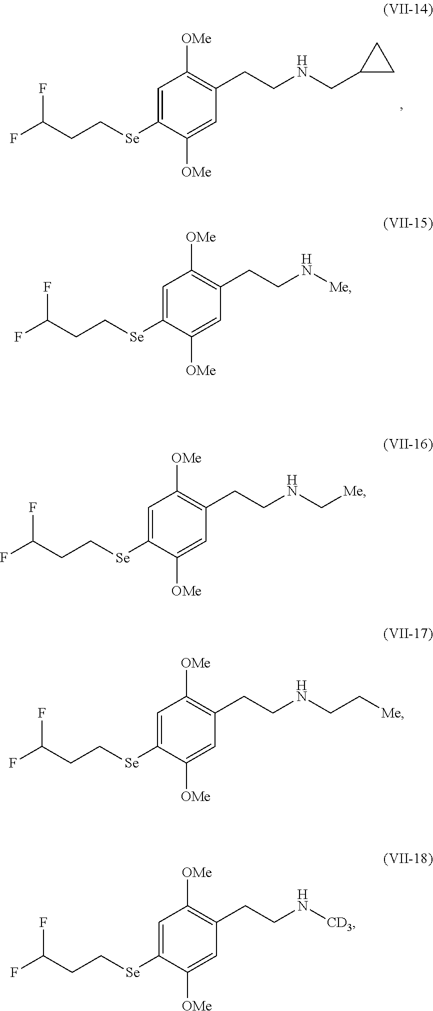 [SMILES: COc1cc([Se]CCC(F)F)c(OC)cc1CCNCC1CC1 ; CNCCc1cc(OC)c([Se]CCC(F)F)cc1OC ; CCNCCc1cc(OC)c([Se]CCC(F)F)cc1OC ; CCCNCCc1cc(OC)c([Se]CCC(F)F)cc1OC ; [2H]C([2H])([2H])NCCc1cc(OC)c([Se]CCC(F)F)cc1OC] 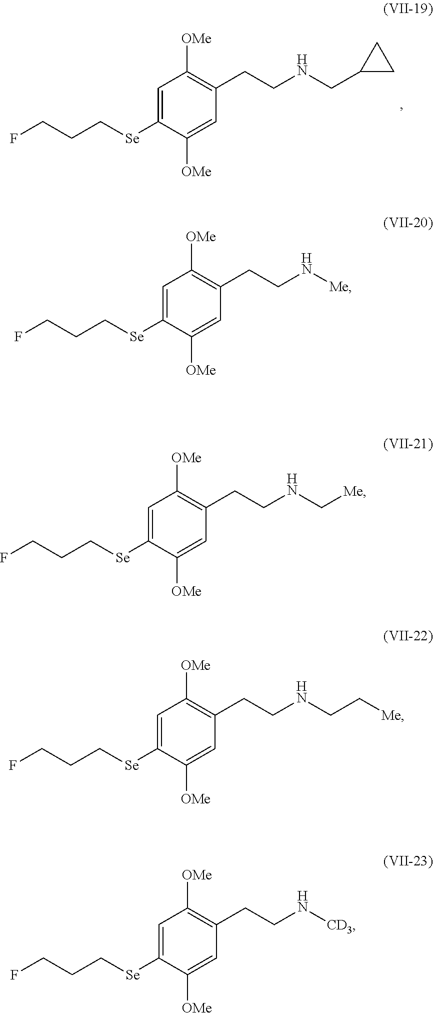 [SMILES: COc1cc([Se]CCCF)c(OC)cc1CCNCC1CC1 ; CNCCc1cc(OC)c([Se]CCCF)cc1OC ; CCNCCc1cc(OC)c([Se]CCCF)cc1OC ; CCCNCCc1cc(OC)c([Se]CCCF)cc1OC ; [2H]C([2H])([2H])NCCc1cc(OC)c([Se]CCCF)cc1OC] 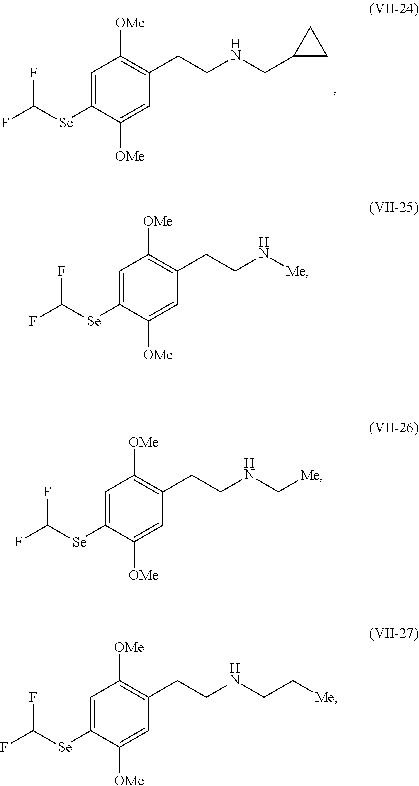 [SMILES: COc1cc([Se]C(F)F)c(OC)cc1CCNCC1CC1 ; CNCCc1cc(OC)c([Se]C(F)F)cc1OC ; CCNCCc1cc(OC)c([Se]C(F)F)cc1OC ; CCCNCCc1cc(OC)c([Se]C(F)F)cc1OC] 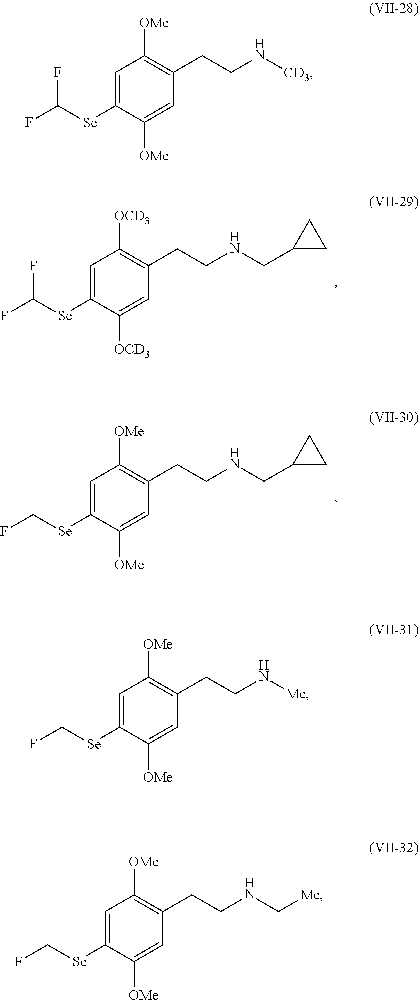 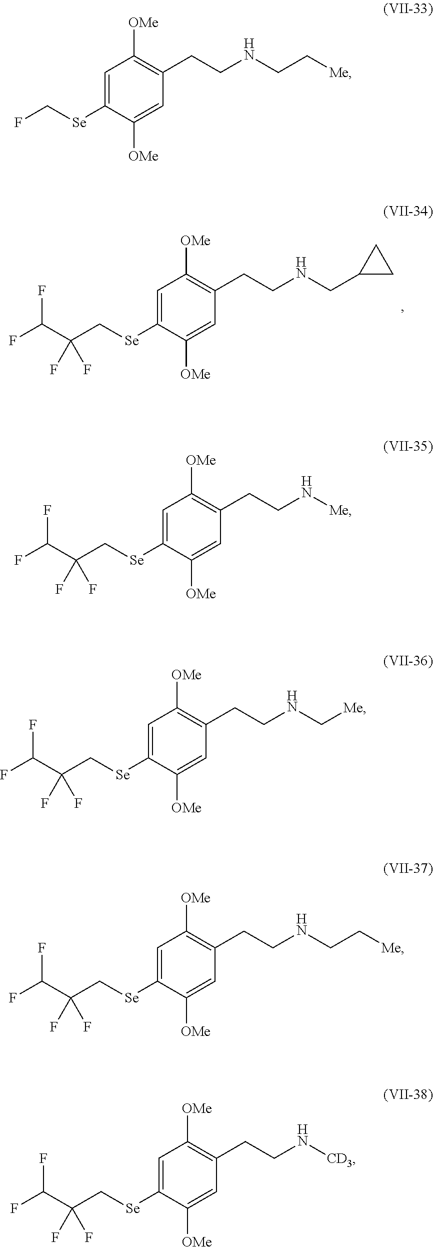 [SMILES: CCCNCCc1cc(OC)c([Se]CF)cc1OC ; COc1cc([Se]CC(F)(F)C(F)F)c(OC)cc1CCNCC1CC1 ; CNCCc1cc(OC)c([Se]CC(F)(F)C(F)F)cc1OC ; CCNCCc1cc(OC)c([Se]CC(F)(F)C(F)F)cc1OC ; CCCNCCc1cc(OC)c([Se]CC(F)(F)C(F)F)cc1OC ; [2H]C([2H])([2H])NCCc1cc(OC)c([Se]CC(F)(F)C(F)F)cc1OC] 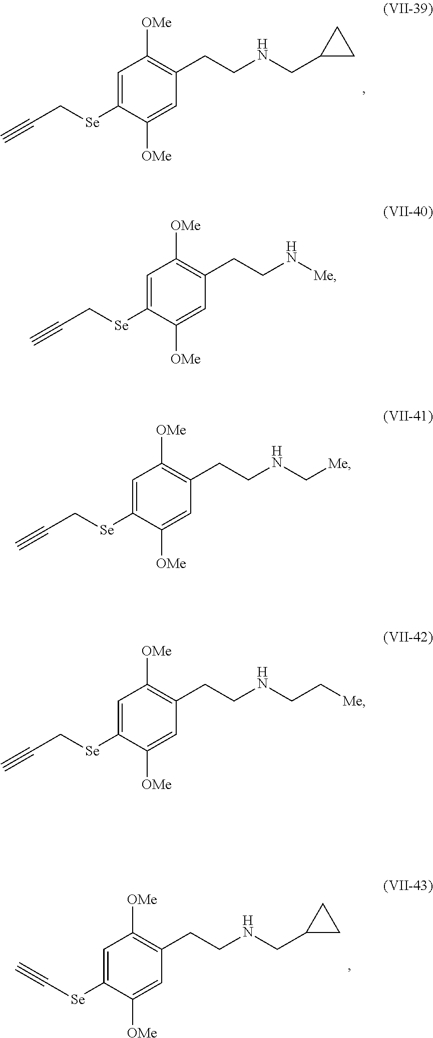 [SMILES: C#CC[Se]c1cc(OC)c(CCNCC2CC2)cc1OC ; C#CC[Se]c1cc(OC)c(CCNC)cc1OC ; C#CC[Se]c1cc(OC)c(CCNCC)cc1OC ; C#CC[Se]c1cc(OC)c(CCNCCC)cc1OC ; C#C[Se]c1cc(OC)c(CCNCC2CC2)cc1OC] 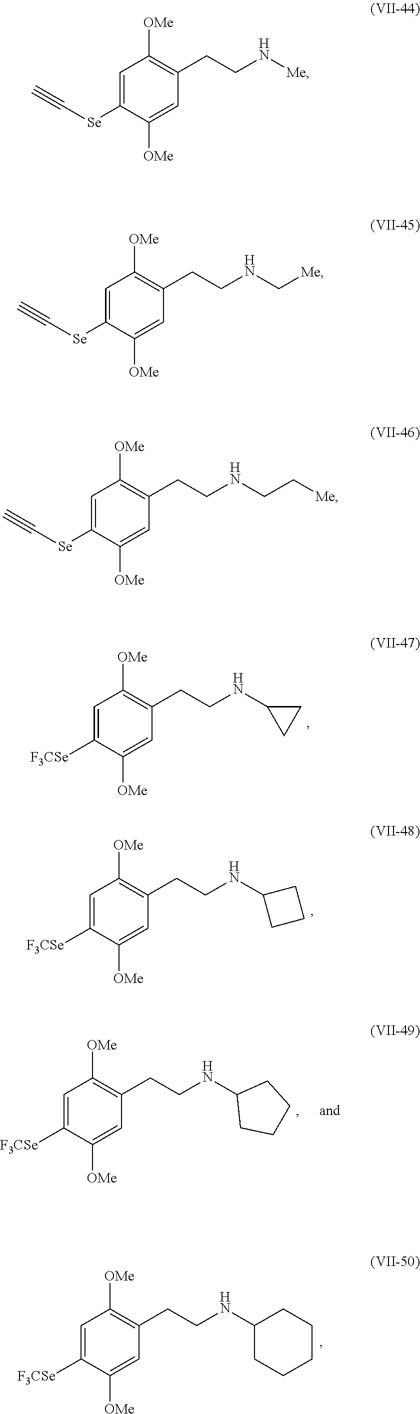
or a pharmaceutically acceptable salt, solvate, or prodrug thereof.

103-104. (canceled)
